(19) United States
(12) Patent Application Publication
(10) Pub. No.: US 2009/0257979 A1
(43) Pub. Date: Oct. 15, 2009

(54) Novel Inhibitors of Hepatitis C Virus Replication

(75) Inventors: Leonid Beigelman, San Mateo, CA (US); Guangyi Wang, Carlsbad, CA (US); Brad O. Buckman, Oakland, CA (US); Antitsa Dimitrova Stoycheva, Half Moon Bay, CA (US)

Correspondence Address: KNOBBE MARTENS OLSON & BEAR LLP, 2040 MAIN STREET, FOURTEENTH FLOOR, IRVINE, CA, 92614, US

(73) Assignee: INTERMUNE, INC., Brisbane, CA (US)

(21) Appl. No.: 12/423,720

(22) Filed: Apr. 14, 2009

Related U.S. Application Data
(60) Provisional application No. 61/045,219, filed on Apr. 15, 2008.
(60) Provisional application No. 61/045,214, filed on Apr. 15, 2008.
(60) Provisional application No. 61/109,856, filed on Oct. 30, 2008.
(60) Provisional application No. 61/117,916, filed on Nov. 25, 2008.
(60) Provisional application No. 61/148,337, filed on Jan. 29, 2009.

(51) Int. Cl. A61K 31/5415; C07D 285/22; C12N 9/99; A61K 38/21; A61P 31/18; A61P 31/20

(52) U.S. Cl. 424/85.5; 544/12; 514/223.2; 435/184; 424/85.7

(57) ABSTRACT

The embodiments provide compounds of the general Formula I, as well as compositions, including pharmaceutical compositions, comprising a subject compound. The embodiments further provide treatment methods, including methods of treating a hepatitis C virus infection and methods of treating liver fibrosis, the methods generally involving administering to an individual in need thereof an effective amount of a subject compound or composition.

### RELATED APPLICATIONS

This application claims the benefit of U.S. Provisional Application Nos. 61/045,219, filed Apr. 15, 2008; 61/045,214, filed Apr. 15, 2008; 61/109,856, filed Oct. 30, 2008; 61/117,916, filed Nov. 25, 2008; and 61/148,337, filed Jan. 29, 2009; all of which are incorporated herein by reference in their entirety.

### BACKGROUND

1. Field

The present application relates to compounds, processes for their synthesis, compositions and methods for the treatment of hepatitis C virus (HCV) infection.

2. Description of the Related Art

Hepatitis C virus (HCV) infection is the most common chronic blood borne infection in the United States. Although the numbers of new infections have declined, the burden of chronic infection is substantial, with Centers for Disease Control estimates of 3.9 million (1.8%) infected persons in the United States. Chronic liver disease is the tenth leading cause of death among adults in the United States, and accounts for approximately 25,000 deaths annually, or approximately 1% of all deaths. Studies indicate that 40% of chronic liver disease is HCV-related, resulting in an estimated 8,000-10,000 deaths each year. HCV-associated end-stage liver disease is the most frequent indication for liver transplantation among adults.

Antiviral therapy of chronic hepatitis C has evolved rapidly over the last decade, with significant improvements seen in the efficacy of treatment. Nevertheless, even with combination therapy using pegylated IFN-α plus ribavirin, 40% to 50% of patients fail therapy, i.e., are nonresponders or relapsers. These patients currently have no effective therapeutic alternative. In particular, patients who have advanced fibrosis or cirrhosis on liver biopsy are at significant risk of developing complications of advanced liver disease, including ascites, jaundice, variceal bleeding, encephalopathy, and progressive liver failure, as well as a markedly increased risk of hepatocellular carcinoma.

The high prevalence of chronic HCV infection has important public health implications for the future burden of chronic liver disease in the United States. Data derived from the National Health and Nutrition Examination Survey (NHANES III) indicate that a large increase in the rate of new HCV infections occurred from the late 1960s to the early 1980s, particularly among persons between 20 to 40 years of age. It is estimated that the number of persons with long-standing HCV infection of 20 years or longer could more than quadruple from 1990 to 2015, from 750,000 to over 3 million. The proportional increase in persons infected for 30 or 40 years would be even greater. Since the risk of HCV-related chronic liver disease is related to the duration of infection, with the risk of cirrhosis progressively increasing for persons infected for longer than 20 years, this will result in a substantial increase in cirrhosis-related morbidity and mortality among patients infected between the years of 1965-1985.

HCV is an enveloped positive strand RNA virus in the Flaviviridae family. The single strand HCV RNA genome is approximately 9500 nucleotides in length and has a single open reading frame (ORF) encoding a single large polyprotein of about 3000 amino acids. In infected cells, this polyprotein is cleaved at multiple sites by cellular and viral proteases to produce the structural and non-structural (NS) proteins of the virus. In the case of HCV, the generation of mature nonstructural proteins (NS2, NS3, NS4, NS4A, NS4B, NS5A, and NS5B) is effected by two viral proteases. The first viral protease cleaves at the NS2-NS3 junction of the polyprotein. The second viral protease is serine protease contained within the N-terminal region of NS3 (herein referred to as “NS3 protease”). NS3 protease mediates all of the subsequent cleavage events at sites downstream relative to the position of NS3 in the polyprotein (i.e., sites located between the C-terminus of NS3 and the C-terminus of the polyprotein). NS3 protease exhibits activity both in cis, at the NS3—NS4 cleavage site, and in trans, for the remaining NS4A-NS4B, NS4B-NS5A, and NS5A-NS5B sites. The NS4A protein is believed to serve multiple functions, acting as a cofactor for the NS3 protease and possibly assisting in the membrane localization of NS3 and other viral replicase components. Apparently, the formation of the complex between NS3 and NS4A is necessary for NS3-mediated processing events and enhances proteolytic efficiency at all sites recognized by NS3. The NS3 protease also exhibits nucleoside triphosphatase and RNA helicase activities.

NS5B is an RNA-dependent RNA polymerase involved in the replication of HCV RNA. There are two main mechanisms of inhibiting the NS5B polymerase. The first involves a phosphorylated nucleoside inhibitor can be accepted as a substrate by the NS5B polymerase as a modified nucleotide. The incorporation of the modified nucleotide in the nascent RNA chain can terminate the growth of the RNA polymer chain. These inhibitors are generally synthesized in the non-phosphorylated form as prodrugs, and are converted to the active triphosphate form by cellular kinases in the cytoplasm of infected cells. The second mechanism of action involves a non-nucleoside inhibitor that inhibits the NS5B polymerase at a stage preceding the elongation reaction. Several different binding sites for non-nucleoside inhibitors exist on the RNA-dependent RNA-polymerase surface.

### SUMMARY

The present embodiments provide a compound having the structure of Formula I:

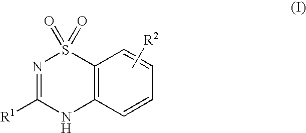

or a pharmaceutically acceptable salt or prodrug thereof wherein R can be selected from:

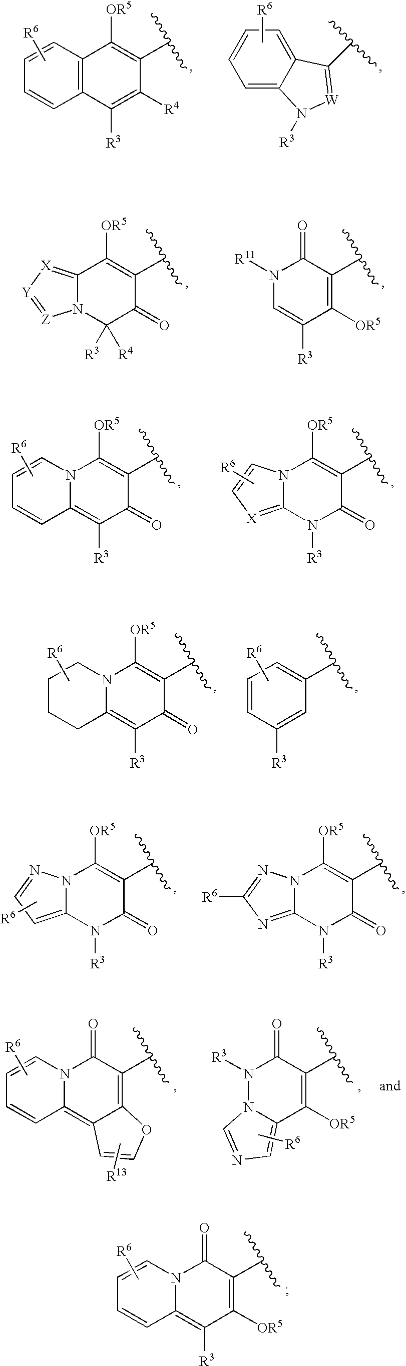

X, Y, and Z can be each N (nitrogen) or CR⁷, wherein each R⁷ can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino; W can be N or CR¹², wherein R¹² can be selected from hydrogen, hydroxyl, optionally substituted alkyl, optionally substituted alkoxy and optionally substituted amino; R² can be present from 0 to 4 times, wherein each R² can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted amino, and —NH(SO₂R⁸), each R⁸ can be independently selected from optionally substituted alkyl and optionally substituted cycloalkyl; R³ is selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted arylalkyl, and optionally substituted amino; R⁴ can be selected from hydrogen, hydroxyl, optionally substituted alkyl, optionally substituted alkoxy and optionally substituted amino; R⁵ can be selected from hydrogen and optionally substituted alkyl; R⁶ can be present from 0 to 4 times, wherein each R⁶ can be independently selected from halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino; R¹¹ can be selected from an optionally substituted aryl, an optionally substituted heteroaryl, an optionally substituted alicyclyl, an optionally substituted heterocyclyl, an optionally substituted alkyl, an optionally substituted alkenyl, an optionally substituted alkynyl, alkyl-CO—, and alkenyl-CO—; R¹³ can be selected from hydrogen, hydroxyl, optionally substituted alkyl, optionally substituted alkoxy and optionally substituted amino; and with the proviso that Formula I cannot be

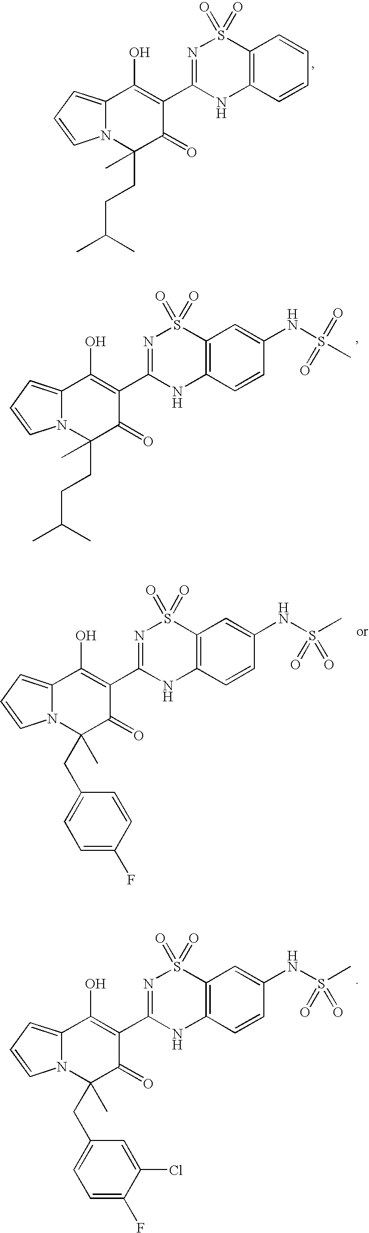

The present embodiments provide for a method of inhibiting NS5B polymerase activity comprising contacting a NS5B polymerase with a compound disclosed herein.

The present embodiments provide for a method of treating hepatitis by modulating NS5B polymerase activity comprising contacting a NS5B polymerase with a compound disclosed herein.

Preferred embodiments provide a pharmaceutical composition comprising: a) a preferred compound; and b) a pharmaceutically acceptable carrier.

Preferred embodiments provide a method of treating a hepatitis C virus infection in an individual, the method comprising administering to the individual an effective amount of a composition comprising a preferred compound.

Preferred embodiments provide a method of treating liver fibrosis in an individual, the method comprising administering to the individual an effective amount of a composition comprising a preferred compound.

Preferred embodiments provide a method of increasing liver function in an individual having a hepatitis C virus infection, the method comprising administering to the individual an effective amount of a composition comprising a preferred compound.

### DETAILED DESCRIPTION OF THE EMBODIMENTS

### Definitions

As used herein, common organic abbreviations are defined as follows:

### Ac Acetyl

Ac₂O Acetic anhydride
aq. Aqueous

### Bn Benzyl

### Bz Benzoyl

BOC or Boc tert-Butoxycarbonyl
Bu n-Butyl
cat. Catalytic

### Cbz Carbobenzyloxy

CDI 1,1′-carbonyldiimidazole
Cy (c-C₆H₁₁Cyclohexyl
° C. Temperature in degrees Centigrade

### d Density

DBU 1,8-Diazabicyclo[5.4.0]undec-7-ene

### DCE 1,2-Dichloroethane

### DCM Dichloromethane

### DIEA Diisopropylethylamine

### DMA Dimethylacetamide

### DMAP N,N-Dimethylaminopyridine

### DME Dimethoxyethane

### DMF N,N′-Dimethylformamide

### DMSO Dimethylsulfoxide

### Et Ethyl

EtOAc Ethyl acetate

### g Gram(s)

h Hour (hours)
HATU 2-(1H-7-azabenzotriazol-1-yl)-1,1,3,3-tetramethyl uronium hexafluorophosphate

### HMPA Hexamethylphosphoramide

HPLC High performance liquid chromatography

### iPr Isopropyl

LCMS Liquid chromatography-mass spectrometry
LDA Lithium diisopropylamide
mCPBA meta-Chloroperoxybenzoic Acid
min minute (minutes)

### MeOH Methanol

### MeCN Acetonitrile

### mL Milliliter(s)

MTBE Methyl tertiary-butyl ether

### NBS N-Bromosuccinimide

NH₄OAc Ammonium acetate
PE:EA Petroleum ether:ethyl acetate
PG Protecting group
Pd/C Palladium on activated carbon
PPSE Polyphosphoric acid trimethylsilyl ester

### ppt Precipitate

RCM Ring closing metathesis
rt or r.t. Room temperature
sBuLi sec-Butylithium

### TEA Triethylamine

TCDI 1,1′-Thiocarbonyl diimidazole
Tert, t tertiary
TFA Trifluoracetic acid

### THF Tetrahydrofuran

TLC Thin-layer chromatography

### TMEDA Tetramethylethylenediamine

### TMS Trimethylsilyl

### μL Microliter(s)

As used herein, the term “hepatic fibrosis,” used interchangeably herein with “liver fibrosis,” refers to the growth of scar tissue in the liver that can occur in the context of a chronic hepatitis infection.

The terms “individual,” “host,” “subject,” and “patient” are used interchangeably herein, and refer to a mammal, including, but not limited to, primates, including simians and humans.

As used herein, the term “liver function” refers to a normal function of the liver, including, but not limited to, a synthetic function, including, but not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

The term “sustained viral response” (SVR; also referred to as a “sustained response” or a “durable response”), as used herein, refers to the response of an individual to a treatment regimen for HCV infection, in terms of serum HCV titer. Generally, a “sustained viral response” refers to no detectable HCV RNA (e.g., less than about 500, less than about 200, or less than about 100 genome copies per milliliter serum) found in the patient's serum for a period of at least about one month, at least about two months, at least about three months, at least about four months, at least about five months, or at least about six months following cessation of treatment.

“Treatment failure patients” as used herein generally refers to HCV-infected patients who failed to respond to previous therapy for HCV (referred to as “non-responders”) or who initially responded to previous therapy, but in whom the therapeutic response was not maintained (referred to as “relapsers”). The previous therapy generally can include treatment with IFN-α monotherapy or IFN-α combination therapy, where the combination therapy may include administration of IFN-α and an antiviral agent such as ribavirin.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “host,” “subject,” and “patient” are used interchangeably herein, and refer to a mammal, including, but not limited to, murines, simians, humans, mammalian farm animals, mammalian sport animals, and mammalian pets.

As used herein, the term “a Type I interferon receptor agonist” refers to any naturally occurring or non-naturally occurring ligand of human Type I interferon receptor, which binds to and causes signal transduction via the receptor. Type I interferon receptor agonists include interferons, including naturally-occurring interferons, modified interferons, synthetic interferons, pegylated interferons, fusion proteins comprising an interferon and a heterologous protein, shuffled interferons; antibody specific for an interferon receptor; non-peptide chemical agonists; and the like.

As used herein, the term “Type II interferon receptor agonist” refers to any naturally occurring or non-naturally occurring ligand of human Type II interferon receptor that binds to and causes signal transduction via the receptor. Type II interferon receptor agonists include native human interferon-γ, recombinant IFN-γ species, glycosylated IFN-γ species, pegylated IFN-γ species, modified or variant IFN-γ species, IFN-γ fusion proteins, antibody agonists specific for the receptor, non-peptide agonists, and the like.

As used herein, the term “a Type III interferon receptor agonist” refers to any naturally occurring or non-naturally occurring ligand of humanIL-28 receptor α (“IL-28R”), the amino acid sequence of which is described by Sheppard, et al., infra., that binds to and causes signal transduction via the receptor.

As used herein, the term “interferon receptor agonist” refers to any Type I interferon receptor agonist, Type II interferon receptor agonist, or Type III interferon receptor agonist.

The term “dosing event” as used herein refers to administration of an antiviral agent to a patient in need thereof, which event may encompass one or more releases of an antiviral agent from a drug dispensing device. Thus, the term “dosing event,” as used herein, includes, but is not limited to, installation of a continuous delivery device (e.g., a pump or other controlled release injectible system); and a single subcutaneous injection followed by installation of a continuous delivery system.

“Continuous delivery” as used herein (e.g., in the context of “continuous delivery of a substance to a tissue”) is meant to refer to movement of drug to a delivery site, e.g., into a tissue in a fashion that provides for delivery of a desired amount of substance into the tissue over a selected period of time, where about the same quantity of drug is received by the patient each minute during the selected period of time.

“Controlled release” as used herein (e.g., in the context of “controlled drug release”) is meant to encompass release of substance (e.g., a Type I or Type III interferon receptor agonist, e.g., IFN-α) at a selected or otherwise controllable rate, interval, and/or amount, which is not substantially influenced by the environment of use. “Controlled release” thus encompasses, but is not necessarily limited to, substantially continuous delivery, and patterned delivery (e.g., intermittent delivery over a period of time that is interrupted by regular or irregular time intervals).

“Patterned” or “temporal” as used in the context of drug delivery is meant delivery of drug in a pattern, generally a substantially regular pattern, over a pre-selected period of time (e.g., other than a period associated with, for example a bolus injection). “Patterned” or “temporal” drug delivery is meant to encompass delivery of drug at an increasing, decreasing, substantially constant, or pulsatile, rate or range of rates (e.g., amount of drug per unit time, or volume of drug formulation for a unit time), and further encompasses delivery that is continuous or substantially continuous, or chronic.

The term “controlled drug delivery device” is meant to encompass any device wherein the release (e.g., rate, timing of release) of a drug or other desired substance contained therein is controlled by or determined by the device itself and not substantially influenced by the environment of use, or releasing at a rate that is reproducible within the environment of use.

By “substantially continuous” as used in, for example, the context of “substantially continuous infusion” or “substantially continuous delivery” is meant to refer to delivery of drug in a manner that is substantially uninterrupted for a pre-selected period of drug delivery, where the quantity of drug received by the patient during any 8 hour interval in the pre-selected period never falls to zero. Furthermore, “substantially continuous” drug delivery can also encompass delivery of drug at a substantially constant, pre-selected rate or range of rates (e.g., amount of drug per unit time, or volume of drug formulation for a unit time) that is substantially uninterrupted for a pre-selected period of drug delivery.

By “substantially steady state” as used in the context of a biological parameter that may vary as a function of time, it is meant that the biological parameter exhibits a substantially constant value over a time course, such that the area under the curve defined by the value of the biological parameter as a function of time for any 8 hour period during the time course (AUC8 hr) is no more than about 20% above or about 20% below, and preferably no more than about 15% above or about 15% below, and more preferably no more than about 10% above or about 10% below, the average area under the curve of the biological parameter over an 8 hour period during the time course (AUC8 hr average). The AUC8 hr average is defined as the quotient (q) of the area under the curve of the biological parameter over the entirety of the time course (AUCtotal) divided by the number of 8 hour intervals in the time course (total/3 days), i.e., q=(AUCtotal)/(total/3 days). For example, in the context of a serum concentration of a drug, the serum concentration of the drug is maintained at a substantially steady state during a time course when the area under the curve of serum concentration of the drug over time for any 8 hour period during the time course (AUC8 hr) is no more than about 20% above or about 20% below the average area under the curve of serum concentration of the drug over an 8 hour period in the time course (AUC8 hr average), i.e., the AUC8 hr is no more than 20% above or 20% below the AUC8 hr average for the serum concentration of the drug over the time course.

The term “alkyl” as used herein refers to a radical of a fully saturated hydrocarbon, including, but not limited to, methyl, ethyl, n-propyl, isopropyl, n-butyl, isobutyl, tert-butyl, n-hexyl,

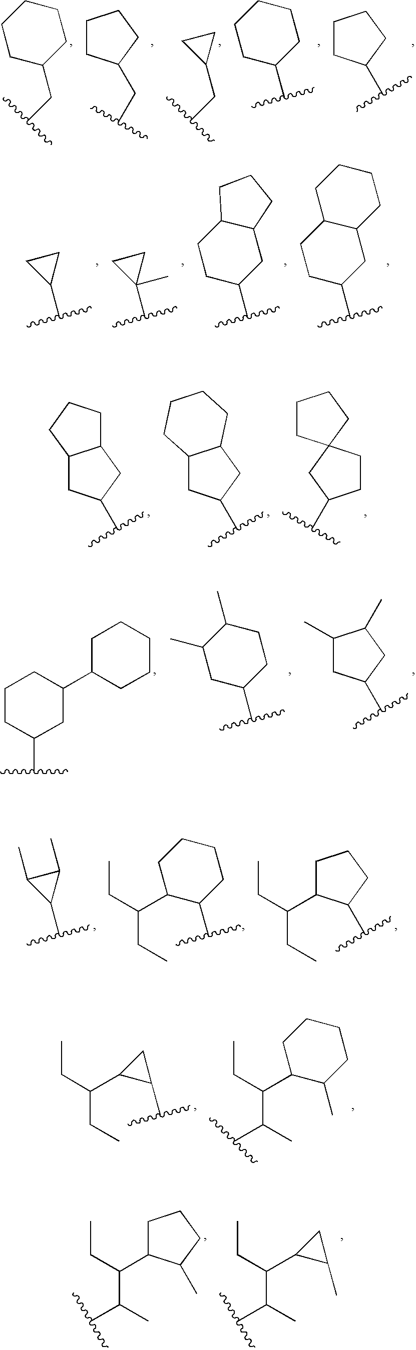

and the like. For example, the term “alkyl” as used herein includes radicals of fully saturated hydrocarbons defined by the following general formula's: the general formula for linear or branched fully saturated hydrocarbons not containing a cyclic structure is CₙH₂ₙ₊₂; the general formula for a fully saturated hydrocarbon containing one ring is CₙH₂ₙ; the general formula for a fully saturated hydrocarbon containing two rings is CₙH₂₍ₙ₋₁₎; the general formula for a saturated hydrocarbon containing three rings is CₙH₂₍ₙ₋₂₎.

The term “halo” used herein refers to fluoro, chloro, bromo, or iodo.

The term “alkoxy” used herein refers to straight or branched chain alkyl radical covalently bonded to the parent molecule through an —O— linkage. Examples of alkoxy groups include, but are not limited to, methoxy, ethoxy, propoxy, isopropoxy, butoxy, n-butoxy, sec-butoxy, t-butoxy and the like.

The term “alkenyl” used herein refers to a monovalent straight or branched chain radical of from two to twenty carbon atoms containing a carbon double bond including, but not limited to, 1-propenyl, 2-propenyl, 2-methyl-1-propenyl, 1-butenyl, 2-butenyl, and the like.

The term “alkynyl” used herein refers to a monovalent straight or branched chain radical of from two to twenty carbon atoms containing a carbon triple bond including, but not limited to, 1-propynyl, 1-butynyl, 2-butynyl, and the like.

The term “aryl” used herein refers to homocyclic aromatic radical whether one ring or multiple fused rings. Examples of aryl groups include, but are not limited to, phenyl, naphthyl, biphenyl, phenanthrenyl, naphthacenyl, and the like.

The term “cycloalkyl” used herein refers to saturated aliphatic ring system radical having three to twenty carbon atoms including, but not limited to, cyclopropyl, cyclopentyl, cyclohexyl, cycloheptyl, and the like.

The term “cycloalkenyl” used herein refers to aliphatic ring system radical having three to twenty carbon atoms having at least one carbon-carbon double bond in the ring. Examples of cycloalkenyl groups include, but are not limited to, cyclopropenyl, cyclopentenyl, cyclohexenyl, cycloheptenyl, and the like.

The term “polycycloalkyl” used herein refers to saturated aliphatic ring system radical having at least two rings that are fused with or without bridgehead carbons. Examples of polycycloalkyl groups include, but are not limited to, bicyclo[4.4.0]decanyl, bicyclo[2.2.1]heptanyl, adamantyl, norbornyl, and the like.

The term “polycycloalkenyl” used herein refers to aliphatic ring system radical having at least two rings that are fused with or without bridgehead carbons in which at least one of the rings has a carbon-carbon double bond. Examples of polycycloalkenyl groups include, but are not limited to, norbornylenyl, 1,1′-bicyclopentenyl, and the like.

The term “polycyclic hydrocarbon” used herein refers to a ring system radical in which all of the ring members are carbon atoms. One or more rings in polycyclic hydrocarbons can be aromatic or can contain less than the maximum number of non-cumulative double bonds. Examples of polycyclic hydrocarbon include, but are not limited to, naphthyl, dihydronaphthyl, indenyl, fluorenyl, and the like.

The term “heterocyclic” or “heterocyclyl” used herein refers to a cyclic ring system radical having at least one non-aromatic ring in which one or more ring atoms are not carbon, namely heteroatom. Examples of heterocyclic groups include, but are not limited to, morpholinyl, tetrahydrofuranyl, dioxolanyl, pyrrolidinyl, pyranyl, and the like.

The term “heteroaryl” used herein refers to a monocyclic or multicyclic aromatic ring system (a ring system with fully delocalized pi-electron system) that contain(s) one or more heteroatoms. In fused ring systems, the one or more heteroatoms may be present in only one of the rings. Examples of heteroaryl groups include, but are not limited to, benzothiazyl, benzoxazyl, quinazolinyl, quinolinyl, isoquinolinyl, quinoxalinyl, pyridinyl, pyrrolyl, oxazolyl, indolyl, and the like.

The term “arylalkyl” used herein refers to one or more aryl groups appended to an alkyl radical. Examples of arylalkyl groups include, but are not limited to, benzyl, phenethyl, phenpropyl, phenbutyl, and the like.

The term “cycloalkylalkyl” used herein refers to one or more cycloalkyl groups appended to an alkyl radical. Examples of cycloalkylalkyl include, but are not limited to, cyclohexylmethyl, cyclohexylethyl, cyclopentylmethyl, cyclopentylethyl, and the like.

The term “heteroarylalkyl” used herein refers to one or more heteroaryl groups appended to an alkyl radical. Examples of heteroarylalkyl include, but are not limited to, pyridylmethyl, furanylmethyl, thiopheneylethyl, and the like.

The term “heterocyclylalkyl” used herein refers to one or more heterocyclyl groups appended to an alkyl radical. Examples of heterocyclylalkyl include, but are not limited to, morpholinylmethyl, morpholinylethyl, morpholinylpropyl, tetrahydrofuranylmethyl, pyrrolidinylpropyl, and the like.

The term “alicyclic” used herein refers to saturated or unsaturated aliphatic ring system radical having one or more ring including, but are not limited to, cyclopropyl, cyclopentyl, cyclohexyl, cycloheptyl, cyclohexenyl, cyclohexadiene and the like.

The term “aryloxy” used herein refers to an aryl radical covalently bonded to the parent molecule through an —O— linkage.

The term “alkylthio” used herein refers to straight or branched chain alkyl radical covalently bonded to the parent molecule through an —S— linkage. Examples of alkoxy groups include, but are not limited to, methoxy, ethoxy, propoxy, isopropoxy, butoxy, n-butoxy, sec-butoxy, t-butoxy and the like.

The term “arylthio” used herein refers to an aryl radical covalently bonded to the parent molecule through an —S— linkage.

The term “alkylamino” used herein refers to nitrogen radical with one or more alkyl groups attached thereto. Thus, monoalkylamino refers to nitrogen radical with one alkyl group attached thereto and dialkylamino refers to nitrogen radical with two alkyl groups attached thereto.

The term “cyanoamino” used herein refers to nitrogen radical with nitrile group attached thereto.

The term “carbamyl” used herein refers to RNHCOO—.

The term “keto” and “carbonyl” used herein refers to C═O.

The term “carboxy” used herein refers to —COOH.

The term “sulfamyl” used herein refers to —SO₂NH₂.

The term “sulfonyl” used herein refers to —SO₂—.

The term “sulfinyl” used herein refers to —SO—.

The term “thiocarbonyl” used herein refers to C═S.

The term “thiocarboxy” used herein refers to CSOH.

The term “cyano” used herein refers to —CN.

The term “hydroxyl” used herein refers to —OH.

The term “nitro” used herein refers to —NO₂.

The term “amino” used herein refers to —NH₂.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

As used herein, a substituted group is derived from the unsubstituted parent structure in which there has been an exchange of one or more hydrogen atoms for another atom or group. When substituted, the substituent group(s) is (are) one or more group(s) individually and independently selected from C₁-C₆ alkyl, C₁-C₆ alkenyl, C₁-C₆ alkynyl, C₃-C₆ cycloalkyl (optionally substituted with halo, alkyl, alkoxy, carboxyl, CN, —SO₂-alkyl, —CF₃, and —OCF₃), C₃-C₆ heterocycloalkyl (e.g., tetrahydrofuryl) (optionally substituted with halo, alkyl, alkoxy, carboxyl, CN, —SO₂-alkyl, —CF₃, and —OCF₃), aryl (optionally substituted with halo, alkyl, alkoxy, carboxyl, CN, —SO₂-alkyl, —CF₃, and —OCF₃), heteroaryl (optionally substituted with, alkyl, alkoxy, carboxyl, CN, —SO₂-alkyl, —CF₃, and —OCF₃), halo (e.g., chloro, bromo, iodo and fluoro), cyano, hydroxy, C₁-C₆ alkoxy, aryloxy, sulfhydryl (mercapto), C₁-C₆ alkylthio, arylthio, mono- and di-(C₁-C₆)alkyl amino, quaternary ammonium salts, amino(C₁-C₆)alkoxy, hydroxy(C₁-C₆)alkylamino, amino(C₁-C₆)alkylthio, cyanoamino, nitro, carbamyl, keto (oxo), carbonyl, carboxy, glycolyl, glycyl, hydrazino, guanyl, sulfamyl, sulfonyl, sulfinyl, thiocarbonyl, thiocarboxy, and combinations thereof. The protecting groups that can form the protective derivatives of the above substituents are known to those of skill in the art and can be found in references such as Greene and Wuts Protective Groups in Organic Synthesis; John Wiley and Sons: New York, 1999. Wherever a substituent is described as “optionally substituted” that substituent can be substituted with the above substituents.

Asymmetric carbon atoms may be present in the compounds described. All such isomers, including diastereomers and enantiomers, as well as the mixtures thereof are intended to be included in the scope of the recited compound. In certain cases, compounds can exist in tautomeric forms. All tautomeric forms are intended to be included in the scope. Likewise, when compounds contain an alkenyl or alkenylene group, there exists the possibility of cis- and trans-isomeric forms of the compounds. Both cis- and trans-isomers, as well as the mixtures of cis- and trans-isomers, are contemplated. Thus, reference herein to a compound includes all of the aforementioned isomeric forms unless the context clearly dictates otherwise.

Various forms are included in the embodiments, including polymorphs, solvates, hydrates, conformers, salts, and prodrug derivatives. A polymorph is a composition having the same chemical formula, but a different structure. A solvate is a composition formed by salvation (the combination of solvent molecules with molecules or ions of the solute). A hydrate is a compound formed by an incorporation of water. A conformer is a structure that is a conformational isomer. Conformational isomerism is the phenomenon of molecules with the same structural formula but different conformations (conformers) of atoms about a rotating bond. Salts of compounds can be prepared by methods known to those skilled in the art. For example, salts of compounds can be prepared by reacting the appropriate base or acid with a stoichiometric equivalent of the compound. A prodrug is a compound that undergoes biotransformation (chemical conversion) before exhibiting its pharmacological effects. For example, a prodrug can thus be viewed as a drug containing specialized protective groups used in a transient manner to alter or to eliminate undesirable properties in the parent molecule. Thus, reference herein to a compound includes all of the aforementioned forms unless the context clearly dictates otherwise.

Where a range of values is provided, it is understood that each intervening value, to the tenth of the unit of the lower limit unless the context clearly dictates otherwise, between the upper and lower limit of that range and any other stated or intervening value in that stated range is encompassed within the embodiments. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the embodiments.

Unless defined otherwise, all technical and scientific terms used herein have the same meaning as commonly understood by one of ordinary skill in the art to which the embodiments belong. Although any methods and materials similar or equivalent to those described herein can also be used in the practice or testing of the embodiments, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The present embodiments provide compounds of Formula I, as well as pharmaceutical compositions and formulations comprising any compound of Formula I. A subject compound is useful for treating HCV infection and other disorders, as discussed below.

The embodiments provide a compound having the structure of Formula I:

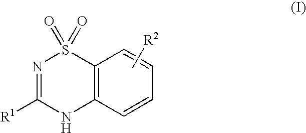

or a pharmaceutically acceptable salt or prodrug thereof; wherein R can be selected from:

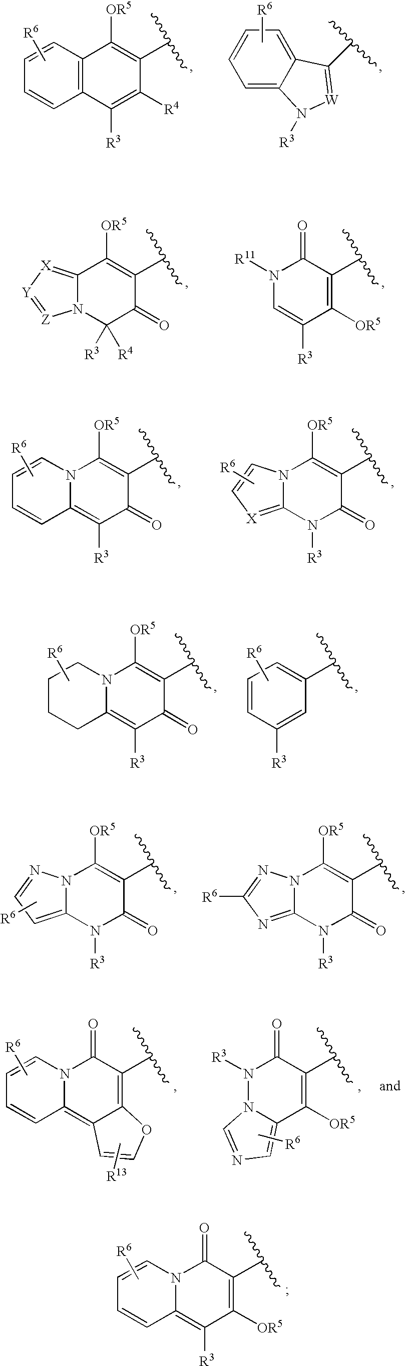

X, Y, and Z are each N or CR⁷, wherein each R⁷ can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino; W can be N (nitrogen) or CR¹², wherein R¹² can be selected from hydrogen, hydroxyl, optionally substituted alkyl, optionally substituted alkoxy and optionally substituted amino; R² can be present from 0 to 4 times, wherein each R² can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted amino, and —NH(SO₂R⁸), each R⁸ can be independently selected from optionally substituted alkyl and optionally substituted cycloalkyl; R³ can be selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted arylalkyl, and optionally substituted amino; R⁴ can be selected from hydrogen, hydroxyl, optionally substituted alkyl, optionally substituted alkoxy and optionally substituted amino; R⁵ can be selected from hydrogen and optionally substituted alkyl; R⁶ can be present from 0 to 4 times, wherein each R⁶ can be independently selected from halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino; R¹¹ can be selected from an optionally substituted aryl, an optionally substituted heteroaryl, an optionally substituted alicyclyl, an optionally substituted heterocyclyl, an optionally substituted alkyl, an optionally substituted alkenyl, an optionally substituted alkynyl, alkyl-CO—, and alkenyl-CO—; and R¹³ can be selected from hydrogen, hydroxyl, optionally substituted alkyl, optionally substituted alkoxy and optionally substituted amino with the proviso that Formula I cannot be

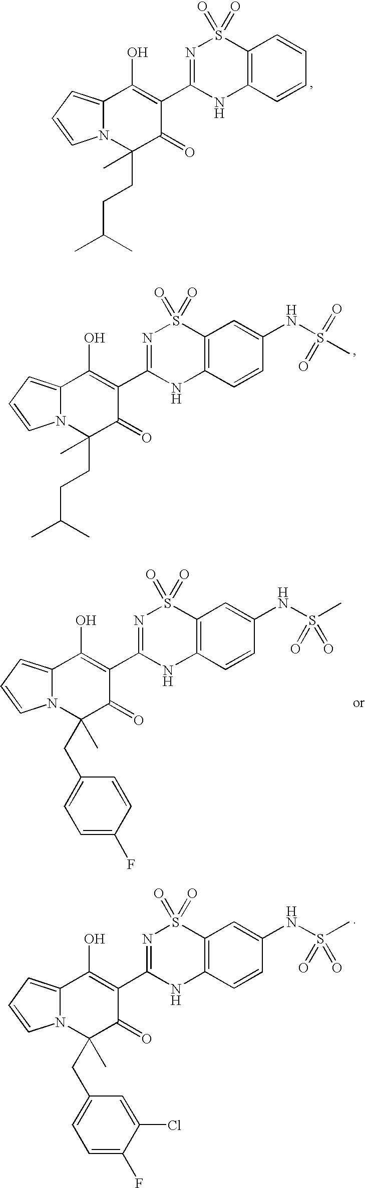

In some embodiments, when X, Y and Z are all CR⁷ and R⁷ is hydrogen, R³ and R⁴ cannot both be optionally substituted alkyl. In some embodiments, when X, Y and Z are all CH, R³ and R⁴ cannot both be alkyl. In some embodiments, when X, Y and Z are all CR⁷ and R⁷ is hydrogen, R⁴ cannot both be optionally substituted alkyl. In some embodiments, when X, Y and Z are all CR⁷ and R⁷ is hydrogen, R³ cannot be an optionally substituted arylalkyl; and R⁴ cannot both be optionally substituted alkyl.

In preferred embodiments, embodiments provide compounds of Formula I, in which R³ is —NR⁹R¹⁰, wherein R⁹ and R¹⁰ are independently selected from hydrogen and optionally substituted alkyl.

In preferred embodiments, embodiments provide compounds of Formula I, in which R³ is selected from halogen and optionally substituted alkyl. In other preferred embodiments, embodiments provide compounds of Formula I, in which R³ is an optionally substituted arylalkyl. In still other preferred embodiments, embodiments provide compounds of Formula I, in which R³ is an optionally substituted heteroarylalkyl.

In preferred embodiments, embodiments provide compounds of Formula I, in which R⁶ is not present.

In preferred embodiments, embodiments provide compounds of Formula I, in which R² is not present.

In an embodiment, a compound of Formula I can have the structure of Formula I-1:

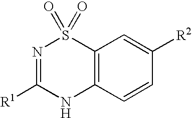

wherein R¹ and R² are described above and with the same provisos described above with respect to Formula I.

Another embodiment provides a compound having the structure of Formula Ia:

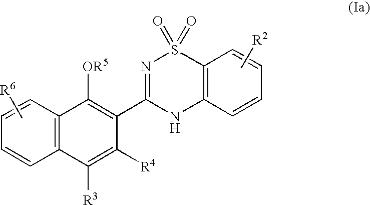

or a pharmaceutically acceptable salt or prodrug thereof, wherein R² can be present from 0 to 4 times, each R² can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted amino, and —NH(SO₂R⁸), and each R⁸ can be independently selected from optionally substituted alkyl and optionally substituted cycloalkyl; R³ can be selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted arylalkyl, and optionally substituted amino; R⁴ can be selected from hydrogen, hydroxyl, optionally substituted alkyl, optionally substituted alkoxy and optionally substituted amino; R⁵ can be selected from hydrogen and optionally substituted alkyl; and R⁶ can be present from 0 to 4 times, wherein each R⁶ can be independently selected from halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino.

In some embodiments in a compound of Formula Ia, R² can be present 0 times. In other embodiments, R² can be —NH(SO₂R⁸), wherein each R⁸ can be independently selected from optionally substituted alkyl and optionally substituted cycloalkyl. In an embodiment, when R² is —NH(SO₂R⁸), R⁸ is an optionally substituted alkyl, such as methyl. In some embodiments, R³ can be an optionally substituted alkyl. In an embodiment, R³ can be isopentyl. In other embodiments, R³ can be halogen. In some embodiments, R⁴ can be hydroxyl. In some embodiments, R⁵ can be hydrogen. In other embodiments, R⁵ can be an optionally substituted alkyl. In an embodiment, R⁵ can be methyl. In some embodiments, R⁶ can be present 0 times. In some embodiments, R² can be present 0 times; R³ can be halogen; R⁴ can be hydroxyl; R⁵ can be hydrogen; and R⁶ can be present 0 times. In other embodiments, R² can be —NH(SO₂R⁸); R³ can be isopentyl; R⁴ can be hydroxyl; R⁵ can be hydrogen or an optionally substituted alkyl; and R⁶ can be present 0 times. In some embodiments, R² can be —NH(SO₂R⁸) and positioned at the same position as shown in Formula I-1.

Another embodiment provides a compound having the structure of Formula Ib:

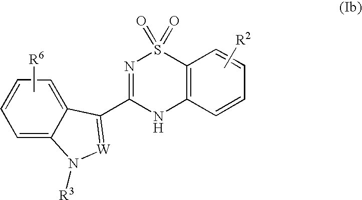

or a pharmaceutically acceptable salt or prodrug thereof, wherein W can be N or CR¹², wherein R¹² can be selected from hydrogen, hydroxyl, optionally substituted alkyl, optionally substituted alkoxy and optionally substituted amino; R² can be present from 0 to 4 times, wherein each R² can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted amino, and —NH(SO₂R⁸), each R⁸ can be independently selected from optionally substituted alkyl and optionally substituted cycloalkyl; R³ can be selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted arylalkyl, and optionally substituted amino; and R⁶ can be present from 0 to 4 times, wherein each R⁶ can be independently selected from halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino.

In preferred embodiments, embodiments provide compounds of Formula Ib, in which R³ is selected from halogen and optionally substituted alkyl.

In preferred embodiments, embodiments provide compounds of Formula Ib, in which R⁶ is selected from halogen, hydroxyl, optionally substituted alkoxy and optionally substituted alkyl.

In preferred embodiments, embodiments provide compounds of Formula Ib, in which R² is not present.

In some embodiments for a compound of Formula Ib, W can be N (nitrogen). In other embodiments, W can be CR¹², wherein R¹² can be selected from hydrogen, hydroxyl, optionally substituted alkyl, optionally substituted alkoxy and optionally substituted amino. In an embodiment, R¹² can be hydrogen.

In some embodiments, R² can be present 0 times in a compound of Formula Ib. In other embodiments, R² can be —NH(SO₂R⁸), each R⁸ can be independently selected from optionally substituted alkyl and optionally substituted cycloalkyl. In an embodiment, R⁸ is an optionally substituted alkyl (for example, methyl). In some embodiments, R³ can be an optionally substituted alkyl. In an embodiment, R³ can be isopentyl. In some embodiments, R⁶ can be present 0 times. In other embodiments, R⁶ can be present 1 time, wherein each R⁶ is independently selected from the group consisting of halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino. In some embodiments, R⁶ can be independently selected from halogen, hydroxy, optionally substituted alkyl, and optionally substituted alkoxy.

In some embodiments, R¹ can have a structure selected from:

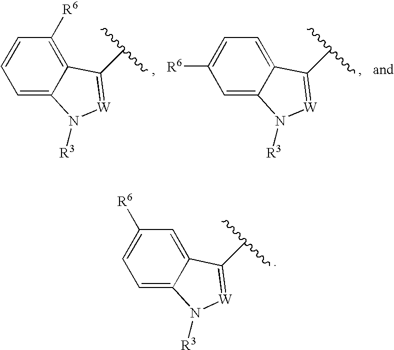

In some embodiments for a compound of Formula Ib, W can be N; R² can be present 0 times; R³ can be an optionally substituted alkyl; and R⁶ can be present from 0 to 1 times. In other embodiments for a compound of Formula Ib, W can be N; R² can be —NH(SO₂R⁸); R³ can be an optionally substituted alkyl; and R⁶ can be present from 0 to 1 times In some embodiments described in the present paragraph, when R⁶ is present, R⁶ can be independently selected from halogen, hydroxy, optionally substituted alkyl, and optionally substituted alkoxy. In some embodiments, R² can be —NH(SO₂R⁸) and positioned at the same position as shown in Formula I-1.

Another embodiment provides a compound having the structure of Formula Ic:

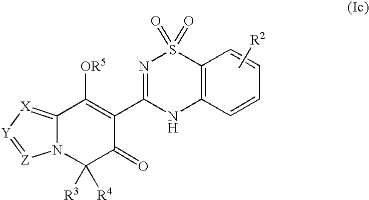

or a pharmaceutically acceptable salt or prodrug thereof, wherein X, Y, and Z can be each N or CR⁷, wherein each R⁷ can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino; R² can be present from 0 to 4 times, wherein each R² can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted amino, and —NH(SO₂R⁸), each R⁸ can be independently selected from optionally substituted alkyl and optionally substituted cycloalkyl; R³ can be selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted arylalkyl, and optionally substituted amino; R⁴ can be selected from hydrogen, hydroxyl, optionally substituted alkyl, optionally substituted alkoxy and optionally substituted amino; and R⁵ can be selected from hydrogen and optionally substituted alkyl, with the proviso that Formula Ic cannot be

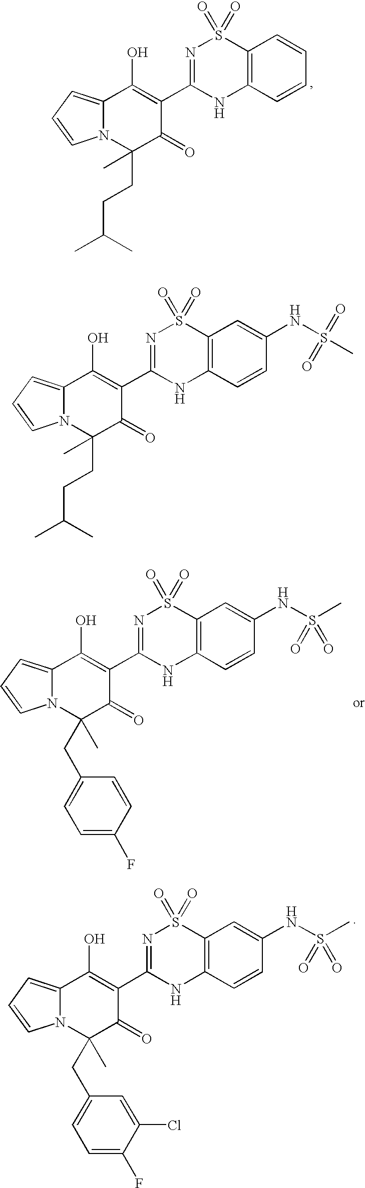

In some embodiments, X, Y, and Z are all CR⁷. In some embodiments, X is N when Y and Z are each CR⁷. In some embodiments, X and Z are each CR⁷ when Y is N. In some embodiments, X and Z are each N when Y is CR⁷. In some embodiments, X and Y are each CR⁷ when Z is N. In some of the embodiments of the present paragraph, R⁷ can be hydrogen.

In some embodiments, when X, Y and Z are all CR⁷ and R⁷ is hydrogen, R³ and R⁴ cannot both be optionally substituted alkyl. In some embodiments, when X, Y and Z are all CH, R³ and R⁴ cannot both be alkyl. In some embodiments, when X, Y and Z are all CR⁷ and R⁷ is hydrogen, R⁴ cannot both be alkyl. In some embodiments, when X, Y and Z are all CR⁷ and R⁷ is hydrogen, R³ cannot be an optionally substituted arylalkyl; and R⁴ cannot both be alkyl.

In some embodiments, R² can be present 0 times in a compound of Formula Ic. In other embodiments, R² can be —NH(SO₂R⁸), wherein each R⁸ can be independently selected from optionally substituted alkyl and optionally substituted cycloalkyl. In embodiment, R⁸ can be an optionally substituted alkyl, for example, methyl. In some embodiments, R³ can be an optionally substituted alkyl (for example, isopentyl). In some embodiments, R⁴ can be an optionally substituted alkyl. In an embodiment, R⁴ can be methyl. In some embodiments, R⁵ can be hydrogen.

In some embodiments, R² can be present 0 times; R³ can be an optionally substituted alkyl; R⁴ can be an optionally substituted alkyl and R⁵ can be hydrogen. In other embodiments, In some embodiments, R² can be —NH(SO₂R⁸); R³ can be an optionally substituted alkyl; R⁴ can be an optionally substituted alkyl and R⁵ can be hydrogen. In some embodiments described in this paragraph, X is N when Y and Z are each CR⁷. In other embodiments described in this paragraph, X and Z are each CR⁷ when Y is N. In still other embodiments described in this paragraph, X and Z are each N when Y is CR⁷. In yet still other embodiments described in this paragraph, X and Y are each CR⁷ when Z is N. In some embodiments, R² can be —NH(SO₂R⁸) and positioned at the same position as shown in Formula I-1.

Another embodiment provides a compound having the structure of Formula Id:

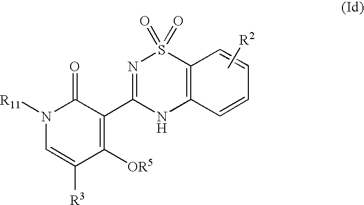

or a pharmaceutically acceptable salt or prodrug thereof, wherein R² can be present from 0 to 4 times, wherein each R² can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted amino, and —NH(SO₂R⁸), each R⁸ can be independently selected from optionally substituted alkyl and optionally substituted cycloalkyl; R³ can be selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted arylalkyl, and optionally substituted amino; R⁵ can be selected from hydrogen and optionally substituted alkyl; and R¹¹ can be selected from an optionally substituted aryl, an optionally substituted heteroaryl, an optionally substituted alicyclyl, an optionally substituted heterocyclyl, an optionally substituted alkyl, an optionally substituted alkenyl, an optionally substituted alkynyl, alkyl-CO—, and alkenyl-CO—.

In some embodiments, R² can be —NH(SO₂R⁸), wherein each R⁸ is independently selected from optionally substituted alkyl and optionally substituted cycloalkyl for a compound of Formula Id. In some embodiments, including those described in the present paragraph, R³ can be optionally substituted alkyl (such as isopentyl). In embodiment, R⁵ can be hydrogen. In some embodiments, R¹¹ can be an optionally substituted heteroaryl, for example, thiazole. In an embodiment, R² can be —NH(SO₂R⁸); R³ can be optionally substituted alkyl; and R⁵ can be hydrogen. In some embodiments, R² can be —NH(SO₂R⁸) and positioned at the same position as shown in Formula I-1.

Another embodiment provides a compound having the structure of Formula Ie:

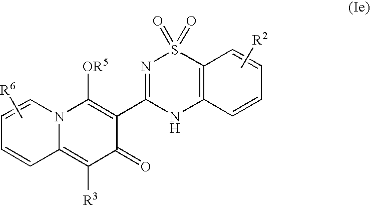

or a pharmaceutically acceptable salt or prodrug thereof, wherein R² can be present from 0 to 4 times, each R² can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted amino, and —NH(SO₂R⁸), and each R⁸ can be independently selected from optionally substituted alkyl and optionally substituted cycloalkyl; R³ can be selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted arylalkyl, and optionally substituted amino; R⁵ can be selected from hydrogen and optionally substituted alkyl; and R⁶ can be present from 0 to 4 times, wherein each R⁶ can be independently selected from halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino.

In some embodiments, R² can be —NH(SO₂R⁸), wherein each R⁸ is independently selected from optionally substituted alkyl and optionally substituted cycloalkyl for a compound of Formula Ie. In an embodiment, R² can be —NH(SO₂R⁸) and R⁸ can be an optionally substituted alkyl (for example, methyl) or an optionally substituted cycloalkyl (for example, cyclopropyl). In some embodiments, R³ can be an optionally substituted alkyl, such as isopentyl. In other embodiments, R³ can be an optionally substituted alkyl substituted with a C₃₋₆ cycloalkyl. In an embodiment, R³ can be an ethyl group substituted with a cyclopropyl group. In other embodiments, R³ can be an optionally substituted arylalkyl. One example of a suitable optionally substituted arylalkyl is an optionally substituted benzyl group. In some embodiments, the optionally substituted arylalkyl can be substituted with a substituent selected from halogen, sulfonyl, alkoxy, mono-(C₁-C₆)alkyl amino and di-(C₁-C₆)alkyl amino. For example, R³ can be a benzyl group substituted at the para, meta, and/or ortho positions with a substituent selected from halogen, sulfonyl, alkoxy, mono-(C₁-C₆)alkyl amino and di-(C₁-C₆)alkyl amino. In an embodiment, R³ can be a para-substituted benzyl group. In another embodiment, R³ can be a meta-substituted benzyl group. In still another embodiment, R³ can be an ortho-substituted benzyl group. In yet still another embodiment, R³ can be a di-substituted benzyl group. In some embodiments, R³ can be an optionally substituted heteroarylalkyl. When R³ is an optionally substituted heteroarylalkyl, the heteroaryl group of an optionally substituted heteroarylalkyl can be selected from an optionally substituted furyl, an optionally substituted thiophene and an optionally substituted pyrrolyl. In an embodiment, the optionally substituted pyrrolyl can be an alkyl-substituted pyrrolyl. In some embodiments, including those described in this paragraph, R⁵ can be hydrogen. Additionally, in some embodiments, including described in the present paragraph, R⁶ can be present 0 times. In other embodiments, including described in the present paragraph, R⁶ can be present 1 time, wherein each R⁶ can be independently selected from halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino. For example, R⁶ can be present 1 time and be independently selected from halogen and an optionally substituted alkyl (for example, methyl). In some embodiments, R can be —NH(SO₂R⁸) and positioned at the same position as shown in Formula I-1.

Another embodiment provides a compound having the structure of Formula If:

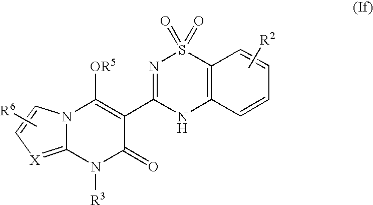

or a pharmaceutically acceptable salt or prodrug thereof, wherein X can be N or CR⁷, each R⁷ can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino; R² can be present from 0 to 4 times, wherein each R² can be independently selected from halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted amino, and —NH(SO₂R⁸); R³ can be selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted arylalkyl, and optionally substituted amino; R⁵ can be selected from hydrogen and optionally substituted alkyl; R⁶ can be present from 0 to 2 times, wherein each R⁶ can be independently selected from halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino; and each R⁸ can be independently selected from optionally substituted alkyl and optionally substituted cycloalkyl.

In some embodiments, in a compound of Formula If, X can be N (nitrogen). In other embodiments, X can be CR⁷; wherein each R⁷ can be hydrogen or an optionally substituted alkyl. In some embodiments, R² can be present 0 times. In other embodiments, R² can be —NH(SO₂R⁸), each R⁸ is independently selected from the group consisting of optionally substituted alkyl and optionally substituted cycloalkyl. In an embodiment, R⁸ can be an optionally substituted alkyl, such as methyl. In some embodiments, including those described in the present paragraph, R³ can be an optionally substituted alkyl. In an embodiment, R³ can be isopentyl. In some embodiments, R⁵ can be hydrogen. In some embodiments, R⁶ can be present 0 times. In an embodiment, X can be N; R² can be present 0 times; R³ can be an optionally substituted alkyl; R⁵ can be hydrogen; and R⁶ can be present 0 times. In another embodiment, X can be N; R² can —NH(SO₂R⁸); R³ can be an optionally substituted alkyl; R⁵ can be hydrogen; and R⁶ can be present 0 times. In some embodiments, R² can be —NH(SO₂R⁸) and positioned at the same position as shown in Formula I-1.

Another embodiment provides a compound having the structure of Formula Ig:

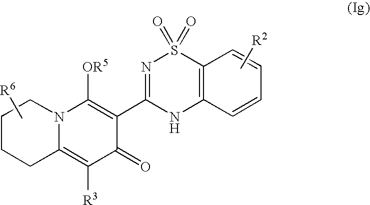

or a pharmaceutically acceptable salt or prodrug thereof, wherein R² can be present from 0 to 4 times, each R² can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted amino, and —NH(SO₂R⁸), and each R⁸ can be independently selected from optionally substituted alkyl and optionally substituted cycloalkyl; R³ can be selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted arylalkyl, and optionally substituted amino; R⁵ can be selected from hydrogen and optionally substituted alkyl; and R⁶ can be present from 0 to 4 times, wherein each R⁶ can be independently selected from halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino.

In some embodiments, R² can be —NH(SO₂R⁸), wherein each R⁸ is independently selected from optionally substituted alkyl and optionally substituted cycloalkyl for a compound of Formula Ig. When R² is —NH(SO₂R⁸), in some embodiments, R⁸ can be an optionally substituted alkyl, such as methyl. In some embodiments, for a compound of Formula Ig, R³ can be an optionally substituted alkyl. In an embodiment, R³ can be isopentyl. In some embodiments, including those described in the present paragraph, R⁵ can be hydrogen. In some embodiments, R² can be —NH(SO₂R⁸) and positioned at the same position as shown in Formula I-1.

Another embodiment provides a compound having the structure of Formula Ih:

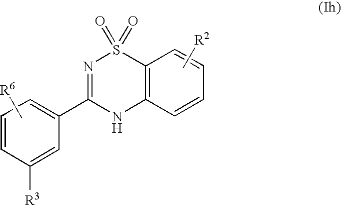

or a pharmaceutically acceptable salt or prodrug thereof, wherein R² can be present from 0 to 4 times, each R² can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted amino, and —NH(SO₂R⁸), and each R⁸ can be independently selected from optionally substituted alkyl and optionally substituted cycloalkyl; R³ can be selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted arylalkyl, and optionally substituted amino; and R⁶ can be present from 0 to 4 times, wherein each R⁶ can be independently selected from halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino.

In some embodiments, R² can be —NH(SO₂R⁸), wherein each R⁸ is independently selected from optionally substituted alkyl and optionally substituted cycloalkyl for a compound of Formula Ih. When R² is —NH(SO₂R⁸), in some embodiments, R⁸ can be an optionally substituted alkyl, such as methyl. In some embodiments, R³ can be a haloalkyl, including a mono-haloalkyl, di-haloalkyl or tri-haloalkyl. In an embodiment, R³ can be trifluoromethyl. In some embodiments, including those of this paragraph, R can be present 0 times. In some embodiments, R² can be —NH(SO₂R⁸) and positioned at the same position as shown in Formula I-1.

An embodiment provides a compound having the structure of Formula II:

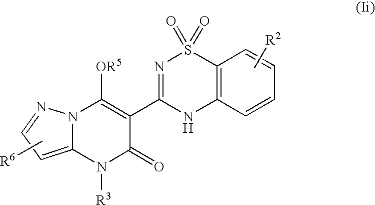

or a pharmaceutically acceptable salt or prodrug thereof, R² can be present from 0 to 4 times, wherein each R² can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted amino, and —NH(SO₂R⁸), each R⁸ can be independently selected from optionally substituted alkyl and optionally substituted cycloalkyl; R³ can be selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted arylalkyl, optionally substituted heteroarylalkyl, and optionally substituted amino; R⁵ can be selected from hydrogen and optionally substituted alkyl; and R⁶ can be present from 0 to 4 times, wherein each R⁶ can be independently selected from halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino.

In some embodiments, R² can be —NH(SO₂R⁸), wherein each R⁸ is independently selected from optionally substituted alkyl and optionally substituted cycloalkyl for a compound of Formula II. When R² is —NH(SO₂R⁸), in some embodiments, R⁸ can be an optionally substituted alkyl. In an embodiment, R⁸ can be methyl. In some embodiments, R³ can be an optionally substituted alkyl, for example, isopentyl. In other embodiments, R³ can be an optionally substituted arylalkyl. An example of a suitable optionally substituted arylalkyl is an optionally substituted benzyl group. In some embodiments, when R³ is an optionally substituted arylalkyl, the optionally substituted arylalkyl can be substituted with a substituent selected from the group consisting of halogen, sulfonyl, alkoxy, mono-(C₁-C₆)alkyl amino and di-(C₁-C₆)alkyl amino. In an embodiment, when the optionally substituted arylalkyl is an optionally substituted benzyl group, the aforementioned substituents can be present at the para, meta and/or ortho position(s). In some embodiments, the optionally substituted arylalkyl is a para-substituted benzyl group, for example, a para-substituted benzyl group substituted with a halogen. In some embodiments, including those described in this paragraph, R⁵ can be hydrogen. In some embodiments, R⁶ can be present 0 times. In other embodiments, R⁶ can be present 1 time, wherein each R⁶ can be independently selected from halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino. In an embodiments, R⁶ can be an optionally substituted alkyl (for example, methyl) or an optionally substituted cycloalkyl (for example, cyclopropyl). In some embodiments, R² can be —NH(SO₂R⁸), R³ can be an optionally substituted alkyl or an optionally substituted arylalkyl; R⁵ can be hydrogen; and R⁶ can be present 0 to 1 times. In some embodiments, R² can be —NH(SO₂R⁸) and positioned at the same position as shown in Formula I-1.

Another embodiment provides a compound having the structure of Formula Ij:

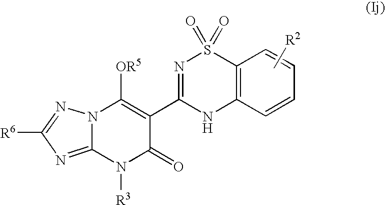

or a pharmaceutically acceptable salt or prodrug thereof, R² can be present from 0 to 4 times, wherein each R² can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted amino, and —NH(SO₂R⁸), each R⁸ can be independently selected from optionally substituted alkyl and optionally substituted cycloalkyl; R³ can be selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted arylalkyl, optionally substituted heteroarylalkyl, and optionally substituted amino; R⁵ can be selected from hydrogen and optionally substituted alkyl; and R⁶ can be present from 0 to 4 times, wherein each R⁶ can be independently selected from halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino.

In some embodiments, a compound having the structure of Formula Ij can be the following: R² can be —NH(SO₂R⁸), wherein each R⁸ is independently selected from optionally substituted alkyl and optionally substituted cycloalkyl. In an embodiment, R² can be —NH(SO₂R⁸); and R⁸ can be an optionally substituted alkyl (e.g., methyl). In some embodiments, R³ can be an optionally substituted alkyl in a compound of Formula Ij. In an embodiment, the optionally substituted alkyl of R³ can be isopentyl. In some embodiments, including those described in the present paragraph, R⁵ can be hydrogen. In some embodiments, in a compound of Formula Ij, R⁶ can be present 0 times. In an embodiment, R² can be —NH(SO₂R⁸), R³ can be an optionally substituted alkyl; R⁵ can be hydrogen; and can be present 0 times. In some embodiments, R² can be —NH(SO₂R⁸) and positioned at the same position as shown in Formula I-1.

An embodiment provides a compound having the structure of Formula Ik:

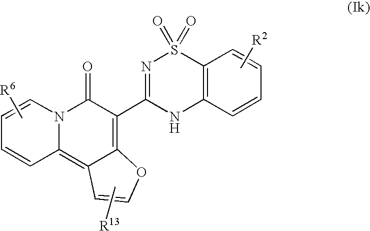

or a pharmaceutically acceptable salt or prodrug thereof, R² can be present from 0 to 4 times, wherein each R² can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted amino, and —NH(SO₂R⁸), each R⁸ can be independently selected from optionally substituted alkyl and optionally substituted cycloalkyl; R⁶ can be present from 0 to 4 times, wherein each R⁶ is independently selected from halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino; and R¹³ can be selected from hydrogen, hydroxyl, optionally substituted alkyl, optionally substituted alkoxy and optionally substituted amino.

In some embodiments, R² can be —NH(SO₂R⁸), wherein each R⁸ can be independently selected from optionally substituted alkyl and optionally substituted cycloalkyl in a compound of Formula Ik. In an embodiment, R⁸ can be an optionally substituted alkyl, for example, methyl. In some embodiments, R⁶ can be present 0 times in a compound of Formula Ik. In an embodiment, R² can be —NH(SO₂R⁸), and R⁶ can be present 0 times in a compound of formula Ik. In some embodiments, including those described in this paragraph, R¹³ can be an optionally substituted alkyl, such as methyl. In some embodiments, R² can be —NH(SO₂R⁸) and positioned at the same position as shown in Formula I-1.

An embodiment provides a compound having the structure of Formula Il:

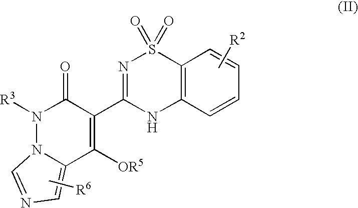

or a pharmaceutically acceptable salt or prodrug thereof, R² can be present from 0 to 4 times, wherein each R² can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted amino, and —NH(SO₂R⁸), each R⁸ can be independently selected from optionally substituted alkyl and optionally substituted cycloalkyl; R³ can be selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted arylalkyl, optionally substituted heteroarylalkyl, and optionally substituted amino; R⁵ can be selected from hydrogen and optionally substituted alkyl; and R⁶ can be present from 0 to 4 times, wherein each R⁶ is independently selected from halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino.

In some embodiments, R² can be —NH(SO₂R⁸), wherein each R⁸ can be independently selected from optionally substituted alkyl and optionally substituted cycloalkyl in a compound of Formula Il. In an embodiment, R⁸ can be an optionally substituted alkyl, for example, methyl. In some embodiments, R³ can be an optionally substituted alkyl (for example, methyl). In an embodiment, R⁵ can be hydrogen. In some embodiments, R⁶ can be present 0 times. In an embodiment, R² can be —NH(SO₂R⁸), R³ can be an optionally substituted alkyl; R⁵ can be hydrogen and R⁶ can be present 0 times. In some embodiments, R² can be —NH(SO₂R⁸) and positioned at the same position as shown in Formula I-1.

Another embodiment provides a compound having the structure of Formula Im:

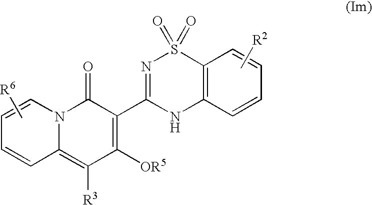

or a pharmaceutically acceptable salt or prodrug thereof, R² can be present from 0 to 4 times, wherein each R² can be independently selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted amino, and —NH(SO₂R⁸), each R⁸ can be independently selected from optionally substituted alkyl and optionally substituted cycloalkyl; R³ can be selected from hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted arylalkyl, optionally substituted heteroarylalkyl, and optionally substituted amino; R⁵ can be selected from hydrogen and optionally substituted alkyl; and R⁶ can be present from 0 to 4 times, wherein each R⁶ is independently selected from halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino.

In some embodiments, R² can be —NH(SO₂R⁸), wherein each R⁸ can be independently selected from optionally substituted alkyl and optionally substituted cycloalkyl in a compound of Formula Im. In an embodiment, R⁸ can be an optionally substituted alkyl, for example, methyl. In some embodiments, R³ can be an optionally substituted alkyl. For example, R³ can be an optionally substituted alkyl substituted by a C₃₋₆ cycloalkyl (for example, cyclohexyl). In an embodiment, R⁵ can be hydrogen. In some embodiments, R⁶ can be present 0 times. In an embodiment, R² can be —NH(SO₂R⁸), R³ can be an optionally substituted alkyl; R⁵ can be hydrogen and R⁶ can be present 0 times. In some embodiments, R² can be —NH(SO₂R⁸) and positioned at the same position as shown in Formula I-1.

Preferred embodiments provide a compound having one of the following formulas:

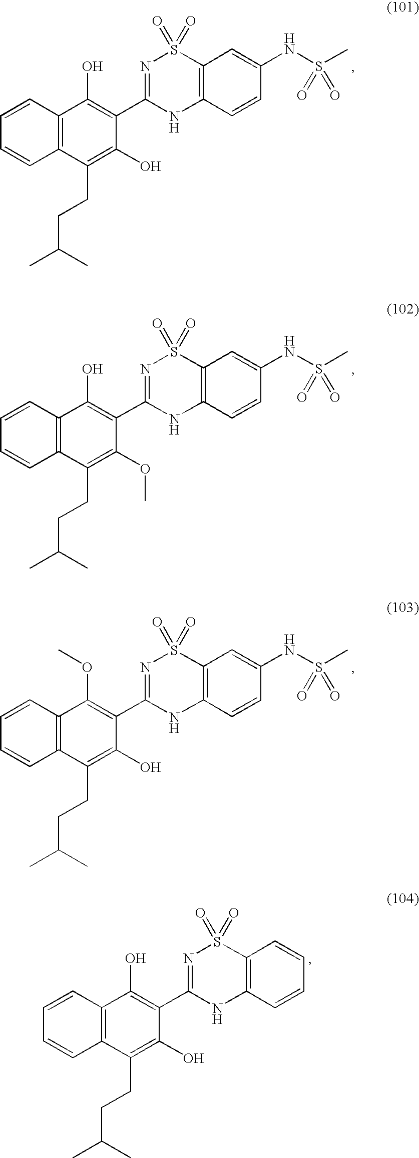

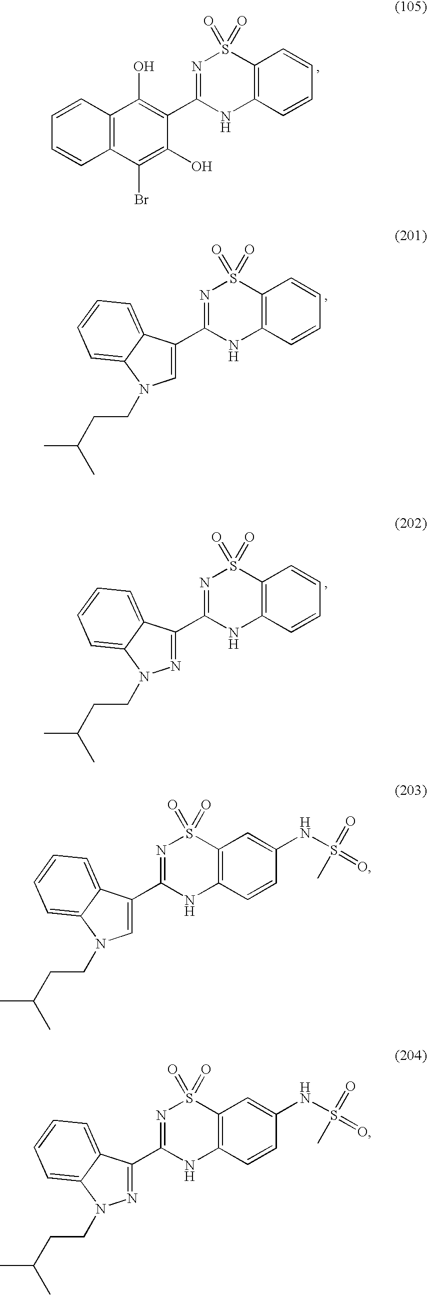

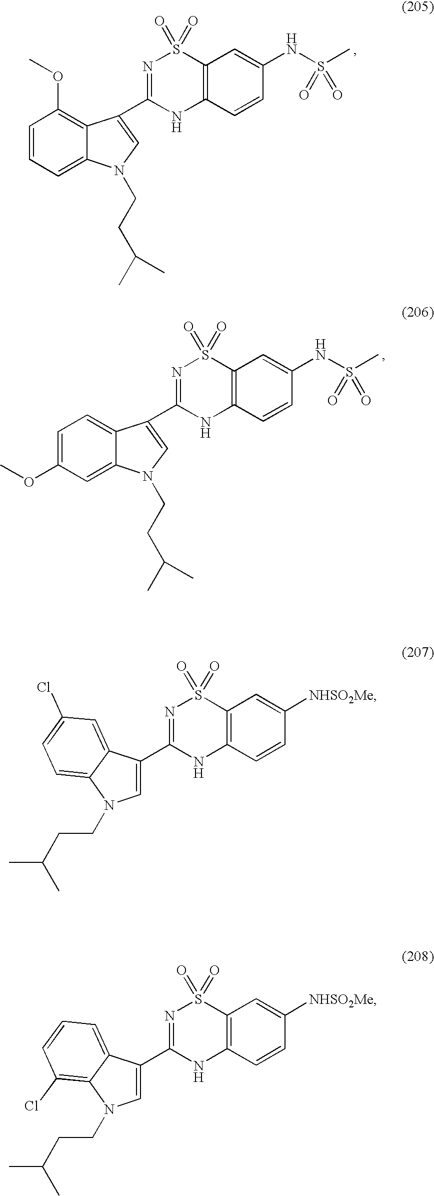

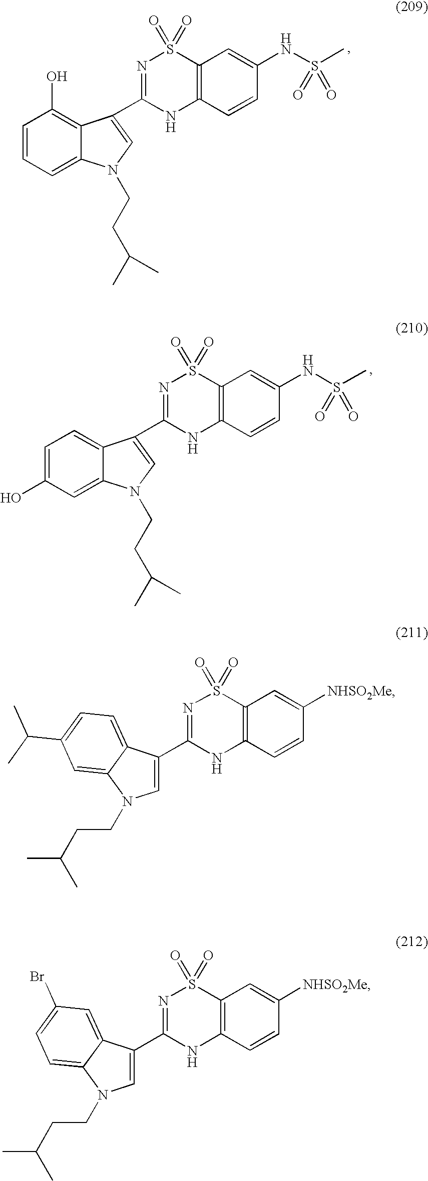

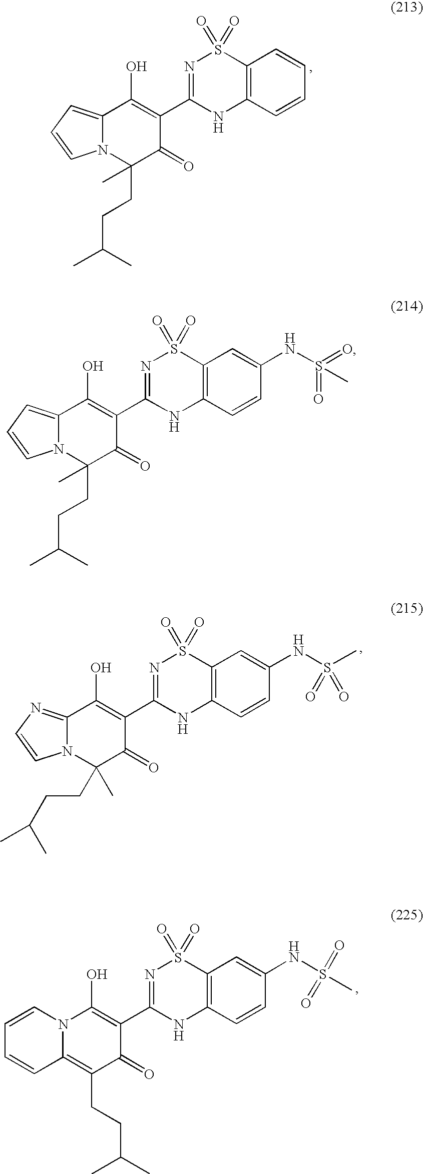

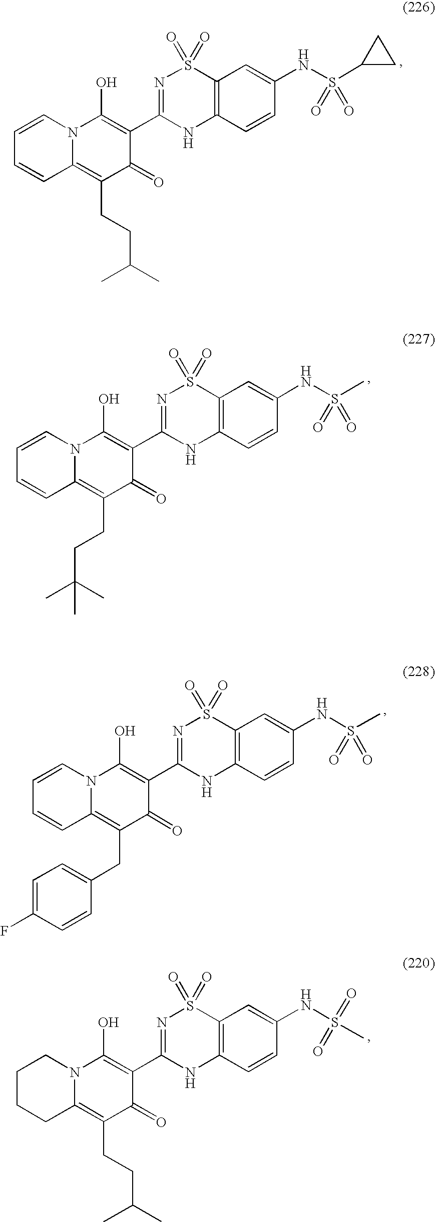

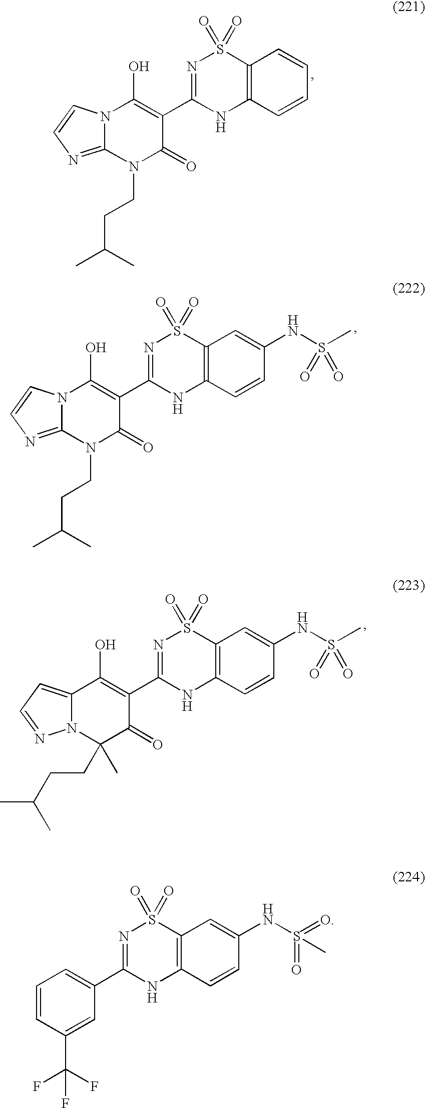

Additional preferred embodiments provide a compound having one of the following formulas:

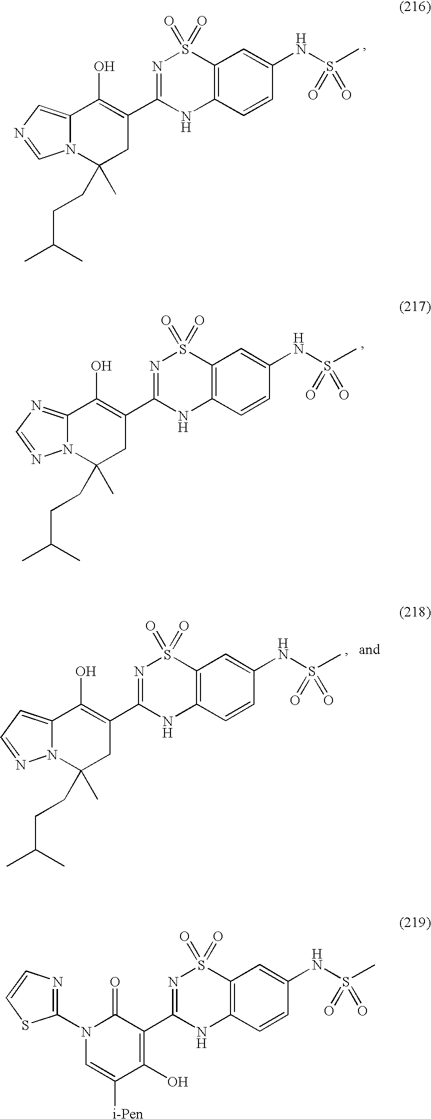

Still further preferred embodiments provide a compound having one of the following formulas:

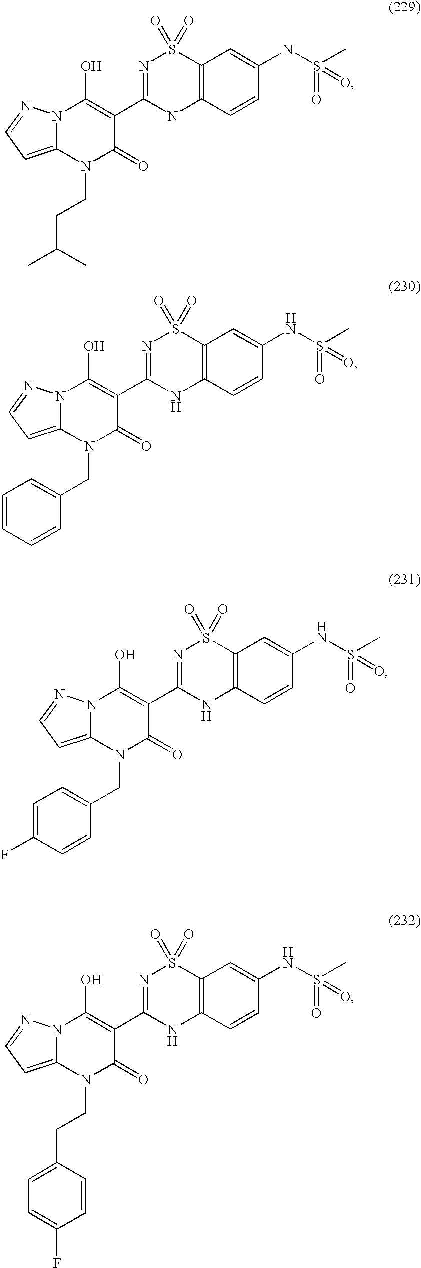

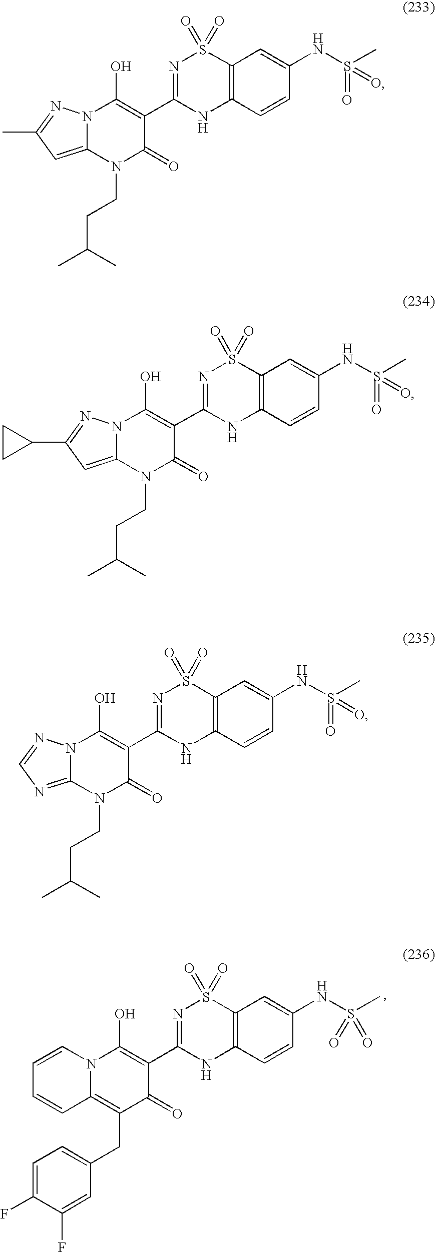

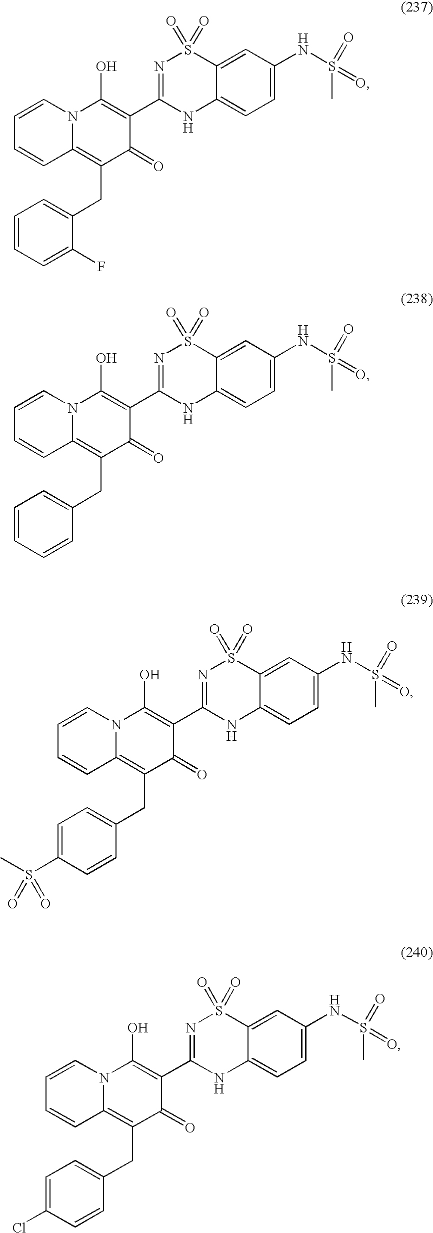

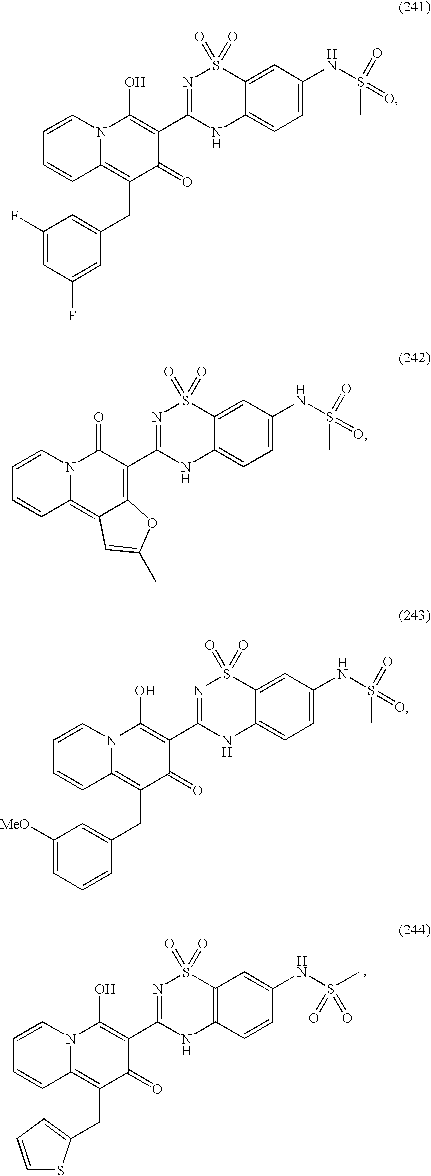

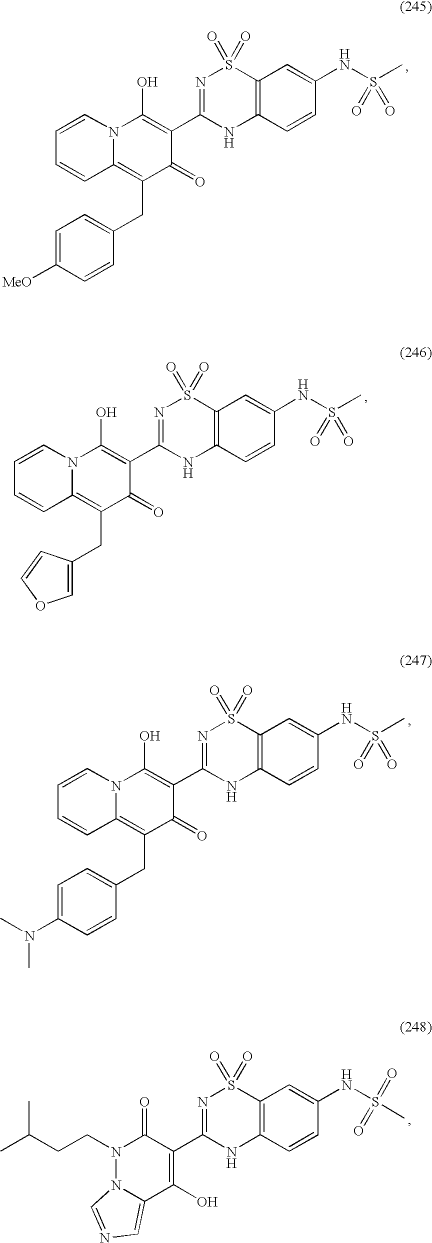

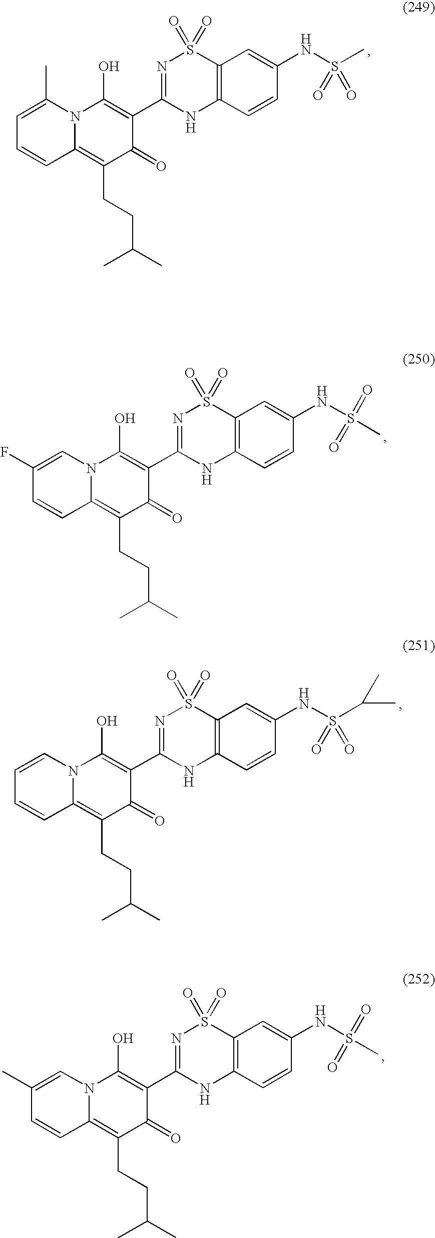

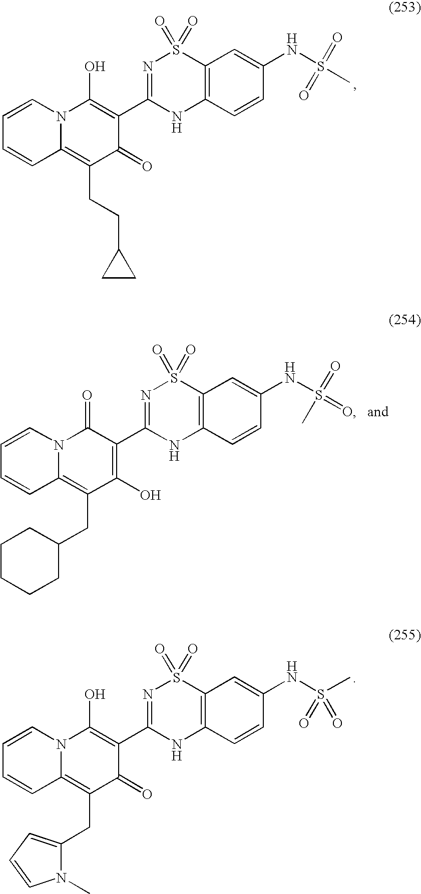

All the embodiments described above intend to include all isomers and tautomers of the represented structural formula.

### Compositions

The present embodiments further provide compositions, including pharmaceutical compositions, comprising compounds of the general Formula I.

A subject pharmaceutical composition comprises a subject compound; and a pharmaceutically acceptable excipient. A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20^{th} edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

The present embodiments provide for a method of inhibiting NS5B polymerase activity comprising contacting a NS5B polymerase with a compound disclosed herein.

The present embodiments provide for a method of treating hepatitis by modulating NS5B polymerase comprising contacting a NS5B polymerase with a compound disclosed herein.

Preferred compounds of Formula I include Compound Numbers 101-105, 201-216, 217-218, 219, 220-247, 248-255.

Preferred embodiments provide a method of treating a hepatitis C virus infection in an individual, the method comprising administering to the individual an effective amount of a composition comprising a preferred compound.

Preferred embodiments provide a method of treating liver fibrosis in an individual, the method comprising administering to the individual an effective amount of a composition comprising a preferred compound.

Preferred embodiments provide a method of increasing liver function in an individual having a hepatitis C virus infection, the method comprising administering to the individual an effective amount of a composition comprising a preferred compound.

In many embodiments, a subject compound inhibits the enzymatic activity of a hepatitis virus C(HCV) NS5B polymerase. Whether a subject compound inhibits HCV NS5B polymerase can be readily determined using any known method. Typical methods involve a determination of whether NS5B polymerase-mediated RNA replication is inhibited in the presence of the agent. In many embodiments, a subject compound inhibits NS5B polymerase activity by at least about 10%, at least about 15%, at least about 20%, at least about 25%, at least about 30%, at least about 40%, at least about 50%, at least about 60%, at least about 70%, at least about 80%, or at least about 90%, or more, compared to the enzymatic activity of NS5B in the absence of the compound.

In many embodiments, a subject compound inhibits enzymatic activity of an HCV NS5B polymerase with an IC₅₀ of less than about 50 μM, e.g., a subject compound inhibits an HCV NS5B polymerase with an IC₅₀ of less than about 40 μM, less than about 25 μM, less than about 10 μM, less than about 1 μM, less than about 100 nM, less than about 80 nM, less than about 60 nM, less than about 50 nM, less than about 25 nM, less than about 10 nM, or less than about 1 nM, or less.

In many embodiments, a subject compound inhibits the enzymatic activity of a hepatitis virus C(HCV) NS5B polymerase. Whether a subject compound inhibits HCV NS5B polymerase can be readily determined using any known method. In many embodiments, a subject compound inhibits NS5B enzymatic activity by at least about 10%, at least about 15%, at least about 20%, at least about 25%, at least about 30%, at least about 40%, at least about 50%, at least about 60%, at least about 70%, at least about 80%, or at least about 90%, or more, compared to the enzymatic activity of NS5B in the absence of the compound.

In many embodiments, a subject compound inhibits HCV viral replication. For example, a subject compound inhibits HCV viral replication by at least about 10%, at least about 15%, at least about 20%, at least about 25%, at least about 30%, at least about 40%, at least about 50%, at least about 60%, at least about 70%, at least about 80%, or at least about 90%, or more, compared to HCV viral replication in the absence of the compound. Whether a subject compound inhibits HCV viral replication can be determined using methods known in the art, including an in vitro viral replication assay.

### Treating a Hepatitis Virus Infection

The methods and compositions described herein are generally useful in treatment of an of HCV infection.

Whether a subject method is effective in treating an HCV infection can be determined by a reduction in viral load, a reduction in time to seroconversion (virus undetectable in patient serum), an increase in the rate of sustained viral response to therapy, a reduction of morbidity or mortality in clinical outcomes, or other indicator of disease response.

In general, an effective amount of a compound of Formula I, and optionally one or more additional antiviral agents, is an amount that is effective to reduce viral load or achieve a sustained viral response to therapy.

Whether a subject method is effective in treating an HCV infection can be determined by measuring viral load, or by measuring a parameter associated with HCV infection, including, but not limited to, liver fibrosis, elevations in serum transaminase levels, and necroinflammatory activity in the liver. Indicators of liver fibrosis are discussed in detail below.

The method involves administering an effective amount of a compound of Formula I, optionally in combination with an effective amount of one or more additional antiviral agents. In some embodiments, an effective amount of a compound of Formula I, and optionally one or more additional antiviral agents, is an amount that is effective to reduce viral titers to undetectable levels, e.g., to about 1000 to about 5000, to about 500 to about 1000, or to about 100 to about 500 genome copies/mL serum. In some embodiments, an effective amount of a compound of Formula I, and optionally one or more additional antiviral agents, is an amount that is effective to reduce viral load to lower than 100 genome copies/mL serum.

In some embodiments, an effective amount of a compound of Formula I, and optionally one or more additional antiviral agents, is an amount that is effective to achieve a 1.5-log, a 2-log, a 2.5-log, a 3-log, a 3.5-log, a 4-log, a 4.5-log, or a 5-log reduction in viral titer in the serum of the individual.

In many embodiments, an effective amount of a compound of Formula I, and optionally one or more additional antiviral agents, is an amount that is effective to achieve a sustained viral response, e.g., non-detectable or substantially non-detectable HCV RNA (e.g., less than about 500, less than about 400, less than about 200, or less than about 100 genome copies per milliliter serum) is found in the patient's serum for a period of at least about one month, at least about two months, at least about three months, at least about four months, at least about five months, or at least about six months following cessation of therapy.

As noted above, whether a subject method is effective in treating an HCV infection can be determined by measuring a parameter associated with HCV infection, such as liver fibrosis. Methods of determining the extent of liver fibrosis are discussed in detail below. In some embodiments, the level of a serum marker of liver fibrosis indicates the degree of liver fibrosis.

As one non-limiting example, levels of serum alanine aminotransferase (ALT) are measured, using standard assays. In general, an ALT level of less than about 45 international units is considered normal. In some embodiments, an effective amount of a compound of Formula I, and optionally one or more additional antiviral agents, is an amount effective to reduce ALT levels to less than about 45 IU/mL serum.

A therapeutically effective amount of a compound of Formula I, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Methods of measuring serum markers include immunological-based methods, e.g., enzyme-linked immunosorbent assays (ELISA), radioimmunoassays, and the like, using antibody specific for a given serum marker.

In many embodiments, an effective amount of a compound of Formula I and an additional antiviral agent is a synergistic amount. As used herein, a “synergistic combination” or a “synergistic amount” of a compound of Formula I and an additional antiviral agent is a combined dosage that is more effective in the therapeutic or prophylactic treatment of an HCV infection than the incremental improvement in treatment outcome that could be predicted or expected from a merely additive combination of (i) the therapeutic or prophylactic benefit of the compound of Formula I when administered at that same dosage as a monotherapy and (ii) the therapeutic or prophylactic benefit of the additional antiviral agent when administered at the same dosage as a monotherapy.

In some embodiments, a selected amount of a compound of Formula I and a selected amount of an additional antiviral agent are effective when used in combination therapy for a disease, but the selected amount of the compound of Formula I and/or the selected amount of the additional antiviral agent is ineffective when used in monotherapy for the disease. Thus, the embodiments encompass (1) regimens in which a selected amount of the additional antiviral agent enhances the therapeutic benefit of a selected amount of the compound of Formula I when used in combination therapy for a disease, where the selected amount of the additional antiviral agent provides no therapeutic benefit when used in monotherapy for the disease (2) regimens in which a selected amount of the compound of Formula I enhances the therapeutic benefit of a selected amount of the additional antiviral agent when used in combination therapy for a disease, where the selected amount of the compound of Formula I provides no therapeutic benefit when used in monotherapy for the disease and (3) regimens in which a selected amount of the compound of Formula I and a selected amount of the additional antiviral agent provide a therapeutic benefit when used in combination therapy for a disease, where each of the selected amounts of the compound of Formula I and the additional antiviral agent, respectively, provides no therapeutic benefit when used in monotherapy for the disease. As used herein, a “synergistically effective amount” of a compound of Formula I and an additional antiviral agent, and its grammatical equivalents, shall be understood to include any regimen encompassed by any of (1)-(3) above.

### Fibrosis

The embodiments provides methods for treating liver fibrosis (including forms of liver fibrosis resulting from, or associated with, HCV infection), generally involving administering a therapeutic amount of a compound of Formula I, and optionally one or more additional antiviral agents. Effective amounts of compounds of Formula I, with and without one or more additional antiviral agents, as well as dosing regimens, are as discussed below.

Whether treatment with a compound of Formula I, and optionally one or more additional antiviral agents, is effective in reducing liver fibrosis is determined by any of a number of well-established techniques for measuring liver fibrosis and liver function. Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt (2000) Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the METAVIR, Knodell, Scheuer, Ludwig, and Ishak scoring systems.

The METAVIR scoring system is based on an analysis of various features of a liver biopsy, including fibrosis (portal fibrosis, centrilobular fibrosis, and cirrhosis); necrosis (piecemeal and lobular necrosis, acidophilic retraction, and ballooning degeneration); inflammation (portal tract inflammation, portal lymphoid aggregates, and distribution of portal inflammation); bile duct changes; and the Knodell index (scores of periportal necrosis, lobular necrosis, portal inflammation, fibrosis, and overall disease activity). The definitions of each stage in the METAVIR system are as follows: score: 0, no fibrosis; score: 1, stellate enlargement of portal tract but without septa formation; score: 2, enlargement of portal tract with rare septa formation; score: 3, numerous septa without cirrhosis; and score: 4, cirrhosis.

Knodell's scoring system, also called the Hepatitis Activity Index, classifies specimens based on scores in four categories of histologic features: I. Periportal and/or bridging necrosis; II. Intralobular degeneration and focal necrosis; III. Portal inflammation; and IV. Fibrosis. In the Knodell staging system, scores are as follows: score: 0, no fibrosis; score: 1, mild fibrosis (fibrous portal expansion); score: 2, moderate fibrosis; score: 3, severe fibrosis (bridging fibrosis); and score: 4, cirrhosis. The higher the score, the more severe the liver tissue damage. Knodell (1981) Hepatol. 1:431.

In the Scheuer scoring system scores are as follows: score: 0, no fibrosis; score: 1, enlarged, fibrotic portal tracts; score: 2, periportal or portal-portal septa, but intact architecture; score: 3, fibrosis with architectural distortion, but no obvious cirrhosis; score: 4, probable or definite cirrhosis. Scheuer (1991) J. Hepatol. 13:372.

The Ishak scoring system is described in Ishak (1995) J. Hepatol. 22:696-699. Stage 0, No fibrosis; Stage 1, Fibrous expansion of some portal areas, with or without short fibrous septa; stage 2, Fibrous expansion of most portal areas, with or without short fibrous septa; stage 3, Fibrous expansion of most portal areas with occasional portal to portal (P-P) bridging; stage 4, Fibrous expansion of portal areas with marked bridging (P-P) as well as portal-central (P-C); stage 5, Marked bridging (P-P and/or P-C) with occasional nodules (incomplete cirrhosis); stage 6, Cirrhosis, probable or definite.

The benefit of anti-fibrotic therapy can also be measured and assessed by using the Child-Pugh scoring system which comprises a multicomponent point system based upon abnormalities in serum bilirubin level, serum albumin level, prothrombin time, the presence and severity of ascites, and the presence and severity of encephalopathy. Based upon the presence and severity of abnormality of these parameters, patients may be placed in one of three categories of increasing severity of clinical disease: A, B, or C.

In some embodiments, a therapeutically effective amount of a compound of Formula I, and optionally one or more additional antiviral agents, is an amount that effects a change of one unit or more in the fibrosis stage based on pre- and post-therapy liver biopsies. In particular embodiments, a therapeutically effective amount of a compound of Formula I, and optionally one or more additional antiviral agents, reduces liver fibrosis by at least one unit in the METAVIR, the Knodell, the Scheuer, the Ludwig, or the Ishak scoring system.

Secondary, or indirect, indices of liver function can also be used to evaluate the efficacy of treatment with a compound of Formula I. Morphometric computerized semi-automated assessment of the quantitative degree of liver fibrosis based upon specific staining of collagen and/or serum markers of liver fibrosis can also be measured as an indication of the efficacy of a subject treatment method. Secondary indices of liver function include, but are not limited to, serum transaminase levels, prothrombin time, bilirubin, platelet count, portal pressure, albumin level, and assessment of the Child-Pugh score.

An effective amount of a compound of Formula I, and optionally one or more additional antiviral agents, is an amount that is effective to increase an index of liver function by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the index of liver function in an untreated individual, or to a placebo-treated individual. Those skilled in the art can readily measure such indices of liver function, using standard assay methods, many of which are commercially available, and are used routinely in clinical settings.

Serum markers of liver fibrosis can also be measured as an indication of the efficacy of a subject treatment method. Serum markers of liver fibrosis include, but are not limited to, hyaluronate, N-terminal procollagen III peptide, 7S domain of type IV collagen, C-terminal procollagen I peptide, and laminin. Additional biochemical markers of liver fibrosis include α-2-macroglobulin, haptoglobin, gamma globulin, apolipoprotein A, and gamma glutamyl transpeptidase.

A therapeutically effective amount of a compound of Formula I, and optionally one or more additional antiviral agents, is an amount that is effective to reduce a serum level of a marker of liver fibrosis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to the level of the marker in an untreated individual, or to a placebo-treated individual. Those skilled in the art can readily measure such serum markers of liver fibrosis, using standard assay methods, many of which are commercially available, and are used routinely in clinical settings. Methods of measuring serum markers include immunological-based methods, e.g., enzyme-linked immunosorbent assays (ELISA), radioimmunoassays, and the like, using antibody specific for a given serum marker.

Quantitative tests of functional liver reserve can also be used to assess the efficacy of treatment with an interferon receptor agonist and pirfenidone (or a pirfenidone analog). These include: indocyanine green clearance (ICG), galactose elimination capacity (GEC), aminopyrine breath test (ABT), antipyrine clearance, monoethylglycine-xylidide (MEG-X) clearance, and caffeine clearance.

As used herein, a “complication associated with cirrhosis of the liver” refers to a disorder that is a sequellae of decompensated liver disease, i.e., or occurs subsequently to and as a result of development of liver fibrosis, and includes, but it not limited to, development of ascites, variceal bleeding, portal hypertension, jaundice, progressive liver insufficiency, encephalopathy, hepatocellular carcinoma, liver failure requiring liver transplantation, and liver-related mortality.

A therapeutically effective amount of a compound of Formula I, and optionally one or more additional antiviral agents, is an amount that is effective in reducing the incidence (e.g., the likelihood that an individual will develop) of a disorder associated with cirrhosis of the liver by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80%, or more, compared to an untreated individual, or to a placebo-treated individual.

Whether treatment with a compound of Formula I, and optionally one or more additional antiviral agents, is effective in reducing the incidence of a disorder associated with cirrhosis of the liver can readily be determined by those skilled in the art.

Reduction in liver fibrosis increases liver function. Thus, the embodiments provide methods for increasing liver function, generally involving administering a therapeutically effective amount of a compound of Formula I, and optionally one or more additional antiviral agents. Liver functions include, but are not limited to, synthesis of proteins such as serum proteins (e.g., albumin, clotting factors, alkaline phosphatase, aminotransferases (e.g., alanine transaminase, aspartate transaminase), 5′-nucleosidase, γ-glutaminyltranspeptidase, etc.), synthesis of bilirubin, synthesis of cholesterol, and synthesis of bile acids; a liver metabolic function, including, but not limited to, carbohydrate metabolism, amino acid and ammonia metabolism, hormone metabolism, and lipid metabolism; detoxification of exogenous drugs; a hemodynamic function, including splanchnic and portal hemodynamics; and the like.

Whether a liver function is increased is readily ascertainable by those skilled in the art, using well-established tests of liver function. Thus, synthesis of markers of liver function such as albumin, alkaline phosphatase, alanine transaminase, aspartate transaminase, bilirubin, and the like, can be assessed by measuring the level of these markers in the serum, using standard immunological and enzymatic assays. Splanchnic circulation and portal hemodynamics can be measured by portal wedge pressure and/or resistance using standard methods. Metabolic functions can be measured by measuring the level of ammonia in the serum.

Whether serum proteins normally secreted by the liver are in the normal range can be determined by measuring the levels of such proteins, using standard immunological and enzymatic assays. Those skilled in the art know the normal ranges for such serum proteins. The following are non-limiting examples. The normal level of alanine transaminase is about 45 IU per milliliter of serum. The normal range of aspartate transaminase is from about 5 to about 40 units per liter of serum. Bilirubin is measured using standard assays. Normal bilirubin levels are usually less than about 1.2 mg/dL. Serum albumin levels are measured using standard assays. Normal levels of serum albumin are in the range of from about 35 to about 55 g/L. Prolongation of prothrombin time is measured using standard assays. Normal prothrombin time is less than about 4 seconds longer than control.

A therapeutically effective amount of a compound of Formula I, and optionally one or more additional antiviral agents, is one that is effective to increase liver function by at least about 10%, at least about 20%, at least about 30%, at least about 40%, at least about 50%, at least about 60%, at least about 70%, at least about 80%, or more. For example, a therapeutically effective amount of a compound of Formula I, and optionally one or more additional antiviral agents, is an amount effective to reduce an elevated level of a serum marker of liver function by at least about 10%, at least about 20%, at least about 30%, at least about 40%, at least about 50%, at least about 60%, at least about 70%, at least about 80%, or more, or to reduce the level of the serum marker of liver function to within a normal range. A therapeutically effective amount of a compound of Formula I, and optionally one or more additional antiviral agents, is also an amount effective to increase a reduced level of a serum marker of liver function by at least about 10%, at least about 20%, at least about 30%, at least about 40%, at least about 50%, at least about 60%, at least about 70%, at least about 80%, or more, or to increase the level of the serum marker of liver function to within a normal range.

### Dosages, Formulations, and Routes of Administration

In the subject methods, the active agent(s) (e.g., compound of Formula I, and optionally one or more additional antiviral agents) may be administered to the host using any convenient means capable of resulting in the desired therapeutic effect. Thus, the agent can be incorporated into a variety of formulations for therapeutic administration. More particularly, the agents of the embodiments can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

### Formulations

The above-discussed active agent(s) can be formulated using well-known reagents and methods. Compositions are provided in formulation with a pharmaceutically acceptable excipient(s). A wide variety of pharmaceutically acceptable excipients is known in the art and need not be discussed in detail herein. Pharmaceutically acceptable excipients have been amply described in a variety of publications, including, for example, A. Gennaro (2000) “Remington: The Science and Practice of Pharmacy,” 20^{th} edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, an agent is formulated in an aqueous buffer. Suitable aqueous buffers include, but are not limited to, acetate, succinate, citrate, and phosphate buffers varying in strengths from about 5 mM to about 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the formulations may further include a preservative. Suitable preservatives include, but are not limited to, a benzyl alcohol, phenol, chlorobutanol, benzalkonium chloride, and the like. In many cases, the formulation is stored at about 4° C. Formulations may also be lyophilized, in which case they generally include cryoprotectants such as sucrose, trehalose, lactose, maltose, mannitol, and the like. Lyophilized formulations can be stored over extended periods of time, even at ambient temperatures.

As such, administration of the agents can be achieved in various ways, including oral, buccal, rectal, parenteral, intraperitoneal, intradermal, subcutaneous, intramuscular, transdermal, intratracheal, etc., administration. In many embodiments, administration is by bolus injection, e.g., subcutaneous bolus injection, intramuscular bolus injection, and the like.

The pharmaceutical compositions of the embodiments can be administered orally, parenterally or via an implanted reservoir. Oral administration or administration by injection is preferred.

Subcutaneous administration of a pharmaceutical composition of the embodiments is accomplished using standard methods and devices, e.g., needle and syringe, a subcutaneous injection port delivery system, and the like. See, e.g., U.S. Pat. Nos. 3,547,119; 4,755,173; 4,531,937; 4,311,137; and 6,017,328. A combination of a subcutaneous injection port and a device for administration of a pharmaceutical composition of the embodiments to a patient through the port is referred to herein as “a subcutaneous injection port delivery system.” In many embodiments, subcutaneous administration is achieved by bolus delivery by needle and syringe.

In pharmaceutical dosage forms, the agents may be administered in the form of their pharmaceutically acceptable salts, or they may also be used alone or in appropriate association, as well as in combination, with other pharmaceutically active compounds. The following methods and excipients are merely exemplary and are in no way limiting.

For oral preparations, the agents can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The agents can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Furthermore, the agents can be made into suppositories by mixing with a variety of bases such as emulsifying bases or water-soluble bases. The compounds of the embodiments can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may comprise the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the embodiments calculated in an amount sufficient to produce the desired effect in association with a pharmaceutically acceptable diluent, carrier or vehicle. The specifications for the novel unit dosage forms of the embodiments depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

### Other Antiviral or Antifibrotic Agents

As discussed above, a subject method will in some embodiments be carried out by administering an NS5B inhibitor that is a compound of Formula I, and optionally one or more additional antiviral agent(s).

In some embodiments, the method further includes administration of one or more interferon receptor agonist(s). Interferon receptor agonists are described herein.

In other embodiments, the method further includes administration of pirfenidone or a pirfenidone analog. Pirfenidone and pirfenidone analogs are described herein.

Additional antiviral agents that are suitable for use in combination therapy include, but are not limited to, nucleotide and nucleoside analogs. Non-limiting examples include azidothymidine (AZT) (zidovudine), and analogs and derivatives thereof; 2′,3′-dideoxyinosine (DDI) (didanosine), and analogs and derivatives thereof; 2′,3′-dideoxycytidine (DDC) (dideoxycytidine), and analogs and derivatives thereof; 2′,3′-didehydro-2′,3′-dideoxythymidine (D4T) (stavudine), and analogs and derivatives thereof; combivir; abacavir; adefovir dipoxil; cidofovir; ribavirin; ribavirin analogs; and the like.

In some embodiments, the method further includes administration of ribavirin. Ribavirin, 1-β-D-ribofuranosyl-1H-1,2,4-triazole-3-carboxamide, available from ICN Pharmaceuticals, Inc., Costa Mesa, Calif., is described in the Merck Index, compound No. 8199, Eleventh Edition. Its manufacture and formulation is described in U.S. Pat. No. 4,211,771. Some embodiments also involve use of derivatives of ribavirin (see, e.g., U.S. Pat. No. 6,277,830). The ribavirin may be administered orally in capsule or tablet form, or in the same or different administration form and in the same or different route as the NS-3 inhibitor compound. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

In some embodiments, the method further includes administration of ritonavir. Ritonavir, 10-hydroxy-2-methyl-5-(1-methylethyl)-1-[2-(1-methylethyl)-4-thiazolyl]-3,6-dioxo-8,11-bis(phenylmethyl)-2,4,7,12-tetraazamidecan-13-oic acid, 5-thiazolylmethyl ester [5S-(5R*,8R*,10R*,11R*)], available from Abbott Laboratories, is an inhibitor of the protease of the human immunodeficiency virus and also of the cytochrome P450 3A and P450 2D6 liver enzymes frequently involved in hepatic metabolism of therapeutic molecules in man. Because of its strong inhibitory effect on cytochrome P450 3A and the inhibitory effect on cytochrome P450 2D6, ritonavir at doses below the normal therapeutic dosage may be combined with polymerase inhibitors to achieve therapeutic levels of the polymerase inhibitor while reducing the number of dosage units required, the dosing frequency, or both.

Ritonavir's structure, synthesis, manufacture and formulation are described in U.S. Pat. No. 5,541,206 U.S. Pat. No. 5,635,523 U.S. Pat. No. 5,648,497 U.S. Pat. No. 5,846,987 and U.S. Pat. No. 6,232,333. The ritonavir may be administered orally in capsule or tablet or oral solution form, or in the same or different administration form and in the same or different route as the NS5B inhibitor compound. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

In some embodiments, an additional antiviral agent is administered during the entire course of NS5B inhibitor compound treatment. In other embodiments, an additional antiviral agent is administered for a period of time that is overlapping with that of the NS5B inhibitor compound treatment, e.g., the additional antiviral agent treatment can begin before the NS5B inhibitor compound treatment begins and end before the NS5B inhibitor compound treatment ends; the additional antiviral agent treatment can begin after the NS5B inhibitor compound treatment begins and end after the NS5B inhibitor compound treatment ends; the additional antiviral agent treatment can begin after the NS5B inhibitor compound treatment begins and end before the NS5B inhibitor compound treatment ends; or the additional antiviral agent treatment can begin before the NS5B inhibitor compound treatment begins and end after the NS5B inhibitor compound treatment ends.

### Methods of Treatment

### Monotherapies

The NS5B inhibitor compounds described herein may be used in acute or chronic therapy for HCV disease. In many embodiments, the NS5B inhibitor compound is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The NS5B inhibitor compound can be administered 5 times per day, 4 times per day, tid, bid, qd, qod, biw, tiw, qw, qow, three times per month, or once monthly. In other embodiments, the NS5B inhibitor compound is administered as a continuous infusion.

In many embodiments, an NS5B inhibitor compound of the embodiments is administered orally.

In connection with the above-described methods for the treatment of HCV disease in a patient, an NS5B inhibitor compound as described herein may be administered to the patient at a dosage from about 0.01 mg to about 100 mg/kg patient bodyweight per day, in 1 to 5 divided doses per day. In some embodiments, the NS5B inhibitor compound is administered at a dosage of about 0.5 mg to about 75 mg/kg patient bodyweight per day, in 1 to 5 divided doses per day.

The amount of active ingredient that may be combined with carrier materials to produce a dosage form can vary depending on the host to be treated and the particular mode of administration. A typical pharmaceutical preparation can contain from about 5% to about 95% active ingredient (w/w). In other embodiments, the pharmaceutical preparation can contain from about 20% to about 80% active ingredient.

Those of skill will readily appreciate that dose levels can vary as a function of the specific NS5B inhibitor compound, the severity of the symptoms and the susceptibility of the subject to side effects. Preferred dosages for a given NS5B inhibitor compound are readily determinable by those of skill in the art by a variety of means. A preferred means is to measure the physiological potency of a given interferon receptor agonist.

In many embodiments, multiple doses of NS5B inhibitor compound are administered. For example, an NS5B inhibitor compound is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Combination Therapies with Ribavirin

In some embodiments, the methods provide for combination therapy comprising administering an NS5B inhibitor compound as described above, and an effective amount of ribavirin. Ribavirin can be administered in dosages of about 400 mg, about 800 mg, about 1000 mg, or about 1200 mg per day.

One embodiment provides any of the above-described methods modified to include co-administering to the patient a therapeutically effective amount of ribavirin for the duration of the desired course of NS5B inhibitor compound treatment.

Another embodiment provides any of the above-described methods modified to include co-administering to the patient about 800 mg to about 1200 mg ribavirin orally per day for the duration of the desired course of NS5B inhibitor compound treatment. In another embodiment, any of the above-described methods may be modified to include co-administering to the patient (a) 1000 mg ribavirin orally per day if the patient has a body weight less than 75 kg or (b) 1200 mg ribavirin orally per day if the patient has a body weight greater than or equal to 75 kg, where the daily dosage of ribavirin is optionally divided into to 2 doses for the duration of the desired course of NS5B inhibitor compound treatment.

Combination Therapies with Levovirin

In some embodiments, the methods provide for combination therapy comprising administering an NS5B inhibitor compound as described above, and an effective amount of levovirin. Levovirin is generally administered in an amount ranging from about 30 mg to about 60 mg, from about 60 mg to about 125 mg, from about 125 mg to about 200 mg, from about 200 mg to about 300 gm, from about 300 mg to about 400 mg, from about 400 mg to about 1200 mg, from about 600 mg to about 1000 mg, or from about 700 to about 900 mg per day, or about 10 mg/kg body weight per day. In some embodiments, levovirin is administered orally in dosages of about 400, about 800, about 1000, or about 1200 mg per day for the desired course of NS5B inhibitor compound treatment.

Combination Therapies with Viramidine

In some embodiments, the methods provide for combination therapy comprising administering an NS5B inhibitor compound as described above, and an effective amount of viramidine. Viramidine is generally administered in an amount ranging from about 30 mg to about 60 mg, from about 60 mg to about 125 mg, from about 125 mg to about 200 mg, from about 200 mg to about 300 gm, from about 300 mg to about 400 mg, from about 400 mg to about 1200 mg, from about 600 mg to about 1000 mg, or from about 700 to about 900 mg per day, or about 10 mg/kg body weight per day. In some embodiments, viramidine is administered orally in dosages of about 800, or about 1600 mg per day for the desired course of NS5B inhibitor compound treatment.

Combination Therapies with Ritonavir

In some embodiments, the methods provide for combination therapy comprising administering an NS5B inhibitor compound as described above, and an effective amount of ritonavir. Ritonavir is generally administered in an amount ranging from about 50 mg to about 100 mg, from about 100 mg to about 200 mg, from about 200 mg to about 300 mg, from about 300 mg to about 400 mg, from about 400 mg to about 500 mg, or from about 500 mg to about 600 mg, twice per day. In some embodiments, ritonavir is administered orally in dosages of about 300 mg, or about 400 mg, or about 600 mg twice per day for the desired course of NS5B inhibitor compound treatment.

Combination Therapies with Alpha-Glucosidase Inhibitors

Suitable α-glucosidase inhibitors include any of the above-described imino-sugars, including long-alkyl chain derivatives of imino sugars as disclosed in U.S. Patent Publication No. 2004/0110795; inhibitors of endoplasmic reticulum-associated α-glucosidases; inhibitors of membrane bound α-glucosidase; miglitol (Glyset®), and active derivatives, and analogs thereof; and acarbose (Precose®), and active derivatives, and analogs thereof.

In many embodiments, the methods provide for combination therapy comprising administering an NS5B inhibitor compound as described above, and an effective amount of an α-glucosidase inhibitor administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time.

An α-glucosidase inhibitor can be administered 5 times per day, 4 times per day, tid (three times daily), bid, qd, qod, biw, tiw, qw, qow, three times per month, or once monthly. In other embodiments, an α-glucosidase inhibitor is administered as a continuous infusion.

In many embodiments, an α-glucosidase inhibitor is administered orally.

In connection with the above-described methods for the treatment of a flavivirus infection, treatment of HCV infection, and treatment of liver fibrosis that occurs as a result of an HCV infection, the methods provide for combination therapy comprising administering an NS5B inhibitor compound as described above, and an effective amount of α-glucosidase inhibitor administered to the patient at a dosage of from about 10 mg per day to about 600 mg per day in divided doses, e.g., from about 10 mg per day to about 30 mg per day, from about 30 mg per day to about 60 mg per day, from about 60 mg per day to about 75 mg per day, from about 75 mg per day to about 90 mg per day, from about 90 mg per day to about 120 mg per day, from about 120 mg per day to about 150 mg per day, from about 150 mg per day to about 180 mg per day, from about 180 mg per day to about 210 mg per day, from about 210 mg per day to about 240 mg per day, from about 240 mg per day to about 270 mg per day, from about 270 mg per day to about 300 mg per day, from about 300 mg per day to about 360 mg per day, from about 360 mg per day to about 420 mg per day, from about 420 mg per day to about 480 mg per day, or from about 480 mg to about 600 mg per day.

In some embodiments, the methods provide for combination therapy comprising administering an NS5B inhibitor compound as described above, and an effective amount of α-glucosidase inhibitor administered in a dosage of about 10 mg three times daily. In some embodiments, an α-glucosidase inhibitor is administered in a dosage of about 15 mg three times daily. In some embodiments, an α-glucosidase inhibitor is administered in a dosage of about 20 mg three times daily. In some embodiments, an α-glucosidase inhibitor is administered in a dosage of about 25 mg three times daily. In some embodiments, an α-glucosidase inhibitor is administered in a dosage of about 30 mg three times daily. In some embodiments, an α-glucosidase inhibitor is administered in a dosage of about 40 mg three times daily. In some embodiments, an α-glucosidase inhibitor is administered in a dosage of about 50 mg three times daily. In some embodiments, an α-glucosidase inhibitor is administered in a dosage of about 100 mg three times daily. In some embodiments, an α-glucosidase inhibitor is administered in a dosage of about 75 mg per day to about 150 mg per day in two or three divided doses, where the individual weighs 60 kg or less. In some embodiments, an α-glucosidase inhibitor is administered in a dosage of about 75 mg per day to about 300 mg per day in two or three divided doses, where the individual weighs 60 kg or more.

The amount of active ingredient (e.g., α-glucosidase inhibitor) that may be combined with carrier materials to produce a dosage form can vary depending on the host to be treated and the particular mode of administration. A typical pharmaceutical preparation can contain from about 5% to about 95% active ingredient (w/w). In other embodiments, the pharmaceutical preparation can contain from about 20% to about 80% active ingredient.

Those of skill will readily appreciate that dose levels can vary as a function of the specific α-glucosidase inhibitor, the severity of the symptoms and the susceptibility of the subject to side effects. Preferred dosages for a given α-glucosidase inhibitor are readily determinable by those of skill in the art by a variety of means. A typical means is to measure the physiological potency of a given active agent.

In many embodiments, multiple doses of an α-glucosidase inhibitor are administered. For example, the methods provide for combination therapy comprising administering an NS5B inhibitor compound as described above, and an effective amount of α-glucosidase inhibitor administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid), over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Combination Therapies with Thymosin-α

In some embodiments, the methods provide for combination therapy comprising administering an NS5B inhibitor compound as described above, and an effective amount of thymosin-α. Thymosin-α (Zadaxin™) is generally administered by subcutaneous injection. Thymosin-α can be administered tid, bid, qd, qod, biw, tiw, qw, qow, three times per month, once monthly, substantially continuously, or continuously for the desired course of NS5B inhibitor compound treatment. In many embodiments, thymosin-α is administered twice per week for the desired course of NS5B inhibitor compound treatment. Effective dosages of thymosin-α range from about 0.5 mg to about 5 mg, e.g., from about 0.5 mg to about 1.0 mg, from about 1.0 mg to about 1.5 mg, from about 1.5 mg to about 2.0 mg, from about 2.0 mg to about 2.5 mg, from about 2.5 mg to about 3.0 mg, from about 3.0 mg to about 3.5 mg, from about 3.5 mg to about 4.0 mg, from about 4.0 mg to about 4.5 mg, or from about 4.5 mg to about 5.0 mg. In particular embodiments, thymosin-α is administered in dosages containing an amount of 1.0 mg or 1.6 mg.

Thymosin-α can be administered over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. In one embodiment, thymosin-α is administered for the desired course of NS5B inhibitor compound treatment.

Combination Therapies with Interferon(s)

In many embodiments, the methods provide for combination therapy comprising administering an NS5B inhibitor compound as described above, and an effective amount of an interferon receptor agonist. In some embodiments, a compound of Formula I and a Type I or III interferon receptor agonist are co-administered in the treatment methods described herein. Type I interferon receptor agonists suitable for use herein include any interferon-α (IFN-α). In certain embodiments, the interferon-α is a PEGylated interferon-α. In certain other embodiments, the interferon-α is a consensus interferon, such as INFERGEN® interferon alfacon-1. In still other embodiments, the interferon-α is a monoPEG (30 kD, linear)-ylated consensus interferon.

Effective dosages of an IFN-α range from about 3 μg to about 27 μg, from about 3 MU to about 10 MU, from about 90 μg to about 180 μg, or from about 18 μg to about 90 μg. Effective dosages of Infergen® consensus IFN-α include about 3 μg, about 6 μg, about 9 μg, about 12 μg, about 15 μg, about 18 μg, about 21 μg, about 24 μg, about 27 μg, or about 30 μg, of drug per dose. Effective dosages of IFN-α2a and IFN-α2b range from 3 million Units (MU) to 10 MU per dose. Effective dosages of PEGASYS®PEGylated IFN-α2a contain an amount of about 90 μg to 270 μg, or about 180 μg, of drug per dose. Effective dosages of PEG-INTRON®PEGylated IFN-α2b contain an amount of about 0.5 μg to 3.0 μg of drug per kg of body weight per dose. Effective dosages of PEGylated consensus interferon (PEG-CIFN) contain an amount of about 18 μg to about 90 μg, or from about 27 μg to about 60 μg, or about 45 μg, of CIFN amino acid weight per dose of PEG-CIFN. Effective dosages of monoPEG (30 kD, linear)-ylated CIFN contain an amount of about 45 μg to about 270 μg, or about 60 μg to about 180 μg, or about 90 μg to about 120 μg, of drug per dose. IFN-α can be administered daily, every other day, once a week, three times a week, every other week, three times per month, once monthly, substantially continuously or continuously.

In many embodiments, the Type I or Type III interferon receptor agonist and/or the Type II interferon receptor agonist is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. Dosage regimens can include tid, bid, qd, qod, biw, tiw, qw, qow, three times per month, or monthly administrations. Some embodiments provide any of the above-described methods in which the desired dosage of IFN-α is administered subcutaneously to the patient by bolus delivery qd, qod, tiw, biw, qw, qow, three times per month, or monthly, or is administered subcutaneously to the patient per day by substantially continuous or continuous delivery, for the desired treatment duration. In other embodiments, any of the above-described methods may be practiced in which the desired dosage of PEGylated IFN-α (PEG-IFN-α) is administered subcutaneously to the patient by bolus delivery qw, qow, three times per month, or monthly for the desired treatment duration.

In other embodiments, an NS5B inhibitor compound and a Type II interferon receptor agonist are co-administered in the treatment methods of the embodiments. Type II interferon receptor agonists suitable for use herein include any interferon-γ (IFN-γ).

Effective dosages of IFN-γ can range from about 0.5 μg/m² to about 500 g/m², usually from about 1.5 μg/m² to 200 μg/m², depending on the size of the patient. This activity is based on 106 international units (U) per 50 μg of protein. IFN-γ can be administered daily, every other day, three times a week, or substantially continuously or continuously.

In specific embodiments of interest, IFN-γ is administered to an individual in a unit dosage form of from about 25 μg to about 500 μg, from about 50 μg to about 400 μg, or from about 100 μg to about 300 μg. In particular embodiments of interest, the dose is about 200 μg IFN-γ. In many embodiments of interest, IFN-γ1b is administered.

Where the dosage is 200 μg IFN-γ per dose, the amount of IFN-γ per body weight (assuming a range of body weights of from about 45 kg to about 135 kg) is in the range of from about 4.4 μg IFN-γ per kg body weight to about 1.48 μg IFN-γ per kg body weight.

The body surface area of subject individuals generally ranges from about 1.33 m² to about 2.50 m². Thus, in many embodiments, an IFN-γ dosage ranges from about 150 μg/m² to about 20 μg/m². For example, an IFN-γ dosage ranges from about 20 μg/m² to about 30 μg/m², from about 30 μg/m² to about 40 μg/m², from about 40 μg/m² to about 50 μg/m², from about 50 μg/m to about 60 μg/m², from about 60 μg/m² to about 70 μg/m², from about 70 μg/m² to about 80 μg/m², from about 80 μg/m² to about 90 μg/m², from about 90 μg/m² to about 100 μg/m², from about 100 μg/m² to about 110 μg/m², from about 110 μg/m² to about 120 μg/m², from about 120 μg/m² to about 130 μg/m², from about 130 μg/m² to about 140 μg/m², or from about 140 μg/m² to about 150 μg/m². In some embodiments, the dosage groups range from about 25 μg/m² to about 100 μg/m². In other embodiments, the dosage groups range from about 25 μg/m² to about 50 μg/m².

In some embodiments, a Type I or a Type III interferon receptor agonist is administered in a first dosing regimen, followed by a second dosing regimen. The first dosing regimen of Type I or a Type III interferon receptor agonist (also referred to as “the induction regimen”) generally involves administration of a higher dosage of the Type I or Type III interferon receptor agonist. For example, in the case of Infergen® consensus IFN-α (CIFN), the first dosing regimen comprises administering CIFN at about 9 μg, about 15 μg, about 18 μg, or about 27 μg. The first dosing regimen can encompass a single dosing event, or at least two or more dosing events. The first dosing regimen of the Type I or Type III interferon receptor agonist can be administered daily, every other day, three times a week, every other week, three times per month, once monthly, substantially continuously or continuously.

The first dosing regimen of the Type I or Type III interferon receptor agonist is administered for a first period of time, which time period can be at least about 4 weeks, at least about 8 weeks, or at least about 12 weeks.

The second dosing regimen of the Type I or Type III interferon receptor agonist (also referred to as “the maintenance dose”) generally involves administration of a lower amount of the Type I or Type III interferon receptor agonist. For example, in the case of CIFN, the second dosing regimen comprises administering CIFN at a dose of at least about 3 μg, at least about 9 μg, at least about 15 μg, or at least about 18 μg. The second dosing regimen can encompass a single dosing event, or at least two or more dosing events.

The second dosing regimen of the Type I or Type III interferon receptor agonist can be administered daily, every other day, three times a week, every other week, three times per month, once monthly, substantially continuously or continuously.

In some embodiments, where an “induction”/“maintenance” dosing regimen of a Type I or a Type III interferon receptor agonist is administered, a “priming” dose of a Type II interferon receptor agonist (e.g., IFN-γ) is included. In these embodiments, IFN-γ is administered for a period of time from about 1 day to about 14 days, from about 2 days to about 10 days, or from about 3 days to about 7 days, before the beginning of treatment with the Type I or Type III interferon receptor agonist. This period of time is referred to as the “priming” phase.

In some of these embodiments, the Type II interferon receptor agonist treatment is continued throughout the entire period of treatment with the Type I or Type III interferon receptor agonist. In other embodiments, the Type II interferon receptor agonist treatment is discontinued before the end of treatment with the Type I or Type III interferon receptor agonist. In these embodiments, the total time of treatment with Type II interferon receptor agonist (including the “priming” phase) is from about 2 days to about 30 days, from about 4 days to about 25 days, from about 8 days to about 20 days, from about 10 days to about 18 days, or from about 12 days to about 16 days. In still other embodiments, the Type II interferon receptor agonist treatment is discontinued once Type I or a Type III interferon receptor agonist treatment begins.

In other embodiments, the Type I or Type III interferon receptor agonist is administered in single dosing regimen. For example, in the case of CIFN, the dose of CIFN is generally in a range of from about 3 μg to about 15 μg, or from about 9 μg to about 15 μg. The dose of Type I or a Type III interferon receptor agonist is generally administered daily, every other day, three times a week, every other week, three times per month, once monthly, or substantially continuously. The dose of the Type I or Type III interferon receptor agonist is administered for a period of time, which period can be, for example, from at least about 24 weeks to at least about 48 weeks, or longer.

In some embodiments, where a single dosing regimen of a Type I or a Type III interferon receptor agonist is administered, a “priming” dose of a Type II interferon receptor agonist (e.g., IFN-γ) is included. In these embodiments, IFN-γ is administered for a period of time from about 1 day to about 14 days, from about 2 days to about 10 days, or from about 3 days to about 7 days, before the beginning of treatment with the Type I or Type III interferon receptor agonist. This period of time is referred to as the “priming” phase. In some of these embodiments, the Type II interferon receptor agonist treatment is continued throughout the entire period of treatment with the Type I or Type III interferon receptor agonist. In other embodiments, the Type II interferon receptor agonist treatment is discontinued before the end of treatment with the Type I or Type III interferon receptor agonist. In these embodiments, the total time of treatment with the Type II interferon receptor agonist (including the “priming” phase) is from about 2 days to about 30 days, from about 4 days to about 25 days, from about 8 days to about 20 days, from about 10 days to about 18 days, or from about 12 days to about 16 days. In still other embodiments, Type II interferon receptor agonist treatment is discontinued once Type I or a Type III interferon receptor agonist treatment begins.

In additional embodiments, an NS5B inhibitor compound, a Type I or III interferon receptor agonist, and a Type II interferon receptor agonist are co-administered for the desired duration of treatment in the methods described herein. In some embodiments, an NS5B inhibitor compound, an interferon-α, and an interferon-γ are co-administered for the desired duration of treatment in the methods described herein.

Some embodiments provide methods using an amount of a Type I or Type III interferon receptor agonist, a Type II interferon receptor agonist, and an NS5B inhibitor compound, effective for the treatment of HCV infection in a patient. Some embodiments provide methods using an effective amount of an IFN-α, IFN-γ, and an NS5B inhibitor compound in the treatment of HCV infection in a patient. One embodiment provides a method using an effective amount of a consensus IFN-α, IFN-γ and an NS5B inhibitor compound in the treatment of HCV infection in a patient.

In general, an effective amount of a consensus interferon (CIFN) and IFN-γ suitable for use in the methods of the embodiments is provided by a dosage ratio of 1 μg CIFN: 10 μg IFN-γ, where both CIFN and IFN-γ are unPEGylated and unglycosylated species.

An embodiment provides any of the above-described methods modified to use an effective amount of INFERGEN®consensus IFN-α and IFN-γ in the treatment of HCV infection in a patient comprising administering to the patient a dosage of INFERGEN® containing an amount of about 1 μg to about 30 μg, of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 300 μg of drug per dose of IFN-γ, subcutaneously qd, qod, tiw, biw, qw, qow, three times per month, once monthly, or per day substantially continuously or continuously, for the desired duration of treatment with an NS5B inhibitor compound.

Another embodiment provides any of the above-described methods modified to use an effective amount of INFERGEN®consensus IFN-α and IFN-γ in the treatment of virus infection in a patient comprising administering to the patient a dosage of INFERGEN® containing an amount of about 1 μg to about 9 μg, of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 100 μg of drug per dose of IFN-γ, subcutaneously qd, qod, tiw, biw, qw, qow, three times per month, once monthly, or per day substantially continuously or continuously, for the desired duration of treatment with an NS5B inhibitor compound.

Another embodiment provides any of the above-described methods modified to use an effective amount of INFERGEN®consensus IFN-α and IFN-γ in the treatment of virus infection in a patient comprising administering to the patient a dosage of INFERGEN® containing an amount of about 1 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 10 μg to about 50 μg of drug per dose of IFN-γ, subcutaneously qd, qod, tiw, biw, qw, qow, three times per month, once monthly, or per day substantially continuously or continuously, for the desired duration of treatment with an NS5B inhibitor compound.

Another embodiment provides any of the above-described methods modified to use an effective amount of INFERGEN®consensus IFN-α and IFN-γ in the treatment of a virus infection in a patient comprising administering to the patient a dosage of INFERGEN® containing an amount of about 9 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 90 μg to about 100 μg of drug per dose of IFN-γ, subcutaneously qd, qod, tiw, biw, qw, qow, three times per month, once monthly, or per day substantially continuously or continuously, for the desired duration of treatment with an NS5B inhibitor compound.

Another embodiment provides any of the above-described methods modified to use an effective amount of INFERGEN®consensus IFN-α and IFN-γ in the treatment of a virus infection in a patient comprising administering to the patient a dosage of INFERGEN® containing an amount of about 30 μg of drug per dose of INFERGEN®, subcutaneously qd, qod, tiw, biw, qw, qow, three times per month, once monthly, or per day substantially continuously or continuously, in combination with a dosage of IFN-γ containing an amount of about 200 μg to about 300 μg of drug per dose of IFN-γ, subcutaneously qd, qod, tiw, biw, qw, qow, three times per month, once monthly, or per day substantially continuously or continuously, for the desired duration of treatment with an NS5B inhibitor compound.

Another embodiment provides any of the above-described methods modified to use an effective amount of PEGylated consensus IFN-α and IFN-γ in the treatment of a virus infection in a patient comprising administering to the patient a dosage of PEGylated consensus IFN-α (PEG-CIFN) containing an amount of about 4 μg to about 60 μg of CIFN amino acid weight per dose of PEG-CIFN, subcutaneously qw, qow, three times per month, or monthly, in combination with a total weekly dosage of IFN-γ containing an amount of about 30 μg to about 1,000 μg of drug per week in divided doses administered subcutaneously qd, qod, tiw, biw, or administered substantially continuously or continuously, for the desired duration of treatment with an NS5B inhibitor compound.

Another embodiment provides any of the above-described methods modified to use an effective amount of PEGylated consensus IFN-α and IFN-γ in the treatment of a virus infection in a patient comprising administering to the patient a dosage of PEGylated consensus IFN-α (PEG-CIFN) containing an amount of about 18 μg to about 24 μg of CIFN amino acid weight per dose of PEG-CIFN, subcutaneously qw, qow, three times per month, or monthly, in combination with a total weekly dosage of IFN-γ containing an amount of about 100 μg to about 300 μg of drug per week in divided doses administered subcutaneously qd, qod, tiw, biw, or substantially continuously or continuously, for the desired duration of treatment with an NS5B inhibitor compound.

In general, an effective amount of IFN-α2a or 2b or 2c and IFN-γ suitable for use in the methods of the embodiments is provided by a dosage ratio of 1 million Units (MU) IFN-α2a or 2b or 2c: 30 μg IFN-γ, where both IFN-α2a or 2b or 2c and IFN-γ are unPEGylated and unglycosylated species.

Another embodiment provides any of the above-described methods modified to use an effective amount of IFN-α2a or 2b or 2c and IFN-γ in the treatment of a virus infection in a patient comprising administering to the patient a dosage of IFN-α2a, 2b or 2c containing an amount of about 1 MU to about 20 MU of drug per dose of IFN-α2a, 2b or 2c subcutaneously qd, qod, tiw, biw, or per day substantially continuously or continuously, in combination with a dosage of IFN-γ containing an amount of about 30 μg to about 600 μg of drug per dose of IFN-γ, subcutaneously qd, qod, tiw, biw, or per day substantially continuously or continuously, for the desired duration of treatment with an NS5B inhibitor compound.

Another embodiment provides any of the above-described methods modified to use an effective amount of IFN-α2a or 2b or 2c and IFN-γ in the treatment of a virus infection in a patient comprising administering to the patient a dosage of IFN-α2a, 2b or 2c containing an amount of about 3 MU of drug per dose of IFN-α2a, 2b or 2c subcutaneously qd, qod, tiw, biw, or per day substantially continuously or continuously, in combination with a dosage of IFN-γ containing an amount of about 100 μg of drug per dose of IFN-γ, subcutaneously qd, qod, tiw, biw, or per day substantially continuously or continuously, for the desired duration of treatment with an NS5B inhibitor compound.

Another embodiment provides any of the above-described methods modified to use an effective amount of IFN-α2a or 2b or 2c and IFN-γ in the treatment of a virus infection in a patient comprising administering to the patient a dosage of IFN-α2a, 2b or 2c containing an amount of about 10 MU of drug per dose of IFN-α2a, 2b or 2c subcutaneously qd, qod, tiw, biw, or per day substantially continuously or continuously, in combination with a dosage of IFN-γ containing an amount of about 300 μg of drug per dose of IFN-γ, subcutaneously qd, qod, tiw, biw, or per day substantially continuously or continuously, for the desired duration of treatment with an NS5B inhibitor compound.

Another embodiment provides any of the above-described methods modified to use an effective amount of PEGASYS®PEGylated IFN-α2a and IFN-γ in the treatment of a virus infection in a patient comprising administering to the patient a dosage of PEGASYS® containing an amount of about 90 μg to about 360 μg, of drug per dose of PEGASYS®, subcutaneously qw, qow, three times per month, or monthly, in combination with a total weekly dosage of IFN-γ containing an amount of about 30 μg to about 1,000 μg, of drug per week administered in divided doses subcutaneously qd, qod, tiw, or biw, or administered substantially continuously or continuously, for the desired duration of treatment with an NS5B inhibitor compound.

Another embodiment provides any of the above-described methods modified to use an effective amount of PEGASYS®PEGylated IFN-α2a and IFN-γ in the treatment of a virus infection in a patient comprising administering to the patient a dosage of PEGASYS® containing an amount of about 180 μg of drug per dose of PEGASYS®, subcutaneously qw, qow, three times per month, or monthly, in combination with a total weekly dosage of IFN-γ containing an amount of about 100 μg to about 300 μg, of drug per week administered in divided doses subcutaneously qd, qod, tiw, or biw, or administered substantially continuously or continuously, for the desired duration of treatment with an NS5B inhibitor compound.

Another embodiment provides any of the above-described methods modified to use an effective amount of PEG-INTRON®PEGylated IFN-α2b and IFN-γ in the treatment of a virus infection in a patient comprising administering to the patient a dosage of PEG-INTRON® containing an amount of about 0.75 μg to about 3.0 μg of drug per kilogram of body weight per dose of PEG-INTRON®, subcutaneously qw, qow, three times per month, or monthly, in combination with a total weekly dosage of IFN-γ containing an amount of about 30 μg to about 1,000 μg of drug per week administered in divided doses subcutaneously qd, qod, tiw, or biw, or administered substantially continuously or continuously, for the desired duration of treatment with an NS5B inhibitor compound.

Another embodiment provides any of the above-described methods modified to use an effective amount of PEG-INTRON®PEGylated IFN-α2b and IFN-γ in the treatment of a virus infection in a patient comprising administering to the patient a dosage of PEG-INTRON® containing an amount of about 1.5 μg of drug per kilogram of body weight per dose of PEG-INTRON®, subcutaneously qw, qow, three times per month, or monthly, in combination with a total weekly dosage of IFN-γ containing an amount of about 100 μg to about 300 μg of drug per week administered in divided doses subcutaneously qd, qod, tiw, or biw, or administered substantially continuously or continuously, for the desired duration of treatment with an NS5B inhibitor compound.

One embodiment provides any of the above-described methods modified to comprise administering to an individual having an HCV infection an effective amount of an NS5B inhibitor; and a regimen of 9 μg INFERGEN® consensus IFN-α administered subcutaneously qd or tiw, and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS5B inhibitor; and a regimen of 9 μg INFERGEN® consensus IFN-α administered subcutaneously qd or tiw; 50 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS5B inhibitor; and a regimen of 9 μg INFERGEN® consensus IFN-α administered subcutaneously qd or tiw; 100 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS5B inhibitor; and a regimen of 9 μg INFERGEN® consensus IFN-α administered subcutaneously qd or tiw; and 50 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS5B inhibitor; and a regimen of 9 μg INFERGEN® consensus IFN-α administered subcutaneously qd or tiw; and 100 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS5B inhibitor; and a regimen of 9 μg INFERGEN® consensus IFN-α administered subcutaneously qd or tiw; 25 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS5B inhibitor; and a regimen of 9 μg INFERGEN® consensus IFN-α administered subcutaneously qd or tiw; 200 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS5B inhibitor; and a regimen of 9 μg INFERGEN® consensus IFN-α administered subcutaneously qd or tiw; and 25 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS5B inhibitor; and a regimen of 9 μg INFERGEN® consensus IFN-α administered subcutaneously qd or tiw; and 200 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS5B inhibitor; and a regimen of 100 μg monoPEG (30 kD, linear)-ylated consensus IFN-α administered subcutaneously every 10 days or qw, and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS5B inhibitor; and a regimen of 100 μg monoPEG (30 kD, linear)-ylated consensus IFN-α administered subcutaneously every 10 days or qw; 50 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS5B inhibitor; and a regimen of 100 μg monoPEG (30 kD, linear)-ylated consensus IFN-α administered subcutaneously every 10 days or qw; 100 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS5B inhibitor; and a regimen of 100 μg monoPEG (30 kD, linear)-ylated consensus IFN-α administered subcutaneously every 10 days or qw; and 50 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS5B inhibitor; and a regimen of 100 μg monoPEG (30 kD, linear)-ylated consensus IFN-α administered subcutaneously every 10 days or qw; and 100 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS5B inhibitor; and a regimen of 150 μg monoPEG (30 kD, linear)-ylated consensus IFN-α administered subcutaneously every 10 days or qw, and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS5B inhibitor; and a regimen of 150 μg monoPEG (30 kD, linear)-ylated consensus IFN-α administered subcutaneously every 10 days or qw; 50 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS5B inhibitor; and a regimen of 150 μg monoPEG (30 kD, linear)-ylated consensus IFN-α administered subcutaneously every 10 days or qw; 100 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS5B inhibitor; and a regimen of 150 μg monoPEG (30 kD, linear)-ylated consensus IFN-α administered subcutaneously every 10 days or qw; and 50 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS5B inhibitor; and a regimen of 150 μg monoPEG (30 kD, linear)-ylated consensus IFN-α administered subcutaneously every 10 days or qw; and 100 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS5B inhibitor; and a regimen of 200 μg monoPEG (30 kD, linear)-ylated consensus IFN-α administered subcutaneously every 10 days or qw, and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS5B inhibitor; and a regimen of 200 μg monoPEG (30 kD, linear)-ylated consensus IFN-α administered subcutaneously every 10 days or qw; 50 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS5B inhibitor; and a regimen of 200 μg monoPEG (30 kD, linear)-ylated consensus IFN-α administered subcutaneously every 10 days or qw; 100 μg Actimmune® human IFN-γ1b administered subcutaneously tiw; and ribavirin administered orally qd, where the duration of therapy is 48 weeks. In this embodiment, ribavirin is administered in an amount of 1000 mg for individuals weighing less than 75 kg, and 1200 mg for individuals weighing 75 kg or more.

One embodiment provides any of the above-described methods modified to comprise administering to an individual having an HCV infection an effective amount of an NS5B inhibitor; and a regimen of 200 μg monoPEG (30 kD, linear)-ylated consensus IFN-α administered subcutaneously every 10 days or qw; and 50 μg Actimmune® human IFN-γ1b administered subcutaneously tiw, where the duration of therapy is 48 weeks.

One embodiment provides any of the above-described methods modified to comprise administering to an individual having an HCV infection an effective amount of an NS5B inhibitor; and a regimen of 200 μg monoPEG (30 kD, linear)-ylated consensus IFN-α administered subcutaneously every 10 days or qw; and 100 μg Actimmune® human IFN-γ1b administered subcutaneously tiw, where the duration of therapy is 48 weeks.

Any of the above-described methods involving administering an NS5B inhibitor, a Type I interferon receptor agonist (e.g., an IFN-α), and a Type II interferon receptor agonist (e.g., an IFN-γ), can be augmented by administration of an effective amount of a TNF-α antagonist (e.g., a TNF-α antagonist other than pirfenidone or a pirfenidone analog). Exemplary, non-limiting TNF-α antagonists that are suitable for use in such combination therapies include ENBREL®, REMICADE®, and HUMIRA™.

One embodiment provides a method using an effective amount of ENBREL®; an effective amount of IFN-α; an effective amount of IFN-γ; and an effective amount of an NS5B inhibitor in the treatment of an HCV infection in a patient, comprising administering to the patient a dosage ENBREL® containing an amount of from about 0.1 μg to about 23 mg per dose, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, or from about 20 mg to about 23 mg of ENBREL®, subcutaneously qd, qod, tiw, biw, qw, qow, three times per month, once monthly, or once every other month, or per day substantially continuously or continuously, for the desired duration of treatment.

One embodiment provides a method using an effective amount of REMICADE®, an effective amount of IFN-α; an effective amount of IFN-γ; and an effective amount of an NS5B inhibitor in the treatment of an HCV infection in a patient, comprising administering to the patient a dosage of REMICADE® containing an amount of from about 0.1 mg/kg to about 4.5 mg/kg, from about 0.1 mg/kg to about 0.5 mg/kg, from about 0.5 mg/kg to about 1.0 mg/kg, from about 1.0 mg/kg to about 1.5 mg/kg, from about 1.5 mg/kg to about 2.0 mg/kg, from about 2.0 mg/kg to about 2.5 mg/kg, from about 2.5 mg/kg to about 3.0 mg/kg, from about 3.0 mg/kg to about 3.5 mg/kg, from about 3.5 mg/kg to about 4.0 mg/kg, or from about 4.0 mg/kg to about 4.5 mg/kg per dose of REMICADE®, intravenously qd, qod, tiw, biw, qw, qow, three times per month, once monthly, or once every other month, or per day substantially continuously or continuously, for the desired duration of treatment.

One embodiment provides a method using an effective amount of HUMIRA™, an effective amount of IFN-α; an effective amount of IFN-γ; and an effective amount of an NS5B inhibitor in the treatment of an HCV infection in a patient, comprising administering to the patient a dosage of HUMIRA™ containing an amount of from about 0.1 μg to about 35 mg, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, or from about 30 mg to about 35 mg per dose of a HUMIRA™, subcutaneously qd, qod, tiw, biw, qw, qow, three times per month, once monthly, or once every other month, or per day substantially continuously or continuously, for the desired duration of treatment.

Combination Therapies with Pirfenidone

In many embodiments, the methods provide for combination therapy comprising administering an NS5B inhibitor compound as described above, and an effective amount of pirfenidone or a pirfenidone analog. In some embodiments, an NS5B inhibitor compound, one or more interferon receptor agonist(s), and pirfenidone or pirfenidone analog are co-administered in the treatment methods of the embodiments. In certain embodiments, an NS5B inhibitor compound, a Type I interferon receptor agonist, and pirfenidone (or a pirfenidone analog) are co-administered. In other embodiments, an NS5B inhibitor compound, a Type I interferon receptor agonist, a Type II interferon receptor agonist, and pirfenidone (or a pirfenidone analog) are co-administered. Type I interferon receptor agonists suitable for use herein include any IFN-α, such as interferon alfa-2a, interferon alfa-2b, interferon alfacon-1, and PEGylated IFN-α's, such as peginterferon alfa-2a, peginterferon alfa-2b, and PEGylated consensus interferons, such as monoPEG (30 kD, linear)-ylated consensus interferon. Type II interferon receptor agonists suitable for use herein include any interferon-γ.

Pirfenidone or a pirfenidone analog can be administered once per month, twice per month, three times per month, once per week, twice per week, three times per week, four times per week, five times per week, six times per week, daily, or in divided daily doses ranging from once daily to 5 times daily over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

Effective dosages of pirfenidone or a specific pirfenidone analog include a weight-based dosage in the range from about 5 mg/kg/day to about 125 mg/kg/day, or a fixed dosage of about 400 mg to about 3600 mg per day, or about 800 mg to about 2400 mg per day, or about 1000 mg to about 1800 mg per day, or about 1200 mg to about 1600 mg per day, administered orally in one to five divided doses per day. Other doses and formulations of pirfenidone and specific pirfenidone analogs suitable for use in the treatment of fibrotic diseases are described in U.S. Pat. Nos., 5,310,562; 5,518,729; 5,716,632; and 6,090,822.

One embodiment provides any of the above-described methods modified to include co-administering to the patient a therapeutically effective amount of pirfenidone or a pirfenidone analog for the duration of the desired course of NS5B inhibitor compound treatment.

Combination Therapies with TNF-α Antagonists

In many embodiments, the methods provide for combination therapy comprising administering an effective amount of an NS5B inhibitor compound as described above, and an effective amount of TNF-α antagonist, in combination therapy for treatment of an HCV infection.

Effective dosages of a TNF-α antagonist range from 0.1 μg to 40 mg per dose, e.g., from about 0.1 μg to about 0.5 μg per dose, from about 0.5 μg to about 1.0 μg per dose, from about 1.0 μg per dose to about 5.0 μg per dose, from about 5.0 μg to about 10 μg per dose, from about 10 μg to about 20 μg per dose, from about 20 μg per dose to about 30 μg per dose, from about 30 μg per dose to about 40 μg per dose, from about 40 μg per dose to about 50 μg per dose, from about 50 μg per dose to about 60 μg per dose, from about 60 μg per dose to about 70 μg per dose, from about 70 μg to about 80 μg per dose, from about 80 μg per dose to about 100 μg per dose, from about 100 μg to about 150 μg per dose, from about 150 μg to about 200 μg per dose, from about 200 μg per dose to about 250 μg per dose, from about 250 μg to about 300 μg per dose, from about 300 μg to about 400 μg per dose, from about 400 μg to about 500 μg per dose, from about 500 μg to about 600 μg per dose, from about 600 μg to about 700 μg per dose, from about 700 μg to about 800 μg per dose, from about 800 μg to about 900 μg per dose, from about 900 μg to about 1000 μg per dose, from about 1 mg to about 10 mg per dose, from about 10 mg to about 15 mg per dose, from about 15 mg to about 20 mg per dose, from about 20 mg to about 25 mg per dose, from about 25 mg to about 30 mg per dose, from about 30 mg to about 35 mg per dose, or from about 35 mg to about 40 mg per dose.

In some embodiments, effective dosages of a TNF-α antagonist are expressed as mg/kg body weight. In these embodiments, effective dosages of a TNF-α antagonist are from about 0.1 mg/kg body weight to about 10 mg/kg body weight, e.g., from about 0.1 mg/kg body weight to about 0.5 mg/kg body weight, from about 0.5 mg/kg body weight to about 1.0 mg/kg body weight, from about 1.0 mg/kg body weight to about 2.5 mg/kg body weight, from about 2.5 mg/kg body weight to about 5.0 mg/kg body weight, from about 5.0 mg/kg body weight to about 7.5 mg/kg body weight, or from about 7.5 mg/kg body weight to about 10 mg/kg body weight.

In many embodiments, a TNF-α antagonist is administered for a period of about 1 day to about 7 days, or about 1 week to about 2 weeks, or about 2 weeks to about 3 weeks, or about 3 weeks to about 4 weeks, or about 1 month to about 2 months, or about 3 months to about 4 months, or about 4 months to about 6 months, or about 6 months to about 8 months, or about 8 months to about 12 months, or at least one year, and may be administered over longer periods of time. The TNF-α antagonist can be administered tid, bid, qd, qod, biw, tiw, qw, qow, three times per month, once monthly, substantially continuously, or continuously.

In many embodiments, multiple doses of a TNF-α antagonist are administered. For example, a TNF-α antagonist is administered once per month, twice per month, three times per month, every other week (qow), once per week (qw), twice per week (biw), three times per week (tiw), four times per week, five times per week, six times per week, every other day (qod), daily (qd), twice a day (bid), or three times a day (tid), substantially continuously, or continuously, over a period of time ranging from about one day to about one week, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more.

A TNF-α antagonist and an NS5B inhibitor are generally administered in separate formulations. A TNF-α antagonist and an NS5B inhibitor may be administered substantially simultaneously, or within about 30 minutes, about 1 hour, about 2 hours, about 4 hours, about 8 hours, about 16 hours, about 24 hours, about 36 hours, about 72 hours, about 4 days, about 7 days, or about 2 weeks of one another.

One embodiment provides a method using an effective amount of a TNF-α antagonist and an effective amount of an NS5B inhibitor in the treatment of an HCV infection in a patient, comprising administering to the patient a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS5B inhibitor compound.

One embodiment provides a method using an effective amount of ENBREL® and an effective amount of an NS5B inhibitor in the treatment of an HCV infection in a patient, comprising administering to the patient a dosage ENBREL® containing an amount of from about 0.1 μg to about 23 mg per dose, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, or from about 20 mg to about 23 mg of ENBREL®, subcutaneously qd, qod, tiw, biw, qw, qow, three times per month, once monthly, or once every other month, or per day substantially continuously or continuously, for the desired duration of treatment with an NS5B inhibitor compound.

One embodiment provides a method using an effective amount of REMICADE® and an effective amount of an NS5B inhibitor in the treatment of an HCV infection in a patient, comprising administering to the patient a dosage of REMICADE® containing an amount of from about 0.1 mg/kg to about 4.5 mg/kg, from about 0.1 mg/kg to about 0.5 mg/kg, from about 0.5 mg/kg to about 1.0 mg/kg, from about 1.0 mg/kg to about 1.5 mg/kg, from about 1.5 mg/kg to about 2.0 mg/kg, from about 2.0 mg/kg to about 2.5 mg/kg, from about 2.5 mg/kg to about 3.0 mg/kg, from about 3.0 mg/kg to about 3.5 mg/kg, from about 3.5 mg/kg to about 4.0 mg/kg, or from about 4.0 mg/kg to about 4.5 mg/kg per dose of REMICADE®, intravenously qd, qod, tiw, biw, qw, qow, three times per month, once monthly, or once every other month, or per day substantially continuously or continuously, for the desired duration of treatment with an NS5B inhibitor compound.

One embodiment provides a method using an effective amount of HUMIRA™ and an effective amount of an NS5B inhibitor in the treatment of an HCV infection in a patient, comprising administering to the patient a dosage of HUMIRA™ containing an amount of from about 0.1 μg to about 35 mg, from about 0.1 μg to about 1 μg, from about 1 μg to about 10 μg, from about 10 μg to about 100 μg, from about 100 μg to about 1 mg, from about 1 mg to about 5 mg, from about 5 mg to about 10 mg, from about 10 mg to about 15 mg, from about 15 mg to about 20 mg, from about 20 mg to about 25 mg, from about 25 mg to about 30 mg, or from about 30 mg to about 35 mg per dose of a HUMIRA™, subcutaneously qd, qod, tiw, biw, qw, qow, three times per month, once monthly, or once every other month, or per day substantially continuously or continuously, for the desired duration of treatment with an NS5B inhibitor compound.

Combination Therapies with Thymosin-α

In many embodiments, the methods provide for combination therapy comprising administering an effective amount of an NS5B inhibitor compound as described above, and an effective amount of thymosin-α, in combination therapy for treatment of an HCV infection.

Effective dosages of thymosin-α range from about 0.5 mg to about 5 mg, e.g., from about 0.5 mg to about 1.0 mg, from about 1.0 mg to about 1.5 mg, from about 1.5 mg to about 2.0 mg, from about 2.0 mg to about 2.5 mg, from about 2.5 mg to about 3.0 mg, from about 3.0 mg to about 3.5 mg, from about 3.5 mg to about 4.0 mg, from about 4.0 mg to about 4.5 mg, or from about 4.5 mg to about 5.0 mg. In particular embodiments, thymosin-α is administered in dosages containing an amount of 1.0 mg or 1.6 mg.

One embodiment provides a method using an effective amount of ZADAXIN™ thymosin-α and an effective amount of an NS5B inhibitor in the treatment of an HCV infection in a patient, comprising administering to the patient a dosage of ZADAXIN™ containing an amount of from about 1.0 mg to about 1.6 mg per dose, subcutaneously twice per week for the desired duration of treatment with the NS5B inhibitor compound.

Combination Therapies with a TNF-α Antagonist and an Interferon

Some embodiments provide a method of treating an HCV infection in an individual having an HCV infection, the method comprising administering an effective amount of an NS5B inhibitor, and effective amount of a TNF-α antagonist, and an effective amount of one or more interferons.

One embodiment provides any of the above-described methods modified to use an effective amount of IFN-γ and an effective amount of a TNF-α antagonist in the treatment of HCV infection in a patient comprising administering to the patient a dosage of IFN-γ containing an amount of about 10 μg to about 300 μg of drug per dose of IFN-γ, subcutaneously qd, qod, tiw, biw, qw, qow, three times per month, once monthly, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS5B inhibitor compound.

One embodiment provides any of the above-described methods modified to use an effective amount of IFN-γ and an effective amount of a TNF-α antagonist in the treatment of HCV infection in a patient comprising administering to the patient a dosage of IFN-γ containing an amount of about 10 μg to about 100 μg of drug per dose of IFN-γ, subcutaneously qd, qod, tiw, biw, qw, qow, three times per month, once monthly, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS5B inhibitor compound.

Another embodiment provides any of the above-described methods modified to use an effective amount of IFN-γ and an effective amount of a TNF-α antagonist in the treatment of a virus infection in a patient comprising administering to the patient a total weekly dosage of IFN-γ containing an amount of about 30 μg to about 1,000 μg of drug per week in divided doses administered subcutaneously qd, qod, tiw, biw, or administered substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS5B inhibitor compound.

Another embodiment provides any of the above-described methods modified to use an effective amount of IFN-γ and an effective amount of a TNF-α antagonist in the treatment of a virus infection in a patient comprising administering to the patient a total weekly dosage of IFN-γ containing an amount of about 100 μg to about 300 μg of drug per week in divided doses administered subcutaneously qd, qod, tiw, biw, or administered substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS5B inhibitor compound.

One embodiment provides any of the above-described methods modified to use an effective amount of INFERGEN® consensus IFN-α and a TNF-α antagonist in the treatment of HCV infection in a patient comprising administering to the patient a dosage of INFERGEN® containing an amount of about 1 μg to about 30 μg, of drug per dose of INFERGEN®, subcutaneously qd, qod, tiw, biw, qw, qow, three times per month, once monthly, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS5B inhibitor compound.

One embodiment provides any of the above-described methods modified to use an effective amount of INFERGEN® consensus IFN-α and a TNF-α antagonist in the treatment of HCV infection in a patient comprising administering to the patient a dosage of INFERGEN® containing an amount of about 1 μg to about 9 μg, of drug per dose of INFERGEN®, subcutaneously qd, qod, tiw, biw, qw, qow, three times per month, once monthly, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS5B inhibitor compound.

Another embodiment provides any of the above-described methods modified to use an effective amount of PEGylated consensus IFN-α and an effective amount of a TNF-α antagonist in the treatment of a virus infection in a patient comprising administering to the patient a dosage of PEGylated consensus IFN-α (PEG-CIFN) containing an amount of about 4 μg to about 60 μg of CIFN amino acid weight per dose of PEG-CIFN, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS5B inhibitor compound.

Another embodiment provides any of the above-described methods modified to use an effective amount of PEGylated consensus IFN-α and an effective amount of a TNF-α antagonist in the treatment of a virus infection in a patient comprising administering to the patient a dosage of PEGylated consensus IFN-α (PEG-CIFN) containing an amount of about 18 μg to about 24 μg of CIFN amino acid weight per dose of PEG-CIFN, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS5B inhibitor compound.

Another embodiment provides any of the above-described methods modified to use an effective amount of IFN-α2a or 2b or 2c and an effective amount of a TNF-α antagonist in the treatment of a virus infection in a patient comprising administering to the patient a dosage of IFN-α2a, 2b or 2c containing an amount of about 1 MU to about 20 MU of drug per dose of IFN-α2a, 2b or 2c subcutaneously qd, qod, tiw, biw, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS5B inhibitor compound.

Another embodiment provides any of the above-described methods modified to use an effective amount of IFN-α 2a or 2b or 2c and an effective amount of a TNF-α antagonist in the treatment of a virus infection in a patient comprising administering to the patient a dosage of IFN-α 2a, 2b or 2c containing an amount of about 3 MU of drug per dose of IFN-α 2a, 2b or 2c subcutaneously qd, qod, tiw, biw, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS5B inhibitor compound.

Another embodiment provides any of the above-described methods modified to use an effective amount of IFN-α2a or 2b or 2c and an effective amount of a TNF-α antagonist in the treatment of a virus infection in a patient comprising administering to the patient a dosage of IFN-α2a, 2b or 2c containing an amount of about 10 MU of drug per dose of IFN-α2a, 2b or 2c subcutaneously qd, qod, tiw, biw, or per day substantially continuously or continuously, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS5B inhibitor compound.

Another embodiment provides any of the above-described methods modified to use an effective amount of PEGASYS®PEGylated IFN-α2a and an effective amount of a TNF-α antagonist in the treatment of a virus infection in a patient comprising administering to the patient a dosage of PEGASYS® containing an amount of about 90 μg to about 360 μg, of drug per dose of PEGASYS®, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS5B inhibitor compound.

Another embodiment provides any of the above-described methods modified to use an effective amount of PEGASYS®PEGylated IFN-α2a and an effective amount of a TNF-α antagonist in the treatment of a virus infection in a patient comprising administering to the patient a dosage of PEGASYS® containing an amount of about 180 μg, of drug per dose of PEGASYS®, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS5B inhibitor compound.

Another embodiment provides any of the above-described methods modified to use an effective amount of PEG-INTRON®PEGylated IFN-α2b and an effective amount of a TNF-α antagonist in the treatment of a virus infection in a patient comprising administering to the patient a dosage of PEG-INTRON® containing an amount of about 0.75 μg to about 3.0 μg of drug per kilogram of body weight per dose of PEG-INTRON®, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS5B inhibitor compound.

Another embodiment provides any of the above-described methods modified to use an effective amount of PEG-INTRON®PEGylated IFN-α2b and an effective amount of a TNF-α antagonist in the treatment of a virus infection in a patient comprising administering to the patient a dosage of PEG-INTRON® containing an amount of about 1.5 μg of drug per kilogram of body weight per dose of PEG-INTRON®, subcutaneously qw, qow, three times per month, or monthly, in combination with a dosage of a TNF-α antagonist containing an amount of from about 0.1 μg to about 40 mg per dose of a TNF-α antagonist, subcutaneously qd, qod, tiw, or biw, or per day substantially continuously or continuously, for the desired duration of treatment with an NS5B inhibitor compound.

Combination Therapies with Other Antiviral Agents

Other agents such as inhibitors of HCV NS3 helicase are also attractive drugs for combinational therapy, and are contemplated for use in combination therapies described herein. Ribozymes such as Heptazyme and phosphorothioate oligonucleotides which are complementary to HCV protein sequences and which inhibit the expression of viral core proteins are also suitable for use in combination therapies described herein. Additional agents such as inhibitors of the NS3 protease are attractive drugs for combinational therapy, and are contemplated for use in combination therapies described herein.

In some embodiments, the additional antiviral agent(s) is administered during the entire course of treatment with the NS5B inhibitor compound described herein, and the beginning and end of the treatment periods coincide. In other embodiments, the additional antiviral agent(s) is administered for a period of time that is overlapping with that of the NS5B inhibitor compound treatment, e.g., treatment with the additional antiviral agent(s) begins before the NS5B inhibitor compound treatment begins and ends before the NS5B inhibitor compound treatment ends; treatment with the additional antiviral agent(s) begins after the NS5B inhibitor compound treatment begins and ends after the NS5B inhibitor compound treatment ends; treatment with the additional antiviral agent(s) begins after the NS5B inhibitor compound treatment begins and ends before the NS5B inhibitor compound treatment ends; or treatment with the additional antiviral agent(s) begins before the NS5B inhibitor compound treatment begins and ends after the NS5B inhibitor compound treatment ends.

The NS5B inhibitor compound can be administered together with (i.e., simultaneously in separate formulations; simultaneously in the same formulation; administered in separate formulations and within about 48 hours, within about 36 hours, within about 24 hours, within about 16 hours, within about 12 hours, within about 8 hours, within about 4 hours, within about 2 hours, within about 1 hour, within about 30 minutes, or within about 15 minutes or less) one or more additional antiviral agents.

As non-limiting examples, any of the above-described methods featuring an IFN-α regimen can be modified to replace the subject IFN-α regimen with a regimen of monoPEG (30 kD, linear)-ylated consensus IFN-α comprising administering a dosage of monoPEG (30 kD, linear)-ylated consensus IFN-α containing an amount of 100 μg of drug per dose, subcutaneously once weekly, once every 8 days, or once every 10 days for the desired treatment duration with an NS5B inhibitor compound.

As non-limiting examples, any of the above-described methods featuring an IFN-α regimen can be modified to replace the subject IFN-α regimen with a regimen of monoPEG (30 kD, linear)-ylated consensus IFN-α comprising administering a dosage of monoPEG (30 kD, linear)-ylated consensus IFN-α containing an amount of 150 μg of drug per dose, subcutaneously once weekly, once every 8 days, or once every 10 days for the desired treatment duration with an NS5B inhibitor compound.

As non-limiting examples, any of the above-described methods featuring an IFN-α regimen can be modified to replace the subject IFN-α regimen with a regimen of monoPEG (30 kD, linear)-ylated consensus IFN-α comprising administering a dosage of monoPEG (30 kD, linear)-ylated consensus IFN-α containing an amount of 200 μg of drug per dose, subcutaneously once weekly, once every 8 days, or once every 10 days for the desired treatment duration with an NS5B inhibitor compound.

As non-limiting examples, any of the above-described methods featuring an IFN-α regimen can be modified to replace the subject IFN-α regimen with a regimen of INFERGEN® interferon alfacon-1 comprising administering a dosage of INFERGEN® interferon alfacon-1 containing an amount of 9 μg of drug per dose, subcutaneously once daily or three times per week for the desired treatment duration with an NS5B inhibitor compound.

As non-limiting examples, any of the above-described methods featuring an IFN-α regimen can be modified to replace the subject IFN-α regimen with a regimen of INFERGEN® interferon alfacon-1 comprising administering a dosage of INFERGEN® interferon alfacon-1 containing an amount of 15 μg of drug per dose, subcutaneously once daily or three times per week for the desired treatment duration with an NS5B inhibitor compound.

As non-limiting examples, any of the above-described methods featuring an IFN-γ regimen can be modified to replace the subject IFN-γ regimen with a regimen of IFN-γ comprising administering a dosage of IFN-γ containing an amount of 25 μg of drug per dose, subcutaneously three times per week for the desired treatment duration with an NS5B inhibitor compound.

As non-limiting examples, any of the above-described methods featuring an IFN-γ regimen can be modified to replace the subject IFN-γ regimen with a regimen of IFN-γ comprising administering a dosage of IFN-γ containing an amount of 50 μg of drug per dose, subcutaneously three times per week for the desired treatment duration with an NS5B inhibitor compound.

As non-limiting examples, any of the above-described methods featuring an IFN-γ regimen can be modified to replace the subject IFN-γ regimen with a regimen of IFN-γ comprising administering a dosage of IFN-γ containing an amount of 100 μg of drug per dose, subcutaneously three times per week for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 100 μg of drug per dose, subcutaneously once weekly, once every 8 days, or once every 10 days; and (b) administering a dosage of IFN-γ containing an amount of 50 μg of drug per dose, subcutaneously three times per week; for the desired treatment duration with an NS5B inhibitor compound.

As non-limiting examples, any of the above-described methods featuring a TNF antagonist regimen can be modified to replace the subject TNF antagonist regimen with a TNF antagonist regimen comprising administering a dosage of a TNF antagonist selected from the group of: (a) etanercept in an amount of 25 mg of drug per dose subcutaneously twice per week, (b) infliximab in an amount of 3 mg of drug per kilogram of body weight per dose intravenously at weeks 0, 2 and 6, and every 8 weeks thereafter, or (c) adalimumab in an amount of 40 mg of drug per dose subcutaneously once weekly or once every 2 weeks; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 100 μg of drug per dose, subcutaneously once weekly, once every 8 days, or once every 10 days; and (b) administering a dosage of IFN-γ containing an amount of 100 μg of drug per dose, subcutaneously three times per week; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 150 μg of drug per dose, subcutaneously once weekly, once every 8 days, or once every 10 days; and (b) administering a dosage of IFN-γ containing an amount of 50 μg of drug per dose, subcutaneously three times per week; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 150 μg of drug per dose, subcutaneously once weekly, once every 8 days, or once every 10 days; and (b) administering a dosage of IFN-γ containing an amount of 100 μg of drug per dose, subcutaneously three times per week; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 200 μg of drug per dose, subcutaneously once weekly, once every 8 days, or once every 10 days; and (b) administering a dosage of IFN-γ containing an amount of 50 μg of drug per dose, subcutaneously three times per week; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of monoPEG (30 kD, linear)-ylated consensus IFN-α containing an amount of 200 μg of drug per dose, subcutaneously once weekly, once every 8 days, or once every 10 days; and (b) administering a dosage of IFN-γ containing an amount of 100 μg of drug per dose, subcutaneously three times per week; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously three times per week; and (b) administering a dosage of IFN-γ containing an amount of 25 μg of drug per dose, subcutaneously three times per week; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously three times per week; and (b) administering a dosage of IFN-γ containing an amount of 50 μg of drug per dose, subcutaneously three times per week; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously three times per week; and (b) administering a dosage of IFN-γ containing an amount of 100 μg of drug per dose, subcutaneously three times per week; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously once daily; and (b) administering a dosage of IFN-γ containing an amount of 25 μg of drug per dose, subcutaneously three times per week; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously once daily; and (b) administering a dosage of IFN-γ containing an amount of 50 μg of drug per dose, subcutaneously three times per week; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 9 μg of drug per dose, subcutaneously once daily; and (b) administering a dosage of IFN-γ containing an amount of 100 μg of drug per dose, subcutaneously three times per week; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously three times per week; and (b) administering a dosage of IFN-γ containing an amount of 25 μg of drug per dose, subcutaneously three times per week; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously three times per week; and (b) administering a dosage of IFN-γ containing an amount of 50 μg of drug per dose, subcutaneously three times per week; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously three times per week; and (b) administering a dosage of IFN-γ containing an amount of 100 μg of drug per dose, subcutaneously three times per week; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously once daily; and (b) administering a dosage of IFN-γ containing an amount of 25 μg of drug per dose, subcutaneously three times per week; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously once daily; and (b) administering a dosage of IFN-γ containing an amount of 50 μg of drug per dose, subcutaneously three times per week; for the desired treatment duration with an NS5B inhibitor compound.

As non-limiting examples, any of the above-described methods featuring an IFN-α and IFN-γ combination regimen can be modified to replace the subject IFN-α and IFN-γ combination regimen with an IFN-α and IFN-γ combination regimen comprising: (a) administering a dosage of INFERGEN® interferon alfacon-1 containing an amount of 15 μg of drug per dose, subcutaneously once daily; and (b) administering a dosage of IFN-γ containing an amount of 100 μg of drug per dose, subcutaneously three times per week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 100 μg of drug per dose, subcutaneously once weekly, once every 8 days, or once every 10 days; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 100 μg of drug per dose, subcutaneously once weekly, once every 8 days, or once every 10 days; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 150 μg of drug per dose, subcutaneously once weekly, once every 8 days, or once every 10 days; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 150 μg of drug per dose, subcutaneously once weekly, once every 8 days, or once every 10 days; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 200 μg of drug per dose, subcutaneously once weekly, once every 8 days, or once every 10 days; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of monoPEG (30 kD, linear)-ylated consensus IFN-α containing an amount of 200 μg of drug per dose, subcutaneously once weekly, once every 8 days, or once every 10 days; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously three times per week; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously three times per week; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously three times per week; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously once daily; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously once daily; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 9 μg of drug per dose, subcutaneously once daily; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously three times per week; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously three times per week; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously three times per week; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously once daily; (b) administering a dosage of IFN-γ containing an amount of 25 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously once daily; (b) administering a dosage of IFN-γ containing an amount of 50 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α, IFN-γ and TNF antagonist combination regimen can be modified to replace the subject IFN-α, IFN-γ and TNF antagonist combination regimen with an IFN-α, IFN-γ and TNF antagonist combination regimen comprising: (a) administering a dosage of INFERGEN® interferon alfacon-1 containing an amount of 15 μg of drug per dose, subcutaneously once daily; (b) administering a dosage of IFN-γ containing an amount of 100 μg of drug per dose, subcutaneously three times per week; and (c) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of monoPEG (30 kD, linear)-ylated consensus IFN-α containing an amount of 100 μg of drug per dose, subcutaneously once weekly, once every 8 days, or once every 10 days; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of monoPEG (30 kD, linear)-ylated consensus IFN-α containing an amount of 150 μg of drug per dose, subcutaneously once weekly, once every 8 days, or once every 10 days; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of monoPEG (30 kD, linear)-ylated consensus IFN-α containing an amount of 200 μg of drug per dose, subcutaneously once weekly, once every 8 days, or once every 10 days; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of INFERGEN® interferon alfacon-1 containing an amount of 9 μg of drug per dose, subcutaneously once daily or three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-α and TNF antagonist combination regimen can be modified to replace the subject IFN-α and TNF antagonist combination regimen with an IFN-α and TNF antagonist combination regimen comprising: (a) administering a dosage of INFERGEN® interferon alfacon-1 containing an amount of 15 μg of drug per dose, subcutaneously once daily or three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-γ and TNF antagonist combination regimen can be modified to replace the subject IFN-γ and TNF antagonist combination regimen with an IFN-γ and TNF antagonist combination regimen comprising: (a) administering a dosage of IFN-γ containing an amount of 25 μg of drug per dose, subcutaneously three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-γ and TNF antagonist combination regimen can be modified to replace the subject IFN-γ and TNF antagonist combination regimen with an IFN-γ and TNF antagonist combination regimen comprising: (a) administering a dosage of IFN-γ containing an amount of 50 μg of drug per dose, subcutaneously three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods featuring an IFN-γ and TNF antagonist combination regimen can be modified to replace the subject IFN-γ and TNF antagonist combination regimen with an IFN-γ and TNF antagonist combination regimen comprising: (a) administering a dosage of IFN-γ containing an amount of 100 μg of drug per dose, subcutaneously three times per week; and (b) administering a dosage of a TNF antagonist selected from (i) etanercept in an amount of 25 mg subcutaneously twice per week, (ii) infliximab in an amount of 3 mg of drug per kilogram of body weight intravenously at weeks 0, 2 and 6, and every 8 weeks thereafter or (iii) adalimumab in an amount of 40 mg subcutaneously once weekly or once every other week; for the desired treatment duration with an NS5B inhibitor compound.

As non-limiting examples, any of the above-described methods that includes a regimen of monoPEG (30 kD, linear)-ylated consensus IFN-α can be modified to replace the regimen of monoPEG (30 kD, linear)-ylated consensus IFN-α with a regimen of peginterferon alfa-2a comprising administering a dosage of peginterferon alfa-2a containing an amount of 180 μg of drug per dose, subcutaneously once weekly for the desired treatment duration with an NS5B inhibitor compound.

As non-limiting examples, any of the above-described methods that includes a regimen of monoPEG (30 kD, linear)-ylated consensus IFN-α can be modified to replace the regimen of monoPEG (30 kD, linear)-ylated consensus IFN-α with a regimen of peginterferon alfa-2b comprising administering a dosage of peginterferon alfa-2b containing an amount of 1.0 μg to 1.5 μg of drug per kilogram of body weight per dose, subcutaneously once or twice weekly for the desired treatment duration with an NS5B inhibitor compound.

As non-limiting examples, any of the above-described methods can be modified to include administering a dosage of ribavirin containing an amount of 400 mg, 800 mg, 1000 mg or 1200 mg of drug orally per day, optionally in two or more divided doses per day, for the desired treatment duration with an NS5B inhibitor compound.

As non-limiting examples, any of the above-described methods can be modified to include administering a dosage of ribavirin containing (i) an amount of 1000 mg of drug orally per day for patients having a body weight of less than 75 kg or (ii) an amount of 1200 mg of drug orally per day for patients having a body weight of greater than or equal to 75 kg, optionally in two or more divided doses per day, for the desired treatment duration with an NS5B inhibitor compound.

As non-limiting examples, any of the above-described methods can be modified to replace the subject NS5B inhibitor regimen with an NS5B inhibitor regimen comprising administering a dosage of 0.01 mg to 0.1 mg of drug per kilogram of body weight orally daily, optionally in two or more divided doses per day, for the desired treatment duration with the NS5B inhibitor compound.

As non-limiting examples, any of the above-described methods can be modified to replace the subject NS5B inhibitor regimen with an NS5B inhibitor regimen comprising administering a dosage of 0.1 mg to 1 mg of drug per kilogram of body weight orally daily, optionally in two or more divided doses per day, for the desired treatment duration with the NS5B inhibitor compound.

As non-limiting examples, any of the above-described methods can be modified to replace the subject NS5B inhibitor regimen with an NS5B inhibitor regimen comprising administering a dosage of 1 mg to 10 mg of drug per kilogram of body weight orally daily, optionally in two or more divided doses per day, for the desired treatment duration with the NS5B inhibitor compound.

As non-limiting examples, any of the above-described methods can be modified to replace the subject NS5B inhibitor regimen with an NS5B inhibitor regimen comprising administering a dosage of 10 mg to 100 mg of drug per kilogram of body weight orally daily, optionally in two or more divided doses per day, for the desired treatment duration with the NS5B inhibitor compound.

As non-limiting examples, any of the above-described methods featuring an NS3 inhibitor regimen can be modified to replace the subject NS3 inhibitor regimen with an NS3 inhibitor regimen comprising administering a dosage of 0.01 mg to 0.1 mg of drug per kilogram of body weight orally daily, optionally in two or more divided doses per day, for the desired treatment duration with an NS5B inhibitor compound.

As non-limiting examples, any of the above-described methods featuring an NS3 inhibitor regimen can be modified to replace the subject NS3 inhibitor regimen with an NS3 inhibitor regimen comprising administering a dosage of 0.1 mg to 1 mg of drug per kilogram of body weight orally daily, optionally in two or more divided doses per day, for the desired treatment duration with an NS5B inhibitor compound.

As non-limiting examples, any of the above-described methods featuring an NS3 inhibitor regimen can be modified to replace the subject NS3 inhibitor regimen with an NS3 inhibitor regimen comprising administering a dosage of 1 mg to 10 mg of drug per kilogram of body weight orally daily, optionally in two or more divided doses per day, for the desired treatment duration with an NS5B inhibitor compound.

As non-limiting examples, any of the above-described methods featuring an NS3 inhibitor regimen can be modified to replace the subject NS3 inhibitor regimen with an NS3 inhibitor regimen comprising administering a dosage of 10 mg to 100 mg of drug per kilogram of body weight orally daily, optionally in two or more divided doses per day, for the desired treatment duration with an NS5B inhibitor compound.

### Patient Identification

In certain embodiments, the specific regimen of drug therapy used in treatment of the HCV patient is selected according to certain disease parameters exhibited by the patient, such as the initial viral load, genotype of the HCV infection in the patient, liver histology and/or stage of liver fibrosis in the patient.

Thus, some embodiments provide any of the above-described methods for the treatment of HCV infection in which the subject method is modified to treat a treatment failure patient for a duration of 48 weeks.

Other embodiments provide any of the above-described methods for HCV in which the subject method is modified to treat a non-responder patient, where the patient receives a 48 week course of therapy.

Other embodiments provide any of the above-described methods for the treatment of HCV infection in which the subject method is modified to treat a relapser patient, where the patient receives a 48 week course of therapy.

Other embodiments provide any of the above-described methods for the treatment of HCV infection in which the subject method is modified to treat a naïve patient infected with HCV genotype 1, where the patient receives a 48 week course of therapy.

Other embodiments provide any of the above-described methods for the treatment of HCV infection in which the subject method is modified to treat a naïve patient infected with HCV genotype 4, where the patient receives a 48 week course of therapy.

Other embodiments provide any of the above-described methods for the treatment of HCV infection in which the subject method is modified to treat a naïve patient infected with HCV genotype 1, where the patient has a high viral load (HVL), where “HVL” refers to an HCV viral load of greater than 2×10⁶ HCV genome copies per mL serum, and where the patient receives a 48 week course of therapy.

One embodiment provides any of the above-described methods for the treatment of an HCV infection, where the subject method is modified to include the steps of (1) identifying a patient having advanced or severe stage liver fibrosis as measured by a Knodell score of 3 or 4 and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having advanced or severe stage liver fibrosis as measured by a Knodell score of 3 or 4 and then (2) administering to the patient the drug therapy of the subject method for a time period of about 40 weeks to about 50 weeks, or about 48 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per mL of patient serum and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per mL of patient serum and then (2) administering to the patient the drug therapy of the subject method for a time period of about 40 weeks to about 50 weeks, or about 48 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per mL of patient serum and no or early stage liver fibrosis as measured by a Knodell score of 0, 1, or 2 and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV genotype 1 infection and an initial viral load of greater than 2 million viral genome copies per mL of patient serum and no or early stage liver fibrosis as measured by a Knodell score of 0, 1, or 2 and then (2) administering to the patient the drug therapy of the subject method for a time period of about 40 weeks to about 50 weeks, or about 48 weeks.

Another embodiment provides any of the above-described methods for the treatment of an HCV infection, where the subject method is modified to include the steps of (1) identifying a patient having an HCV genotype 1 infection and an initial viral load of less than or equal to 2 million viral genome copies per mL of patient serum and then (2) administering to the patient the drug therapy of the subject method for a time period of about 20 weeks to about 50 weeks, or about 24 weeks to about 48 weeks, or about 30 weeks to about 40 weeks, or up to about 20 weeks, or up to about 24 weeks, or up to about 30 weeks, or up to about 36 weeks, or up to about 48 weeks.

Another embodiment provides any of the above-described methods for the treatment of an HCV infection, where the subject method is modified to include the steps of (1) identifying a patient having an HCV genotype 1 infection and an initial viral load of less than or equal to 2 million viral genome copies per mL of patient serum and then (2) administering to the patient the drug therapy of the subject method for a time period of about 20 weeks to about 24 weeks.

Another embodiment provides any of the above-described methods for the treatment of an HCV infection, where the subject method is modified to include the steps of (1) identifying a patient having an HCV genotype 1 infection and an initial viral load of less than or equal to 2 million viral genome copies per mL of patient serum and then (2) administering to the patient the drug therapy of the subject method for a time period of about 24 weeks to about 48 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 20 weeks to about 50 weeks, or about 24 weeks to about 48 weeks, or about 30 weeks to about 40 weeks, or up to about 20 weeks, or up to about 24 weeks, or up to about 30 weeks, or up to about 36 weeks, or up to about 48 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of about 20 weeks to about 24 weeks.

Another embodiment provides any of the above-described methods for the treatment of an HCV infection, where the subject method is modified to include the steps of (1) identifying a patient having an HCV genotype 2 or 3 infection and then (2) administering to the patient the drug therapy of the subject method for a time period of at least about 24 weeks.

Another embodiment provides any of the above-described methods for the treatment of an HCV infection, where the subject method is modified to include the steps of (1) identifying a patient having an HCV genotype 1 or 4 infection and then (2) administering to the patient the drug therapy of the subject method for a time period of about 24 weeks to about 60 weeks, or about 30 weeks to about one year, or about 36 weeks to about 50 weeks, or about 40 weeks to about 48 weeks, or at least about 24 weeks, or at least about 30 weeks, or at least about 36 weeks, or at least about 40 weeks, or at least about 48 weeks, or at least about 60 weeks.

Another embodiment provides any of the above-described methods for the treatment of an HCV infection, where the subject method is modified to include the steps of (1) identifying a patient having an HCV infection characterized by any of HCV genotypes 5, 6, 7, 8 and 9 and then (2) administering to the patient the drug therapy of the subject method for a time period of about 20 weeks to about 50 weeks.

Another embodiment provides any of the above-described methods for the treatment of an HCV infection, where the subject method is modified to include the steps of (1) identifying a patient having an HCV infection characterized by any of HCV genotypes 5, 6, 7, 8 and 9 and then (2) administering to the patient the drug therapy of the subject method for a time period of at least about 24 weeks and up to about 48 weeks.

### Subjects Suitable for Treatment

Any of the above treatment regimens can be administered to individuals who have been diagnosed with an HCV infection. Any of the above treatment regimens can be administered to individuals who have failed previous treatment for HCV infection (“treatment failure patients,” including non-responders and relapsers).

Individuals who have been clinically diagnosed as infected with HCV are of particular interest in many embodiments. Individuals who are infected with HCV are identified as having HCV RNA in their blood, and/or having anti-HCV antibody in their serum. Such individuals include anti-HCV ELISA-positive individuals, and individuals with a positive recombinant immunoblot assay (RIBA). Such individuals may also, but need not, have elevated serum ALT levels.

Individuals who are clinically diagnosed as infected with HCV include naïve individuals (e.g., individuals not previously treated for HCV, particularly those who have not previously received IFN-α-based and/or ribavirin-based therapy) and individuals who have failed prior treatment for HCV (“treatment failure” patients). Treatment failure patients include non-responders (i.e., individuals in whom the HCV titer was not significantly or sufficiently reduced by a previous treatment for HCV, e.g., a previous IFN-α monotherapy, a previous IFN-α and ribavirin combination therapy, or a previous pegylated IFN-α and ribavirin combination therapy); and relapsers (i.e., individuals who were previously treated for HCV, e.g., who received a previous IFN-α monotherapy, a previous IFN-α and ribavirin combination therapy, or a previous pegylated IFN-α and ribavirin combination therapy, whose HCV titer decreased, and subsequently increased).

In particular embodiments of interest, individuals have an HCV titer of at least about 10⁵, at least about 5×10⁵, or at least about 10⁶, or at least about 2×10⁶, genome copies of HCV per milliliter of serum. The patient may be infected with any HCV genotype (genotype 1, including 1a and 1b, 2, 3, 4, 6, etc. and subtypes (e.g., 2a, 2b, 3a, etc.)), particularly a difficult to treat genotype such as HCV genotype 1 and particular HCV subtypes and quasispecies.

Also of interest are HCV-positive individuals (as described above) who exhibit severe fibrosis or early cirrhosis (non-decompensated, Child's-Pugh class A or less), or more advanced cirrhosis (decompensated, Child's-Pugh class B or C) due to chronic HCV infection and who are viremic despite prior anti-viral treatment with IFN-α-based therapies or who cannot tolerate IFN-α-based therapies, or who have a contraindication to such therapies. In particular embodiments of interest, HCV-positive individuals with stage 3 or 4 liver fibrosis according to the METAVIR scoring system are suitable for treatment with the methods described herein. In other embodiments, individuals suitable for treatment with the methods of the embodiments are patients with decompensated cirrhosis with clinical manifestations, including patients with far-advanced liver cirrhosis, including those awaiting liver transplantation. In still other embodiments, individuals suitable for treatment with the methods described herein include patients with milder degrees of fibrosis including those with early fibrosis (stages 1 and 2 in the METAVIR, Ludwig, and Scheuer scoring systems; or stages 1, 2, or 3 in the Ishak scoring system.).

### NS5B Inhibitors

### Methodology

The HCV polymerase inhibitors can be prepared according to the procedures and schemes shown herein. The numberings in each of the following Preparation of NS5B Inhibitor are meant for that specific scheme only, and should not be construed or confused with the same numberings in other schemes.

### Preparation of NS5B Inhibitors

### Example 1

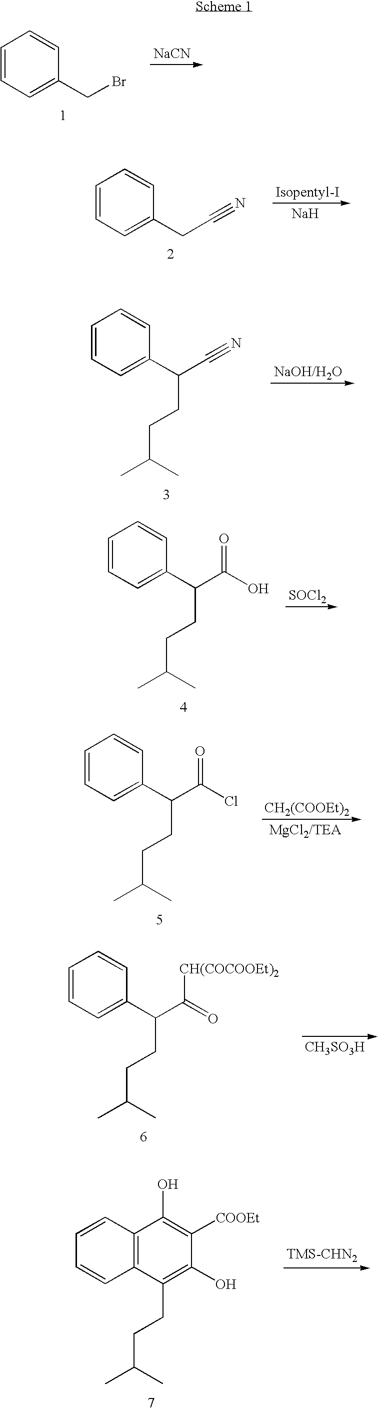

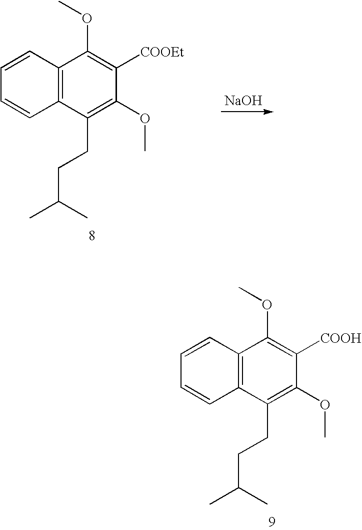

Compound 2, prepared from benzyl bromide and sodium cyanide, was alkylated with 3-methylbutyl iodide to give compound 3. Compound 3 was hydrolyzed to the acid 4, which was converted to the acyl chloride 5. Condensation of compound 5 with diethyl malonate gave compound 6, which cyclized in the presence of methanesulfonic acid to give compound 7. Compound 9 was obtained by methylation of compound 7 with trimethylsilyldiazomethane and subsequent hydrolysis.

### Preparation of alpha-(3-methylbutyl)-alpha-phenylacetonitrile (3)

A mixture of compound 1 (17.1 g, 0.1 mol) and NaCN (5.39 g, 0.11 mol) in 50 mL of ethanol and 300 mL of water was heated (oil bath 98-100° C.) for 5 h. The mixture was cooled, concentrated to remove ethanol, and extracted with ethyl acetate. The organic phase was washed with brine, dried (Na₂SO₄) and concentrated. Distillation gave 9.25 g of compound 2.

Compound 2 (9.25 g, 79.06 mmol) was added to a stirred suspension of 60% NaH/mineral oil (3.48 g, 87 mmol) in 50 mL of anhydrous DMF and 100 mL of toluene at 0° C. under argon. The mixture was stirred at room temperature for 1 h and 10 min at 30° C. After cooling to 0° C., 3-methyl-1-iodobutane (15.7 g, 79.2 mmol) was added, and the resulting mixture stirred at 0° C. for 30 min. The reaction mixture was quenched with water, and diluted with ethyl acetate. The organic phase was washed with brine 3 times, with dilute NaHCO₃, dried (Na₂SO₄), and concentrated. Distillation gave 10.2 g of compound 3.

### Preparation of ethyl 1,3-dihydroxynaphthalene-2-carboxylate (7)

A solution of compound 3 (19.3 g) in ethoxyethanol (150 mL) and 2N NaOH (150 mL) was refluxed overnight, concentrated to a small volume, and dissolved in water. The aqueous solution was extracted with toluene, acidified with 2 N HCl to pH ˜2, extracted again with ethyl acetate 3×. The ethyl acetate extracts were dried (Na₂SO₄) and concentrated to give 18.7 g of compound 4 as liquid.

A solution of compound 4 (18.5 g, 89.8 mmol) and thionyl chloride in 60 mL of 1,2-dichloroethane was refluxed for 4 h and concentrated to dryness. The remaining syrup (compound 5) was co-evaporated with anhydrous toluene and then dried under high vacuum.

To a mixture of magnesium chloride (8.64 g, 89.8 mmol) and diethyl malonate (14.38 g, 89.8 mmol) in 90 mL of anhydrous acetonitrile at 0° C. under argon was added slowly 25 mL (180 mmol) of triethylamine. The resulting mixture was stirred at 0° C. for 30 min. Compound 5 (89.8 mmol the crude obtained above) in 10 mL of anhydrous acetonitrile was added dropwise, and the resulting reaction mixture was stirred at 0° C. for 1 h and at rt overnight. The mixture was cooled with ice, made acidic with 250 mL of 2N HCl, and extracted with EtOAc. The extracts were washed with brine 3 times, dried (Na₂SO₄), and concentrated to give 31 g of the crude 6 as a faint-amber syrup.

The crude compound 6 (15.5 g) was dissolved in 80 mL of methanesulfonic acid, and the solution stood at 30° C. overnight. After cooled, the mixture was poured into 700 mL of ice-water and extracted with EtOAc. The extracts were washed with brine 4 times, dried (Na₂SO₄), and concentrated. Chromatography on silica gel with DCM/hexanes (1:4 to 1:3) gave 9.2 g of 7 as yellow solid.

### Preparation of ethyl 1,3-dimethoxy-4-(3-methylbutyl)naphthalene-2-carboxylate (9)

TMS-diazomethane in hexane (2.0 M, 50 mL) was added to a solution of the crude compound 7 (3.02 g, 10 mmol) in 30 mL of THF and 15 mL of methanol. Under cooling with cold water, 5 mL of diisopropylethylamine was added, and the resulting solution stood at 30° C. overnight. The solution was concentrated to dryness. Chromatography on silica gel with 25-40% DCM in hexanes gave 3.07 g of compound 8 as syrup.

A mixture of compound 8 (3.07 g) in dioxane (90 mL), 2 N NaOH (30 mL) and water (40 mL) was refluxed for 2 days, cooled with ice, and acidified with 2N HCl to pH ˜2, and extracted with EtOAc. The extracts were washed with brine 3 times, dried (Na₂SO₄), and concentrated to give the crude compound 9 as faint-amber syrup. ¹H NMR (CDCl₃) 1.03 (d, J=6.8 Hz, 6H), 1.55 (m, 2H), 1.77 (sept, J=6.8 Hz, 1H), 3.03 (m, 2H), 3.94 (s, 3H), 4.07 (s, 3H), 7.49 (ddd, J=8.0, 1.2 Hz, 1H), (ddd, J=8.4, 1.6 Hz, 1H), 7.98 (d, J=8.4 Hz, 1H), 8.15 (dd, J=8.4, 1.6 Hz, 1H).

### Example 2

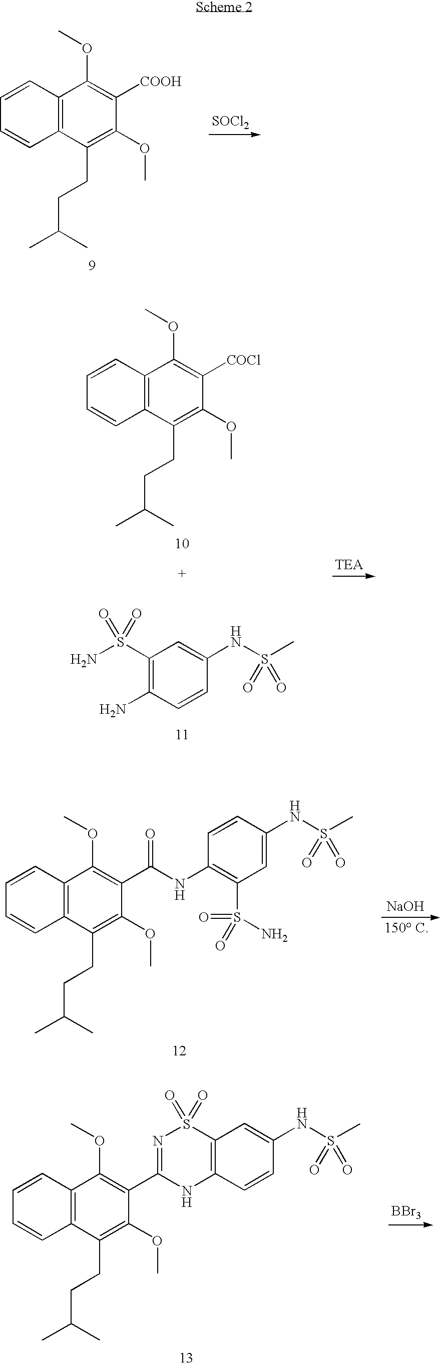

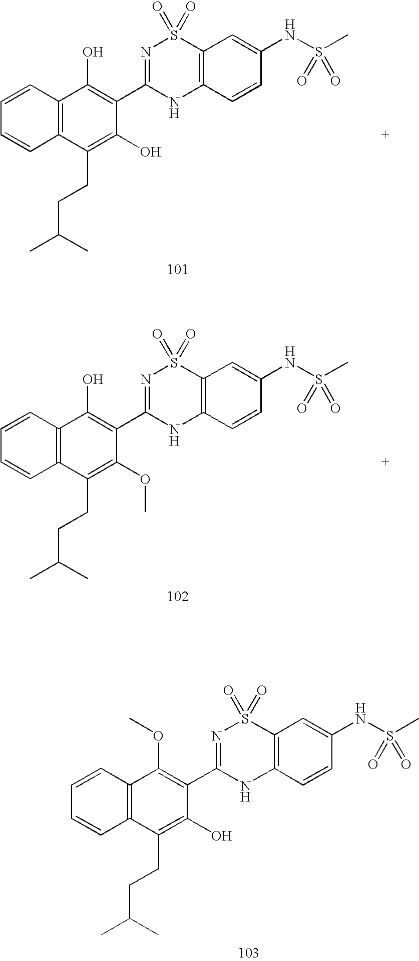

The acyl chloride 10, obtained by reacting Compound 9 with thionyl chloride, was condensed with 6-amino-3-(methanesulfonamido)benzenesulfonamide 11 to give the coupling product compound 12, which was converted to compound 13 by cyclization under vigorous heating condition. Removal of methyl group was effected by treatment of compound 13 with boron tribromide to yield compounds 101, 102 and 103.

### Preparation of 1,3-dimethoxy-2-(1,1-dioxo-6-(methanesulfonamido)-2H-(1,2,4)-benzothiadiazin-3-yl)-4-(3-methylbutyl)naphthalene (13)

A solution of compound 9 (604 mg, 2.0 mmol) and thionyl chloride (0.4 mL in 1,2-dichloromethane (4 mL) was heated at 60° C. overnight, concentrated to dryness, and under high vacuum. The crude compound 10 in anhydrous dimethoxyethane (3 mL) was added to a solution of compound 11 (531 mg, 2 mmol) and pyridine (0.96 mL, 12 mmol) in anhydrous 1,2-diemthoxyethane (20 mL). The mixture was stirred at room temperature overnight and then triethylamine (1 mL) was added. The resulting mixture stirred at rt for 6 h, concentrated to a small volume, and diluted with DCM. Precipitate was filtered and washed with DCM. The filtrate was concentrated and the residue purified on a silica gel column with 10-30% EtOAc in DCM to give 178 mg of compound 12 as white solid.

A solution of compound 12 (170 mg) in 16 mL of 0.25N NaOH was heated in a stainless steel vessel at 150° C. for 3 days, cooled, and neutralized with 2N HCl. The precipitate was filtered and washed with water. The crude was purified by chromatography on silica gel with 10-15% EtOAc/DCM to give 63 mg of compound 13 as a white solid.

### Preparation of 1,3-dihydroxy-2-(1,1-dioxo-6-(methanesulfonamido)-2H-(1,2,4)-benzothiadiazin-3-yl)-4-(3-methylbutyl)naphthalene (101), 2-(1,1-dioxo-6-(methanesulfonamido)-2H-(1,2,4)-benzothiadiazin-3-yl)-1-hydroxy-3-methoxy-4-(3-methylbutyl)naphthalene (102) and 2-(1,1-dioxo-6-(methanesulfonamido)-2H-(1,2,4)-benzothiadiazin-3-yl)-3-hydroxy-1-methoxy-4-(3-methylbutyl)naphthalene (103)

A solution of compound 13 (50 mg, 0.096 mmol) and BBr₃ (1.0 M/DCM, 1.0 mL) in 4 mL of anhydrous 1,2-dichloroethane was heated at 40° C. for 28 h, cooled, concentrated to dryness and co-evaporated with methanol. The crude was purified by chromatography on silica gel with 10-25% acetone in DCM to give a mixture of compounds 102 and 103 and 5.9 mg of 101 as yellow solid. Further purification of compounds 102 and 103 on a silica gel column with 2-7% EtOAc in DCM gave 10.4 mg of compounds 102 and 2.5 mg of 103, both as pale-yellow solid. ¹H NMR of compound 101 (acetone-d₆) δ 1.01 (d, J=6.4 Hz, 6H), 1.49 (m, 2H), 1.75 (sept, J=6.8 Hz, 1H), 3.03 (m, 2H), 3.07 (s, 3H), 7.31 (t, J=7.4, 1H), 7.49 (d, J=8.8 Hz, 1H), 7.56 (t, J=7.4 Hz, 1H), 7.64 (dd, J=8.8, 2.4 Hz, 1H), 7.80 (d, J=2.0 Hz, 1H), 7.83 (d, J=8.8 Hz, 1H), 8.33 (d, J=8.4 Hz, 1H), 8.86 (s, 1H, D₂O exchangeable); ¹H NMR of compound 102 (acetone-d₆) δ 1.06 (d, J=6.4 Hz, 6H), 1.59 (m, 2H), 1.84 (sept, 6.8 Hz, 1H), 3.08 (m, 2H), 3.12 (s, 3H), 3.95 (s, 3H), 7.55 (ddd, J=8.4, 1.2 Hz, 1H), 7.70-7.76 (m, 3H), 7.87 (d, J=2.0 Hz, 1H), 8.02 (d, J=8.8 Hz, 1H), 8.43 (d, J=8.4 Hz, 1H), 8.9 (br, 1H, D₂O exchangeable), 11.7 (br, 1H, D₂O exchangeable, 12.9 (br, 1H, D₂O exchangeable); ¹H NMR of compound 103 (CDCl₃) δ 1.03 (d, J=6.8 Hz, 6H), 1.5 (m, 2H), 1.76 (sept, J=6.8 Hz, 1H), 3.06 (m, 2H), 3.08 (s, 3H), 4.04 (s, 3H), 6.82 (s, br, 1H, D₂O exchangeable), 7.23 (d, J=8.8 Hz, 1H), 7.38 (ddd, J=8.4, 1.2 Hz, 1H), 7.56 (ddd, J=8.4, 1.6 Hz, 1H), 7.70 (ddd, J=8.4 Hz, 2.4 Hz, 1H), 7.72 (d, J=2.4 Hz, 1H), 7.92 (d, J=8.4 hz, 1H), 8.03 (d, J=8.0 Hz, 1H), 11.44 (s, 1H, D₂O exchangeable), 11.54 (s, 1H, D₂O exchangeable).

### Example 3

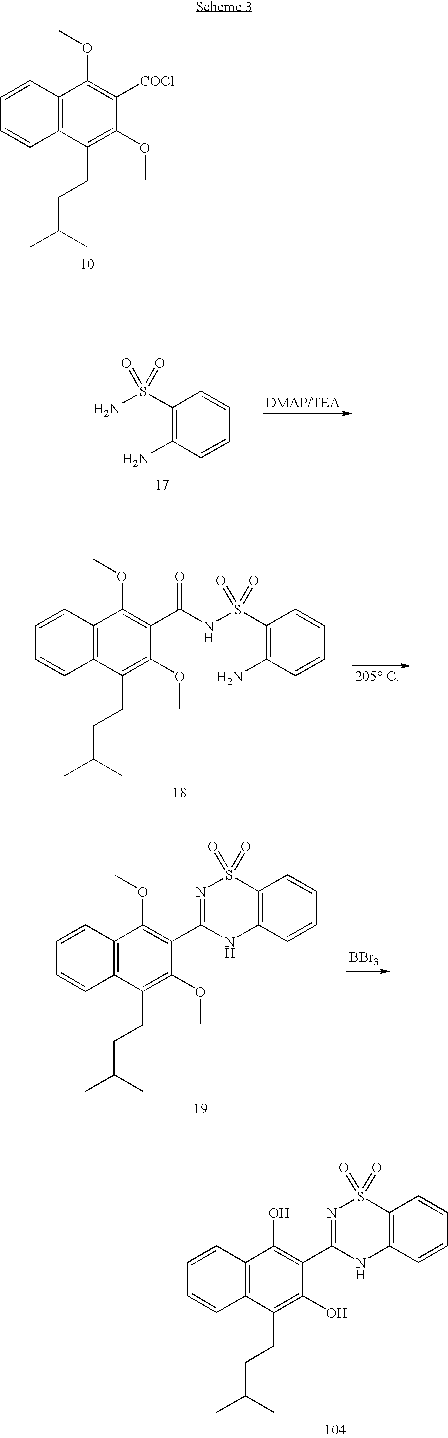

Compound 104 was obtained by heating 19 at 200° C., which was prepared by condensation of 10 with 2-aminobenzenesulfonamide (17) in the presence of DMAP and TEA.

### Preparation of 1,3-dihydroxy-2-(1,1-dioxo-2H-(1,2,4)-benzothiadiazin-3-yl)-4-(3-methylbutyl)naphthalene (104)

A solution of 9 (crude, 9.3 mmol) and thionyl chloride (1.7 mL in 1,2-dichloromethane (15 mL) was heated at 50° C. overnight, concentrated to dryness, and under high vacuum. To a solution of the crude 10 in anhydrous DMF (8 mL) was added a solution of 17 (1.60 g mg, 9.3 mmol), DMAP (227 mg, 1.86 mmol) and TEA (2.6 mL, 18.6 mmol) in anhydrous DMF (8 mL). The resulting mixture was stirred at 30° C. for 30 h, diluted with ethyl acetate, washed with brine, dried (Na₂SO₄) and concentrated. Chromatography on a silica gel with 2-8% EtOAc in DCM gave 1.56 g of 18 as white solid.

Compound 18 (neat, 1.52 g) was heated under argon at 200° C. for 90 min. The resulting residue was cooled and purified by chromatography on silica gel with 4-7% EtOAc in DCM/hexanes (1:1) to give 585 mg of 19 as white solid; ¹H NMR (CDCl₃) δ 1.03 (d, J=6.8 Hz, 6H), 1.51 (m, 2H), 1.77 (sept, J=6.8 Hz, 1H), 2.84 (m, 2H), 3.93 (s, 3H), 4.07 (s, 3H), 7.03 (d, J=8.4 Hz, 1H), 7.38 (dt, J=7.6, 0.8 Hz, 1H), 7.46-7.61 (m, 3H), 7.86 (d, J=8.8 Hz, 1H), 7.97 (d, J=8.0 Hz, 1H), 8.08 (d, J=8.2 Hz, 1H), 8.82 (s, 1H, D₂O exchangeable).

A solution of 19 (390 mg, 0.91 mmol) and BBr₃ (7.3 M/DCM, 1.0 mL) in 12 mL of anhydrous 1,2-dichloroethane was heated at 45° C. for 24 h, cooled, concentrated to dryness, and co-evaporated with methanol. Precipitate in DCM was thoroughly washed with DCM to give 205 mg of 104 as yellow solid. The filtrate was concentrated to dryness and the residue purified by chromatography on silica gel with 2-4% EtOAc in DCM to give additional 62 mg of 104 as yellow solid; ¹H NMR (DMSO-d₆) δ 0.99 (d, J=6.8 Hz, 6H), 1.39 (m, 2H), 1.71 (sept, J=6.8 Hz, 1H), 2.93 (s, 2H), 7.32 (t, J=7.4 Hz, 1H), 7.39 (d, J=8.0 Hz, 1H), 7.45 (dt, J=7.4, 0.8 Hz, 1H), 7.54 (ddd, J=8.4, 1.2 Hz, 1H), 7.67 (ddd, J=8.2, 1.2 Hz, 1H), 7.82 (d, J=8.4 Hz, 1H), 7.84 (dd, J=8.0, 2.0 Hz, 1H), 8.20 (dd, J=8.4, 0.8 Hz, 1H).

### Example 4

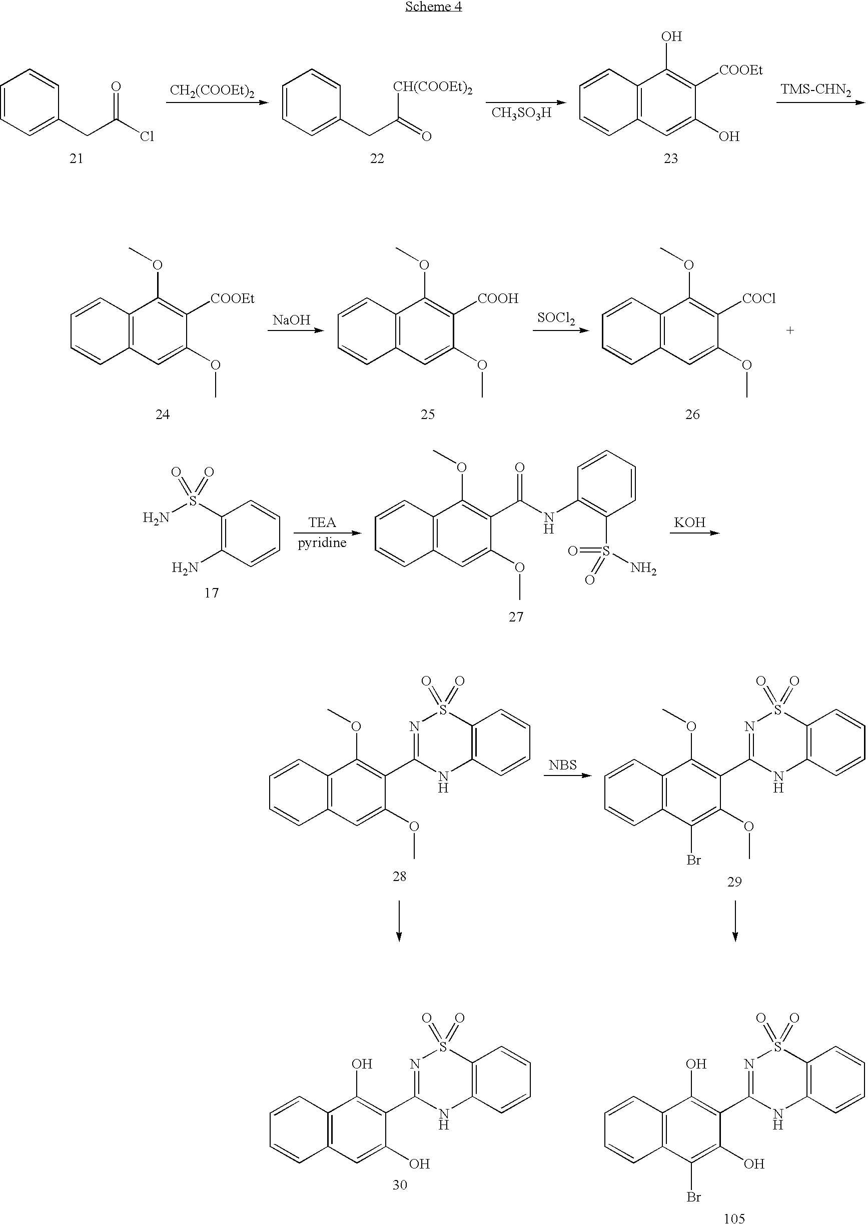

Condensation of 21 with diethyl malonate, followed by cyclization, afforded the naphthalene derivative 23. Methylation of 23 with trimethylsilyldiazomethane and subsequent hydrolysis gave 25. Compound 25 was converted to the acyl chloride 26 and then condensed with 2-aminobenzenesulfonamide (17) to yield 27. Compound 28 was obtained by vigorous heating in the presence of potassium hydroxide. Bromination of 28 with NBS gave the bromonaphthalene derivative 29. Compound 30 and 105 were prepared from 28 and 29, respectively, by treatment with boron tribromide.

### Preparation of ethyl 1,3-dihydroxynaphthalene-2-carboxylate (23)

To a mixture of magnesium chloride (8.64 g, 89.8 mmol) and diethyl malonate (14.38 g, 89.8 mmol) in 90 mL of anhydrous acetonitrile at 0° C. under argon was slowly added triethylamine (25 mL, 180 mmol). The resulting mixture was stirred at 0° C. for 30 min. Commercially available 21 (13.88 g, 89.8 mmol) was added dropwise, and the resulting reaction mixture was stirred at rt overnight. The mixture was cooled with ice, made acidic with 250 mL of 2N HCl, and extracted with EtOAc. The extracts were washed with brine 3×, dried (Na₂SO₄), and concentrated.

The resulting product 22 was dissolved in 150 mL of methanesulfonic acid, and the solution stood at 30° C. for 2 days. After cooled, the mixture was poured into 1400 mL of ice-water, extracted with EtOAc. The extracts were washed with brine 4 times, dried (Na₂SO₄), and concentrated to give 22 g of the crude 23 as syrup.

### Preparation of ethyl 1,3-methoxynaphthalene-2-carboxylate (25)

TMS-dazomethane in hexane (2.0M, 162 mL) was added to a solution of the crude 23 (90 mmol) in 200 mL of THF and 100 mL of methanol. Under cooling with cold water, 12 mL of diisopropylethylamine was added, and the resulting solution stood at rt for 2 days. The solution was concentrated to dryness. Chromatography on silica gel with DCM/hexanes (1:2 to 2:1) gave 12.01 g of 24 as syrup.

A mixture of 24 (8.35 g, 25.3 mmol) in ethoxyethanol (100 mL) and 1 N NaOH (100 mL) was refluxed for 24 h, and diluted with cold water, and extracted once with DCM/hexane mixture. The aqueous phase was made acidic with 2N HCl and extracted with EtOAc. The extracts were washed with brine 3 times, dried (Na₂SO₄), and concentrated. The residue was co-evaporated with xylene 2 times and under high vacuum overnight to give 8.14 g of 25 as faint-amber syrup.

### Preparation of 2-(1,1-dioxo-2H-(1,2,4)-benzothiadiazin-3-yl)-1,3-methoxynaphthalene (28)

A solution of 25 (6.05 g, 26 mmol) and thionyl chloride (5.0 mL in 1,2-dichloromethane (50 mL) was reflux 55° C. overnight (or 60° C., 5 h), concentrated to dryness, and under high vacuum for two hours. The crude 26 in anhydrous DMF (10 mL) was added to a solution of 2-aminobenzenesulfonamide (4.47 g, 26 mmol) in anhydrous DMF (26 mL), followed by addition of triethylamine (7.3 mL, 52 mmol). The mixture was stirred at rt overnight and 40° C. for 5 h. The mixture was diluted with DCM, and the resulting precipitate was filtered and washed with DCM to give 3.3 g of 27 as white solid.

A solution of 27 (1.98 g) in 70 mL of 10% aqueous KOH was heated in a pressure bottle for 3 days, cooled, and neutralized with 2N HCl. The precipitate was filtered and washed with water. The precipitate was extracted with DCM-EtOAc, and then with DCM-MeOH. The extracts were concentrated and crystallized from EtOAc gave 28 as off-white solid. The filtrate was concentrated and chromatographed on silica gel with 2-8% EtOAc in DCM to give another crop of 28. Total yield was 1.06 g as off-white solid; ¹H NMR (DMSO-d₃) δ 3.90 (s, 3H), 3.97 (s, 3H), 7.37 (d, J=8.0 Hz, 1H), 7.37 (s, 1H), 7.47-7.55 (m, 2H), 7.61 (ddd, J=8.2, 1.2 Hz, 1H), 7.73 (ddd, J=8.4, 1.2 Hz, 1H), 7.90 (dd, J=8.0, 1.6 Hz, 1H), 7.95 (d, J=8.0 Hz, 1H), 8.05 (d, J=8.0 Hz, 1H), 12.57 (s, 1H, D₂O exchangeable).

### Preparation of 1,3-dihydroxy-2-(1,1-dioxo-2H-(1,2,4)-benzothiadiazin-3-yl)naphthalene (30)

A solution of 28 (74 mg, 0.20 mmol) and BBr₃ (1.0 M, 1.0 mL) in 1,2-dichloroethane (3 mL) was stirred at 45° C. for 40 h, concentrated, co-evaporated with DCM, and co-evaporated with methanol. Precipitate in acetone was filtered and washed thoroughly with warm acetone to give 31 mg of 30 as yellow solid; ¹H NMR (DMSO-d₃) δ 6.82 (s, 1H), 7.29 (ddd, J=8.4, 1.2 Hz, 1H), 7.42 (d, J=8.4 Hz, 1H), 7.45-7.51 (m, 2H), 7.67 (d, J=8.4 Hz, 1H), 7.70 (m, 1H), 7.87 (dd, J=8.0, 1.2 Hz, 1H), 8.13 (d, J=8.4 Hz, 1H), 10.6 (br), 12.5 (br).

### Preparation of 4-bromo-2-(1,1-dioxo-2H-(1,2,4)-benzothiadiazin-3-yl)-1,3-methoxynaphthalene (29)

A solution of 28 (370 mg, 1.0 mmol), NBS (214 mg, 1.2 mmol), and 50 microliter of concentrated sulfuric acid in 15 mL of anhydrous THF was stirred at rt overnight. The solution was neutralized with 0.5 mL of TEA and concentrated. Chromatography on silica gel with 1-4% EtOAc in DCM gave 432 mg of 29 as white solid; ¹H NMR (CDCl₃) .4.03 (s, 3H), 4.10 (s, 3H), 7.07 (d, J=8.0 Hz, 1H), 7.38 (m, 1H), 7.49-7.56 (m, 2H), 7.62 (m, 1H), 7.95 (d, J=8.0 Hz, 1H), 8.01 (d, J=8.0 Hz, 1H), 8.11 (d, J=8.4 Hz, 1H), 9.15 (s/br, 1H, D₂O exchangeable).

### Preparation of 4-bromo-1,3-dihydroxy-2-(1,1-dioxo-2H-(1,2,4)-benzothiadiazin-3-yl)naphthalene (105)

A solution of 29 (76 mg, 0.17 mmol) and BBr₃ (1.0 M, 1.4 mL) in 1,2-dichloroethane (3.5 mL) was stirred at rt for 4 days, concentrated, co-evaporated with DCM, and co-evaporated with methanol. Precipitate in acetone was filtered and washed thoroughly with warm acetone to give 22 mg of 105 as dark-yellow solid; ¹H NMR (DMSO-d₃) δ 7.35-7.43 (m, 2H), 7.46 (t, J=7.6 Hz, 1H), 7.61-7.70 (m, 2H), 7.83 (d, J=8.0 Hz, 1H), 7.96 (d, J=8.0 Hz, 1H), 8.22 (d, J=8.0 Hz, 1H).

### Example 5

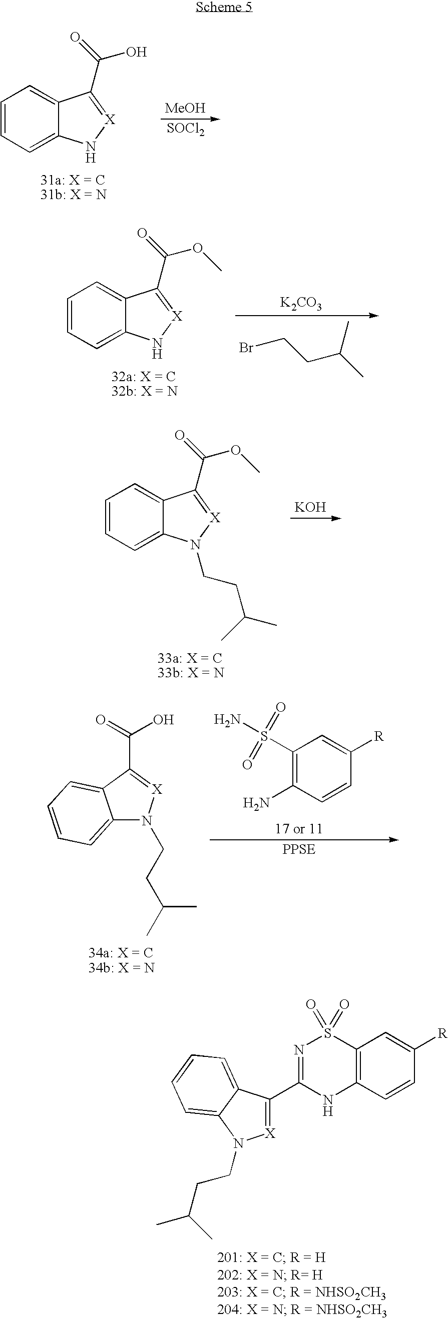

### Preparation of 1H-indazole-3-carboxylic acid methyl ester (32b)

To the methanol solution of 1H-indazole-3-carboxylic acid (31b) (162 mg, 1 mmol) was added SOCl₂ (0.5 mL) and the mixture was stirred at the room temperature for 24 h. After evaporation of the volatiles, the mixture was partitioned between aqueous NaHCO₃ solution and ethyl acetate. The aqueous phase was extracted with ethyl acetate (2×15 mL), and the combined organic layer was dried over sodium sulfate. The volatiles were removed, and the residue was filtered over silica gel to provide 123 mg of 1H-Indazole-3-carboxylic acid methyl ester (32b).

### Preparation of 1-(3-Methyl-butyl)-1H-indole-3-carboxylic acid methyl ester (33a)

A solution of 0.16 g (1.00 mmol) of indole-3-carboxylic acid methyl ester (32a), 0.5 g (4 mmol) of K₂CO₃ and 180 mg (1.2 mmol) of 1-bromo-3-methyl-butane in 3 ml of DMF was stirred at 50° C. overnight. The mixture was partitioned between EtOAc and water. The aqueous phase was extracted with additional EtOAc. The combined organic phases were washed with water, brine and finally dried over Na₂SO₄, then purified by prep-TLC (PE:EA=3:1), to give 0.4 g of 1-(3-Methyl-butyl)-1H-indole-3-carboxylic acid methyl ester (33a).

### Preparation of 1-(3-Methyl-butyl)-1H-indole-3-carboxylic acid (34a)

The 0.4 g of 1-(3-Methyl-butyl)-1H-indole-3-carboxylic acid methyl ester (33a) was dissolved in 10 ml of methanol and 10 ml of 1M KOH and heated to reflux overnight. After most of the methanol was removed in vacuum the residual water phase was diluted to 25 ml, acidified and extracted with 3×25 ml of EtOAc. The combined organic phases were washed with brine and evaporated to give 0.38 g of the crude 1-(3-Methyl-butyl)-1H-indole-3-carboxylic acid (34).

### Preparation of Compounds 201-208

The 0.1 mmol of 1-(3-Methyl-butyl)-1H-indole-3-carboxylic acid (34a), 1-(3-Methyl-butyl)-1H-indazole-3-carboxylic acid (34b) or the derivatives of compound 34a (for example, compounds 34c, 34d, 34e, and 34f) was added to 1 ml of PPSE, and stirred at 160° C. After the acid was solved, the benzenesulfonamide (0.1 mmol) 17 or 11 was added, keep stirring at 160° C. for 1 hour, then poured to ice water, extract with EtOAc, then concentrated the organic layer and purified by Prep-HPLC to obtain the desired compound.

Derivatives of compound 34a used include:

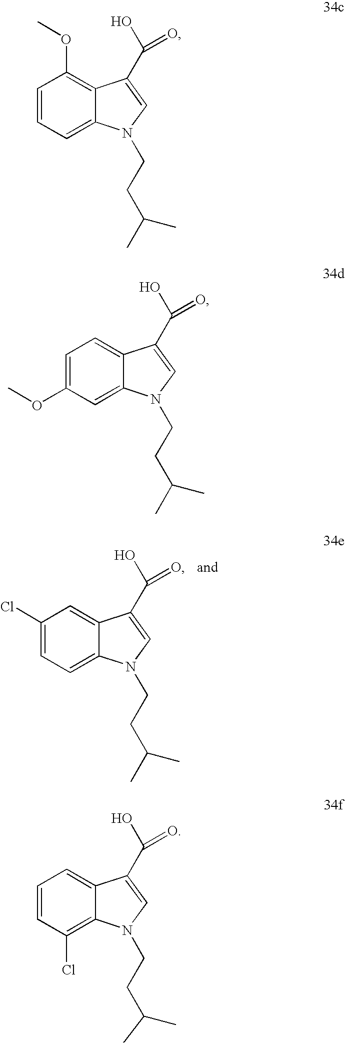

Different benzenesulfonamide used for the reactions are as follows:

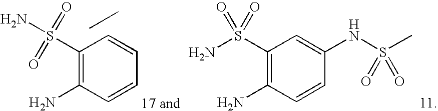

| | | | Benzene- | |
|---|---|---|---|---|
| Compound | Structure | Precursor | sulfonamide | Characterization |
| 201 | 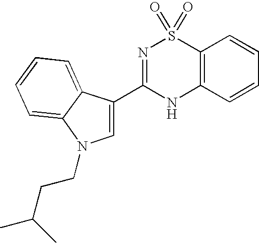 | 34a | 17 | MS-ESI: m/z = 368 [M + 1]⁺ 70% yield White solid |
| 202 | 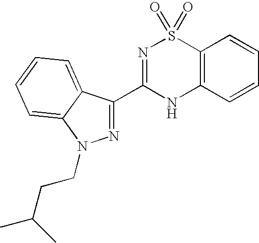 | 34b | 17 | MS-ESI: m/z = 369 [M + 1]⁺ 64% yield White solid |
| 203 | 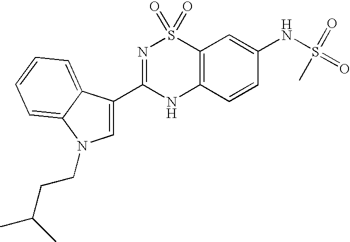 | 34a | 11 | MS-ESI: m/z = 461 [M + 1]⁺ 50% yield White solid |
| 204 | 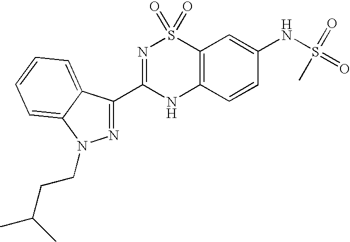 | 34b | 11 | MS-ESI: m/z = 462 [M + 1]⁺¹ 45% yield White solid |
| 205 | 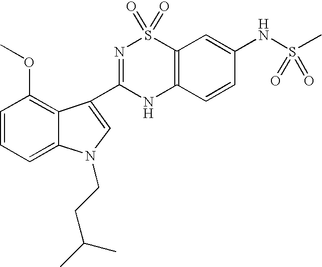 | 34c | 11 | MS-ESI: m/z = 491 [M + 1]⁺ 46.2% yield Gray solid |
| 206 | 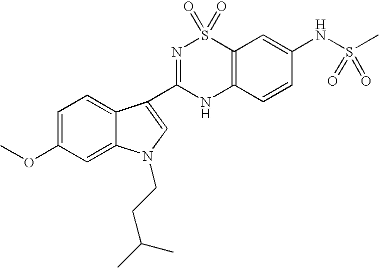 | 34d | 11 | MS-ESI: m/z = 491 [M + 1]⁺ 77.9% yield Yellow solid |
| 207 | 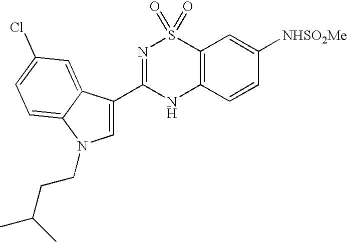 | 34e | 11 | MS-ESI: m/z = 495.2 [M + 1]⁺ 30.2% yield Yellow solid |
| 208 | 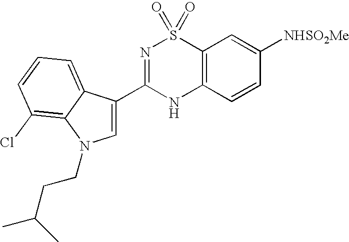 | 34f | 11 | MS-ESI: m/z = 495.2 [M + 1]⁺ 63.3% yield Yellow solid |

### Preparation of Compounds 209 and 210

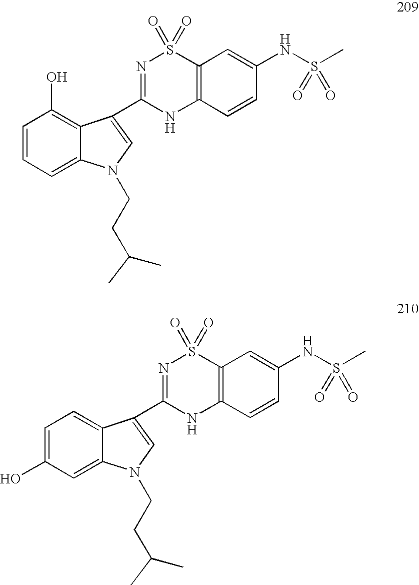

To the mixture of compound 205 in anhydrous CH₂Cl₂ was added 4M BBr₃ (4 eq.) at −40° C. under N₂ atmosphere and then it was warmed to room temperature slowly, and the mixture was stirred at room temperature for about 2-3 hours. Then the mixture was poured into ice/water and the solvent was filtered off and the precipitate was washed with water. The precipitate was collected and dried under freeze drying to give the pure product of compound 209 (83.8% yield) as a yellow solid. MS-ESI: m/z=477 [M+1]⁺.

The same procedure as above was used with compound 206 as the starting material to obtain compound 210 (83.6% yield) as a yellow solid. MS-ESI: m/z=477 [M+1]⁺.

### Example 6

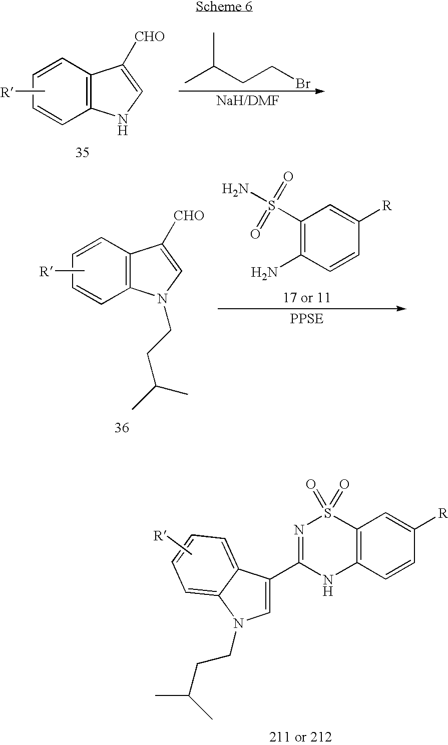

### Preparation of Compound 36

To the solution (DMF: 2 mL) of compound 35 (100 mg, 0.534 mmol) was added NaH (240 mg, 6 mmol) at 0° C. The reaction mixture was stirred for 0.5 h at 0-5° C. And 1-bromo-3-methyl-butane in DMF 1.5 eq□ was added, then the reaction mixture was allowed to come to room temperature and stirred for 3 hours. The mixture was poured into ice/water (30 mL) and extracted with EtOAc, the combined organic lagers were dried (Na₂SO₄), filtered and the solvent was evaporated, and purified on silica gel to give compound 36 (110 mg, 80% yield)

### Preparation of Compounds 211 and 212

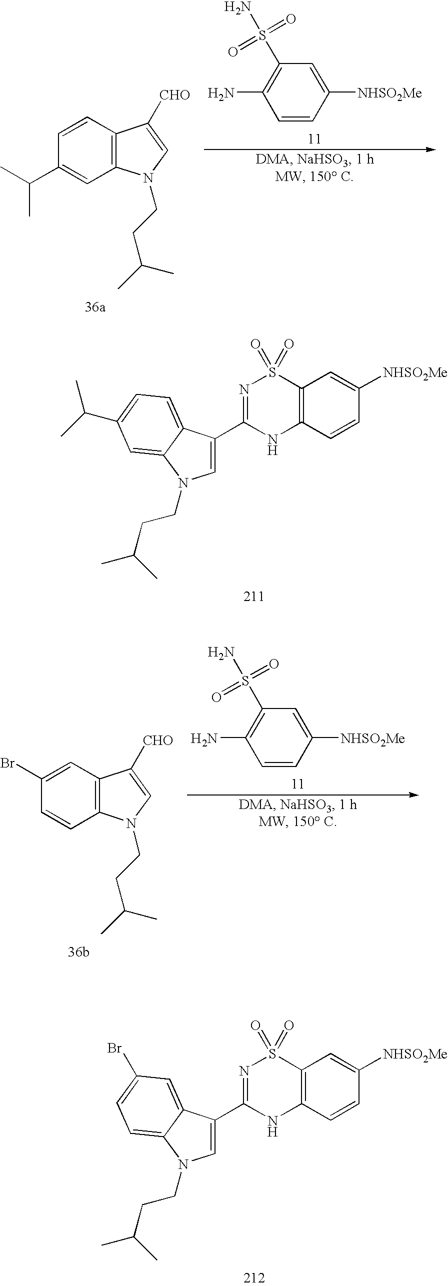

Compound 36a (200 mg, 0.777 mmol), compound 11 (265 mg, 1 mmol) and NaHSO₃ (133 mg, 1.28 mmol) in dimethylacetamide is heated in microwave at 150° C. for 1 h. The reaction mixture was dissolved in EtOAc (200 mL) and washed with brine (4×10 mL), The organic layer was dried over Na₂SO₄ and concentrated, and was purified by TLC (PE:EA=1:3), all by-product moved and only product stay in the bottom. About 30 mg of yellow solid compound 211 was obtained. MS-ESI: m/z=503 [M+1]⁺.

Compound 212 (37% yield, yellow solid) can be obtained using the same procedure with compound 36b as the starting material. MS-ESI: m/z=539.1 [M+1]⁺.

### Preparation of Compound 224

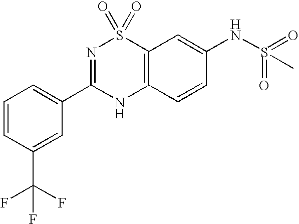

Compound 224 was prepared according to the procedure described for compound 211 shown in Scheme 6 using 3-(trifluoromethyl)benzaldehyde.

### Example 7

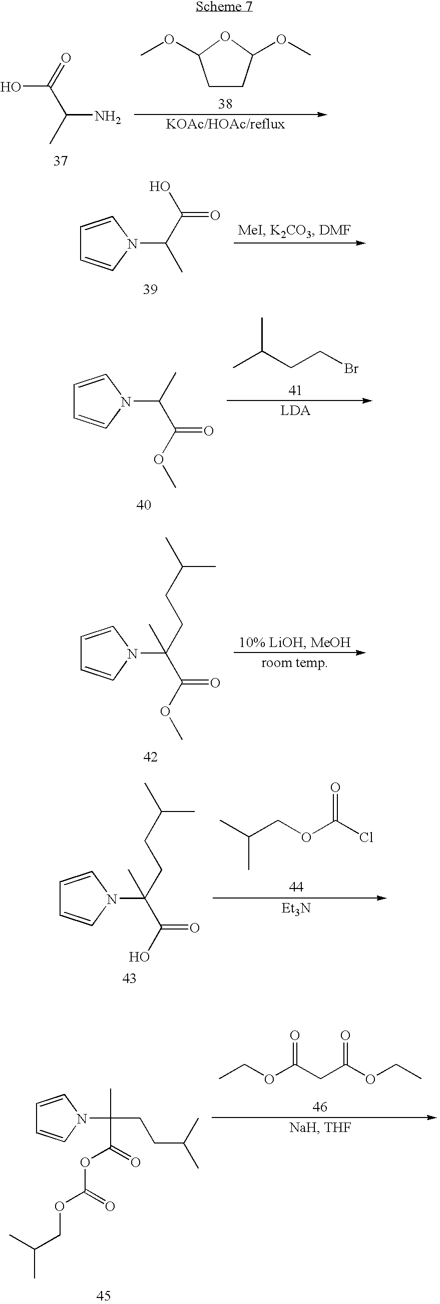

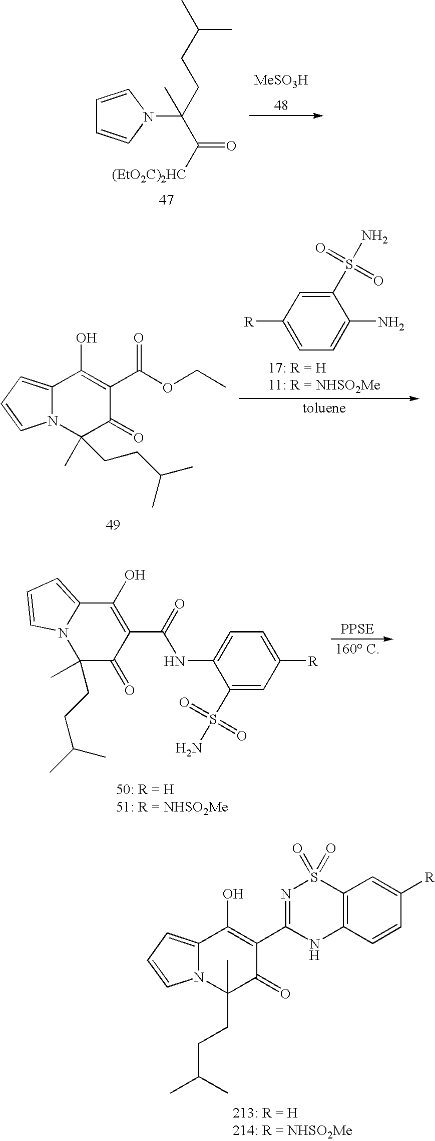

### Preparation of Compound 39

Compound 37 (75 g, 852 mmol) was dissolved in 112 ml of water and added to HOAc (750 ml). The reaction mixture was then heated to reflux and compound 38 (112 g, 852 mmol) was added dropwise. After heating for 4 h, the mixture was cooled and concentrated. The reaction mixture was poured into 1.5 L of water and extracted with ethyl acetate (500 ml×3) and dried over Na₂SO₄. The solvent was removed and the crude product of compound 39 (120 g, purity 83%) was used directly without purification.

### Preparation of Compound 40

Compound 39 (60 g, 432 mmol) and CH₃I (123 g, 866 mmol) was dissolved in 300 ml of DMF, then 119 g of K₂CO₃ was added. The reaction mixture was stirred at room temperature for 18 h. 600 ml of ethyl acetate was then added and the mixture was washed with water (500 ml×3). The organic layer was combined and dried over Na₂SO₄. The solvent was removed and the crude product was purified by column chromatography on silica gel to give compound 40 (35 g, 53%) as a yellow liquid.

### Preparation of Compound 42

To a solution of (i-Pr₂)₂NH (19.8 g, 196.1 mmol) in 50 ml of THF, n-BuLi (78.4 ml, 196.1 mmol) was added dropwise at −78° C. It was slowly raised to −30° C. for 30 min and again cooldown to −78° C. After stirring for 1 h, the solution of compound 40 (20 g, 130.7 mmol) in 50 ml of THF was added and stirred for 1 h. To this solution was added compound 41 (39.5 g, 261.4 mmol) dropwise. The reaction mixture was slowly warmed to room temperature and stirred overnight. The mixture was quenched with 50 ml of water and extracted with ethyl acetate (400 ml×3). The combined organic layer was dried and concentrated. The crude product was purified by column chromatography on silica gel to afford compound 42 (6.4 g, 22%) as a yellow liquid.

### Preparation of Compound 43

To a solution of compound 42 (10 g, 44.7 mmol) in 120 ml of CH₃OH was added 10% aqueous LiOH (120 ml). The reaction mixture was stirred at room temperature for 6 h. 1 N aqueous HCl was added and the mixture was adjusted to PH=3. The mixture was extracted with ethyl acetate (200 ml×3). The combined organic layer was dried and concentrated. The crude product was purified by column chromatography on silica gel to afford compound 43 (5.5 g, 59%) as a black liquid.

### Preparation of Compound 45

Compound 43 (5.5 g, 26.3 mmol) and Et₃N (5.3 g, 52.6 mmol) was dissolved in 50 ml of DCM. Compound 44 (7.2 g, 52.6 mmol) was slowly added at 0° C. and the reaction mixture was stirred for 4 h. The mixture was poured into 100 ml of water and extracted with DCM (50 ml×3). The combined organic layer was dried over Na₂SO₄ and concentrated. The crude product of compound 45 (6.67 g, 82%) was used directly without further purification.

### Preparation of Compound 47

Compound 46 (6.9 g, 43.2 mmol) was dissolved in 40 ml of THF and 3.3 g of NaH was added slowly at 0° C. The mixture was stirred for 0.5 h and compound 45 (6.67 g, 21.59 mmol) was added. The mixture was stirred at room temperature for 2 h. and poured into 100 ml of water. The mixture was extracted with ethyl acetate (50 ml×3). The combined organic layer was dried and concentrated. The crude product was purified by column chromatography on silica gel to afford compound 47 (4.3 g, 57%) as a black liquid.

### Preparation of Compound 49

Compound 47 (2 g, 5.7 mmol) was dissolved in 20 ml of compound 48 and the mixture was stirred at room temperature overnight. The mixture was poured into 100 ml of ice-water and extracted with ethyl acetate (50 ml×3). The combined organic layer was dried and concentrated. The crude product was purified by column chromatography on silica gel to afford compound 49 (0.5 g, 29%) as a liquid.

### Preparation of Compound 50 or 51

To a solution of compound 49 (100 mg, 0.33 mmol) in 5 ml of toluene was added compound 17 or 11 (112 mg, 0.65 mmol). The reaction mixture was heated to 130° C. and stirred for 2 h. The mixture was concentrated and the crude product compound 50 or 51 (0.12 g) was used directly without purification.

### Preparation of Compounds 213 and 214

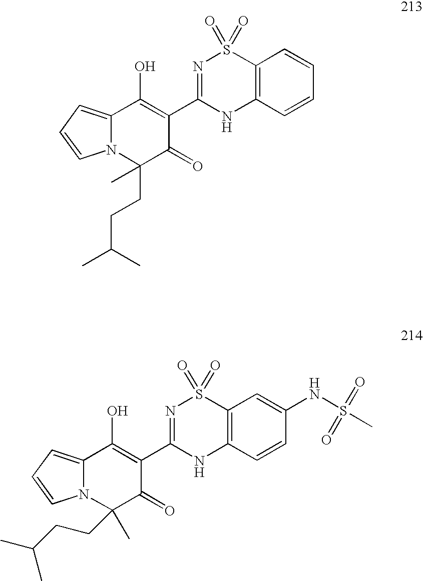

Compound 50 (0.12 g, 278 mmol) was added 5 ml of PPSE and the mixture was heated to 160° C. for 3 h. The mixture was poured into 10 ml of water and extracted with ethyl acetate (30 ml×3). The combined organic layer was dried and concentrated. The crude product was purified by column chromatography on silica gel to afford compound 213 (22 mg, 15%) as a yellow solid. MS-ESI: m/z=414 [M+1]⁺.

The same procedure with compound 51 was used to obtain compound 214 (14% yield) as a yellow solid. MS-ESI: m/z=507 [M+1]⁺.

### Example 8

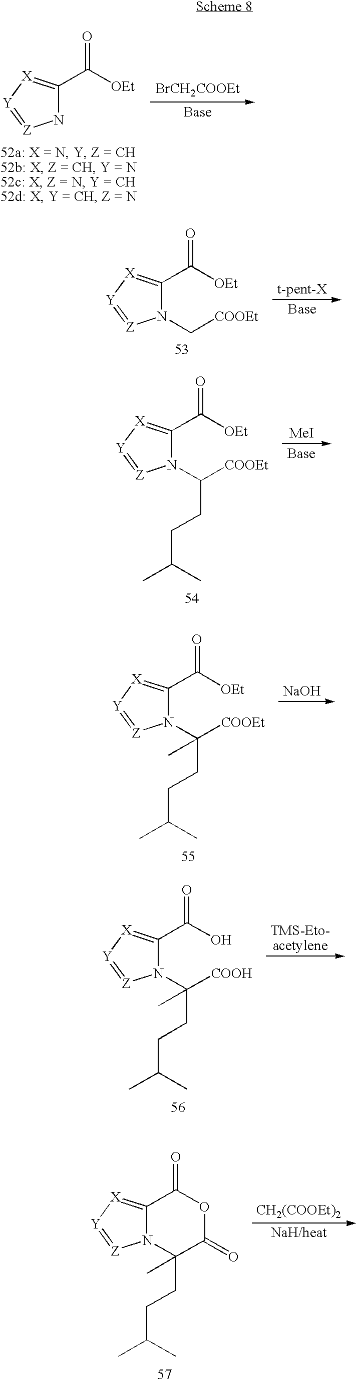

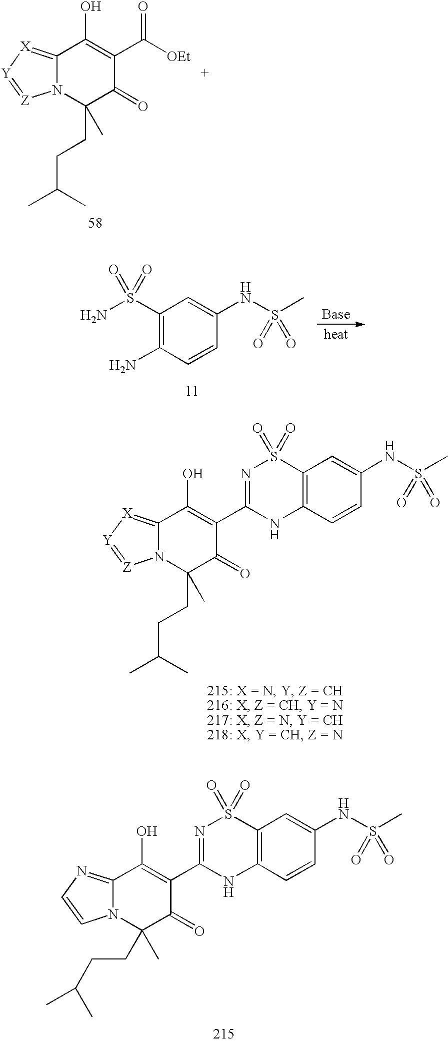

### Preparation of Compound 215

Compound 52 was alkylated in sequence with ethyl bromoacetate, isopentyl iodide, and iodomethane to give compound 55. Hydrolysis of compound 55, followed by a treatment with TMS-Eto-acetylene, gave the cyclic anhydride 57. Reaction of compound 57 with diethyl malonate gave compound 58, which coupled with compound 11 to yield the compound 215.

### Example 9

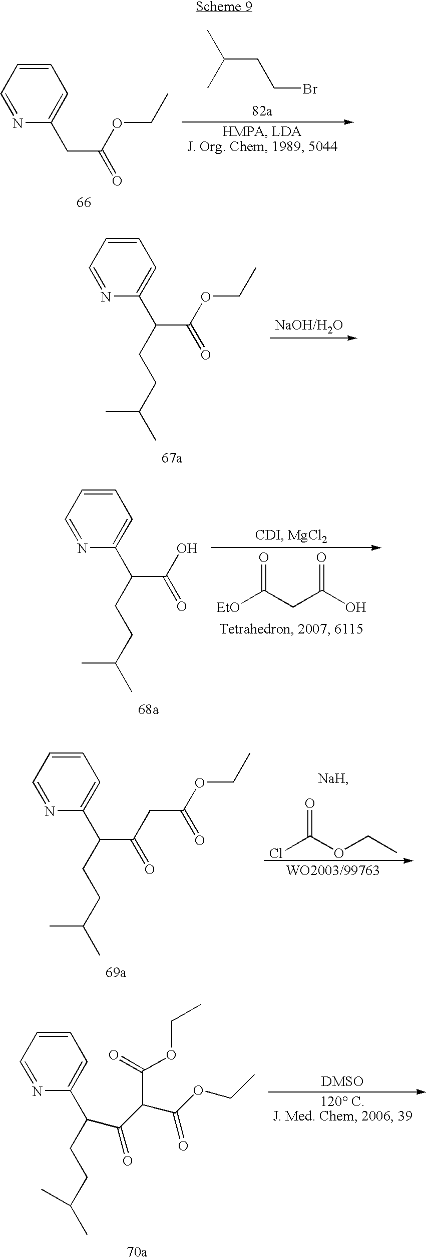

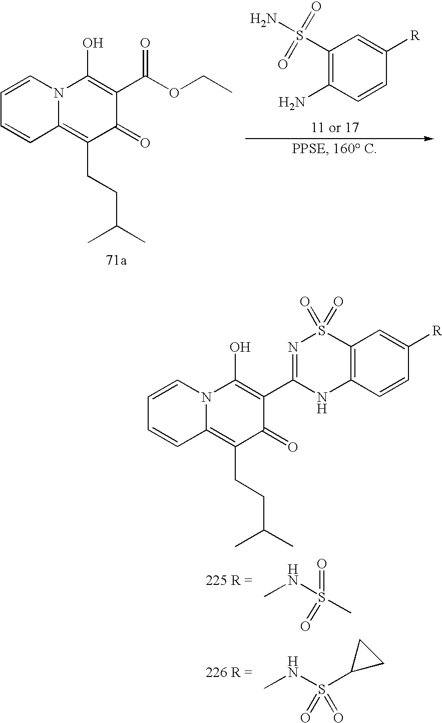

### Preparation of Compound 67a

A solution of 2.5M n-BuLi in hexane (29 mL, 72 mmol) was added dropwise to the solution of diisopropylamine (6.7 g, 67 mmol) in anhydrous THF (150 ml) at −78° C. and stirred for 1 h at this temperature. A solution of compound 66 (10 g, 61 mmol) in anhydrous THF (10 mL) was added dropwise to the mixture at −78° C. After 45 min at this temperature, 1-bromo-3-methylbutane (82a, 10 g, 67 mmol) in tetrahydrofuran (10 mL) was added dropwise to the mixture, followed by HMPA (6.7 g, 37 mmol). The reaction mixture was allowed to warm to room temperature overnight, and then was quenched with water and extracted with ethyl acetate. The organic layer was dried on Na₂SO₄ and concentrated. The product was purified by chromatography to give compound 67a as yellow oil. ¹H NMR (400 MHz, CDCl₃): 0.854 (m, 6H), 1.101 (m, 1H), 1.210 (m, 4H), 1.559 (m, 1H), 1.896 (m, 1H), 2.109 (m, 1H), 3.746 (m, 1H), 4.148 (m, 2H), 7.167 (m, 1H), 7.303 (m, 1H), 7.644 (m, 1H), 8.556 (t, 1H, J=2.4 Hz). MS-ESI: m/z=235.9 [M+1]⁺.

### Preparation of Compound 68a

Compound 67a (1 g, 4.24 mmol) was added to the solution of NaOH (950 mg, 50.12 mmol) in water and stirred at r.t. overnight. Then the mixture was cooled in an ice bath and neutralized with 1N HCl to pH˜4. The solution was freeze-dried to give the mixture of compound 68a and NaCl salt which was used directly for the next step. MS-ESI: m/z=207.9 [M+1]⁺.

### Preparation of Compound 69a

A solution of crude compound 68a (0.42 mmol) in anhydrous THF (1 mL) was cooled in salt-ice bath, and N,N′-carbonyldiimidazole (69 mg, 0.42 mmol) was added in small portions under vigorous stirring. After evolution of gas, the mixture was stirred at room temperature for 3 h and then cooled in an ice bath. To a suspension of monoethyl malonate potassium salt (159 mg, 0.93 mmol) in THF (2 mL) in ice bath was added Et₃N (0.21 mL, 1.44 mmol) followed by anhydrous MgCl₂ (109 mg, 1.15 mmol). The mixture was stirred at room temperature for 3 h, then cooled in salt-ice bath and the above solution of the activated ester previously prepared in THF was added dropwise slowly. The mixture was allowed to stir for 39 h at room temperature, then quenched with aqueous citric acid and extracted with ethyl acetate. The organic layers were washed with saturated NaHCO₃ solution and brine, dried (Na₂SO₄) and concentrated in vacuo and purified by prep-TLC to give compound 69a as yellow oil. ¹H NMR (400 MHz, CDCl₃): 0.875 (t, 6H, J=7 Hz), 1.059 (m, 1H), 1.217 (m, 4H), 1.564 (m, 1H), 1.875 (m, 1H), 2.143 (m, 1H), 3.419 (d, 1H, J=16 Hz), 3.534 (d, 1H, J=16 Hz), 4.002 (t, 1H, J=7.4 Hz), 4.140 (m, 2H), 7.228 (m, 2H), 7.692 (m, 1H), 8.599 (m, 1H). MS-ESI: m/z=277.9 [M+1]⁺.

### Preparation of Compound 70a

Compound 69a (1 g, 3.61 mmol) was dissolved in anhydrous THF (10 mL) and cooled to 0° C. NaH (60% in oil, 187 mg, 4.69 mmol) was added and the mixture was stirred for 45 min at room temperature. After cooling again to 0° C., a solution of ethyl chloroformate (430 mg, 3.97 mmol) in anhydrous THF (0.5 mL) was slowly added with a syringe. The solution was stirring at room temperature for 2 h, treated with water, acidified to pH˜3 by addition of citric acid and extracted with ethyl acetate. The organic layer was dried over Na₂SO₄ and concentrated in vacuo to give crude product 70a, which was used directly for the next step. MS-ESI: m/z=350.1 [M+1]⁺

### Preparation of Compound 71a

The crude compound 70a (3.61 mmol) was dissolved in DMSO (10 mL) and heated to 120° C. for 2.5 h. Then it was poured into water and extracted with ethyl acetate. The organic layer was washed with water, dried on Na₂SO₄ and concentrated in vacuo. The product was purified by prep-TLC to give compound 71a as brown solid. ¹H NMR (400 MHz, CDCl₃): 0.995 (d, 6H, J=6.4 Hz), 1.391 (m, 2H), 1.481 (t, 3H, J=7.2 Hz), 1.673 (m, 1H), 2.746 (m, 2H), 4.504 (q, 2H, J=7.2 Hz), 6.836 (t, 1H, J=6.8 Hz), 7.435 (m, 2H), 9.123 (d, 1H, J=7.6 Hz), 13.526 (s, 1H). MS-ESI: m/z=304.1 [M+1]⁺, m/z=326.0 [M+Na]⁺.

### Preparation of Compound 225

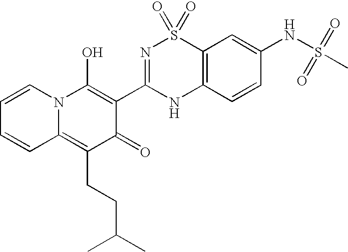

Compound 71a was added at 160° C. to PPSE which and then 2-amino-5-(methylsulfonamido)benzenesulfonamide was added. The solution stirred for 2 h at 160° C. The cooled mixture was poured into water and the precipitate was collected and washed with MeOH for several times. Then it was dried to give compound 225 as a green solid (36.1% yield). ¹H NMR (400 MHz, DMSO): 0.960 (d, 6H, J=6.4 Hz), 1.347 (q, 2H, J=6.4 Hz), 1.661 (m, 1H), 2.782 (t, 2H, J=8 Hz), 3.081 (s, 3H), 7.277 (t, 1H, J=7 Hz), 7.575 (dd, 1H, J₁=2.4 Hz, J₂=6.4 Hz), 7.628 (d, 1H, J=2 Hz), 7.688 (d, 1H, J=9.2 Hz), 7.799 (t, 1H, J=7.4 Hz), 7.856 (d, 1H, J=8.8 Hz), 9.053 (d, 1H, J=7.2 Hz), 10.270 (s, 1H), 14.133 (s, 1H), 14.281 (s, 1H). MS-ESI: m/z=505.1 [M+1]⁺.

### Preparation of Compound 226

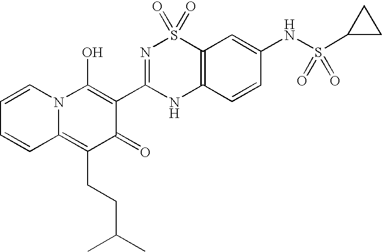

Compound 71a (200 mg, 0.66 mmol) was added at 160° C. to PPSE (3 mL), and then 2-amino-5-(cyclopropanesulfonamido)benzenesulfonamide (192 mg, 0.66 mmol) was added. The solution was stirred for 1.5 h at 160° C. The cooled mixture was poured into water and the precipitate was collected and purified by prep-HPLC (basic column) to give compound 226 as a yellow solid (60.1 mg, yield: 17.2%). ¹H NMR (400 MHz, DMSO): 0.954 (d, 10H, J=6.4 Hz), 1.330 (q, 2H, J=6 Hz), 1.644 (m, 1H), 2.718 (m, 3H), 7.228 (br, 1H), 7.595 (m, 1H), 7.681 (s, 2H), 7.774 (br, 2H), 9.014 (d, 1H, J=6.4 Hz), 10.265 (br, 1H). MS-ESI: m/z=531.1 [M+1]⁺.

### Example 10

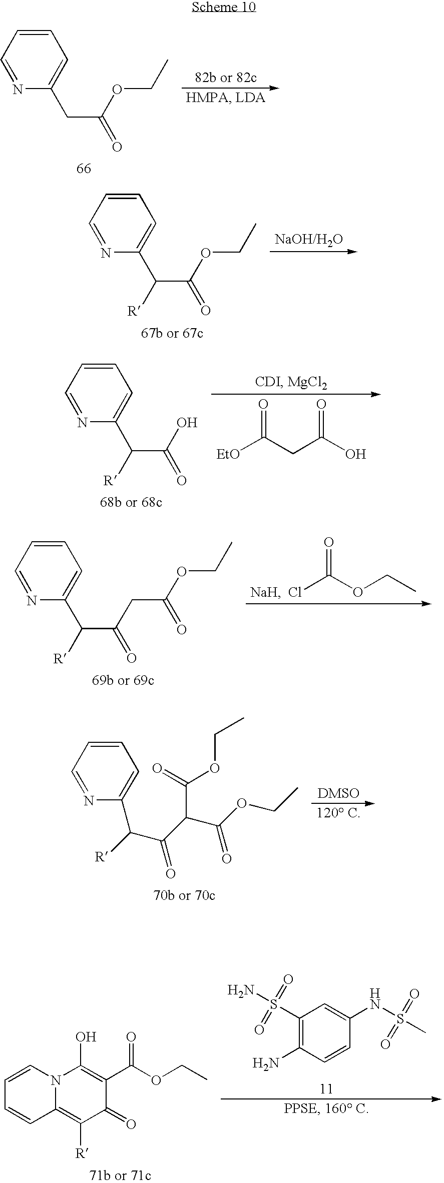

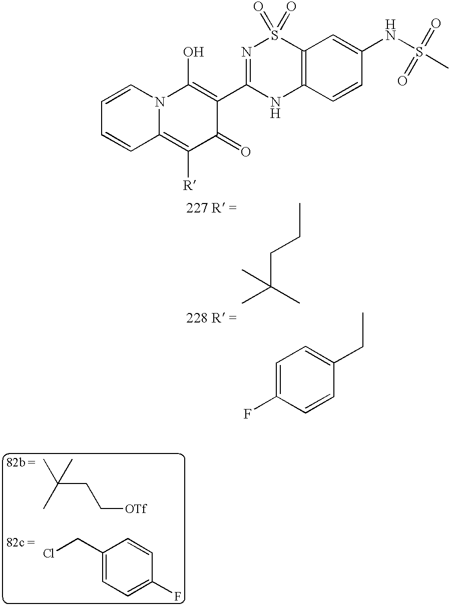

### Preparation of Compound 67b

To a solution of compound 66 (500 mg, 3.0 mmol) in 5 ml dry THF and LiHMDS (3.6 ml, 1M) at −78° C. After 2 h at this temperature, compound 82b (846 mg, 1.2 eq.) in dry THF (1 ml) was added dropwise, followed by HMPA (325.8 mg, 0.6 eq.). The reaction mixture was allowed to warm to room temperature and stirred overnight, then quenched with water and extracted with EtOAc. The organic layer was dried on Na₂SO₄ and concentrated. The crude was purified by chromatography to give compound 67b (465 mg, yield: 66.8%) as yellow oil. MS-ESI: m/z=250.0 [M+1]⁺.

### Preparation of Compound 67c

A solution of 1.0M LiHDMS in THF (0.67 mL, 0.67 mmol) was added dropwise to the solution of compound 66 (100 m g, 0.606 mmol) in anhydrous THF (5.0 ml) at −78° C. and stirred for 2 h at this temperature, and then compound 82c (96.0 m g, 0.67 mmol) was added dropwise to the mixture at −78° C. After 45 min at this temperature, the reaction mixture was allowed to warm to room temperature and stirred overnight, quenched with water and extracted with ethyl acetate. The organic layer was dried over Na₂SO₄ and concentrated. The product was purified by Prep-TLC (EA:PE=1:4) to give compound 67c (48.0m g, yield: 29.0%) as a yellow oil. MS-ESI: m/z=274.1 [M+1]⁺.

### Preparation of Compound 227

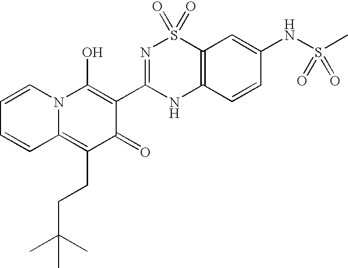

Compounds 68b (MS-ESI: m/z=222.0 [M+1]⁺), 69b (MS-ESI: m/z=292.0 [M+1]⁺), 70b (MS-ESI: m/z=364.0 [M+1]⁺) and 71b (yield: 38.1%, MS-ESI: m/z=318.0 [M+1]⁺) were prepared according to the same procedure for the preparation of 68a, 69a, 70a and 71a, from compound 67b. The same procedure for preparing compound 225 was used to prepare compound 227 from compound 71b as a black solid (yield: 10.4%). ¹H NMR (400 MHz, DMSO): 0.990 (s, 9H), 1.347 (m, 2H), 1.661 (m, 1H), 2.711 (m, 2H), 3.081 (s, 3H), 7.260 (t, 1H, J=6.4 Hz), 7.613 (m, 3H), 7.751 (m, 2H), 9.026 (d, 1H, J=6.4 Hz), 10.273 (s, 1H), 14.105 (s, 1H), 14.243 (s, 1H). MS-ESI: m/z=519.1 [M+1]⁺.

### Preparation of Compound 228

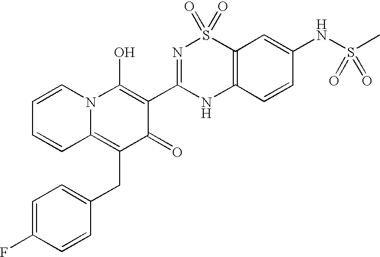

Compounds 68c (MS-ESI: m/z=245.9 [M+1]⁺), 69c (MS-ESI: m/z=315.9 [M+1]⁺), 70c (MS-ESI: m/z=388.1 [M+1]⁺) and 71c (yield: 16.7%, MS-ESI: m/z=342.0 [M+1]⁺) were prepared according to the same procedure for the preparation of 68a, 69a, 70a and 71a, from compound 67c. The same procedure for preparing compound 225 was used to prepare compound 228 from compound 71c as a dark green solid (yield: 16.7%). ¹H NMR (400 MHz, DMSO): 3.170 (S, 3H), 4.0 (S, 2H), 7.153 (t, 2H, J=8.4 Hz), 7.373 (m, 3H), 21 (s, 3H), 7.701 (m, 2H), 7.795 (m, 1H), 7.878 (m, 1H), 7.987 (m, 1H), 9.187 (d, 1H, J=7.2 Hz), 10.352 (s, 1H), 14.315 (s, 1H), 14.348 (s, 1H). MS-ESI: m/z=543.1 [M+1]⁺.

### Example 11

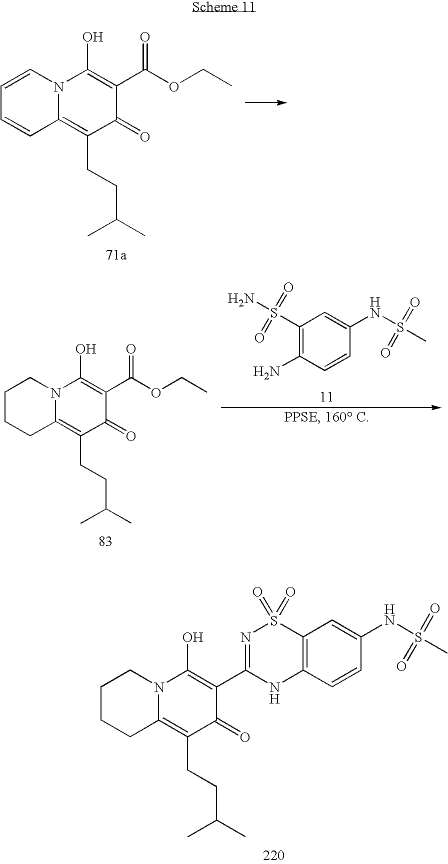

### Preparation of Compound 83

A mixture of compound 71a (50 mg, 0.17 mmol) and 10% Pd/C (20 mg) in acetic acid (5 mL) was stirred under 15 psi of H₂ at 60° C. for 4 hour. Then the mixture was cooled to r.t. and Pd/C was filtered off. The solvent was removed under vacuum to give the pure product as brown oil without further purification (37 mg, yield: 71.2%). MS-ESI: m/z=307.9 [M+1]⁺.

### Preparation of Compound 220

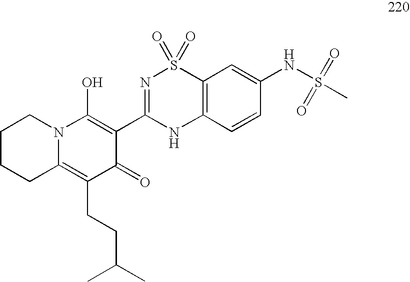

The same procedure for preparing compound 216 was used to prepare compound 220 from compound 83 as a gray solid (yield: 10%). ¹H NMR (400 MHz, DMSO): 0.930 (d, 6H, J=6.4 Hz), 1.282 (t, 2H, J=3.2 Hz), 1.595 (t, 1H, J=6.4 Hz), 1.775 (d, 2H, J=6.4 Hz), 1.853 (d, 2H, J=5.6 Hz), 2.490 (m, 2H), 2.900 (d, 2H, J=5.6 Hz), 3.067 (s, 3H), 3.970 (t, 2H, J=5.6 Hz), 7.549 (d, 1H, J=8.4 Hz), 7.624 (t, 2H, J=5.6 Hz), 10.241 (s, 1H), 14.252 (s, 1H), 14.707 (s, 1H). MS-ESI: m/z=509.0 [M+1]⁺.

### Example 12

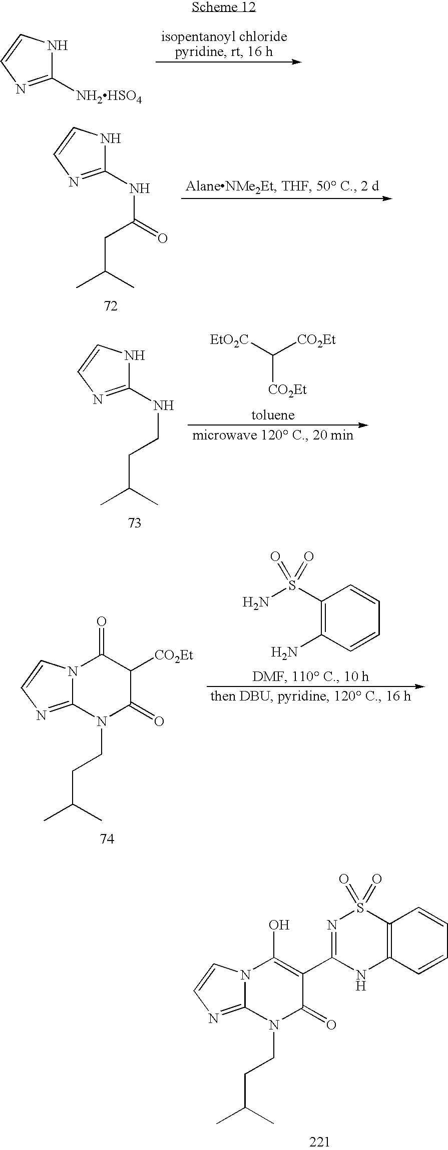

### Preparation of N-(1H-Imidazol-2-yl)-3-methyl-butyramide (72)

To a stirred suspension of 2-aminoimidazole hydrogen sulphate (5.80 g, 22.0 mmol) in dry pyridine (28 mL) was added isovaleryl chloride (2.64 mL, 22.2 mmol, d 0.989) and the brown suspension stirred at rt overnight before being poured into water (200 mL). The mixture was filtered and the solid washed with further water (50 mL) and air-dried to afford the title compound 72 as an off-white solid (1.66 g, 45%). ¹H NMR (250 MHz, DMSO-d₆) δ 11.51 (bs, 1H), 11.02 (bs, 1H), 6.68 (s, 2H), 2.19 (d, 2H), 2.06 (spt, 1H), 0.90 (d, 6H).

### Preparation of (1H-Imidazol-2-yl)-(3-methyl-butyl)-amine (73)

To a stirred suspension of amide 72 (1.80 g, 10.8 mmol) in dry THF at 4° C. under a nitrogen atmosphere, was added cautiously by syringe a solution of alane dimethylethylamine complex in toluene (64 mL, 0.5M, 32 mmol). (CAUTION: significant gas evolution occurred during the first third of addition). After the addition was completed, the suspension was allowed to warm to rt, then heated with stirring at 50° C. for 2d. The mixture was cooled to 4° C. and quenched by careful addition of water-saturated THF (10 mL), water (50 mL) and 10% w/v sodium potassium tartrate (50 mL). The mixture was extracted with EtOAc (3×100 mL) and the combined organic layers washed with saturated brine (30 mL), dried (Na₂SO₄), filtered and evaporated. The crude residue was purified by flash chromatography (silica, eluting with 20% EtOAc in heptane, 50% EtOAc in heptane, neat EtOAc, and 10% MeOH in EtOAc containing aqueous ammonia) to afford the title compound 73 as a red oil (896 mg, 54%). ¹H NMR (250 MHz, CDCl₃) δ 6.56 (s, 2H), 3.17 (t, 2H), 1.58 (spt, 1H), 1.39 (qd, 2H) 0.83 (d, 6H). MS m/e 154 (MH⁺).

### Preparation of 8-(3-Methyl-butyl)-5,7-dioxo-5,6,7,8-tetrahydro-imidazo[1,2-a]pyrimidine-6-carboxylic acid ethyl ester (74)

A microwave tube containing amine 73 (100 mg, 0.653 mmol), triethyl methanetricarboxylate (151 mg, 0.653 mmol), toluene (2.0 mL) and a stirrer bar was sealed and irradiated in a CEM Discover microwave (150W, 140° C., 10 minute ramp time, 20 minute hold time). The mixture was purified by flash chromatography (silica, eluting with 100% DCM followed by 5% MeOH in DCM then 10% MeOH in DCM) to afford the title compound 74 as a dark green oil which was judged pure enough to use in the subsequent step (58 mg, 30%). ¹H NMR (500 MHz, CD₃OD) δ 7.51 (d, 1H), 7.19 (d, 1H), 4.18 (q, 2H), 3.96 (t, 2H), 1.61 (spt, 1H), 1.48 (m, 2H), 1.23 (t, 3H), 0.90 (d, 6H). MS m/e 294 (MH⁺).

### Preparation of 6-(1,1-Dioxo-1,4-dihydro-1lambda*6*-benzo[1,2,4]thiadiazin-3-yl)-5-hydroxy-8-(3-methyl-butyl)-8H-imidazo[1,2-a]pyrimidin-7-one (221)

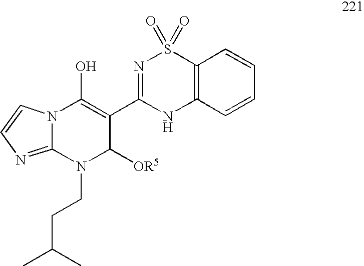

To a solution of ester 74 (58 mg, 0.198 mmol) in dry DMF (1 mL) was added 2-aminobenzenesulfonamide (36 mg, 0.207 mmol) and the solution stirred at 100° C. for 10 h. The solvent was evaporated under reduced pressure and replaced with dry pyridine (2 mL). DBU (136 mg, 0.895 mmol) was added and the dark green solution heated at 120° C. for 16 h. The solvent was evaporated and the brown oil dissolved in MeOH and purified by preparative HPLC (high pH method). Evaporation of product-containing fractions under reduced pressure afforded the title compound 221 as a white solid (5.1 mg, 6%). ¹H NMR (250 MHz, CD₃OD) δ 7.77 (d, 1H), 7.60 (dd, 1H), 7.45 (d, 1H), 7.37 (d, 1H), 7.29 (dd, 1H), 6.93 (d, 1H), 4.14 (m, 2H), 1.72-1.60 (m, 3H), 1.00 (d, 6H). MS m/e 402 (MH⁺).

### Preparation of N-{3-[5-Hydroxy-8-(3-methyl-butyl)-7-oxo-7,8-dihydro-imidazo[1,2-a]pyrimidin-6-yl]-1,1-dioxo-1,4-dihydro-1lambda*6*-benzo[1,2,4]thiadiazin-7-yl}-methanesulfonamide (222)

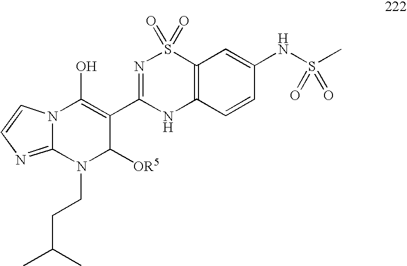

To a stirred solution of ester 74 (70 mg, 0.239 mmol) in dry pyridine (2 mL) was added 2-amino-5-methanesulfonylaminobenzenesulfonamide (76 mg, 0.288 mmol) and the solution stirred at 110° C. for 3 h. DBU (110 mg, 0.717 mmol) was added and the dark grey solution heated at 110° C. for 16 h. After cooling to rt, the solution was evaporated under reduced pressure, and the brown residue was partitioned between 0.1M citric acid solution (25 mL) and ethyl acetate (3×25 mL). The combined organic layers were dried (Na₂SO₄) and concentrated in vacuo to give a brown oil. MeOH was added and the solution stirred for 10 minutes. The resulting suspension was filtered and the brown solid dissolved in DMSO and purified by preparative HPLC (high pH method). Evaporation of product-containing fractions under reduced pressure afforded the title compound 222 as a brown solid (1.5 mg, 2%). ¹H NMR (500 MHz, CD₃OD) δ 7.57 (s, 1H), 7.41 (d, 1H), 7.36 (s, 1H), 7.21 (d, 1H), 6.84 (s, 1H), 4.04-4.07 (m, 2H), 2.90 (s, 3H), 1.60-1.65 (m, 1H), 1.50-1.55 (m, 2H), 0.90 (d, 6H). MS m/e 493 (M−1⁻).

### Example 13

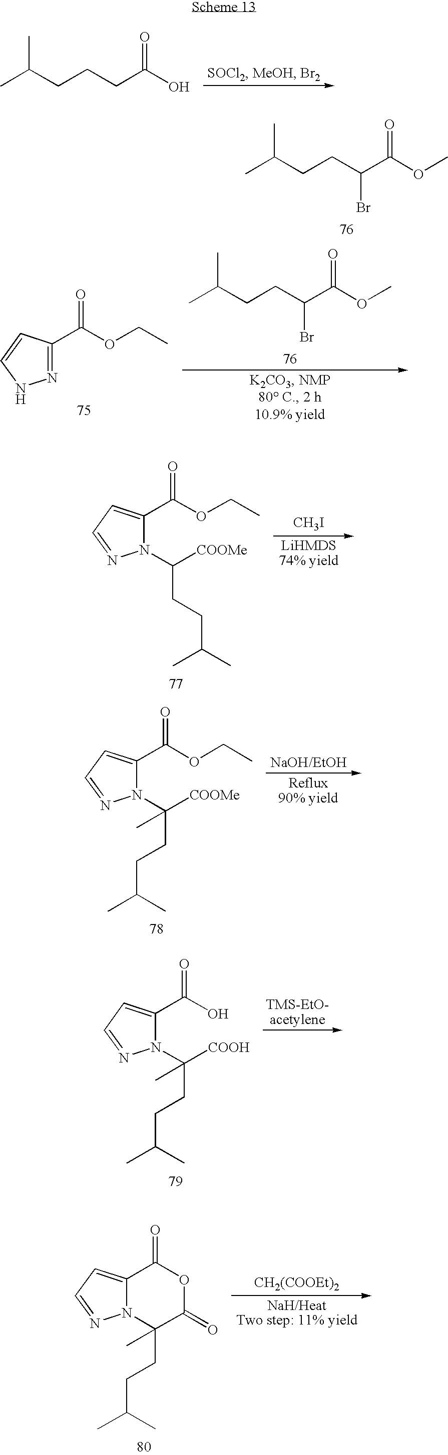

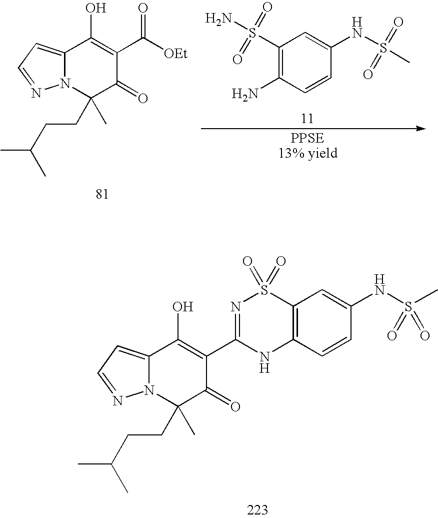

Preparation of Compound 77 Compound 75 was prepared according to J. Heterocycl. Chem., 2003, 487. Compound 75 (10 g, 71.4 mmol), compound 76 (20 g, 89.2 mmol) and K₂CO₃ (12.328 g, 89.2 mmol) were dissolved in NMP (60 ml), and the mixture was heated to 80° C. for 2 h. The reaction mixture was allowed to cool and diluted with a mixture of ethyl acetate: water=3:1 (500 ml). The water layer was washed with EtOAc (3×100 ml), and the organic layers were dried over Na₂SO₄, and concentrated in vacuo. The residue was purified on silica gel (PE:EA=100:1 to 20:1) to give compound 77 as yellow oil (2.2 g, 10.9%). ¹H NMR (400 MHz, CDCl₃): 0.860 (q, 6H, J=4 Hz), 0.978 (m, 1H), 1.213 (m, 1H), 1.372 (t, 3H, J=7.2 Hz), 1.555 (m, 2H), 2.329 (m, 2H), 3.709 (s, 3H), 4.334 (q, 2H, J=7.2 Hz), 5.907 (q, 1H, J=5.2 Hz), 6.896 (d, 1H, J=2 Hz), 7.587 (d, 1H, J=2 Hz). MS-ESI: m/z=283.1 [M+1]⁺.

### Preparation of Compound 78

Compound 77 (2.2 g, 7.792 mmol), was dissolved in THF (15 ml), the temperature was allowed to cool to −78° C., and LiHMDS (1 M in THF, 11.67 ml) was added drop-wise. The reaction mixture was allowed to stir at −78° C. for 1 h. Then CH₃I (2.212 g, 15.584 mmol) was added slowly. The reaction mixture was stirred at −78° C. for 4 h. When the temperature was allowed to r.t, the mixture was poured into water, and extracted with EtOAc, the combined organic lagers were dried (Na₂SO₄), filtered and the solvent was evaporated, and purified on silica gel (only PE as elute) to give compound 78 as yellow oil (1.7 g, 74%). ¹H NMR (400 MHz, CDCl₃): 0.542 (m, 1H), 0.806 (q, 6H, J=6.8 Hz), 1.227 (m, 1H), 1.333 (t, 3H, J=7.2 Hz), 1.435 (m, 1H), 1.855 (s, 3H), 2.315 (m, 2H), 3.693 (s, 3H), 4.282 (q, 2H, J=7.2 Hz), 6.957 (d, 1H, J=2 Hz), 7.496 (d, 1H, J=2 Hz). MS-ESI: m/z=296.9 [M+1]⁺.

### Preparation of Compound 79

Compound 78 (0.7 g, 2.362 mmol), was dissolved in EtOH (10 ml), then was added NaOH (0.945 g, 23.62 mmol) in H₂O (3 ml). The reaction mixture was refluxed for 4 h. When the mixture was cooled, it was acidified with 3 M HCl until PH=2. Then the mixture was extracted with EtOAc, the combined organic lagers were dried (Na₂SO₄), filtered and the solvent was evaporated to obtain compound 79 as solid (0.54 g, 90%). MS-ESI: m/z=255.0 [M+1]⁺.

### Preparation of Compound 80

To compound 79 (540 mg, 2.124 mmol) in a solvent of (CH₂)₂Cl₂ (10 ml) was added compound TMS-EtO-acetylene (0.453 g, 3.185 mmol). And then the mixture was stirred at 70° C. for 3 days. After concentration of the reaction mixture by rotary evaporator, compound 80 was obtained (used directly in the next step). ¹H NMR (400 MHz, CDCl₃): 0.502 (m, 1H), 0.787 (q, 6H, J=6.4 Hz), 0.977 (m, 1H), 1.439 (m, 1H), 1.967 (s, 3H), 2.292 (m, 2H), 7.138 (d, 1H, J=2 Hz), 7.785 (d, 1H, J=2 Hz).

### Preparation of Compound 81

To a slurry of NaH (60%, 425 mg, 10.625 mmol) in 2 ml anhydrous DMA at 10° C. under N₂ was added diethyl malonate (0.68 g, 4.25 mmol) drop-wise. The mixture was stirred at ambient temperature for 30 min, treated with compound 80 (crude, 2.124 mmol by theoretical weight), and heated at 120° C. for 4 h. The mixture was cooled to ambient temperature and partitioned between ethyl acetate and cold water adjusting the PH to 3 with 3M HCl. The organic layers were dried (Na₂SO₄), filtered and was concentrated under vacuum. The residue was purified by TLC (DCM: MeOH=9:2) to obtain the desired compound 81 (70 mg, 11% in two steps). ¹H NMR (400 MHz, MeOD): 0.338 (m, 1H), 0.767 (q, 6H, J=6.4 Hz), 0.980 (m, 1H), 1.348 (m, 4H), 1.688 (s, 3H), 2.207 (m, 2H), 4.285 (q, 2H, J=7.2 Hz), 6.727 (s, 1H), 7.624 (s, 1H). MS-ESI: m/z=306.9 [M+1]⁺.

### Preparation of Compound 223

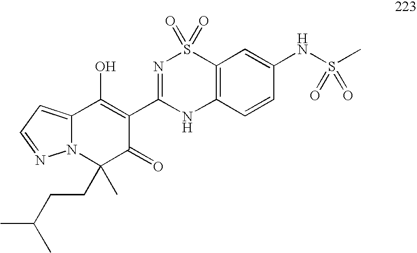

Compound 81 (70 mg, 0.251 mmol) was added at 160° C. to PPSE (4 ml). The mixture becomes clear within a few minutes. 2-amino-5-(methylsulfonamido)benzenesulfonamide (1e.q) was added and the solution stirred for 1.5 h at 160° C. The cooled mixture was poured in ice/water, and extracted with EtOAc. The combined organic lagers were dried (Na₂SO₄), filtered and the solvent was evaporated, and purified by TLC (EA) to obtain compound 223 (15 mg, 13%). ¹H NMR (400 MHz, MeOD): 0.363 (m, 1H), 0.772 (q, 6H, J=6.4 Hz), 0.972 (m, 1H), 1.373 (m, 1H), 1.743 (s, 3H), 2.188 (m, 1H), 2.317 (m, 1H), 3.018 (s, 3H), 6.799 (d, 1H, J=1.6 Hz), 7.320 (d, 1H, J=8.8 Hz), 7.527 (d, 1H, J=2.4 Hz), 7.659 (d, 1H, J=2 Hz), 7.693 (d, 1H, J=2.4 Hz). MS-ESI: m/z=508.0 [M+1]⁺.

### Example 14

A general synthetic scheme for the preparation of polymerase inhibitors, described in this section is illustrated in Scheme 14 below and exemplified by the following description of the synthesis of compound 229.

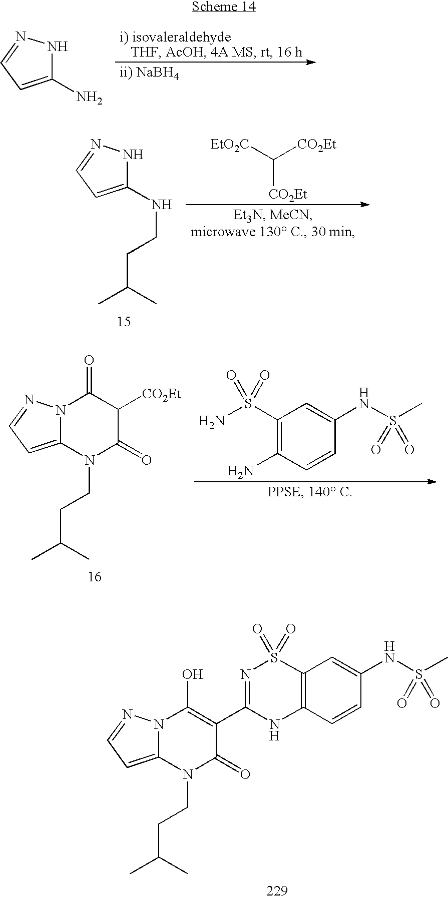

### Preparation of (3-Methyl-butyl)-(2H-pyrazol-3-yl)-amine 15

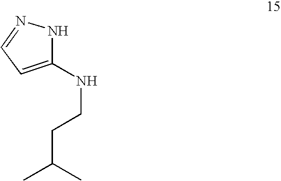

To a stirred solution of 3-aminopyrazole (2.75 g, 33.1 mmol) in THF (40 ml) was added isovaleraldehyde (3.11 g, 36.2 mmol) and acetic acid (2.18 g, 36.3 mmol) and 4 Å molecular sieves. After 30 min, sodium borohydride (1.37 g, 36.0 mmol) was added in portions over 20 min and the mixture stirred for 3 h. Water (15 mL) was added and the pH raised to 14 with 1M NaOH. The mixture was extracted with EtOAc (3×50 ml), the combined organic layers dried (Na₂SO₄), the mixture filtered and the filtrate concentrated in vacuo. The resultant oil was chromatographed (silica: eluting with 50% EtOAc in heptane followed by 100% EtOAc, then 5% MeOH in EtOAc) to afford the title compound as a yellow oil (410 mg, 8%); ¹H NMR (250 MHz, CDCl₃) δ 6.56 (s, 2H), 3.17 (t, 2H), 1.58 (m, 1H), 1.40 (q, 2H), 0.82 (d, 6H); MS m/e 154 (MH)⁺.

### Preparation of benzyl-(2H-pyrazol-3-yl)-amine 18

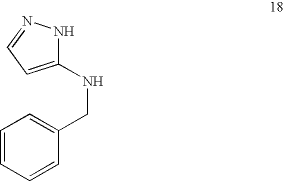

Pyrazole amine 18 was prepared according to the procedure described for pyrazole amine 15, except that benzaldehyde was used instead of isovaleraldehyde. Molecular sieves and acetic acid were omitted from the reaction mixture; 86%; MS m/e 174 (MH)⁺.

### Preparation of (4-Fluoro-benzyl)-(2H-pyrazol-3-yl)-amine 19

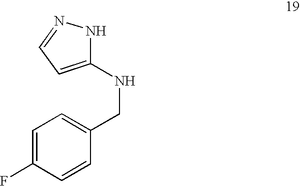

Pyrazole amine 19 was prepared according to the procedure described for pyrazole amine 15, except that 4-fluorobenzaldehyde was used instead of isovaleraldehyde. Molecular sieves and acetic acid were omitted from the reaction mixture; 46%, MS m/e 192 (MH)⁺.

### Preparation of [2-(4-Fluoro-phenyl)-ethyl]-(2H-pyrazol-3-yl)-amine 20

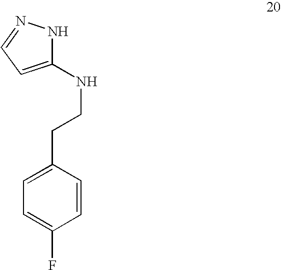

Pyrazole amine 20 was prepared according to the procedure described for pyrazole amine 15, except that 4-fluorophenethylaldehyde was used instead of isovaleraldehyde. Molecular sieves and acetic acid were omitted from the reaction mixture; 73%; MS m/e 206 (MH)⁺.

### Preparation of (3-Methyl-butyl)-(5-methyl-2H-pyrazol-3-yl)-amine 21

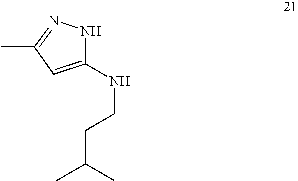

Pyrazole amine 21 was prepared according to the procedure described for 15, except that 5-amino-3-methylpyrazole was used instead of 3-aminopyrazole. Molecular sieves and acetic acid were omitted from the reaction mixture; 26%; MS m/e 168 (MH)⁺.

### Preparation of (5-Cyclopropyl-2H-pyrazol-3-yl)-(3-methyl-butyl)-amine 22

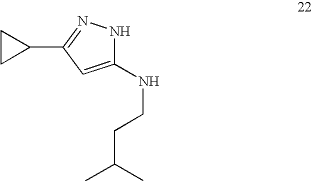

Pyrazole amine 22 was prepared according to the procedure described for pyrazole amine 15, except that 5-amino-3-cyclopropyl-pyrazole was used instead of 3-aminopyrazole. Molecular sieves and acetic acid were omitted from the reaction mixture; 25% MS m/e 194 (MH)⁺.

### Preparation of 4-(3-Methyl-butyl)-5,7-dioxo-4,5,6,7-tetrahydro-pyrazolo[1,5-a]pyrimidine-6-carboxylic acid ethyl ester 16

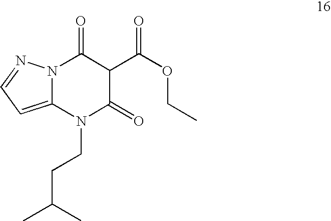

A solution of amine 15 (140 mg, 0.915 mmol) in acetonitrile (3 mL) was placed in a microwave tube containing a stirrer bar. Et₃N (0.300 ml) and triethylmethane tricarboxylate (270 mg, 1.16 mmol) were added, the tube sealed and the solution irradiated in a CEM Discover microwave (130° C., 30 min, 150 W). The solution was concentrated in vacuo and the orange oil chromatographed (silica, eluting with neat DCM followed by 4% MeOH in DCM) to afford the title compound as an orange oil (163 mg, 61%); MS (−ive ion) m/e 292 (M−1)⁻.

### Preparation of 4-Benzyl-5,7-dioxo-4,5,6,7-tetrahydro-pyrazolo[1,5-a]pyrimidine-6-carboxylic acid ethyl ester 23

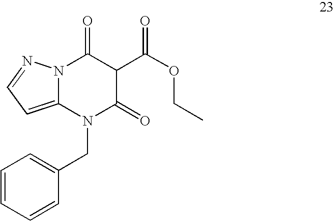

The title compound was prepared according to the procedure described for 16, except that amine 18 was used as the cyclisation substrate; 79%; MS m/e 314 (MH)⁺.

### Preparation of 4-(4-Fluoro-benzyl)-5,7-dioxo-4,5,6,7-tetrahydro-pyrazolo[1,5-a]pyrimidine-6-carboxylic acid ethyl ester 24

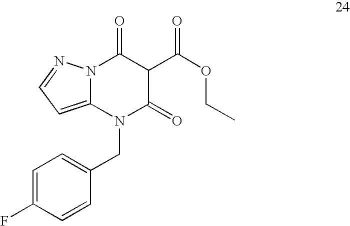

The title compound was prepared according to the procedure described for 16, except that amine 19 was used as the cyclisation substrate; 63%; MS m/e 332 (MH)⁺.

### Preparation of 4-[2-(4-Fluoro-phenyl)-ethyl]-5,7-dioxo-4,5,6,7-tetrahydro-pyrazolo[1,5-a]pyrimidinecarboxylic acid ethyl ester 25

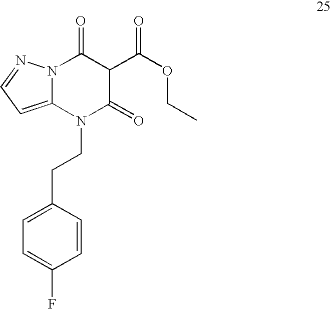

The title compound was prepared according to the procedure described for 16, except that amine 20 was used as the cyclisation substrate; 17%; MS m/e 346 (MH)⁺.

### Preparation of 2-Methyl-4-(3-methyl-butyl)-5,7-dioxo-4,5,6,7-tetrahydro-pyrazolo[1,5-a]pyrimidine-6-carboxylic acid ethyl ester 26

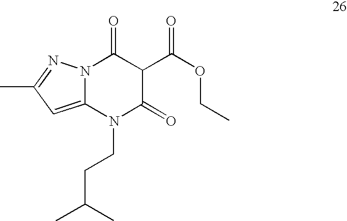

The title compound was prepared according to the procedure described for 16, except that amine 21 was used as the cyclisation substrate; 50%; MS m/e 308 (MH)⁺.

### Preparation of 2-Cyclopropyl-4-(3-methyl-butyl)-5,7-dioxo-4,5,6,7-tetrahydro-pyrazolo[1,5-a]pyrimidine-6-carboxylic acid ethyl ester 27

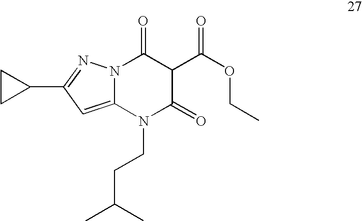

The title compound was prepared according to the procedure described for 16, except that amine 22 was used as the cyclisation substrate; 90%; MS m/e 334 (MH)⁺.

### Preparation of N-{3-[7-Hydroxy-4-(3-methyl-butyl)-5-oxo-4,5-dihydro-pyrazolo[1,5-a]pyrimidin-6-yl]-1,1-dioxo-1,4-dihydro-1lambda*6*-benzo[1,2,4]thiadiazin-7-yl}-methanesulfonamide 229

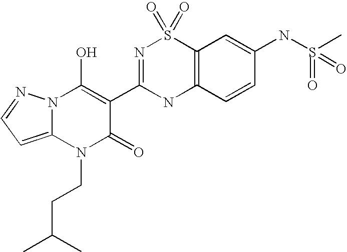

To a mixture of ester 16 (84 mg, 0.287 mmol) and 2-amino-5-methanesulfonylaminobenzenesulfonamide (84 mg, 0.316 mmol) was added polyphosphoric acid trimethylsilyl ester (PPSE, 2.0 mL) and the brown suspension heated at 140° C. for 2 h. The mixture was cooled to 40° C., water (10 mL) added and the brown mass stirred until a filterable mixture formed. The mixture was filtered and the brown solid dissolved in 80% DMSO in MeOH and filtered. The filtrate was submitted to reverse-phase chromatography (high pH method) to afford 229 as an off-white solid; (4.1 mg, 3%); ¹H NMR (500 MHz, DMSO-d₆) δ 9.92 (s, 1H), 7.67 (s, 1H), 7.48 (s, 1H), 7.44 (d, 1H), 7.33 (d, 1H), 5.92 (s, 1H), 3.89 (t, 2H), 3.00 (s, 3H), 1.65 (m, 1H), 1.48 (m, 2H), 0.94 (d, 6H); MS (−ive ion) m/e 493 (M−1)⁻.

### Preparation of N-[3-(4-Benzyl-7-hydroxy-5-oxo-4,5-dihydro-pyrazolo[1,5-a]pyrimidin-6-yl)-1,1-dioxo-1,4-dihydro-1lambda*6*-benzo[1,2,4]thiadiazin-7-yl]-methanesulfonamide 230

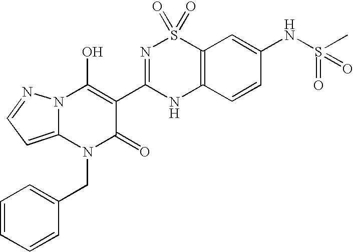

Compound 230 was prepared according to the procedure described for compound 229, except that ester 23 was used and final HPLC purification was carried out via the low pH method; 5%; ¹H NMR (500 MHz, CD₃OD) δ 7.74 (s, 1H), 7.68 (s, 1H), 7.54 (d, 1H), 7.45 (d, 1H), 7.30-7.19 (m, 5H), 6.01 (s, 1H), 5.21 (s, 2H), 2.98 (s, 3H); MS (−ive ion) m/e 513 (M−1)⁻.

### Preparation of N-{3-[4-(4-Fluoro-benzyl)-7-hydroxy-5-oxo-4,5-dihydro-pyrazolo[1,5-a]pyrimidin-6-yl]-1,1-dioxo-1,4-dihydro-1lambda*6*-benzo[1,2,4]thiadiazin-7-yl}-methanesulfonamide 231

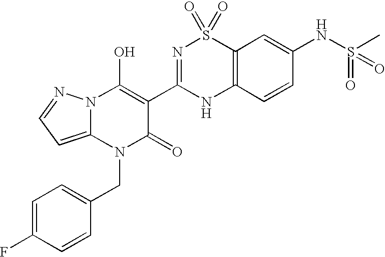

Compound 231 was prepared according to the procedure described for compound 229, except that ester 24 was used and final HPLC purification was carried out via the low pH method; 1%; ¹H NMR (500 MHz, CD₃OD) 7.58 (m, 2H), 7.39 (d, 1H), 7.28 (m, 1H), 7.20 (d, 1H), 7.09 (s, 1H), 6.93 (m, 2H), 6.74 (m, 1H), 6.60 (d, 1H), 5.09 (s, 2H), 2.90 (s, 3H); MS m/e 533 (MH)⁺.

### Preparation of N-(3-{4-[2-(4-Fluoro-phenyl)-ethyl]-7-hydroxy-5-oxo-4,5-dihydro-pyrazolo[1,5-a]pyrimidin-6-yl}-1,1-dioxo-1,4-dihydro-1lambda*6*-benzo[1,2,4]thiadiazin-7-yl)-methanesulfonamide 232

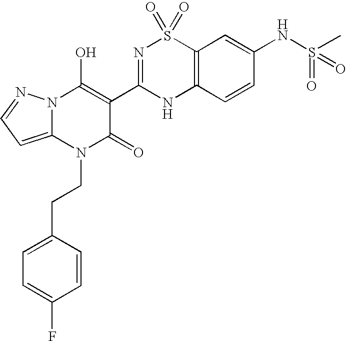

Compound 232 was prepared according to the procedure described for compound 229, except that ester 25 was used and final HPLC purification was carried out via the low pH method; 1%; ¹H NMR (500 MHz, DMSO-d₆); 13.95 (s, 1H), 9.94 (s, 1H), 7.69 (s, 1H), 7.50 (s, 1H), 7.45 (d, 1H), 7.38-7.34 (m, 3H), 7.10 (dd, 2H), 6.00 (s, 1H), 4.08 (t, 2H), 3.02 (s, 3H), 2.93 (t, 2H); MS m/e 547 (MH)⁺.

### Preparation of N-{3-[7-Hydroxy-2-methyl-4-(3-methyl-butyl)-5-oxo-4,5-dihydro-pyrazolo[1,5-a]pyrimidin-6-yl]-1,1-dioxo-1,4-dihydro-1lambda*6*-benzo[1,2,4]thiadiazin-7-yl}-methanesulfonamide 233

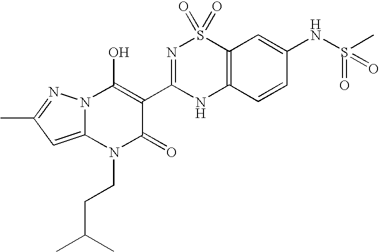

Compound 233 was prepared according to the procedure described for compound 229, except that ester 26 was used; (4%); ¹H NMR (500 MHz, DMSO-d₆) 7.48 (s, 1H), 7.41 (d, 2H), 7.29 (d, 2H), 5.72 (s, 1H), 3.84 (t, 2H), 3.00 (s, 3H), 2.22 (s, 3H), 1.67-1.62 (m, 1H), 1.52-1.47 (m, 2H), 0.94 (d, 6H); MS (−ive ion) m/e 507 (M−1)⁻.

### Preparation of N-{3-[2-cyclopropyl-7-Hydroxy-4-(3-methyl-butyl)-5-oxo-4,5-dihydro-pyrazolo[1,5-a]pyrimidin-6-yl]-1,1-dioxo-1,4-dihydro-1lambda*6*-benzo[1,2,4]thiadiazin-7-yl}-methanesulfonamide 234

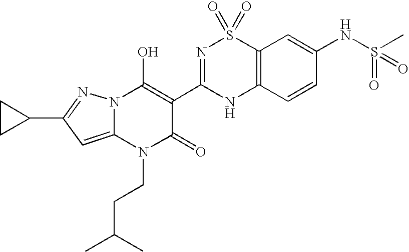

Compound 234 was prepared according to the procedure described for thiadiazine 229, except that ester 27 was used; (1%); insufficient material for NMR; MS (−ive ion) m/e 533 (M−1)⁻.

### Example 15

A general synthetic scheme for the preparation of polymerase inhibitors, described in this section is illustrated in Scheme 15 below and exemplified by the following description of the synthesis of compound 235

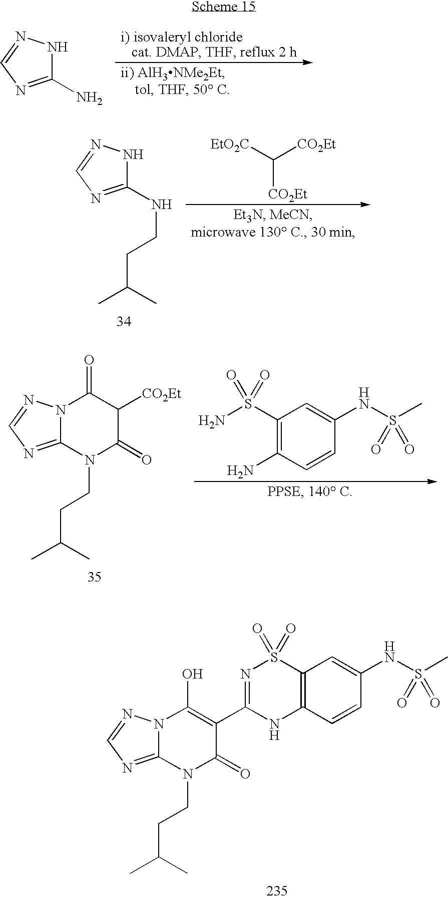

### Preparation of (3-Methyl-butyl)-(2H-[1,2,4]triazol-3-yl)-amine 34

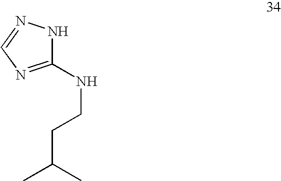

A mixture of 3-aminotriazole (5.00 g, 59.5 mmol) and isovaleryl chloride (7.14 g, 59.5 mmol) containing a crystal of DMAP was refluxed in THF (250 ml) for 2 h. The mixture was cooled and filtered. The solid was washed with more THF (2×25 ml) to afford amide 33 as a white solid (7.68, 78%); MS m/e 169 (MH)⁺

To a suspension of amide 33 (1.50 g, 8.93 mmol) in THF (30 ml) at 4° C. was added a solution of alane dimethylethylamine complex in toluene (0.5M, 53 ml, 26.5 mmol) over 20 min. The reaction mixture was then stirred at 45° C. for 2d. After cooling in ice, the mixture was quenched by the sequential addition of 10% water in THF, saturated Rochelle's salts and water. The mixture was extracted with EtOAc (3×100 ml), the combined organic layers dried (Na₂SO₄), and the mixture filtered. The filtrate was concentrated in vacuo and chromatographed (silica: eluent 50% EtOAc in heptane, neat EtOAc then 10% MeOH in EtOAc) to afford the title compound as a yellow oil; 240 mg (17%); MS m/e 155 (MH)⁺

### Preparation of 4-(3-Methyl-butyl)-5,7-dioxo-4,5,6,7-tetrahydro-[1,2,4]triazolo[1,5-a]pyrimidine-6-carboxylic acid ethyl ester 35

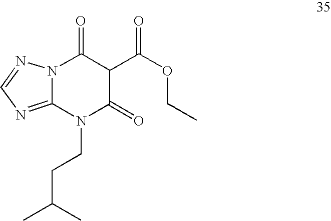

The title compound was prepared according to the procedure described for compound 16 of Scheme 14 except that amine 34 was used; 25%; ¹H NMR (500 MHz, CD₃OD) δ 7.89 (s, 1H), 4.24 (q, 2H), 3.98 (q, 2H), 1.62-1.40 (m, 3H), 1.23 (t, 3H), 0.84 (d, 6H).

### Preparation of N-{3-[7-Hydroxy-4-(3-methyl-butyl)-5-oxo-4,5-dihydro-[1,2,4]triazolo[1,5-a]pyrimidin-6-yl]-1,1-dioxo-1,4-dihydro-1lambda*6*-benzo[1,2,4]thiadiazin-7-yl}-methanesulfonamide 235

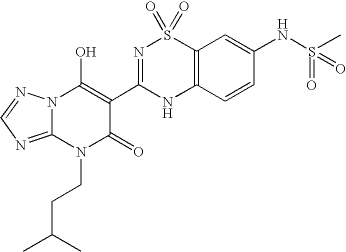

Compound 235 was prepared according to the procedure described for compound 229, except that ester 35 was used and the final HPLC purification was run using the low pH method, affording the title compound as an off-white solid; 3%; ¹H NMR (500 MHz, CD₃OD) δ 7.98 (s, 1H), 7.69 (s, 1H), 7.53 (d, 1H), 7.34 (d, 1H), 4.19 (t, 2H), 1.74-1.64 (m, 3H), 1.02 (d, 6H); MS m/e 496 (MH)⁺.

### Example 16

A general synthetic scheme for the preparation of polymerase inhibitors, described in this section is illustrated in Scheme 16 below and exemplified by the following description of the synthesis of compound 236:

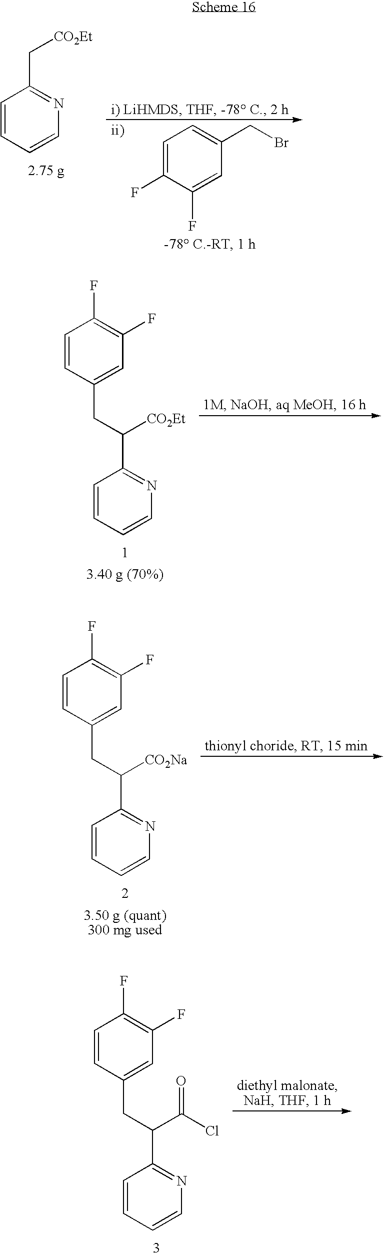

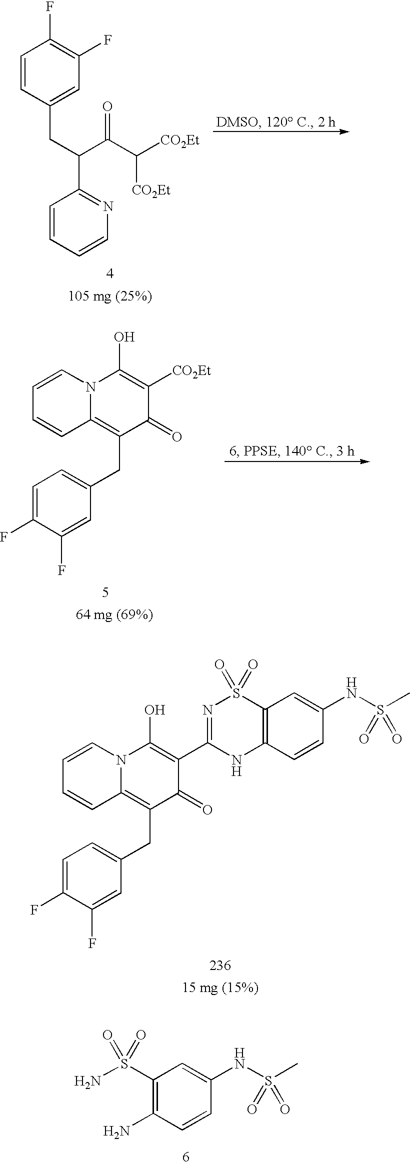

### Preparation of 3-(3,4-Difluoro-phenyl)-2-pyridin-2-yl-propanoic acid ethyl ester 1

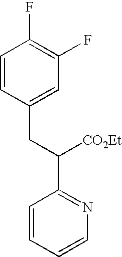

To a stirred solution of 2-pyridylacetic acid, ethyl ester (2.75 g, 16.7 mmol) in THF (70 mL) at −78° C. under nitrogen was added, dropwise via syringe over 15 min, a solution of lithium bis(trimethylsilyl)amide (1M in THF, 16.7 mL, 16.7 mmol) and the solution stirred for 2 h at this temperature, whereupon a white precipitate formed. Neat 3,4-difluorobenzyl bromide was added via syringe and the mixture allowed to warm to RT with stirring. After 1 h at RT, water (30 mL) was added and the mixture extracted with EtOAc (3×30 mL). The combined organic extracts were dried (Na₂SO₄), the mixture filtered and the filtrate evaporated to dryness to afford an orange oil which was chromatographed (silica, eluent 20% EtOAc in heptane) giving compound 236 as a yellow oil (3.40 g, 70%); MS m/e 292 (MH)⁺.

### Preparation of 3-(3,4-Difluoro-phenyl)-2-pyridin-2-yl-propanoic acid, sodium salt 2

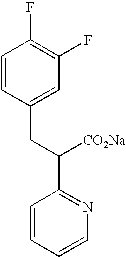

To a stirred solution of ester 1 (3.40 g, 11.7 mmol) in MeOH (25 mL) at rt was added an aqueous solution of sodium hydroxide (1M, 11.7 mL, 11.7 mmol) and the cloudy mixture stirred for 5 h or until complete hydrolysis had occurred (determined by LCMS). The solvents were evaporated in vacuo, and residual solvent removed by threefold azeotrope with DCM to afford the title compound as a white solid; (3.50 g, quant.); MS m/e 264 (MH)⁺.

### Preparation of 2-[3-(3,4-Difluoro-phenyl)-2-pyridin-2-yl-propionyl]-malonic acid diethyl ester 4

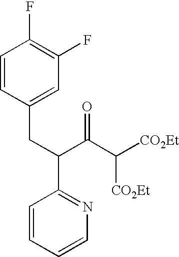

Thionyl chloride (2 mL) was added to solid sodium salt 2 (300 mg, 1.05 mmol) and the mixture stirred for 15 min until a red solution formed. The thionyl chloride was evaporated and the residue azeotroped with anhydrous THF three times to afford the acid chloride 3.

To a solution of diethyl malonate (336 mg, 2.10 mmol) in anhydrous THF at 4° C., was added sodium hydride (60% by weight in mineral oil, 84 mg, 2.10 mmol) in portions and the mixture stirred until hydrogen evolution ceased. To this solution was added dropwise a solution of 3 in anhydrous THF and the red solution stirred for 1 h. A solution of citric acid (10 mL, 10% w/v) was added and the mixture extracted with EtOAc (3×15 mL). The combined organic extracts were dried (Na₂SO₄), the mixture filtered and the filtrate evaporated to dryness to afford a red oil which was chromatographed (silica, eluent 25% EtOAc in heptane) to afford the title compound as a red oil; 105 mg, 25%; MS m/e 406 (MH)⁺.

### Preparation of 1-(3,4-Difluoro-benzyl)-4-hydroxy-2-oxo-2H-quinolizine-3-carboxylic acid ethyl ester 5

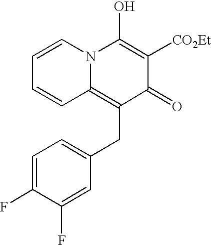

A stirred solution of diester 4 (105 mg, 0.258 mmol) in DMSO (2 mL) was heated at 120° C. for 2 h. The solution was allowed to cool, water added (5 mL) and the mixture extracted with EtOAc (3×15 mL). The combined organic extracts were washed with water (4×5 mL), dried (Na₂SO₄), the mixture filtered and the filtrate evaporated to dryness to afford an orange solid which was chromatographed (silica, eluent 25% EtOAc in heptane rising to 50% EtOAc in heptane) to afford the title compound as a yellow solid; 64 mg, 69%; MS m/e 406 (MH)⁺.

### Preparation of N-{3-[1-(3,4-Difluoro-benzyl)-4-hydroxy-2-oxo-2H-quinolizin-3-yl]-1,1-dioxo-1,4-dihydro-1lambda′6′-benzo[1,2,4]thiadiazin-7-yl}-methanesulfonamide 236

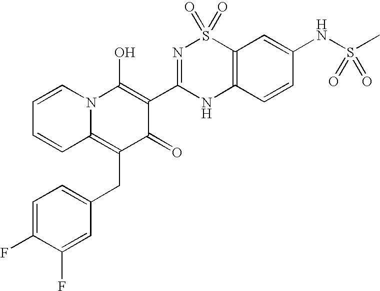

To a mixture of ester 5 (64 mg, 0.178 mmol) in PPSE (1-2 mL) was added aminosulfonamide 6 (Dragovich et al., Synth. Commun. (2008) 38 1909-16, 47 mg, 0.178 mmol). The mixture was heated with stirring at 140° C. for 3 h, during which a brown solution formed. The solution was allowed to coo to rtl, water (8 mL) was added and the mixture agitated with a spatula to allow complete dissolution of the PPSE. The mixture was filtered and the resultant brown solid washed with more water (2×5 mL) and air dried. The solid (about 70 mg) was dissolved in 20% MeOH in DMSO and purified by preparative HPLC (high pH method) to afford compound 236 as a yellow solid; 15 mg, 15%; ¹H NMR (500 MHz, DMSO-d₆) δ10.28 (s, 1H), 9.13 (d, 1H), 7.91 (d, 1H), 7.82-7.75 (m, 1H), 7.73 (d, 1H), 7.65 (s, 1H), 7.61 (d, 1H), 7.38-7.28 (m, 3H), 7.13-7.08 (m, 1H), 4.22 (s, 2H), 3.10 (s, 3H); MS (−ive ion) m/e 559 (M−1)⁻.

### Preparation of 3-(2-Fluoro-phenyl)-2-pyridin-2-yl-propanoic acid ethyl ester 8

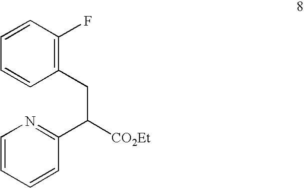

Compound 8 was prepared in a similar manner to 1; 80%; MS m/e 274 (MH)⁺.

### Preparation of 3-Phenyl-2-pyridin-2-yl-propanoic acid ethyl ester 9

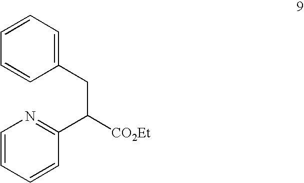

Compound 9 was prepared in a similar manner to 1; 70%; MS m/e 256 (MH)⁺.

### Preparation of 3-(4-Methanesulfonyl-phenyl)-2-pyridin-2-yl-propanoic acid ethyl ester 10

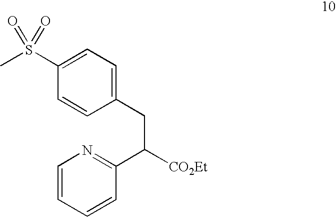

Compound 10 was prepared in a similar manner to 1; 49%; MS m/e 334 (MH)⁺.

### Preparation of 3-(4-Chloro-phenyl)-2-pyridin-2-yl-propanoic acid ethyl ester 11

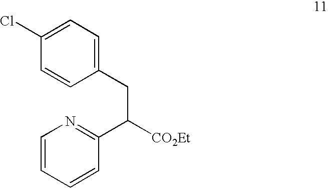

Compound 11 was prepared in a similar manner to 1; 92%; MS m/e 290, 292 (MH)⁺.

### Preparation of 3-(3,5-Difluoro-phenyl)-2-pyridin-2-yl-propanoic acid ethyl ester 12

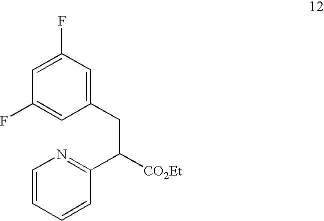

Compound 12 was prepared in a similar manner to 1; 92%; MS m/e 292 (MH)⁺.

### Preparation of 2-Pyridin-2-yl-pent-4-ynoic acid ethyl ester 13

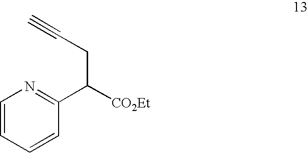

Compound 13 was prepared in a similar manner to 1; 66%; MS m/e 204 (MH)⁺.

### Preparation of 3-(3-Methoxy-phenyl)-2-pyridin-2-yl-propionic acid ethyl ester 14

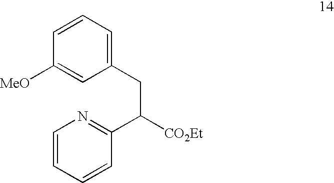

Compound 14 was prepared in a similar manner to 1; 77%; MS m/e 286 (MH)⁺.

### Preparation of 3-(2-Fluoro-phenyl)-2-pyridin-2-yl-propanoic acid, sodium salt 15

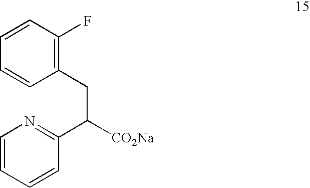

Compound 15 was prepared in a similar manner to 2; 97%; ¹H NMR (500 MHz, MeOD) δ 8.38 (d, 1H), 7.73-7.66 (m, 1H), 7.48-7.44 (m, 1H), 7.20-7.09 (m, 3H), 6.98-6.90 (m, 2H), 4.05-3.99 (m, 1H), 3.48-3.42 (m, 1H), 3.28-3.20 (m, 1H); MS m/e 246 (MH)⁺.

### Preparation of 3-Phenyl-2-pyridin-2-yl-propanoic acid, sodium salt 16

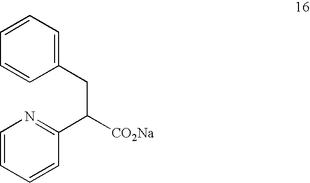

Compound 16 was prepared in a similar manner to 2; 100%; MS m/e 228 (MH)⁺.

### Preparation of 3-(4-Methanesulfonyl-phenyl)-2-pyridin-2-yl-propanoic acid, sodium salt 17

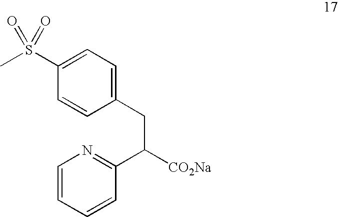

Compound 17 was prepared in a similar manner to 2; 100%; MS m/e 306 (MH)⁺.

### Preparation of 3-(4-Chloro-phenyl)-2-pyridin-2-yl-propanoic acid, sodium salt 18

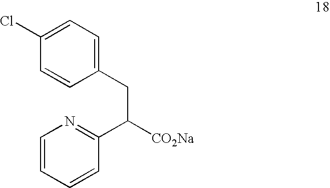

Compound 18 was prepared in a similar manner to 2; 100%; MS m/e 262, 264 (MH)⁺.

### Preparation of 3-(3,5-Difluoro-phenyl)-2-pyridin-2-yl-propanoic acid, sodium salt 19

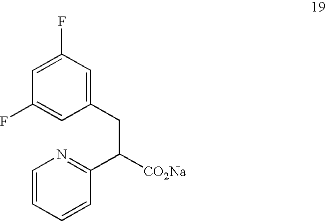

Compound 19 was prepared in a similar manner to 2; 100%; MS m/e 264; (MH)⁺.

### Preparation of 2-Pyridin-2-yl-pent-4-ynoic acid, sodium salt 20

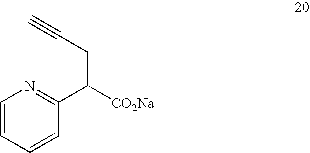

Compound 20 was prepared in a similar manner to 2; 100%; MS m/e 176 (MH)⁺.

### Preparation of 3-(3-Methoxy-phenyl)-2-pyridin-2-yl-propionic acid, sodium salt 21

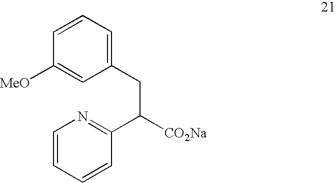

Compound 21 was prepared in a similar manner to 2; 100%; MS m/e 258 (MH)⁺.

### Preparation of 2-[3-(2-Fluoro-phenyl)-2-pyridin-2-yl-propionyl]-malonic acid diethyl ester 22

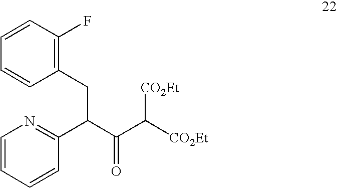

Compound 22 was prepared in a similar manner to 4; 57%; MS m/e 388 (MH)⁺.

### Preparation of 2-(3-Phenyl-2-pyridin-2-yl-propionyl)-malonic acid diethyl ester 23

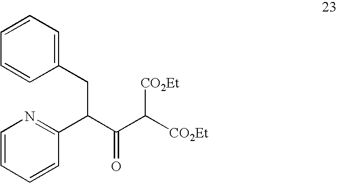

Compound 23 was prepared in a similar manner to 4; used crude in next step; MS m/e 370 (MH)⁺.

### Preparation of 2-[3-(4-Methanesulfonyl-phenyl)-2-pyridin-2-yl-propionyl]-malonic acid diethyl ester 24

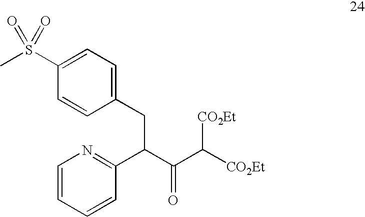

Compound 24 was prepared in a similar manner to 4; used crude in next step; MS m/e 448 (MH)⁺.

### Preparation of 2-[3-(4-Chloro-phenyl)-2-pyridin-2-yl-propionyl]-malonic acid diethyl ester 25

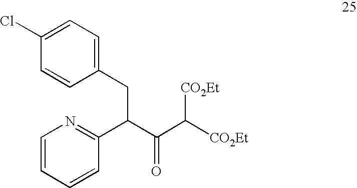

Compound 25 was prepared in a similar manner to 4; used crude in next step; MS m/e 404, 406 (MH)⁺.

### Preparation of 2-[3-(3,5-Difluoro-phenyl)-2-pyridin-2-yl-propionyl]-malonic acid diethyl ester 26

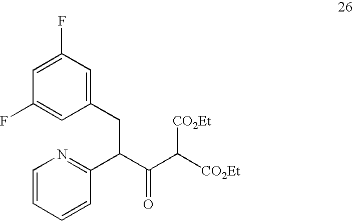

Compound 26 was prepared in a similar manner to 4; used crude in next step; MS m/e 406 (MH)⁺.

### Preparation of 2-(2-Pyridin-2-yl-pent-4-ynoyl)-malonic acid diethyl ester 27

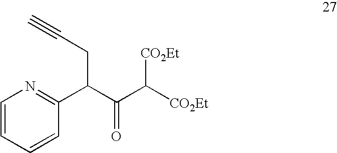

Compound 27 was prepared in a similar manner to 4; used crude in next step; MS m/e 318 (MH)⁺.

### Preparation of 2-[3-(3-Methoxy-phenyl)-2-pyridin-2-yl-propionyl]-malonic acid diethyl ester 28

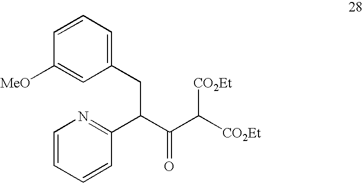

Compound 28 was prepared in a similar manner to 4; used crude in next step; MS m/e 400 (MH)⁺.

### Preparation of 1-(2-Fluoro-benzyl)-4-hydroxy-2-oxo-2H-quinolizine-3-carboxylic acid ethyl ester 29

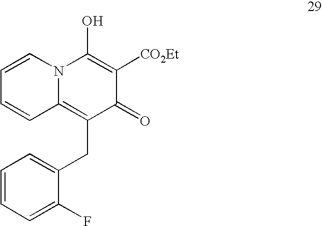

Compound 29 was prepared in a similar manner to 5; 22%; ¹H NMR (250 MHz, CDCl₃) δ 9.09 (d, 1H), 7.45-7.31 (m, 2H), 7.14-7.05 (m, 1H), 7.05-6.96 (m, 3H), 6.88-6.76 (m, 1H), 4.45 (q, 2H), 4.10 (s, 2H), 1.42 (t, 3H); MS m/e 342 (MH)⁺.

### Preparation of 1-Benzyl-4-hydroxy-2-oxo-2H-quinolizine-3-carboxylic acid ethyl ester 30

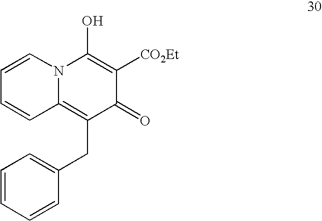

Compound 30 was prepared in a similar manner to 5; 4% from 16; MS m/e 324 (MH)⁺.

### Preparation of 4-Hydroxy-1-(4-Methanesulfonyl-benzyl)-2-oxo-2H-quinolizine-3-carboxylic acid ethyl ester 31

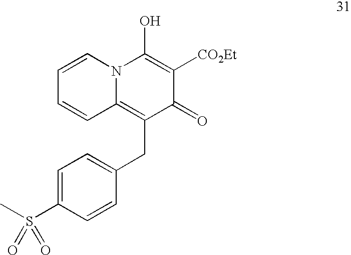

Compound 31 was prepared in a similar manner to 5; 13%; MS m/e (−ive ion) 400 (M−1)⁻.

### Preparation of 1-(4-Chloro-benzyl)-4-hydroxy-2-oxo-2H-quinolizine-3-carboxylic acid ethyl ester 32

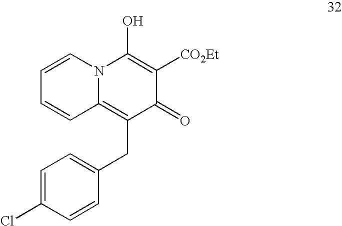

Compound 32 was prepared in a similar manner to 5; 22%; MS m/e 358, 360 (MH)⁺.

### Preparation of 1-(3,5 Difluoro-benzyl)-4-hydroxy-2-oxo-2H-quinolizine-3-carboxylic acid ethyl ester 33

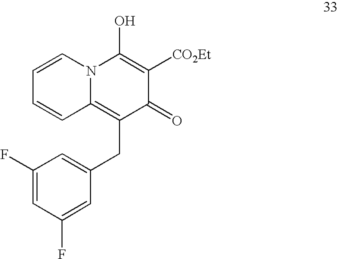

Compound 33 was prepared in a similar manner to 5; 23%; MS m/e 360 (MH)⁺.

### Preparation of 4-Hydroxy-2-oxo-1-prop-2-ynyl-2H-quinolizine-3-carboxylic acid ethyl ester 34

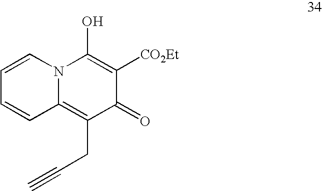

Compound 34 was prepared in a similar manner to 5; 17% from 20; MS m/e 272 (MH)⁺.

### Preparation of 4-Hydroxy-1-(3-methoxy-benzyl)-2-oxo-2H-quinolizine-3-carboxylic acid ethyl ester 35

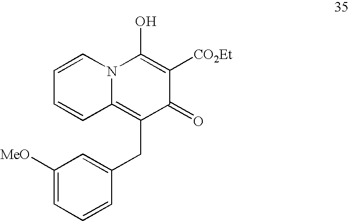

### Preparation of N-{3-[1-(2-Fluoro-benzyl)-4-hydroxy-2-oxo-2H-quinolizin-3-yl]-1,1-dioxo-1,4-dihydro-1lambda′6′-benzo[1,2,4]thiadiazin-7-yl}-methanesulfonamide 237

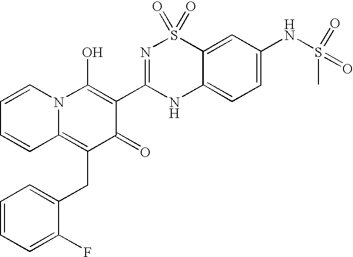

Compound 237 was prepared in a similar manner to 236; 12%; ¹H NMR (500 MHz, DMSO-d₆) δ10.32-10.20 (m, 1H), 9.20-9.05 (m, 1H), 7.90-7.55 (m, 4H), 7.36-7.15 (m, 3H), 7.05-6.97 (m, 2H), 4.20 (s, 2H), 3.08 (s, 3H); MS m/e 543 (MH)⁺.

### Preparation of N-[3-(1-Benzyl-4-hydroxy-2-oxo-2H-quinolizin-3-yl)-1,1-dioxo-1,4-dihydro-1lambda′6′-benzo[1,2,4]thiadiazin-7-yl]-methanesulfonamide 238

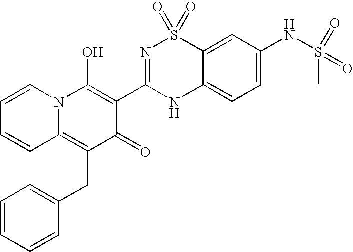

Compound 238 was prepared in a similar manner to 236; 11%; ¹H NMR (500 MHz, DMSO-d₆) δ 10.28 (s, 1H), 9.12 (d, 1H), 7.91 (d, 1H), 7.82 (dd, 1H), 7.73 (d, 1H), 7.65 (s, 1H), 7.60 (d, 1H), 7.32 (dd, 1H), 7.26 (m, 4H), 7.17 (m, 1H), 4.23 (s, 2H), 3.10 (s, 3H); MS (−ive ion) m/e 523 (M−1)⁻.

### Preparation of N-{3-[4-hydroxy-1-(4-methanesulfonyl-benzyl)-2-oxo-2H-quinolizin-3-yl]-1,1-dioxo-1,4-dihydro-1lambda′6′-benzo[1,2,4]thiadiazin-7-yl}-methanesulfonamide 239

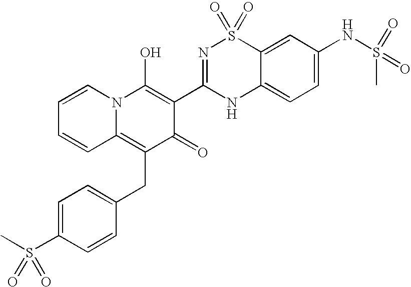

Compound 239 was prepared in a similar manner to 236; 4%; ¹H NMR (500 MHz, DMSO-d₆) δ 10.29 (s, 1H), 9.15 (d, 1H), 7.93 (d, 1H), 7.81-7.70 (m, 4H), 7.64 (s, 1H), 7.60 (d, 1H), 7.53 (d, 2H), 7.33 (d, 1H), 4.35 (s, 2H), 3.16 (s, 3H), 3.10 (s, 3H); MS (−ive ion) m/e 601 (M−1)⁻.

### Preparation of N-{3-[1-(4-Chloro-benzyl)-4-hydroxy-2-oxo-2H-quinolizin-3-yl]-1,1-dioxo-1,4-dihydro-1lambda′6′-benzo[1,2,4]thiadiazin-7-yl}-methanesulfonamide 240

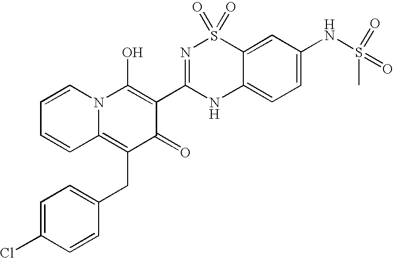

Compound 240 was prepared in a similar manner to 236; 26%; ¹H NMR (500 MHz, DMSO-d₆) δ 10.29 (s, 1H), 9.10 (d, 1H), 7.89 (d, 1H), 7.80 (dd, 1H), 7.72 (d, 1H), 7.64 (s, 1H), 7.60 (d, 1H), 7.32-7.29 (m, 5H), 4.20 (s, 2H), 3.10 (s, 3H); MS (−ive ion) m/e 557, 559 (M−1)⁻.

### Preparation of N-{3-[1-(3,5-Difluoro-benzyl)-4-hydroxy-2-oxo-2H-quinolizin-3-yl]-1,1-dioxo-1,4-dihydro-1lambda′6′-benzo[1,2,4]thiadiazin-7-yl}-methanesulfonamide 241

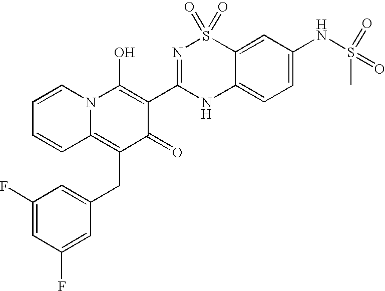

Compound 241 was prepared in a similar manner to 236; 16%; ¹H NMR (500 MHz, DMSO-d₆) δ 10.29 (s, 1H), 9.12 (bs, 1H), 7.89-7.85 (m, 2H), 7.70 (bs, 1H), 7.64-7.55 (m, 2H), 7.33 (bs, 1H), 7.03-6.85 (m, 3H), 4.25 (s, 2), 3.09 (s, 3H); MS (−ive ion) m/e 559 (M−1)⁻.

### Preparation of N-[3-(2-methyl-5-oxo-5H-3-oxa-5^{a}-aza-cyclopenta[a]naphthalen-4-yl-1,1-dioxo-1,4-dihydro-1lambda′6′-benzo[1,2,4]thiadiazin-7-yl)-methanesulfonamide 242

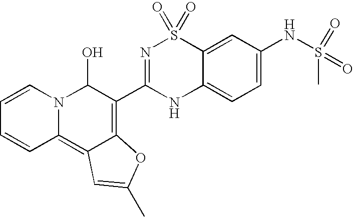

Compound 242 was prepared in a similar manner to 236, cyclisation to form the furan occurring under the reaction conditions; 4%; ¹H NMR (500 MHz, DMSO-d₆) δ 9.38 (d, 1H), 8.39 (d, 1H), 8.12 (dd, 1H), 7.64 (dd, 1H), 7.60-7.53 (m, 3H), 7.23 (s, 1H), 3.06 (s, 3H), 2.55 (s, 3H); MS (−ive ion) m/e 471 (M−1)⁻.

### Preparation of N-{3-[4-Hydroxy-1-(3-methoxy-benzyl)-2-oxo-2H-quinolizin-3-yl]-1,1-dioxo-1,4-dihydro-1lambda*6*-benzo[1,2,4]thiadiazin-7-yl}-methanesulfonamide 243

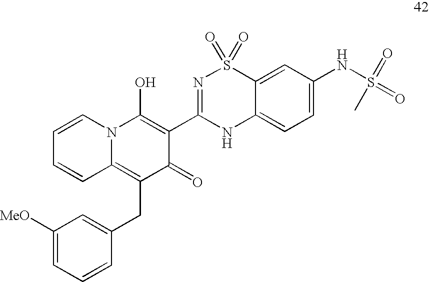

Compound 243 was prepared in a similar manner to 236; 32%; ¹H NMR (500 MHz, DMSO-d₆) δ 14.26 (s, 2H), 10.29 (s, 1H), 9.10 (s, 1H), 7.88-7.55 (m, 5H), 7.31 (s, 1H), 7.18-7.17 (m, 1H), 6.84-6.55 (m, 3H), 4.19 (s, 2H), 3.70 (s, 3H), 3.20 (s, 3H); MS (−ive ion) m/e 553 (M−1)⁻.

### Example 17

A general synthetic scheme for the preparation of polymerase inhibitors, described in this section is illustrated in Scheme 17 below and exemplified by the following description of the synthesis of compound 244.

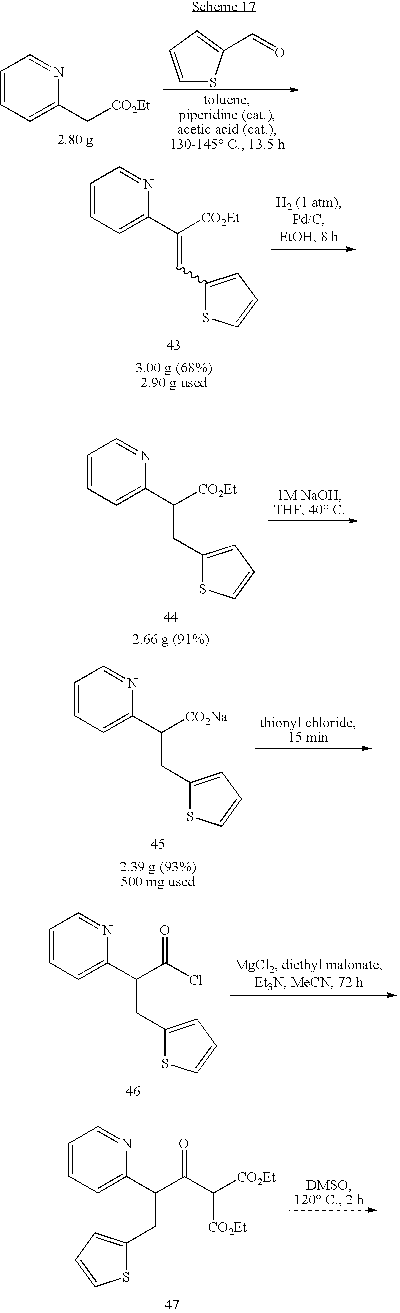

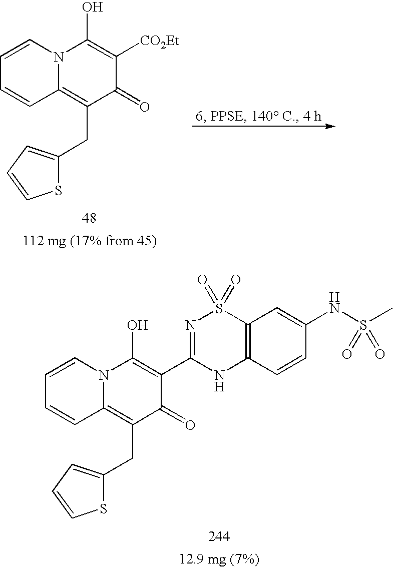

### Preparation of 2-Pyridin-2-yl-3-thiphen-2-yl-acrylic acid ethyl ester 43

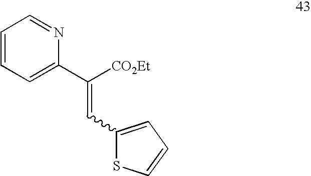

A mixture of ethyl 2-pyridylacetate (2.80 g, 17.0 mmol), thiophene-2-carboxaldehyde (2.00 g, 17.8 mmol), piperidine (70 mg, 0.823 mmol), glacial acetic acid (210 mg, mmol) in toluene (15 mL) was heated to reflux under Dean-Stark conditions for 1.5 h. Heating was continued at 130° C. for 12 h. The solution was cooled to rt, diluted with EtOAc (30 mL) and washed with saturated sodium carbonate solution (2×5 mL). The organic layer was dried (Na₂SO₄), the mixture filtered the filtrate evaporated to dryness. The residue was chromatographed on silica with dry-loading (eluent: 100% heptane to 50% EtOAc in heptane to afford the title compound as a yellow oil; 3.00 g, 68%; MS m/e 260 (MH)⁺.

### Preparation of 2-Pyridin-2-yl-3-thiphen-2-yl-propanoic acid ethyl ester 44

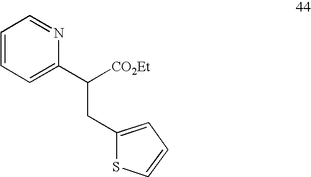

To a solution of olefin 43 (2.90 g, 11.2 mmol) in EtOH (50 mL) was added 10% palladium on charcoal (50% wet, 1.00 g) and the mixture stirred while being purged repeatedly with nitrogen. The mixture was then stirred under an atmosphere of hydrogen (1 atm) for 8 h, the atmosphere replaced with nitrogen, and the mixture filtered through Celite with the filtrate evaporated to dryness to afford the title compound as a green oil which was used without further purification; 2.66 g, 91%; MS m/e 262 (MH)⁺.

### Preparation of 2-Pyridin-2-yl-3-thiphen-2-yl-propanoic acid, sodium salt 45

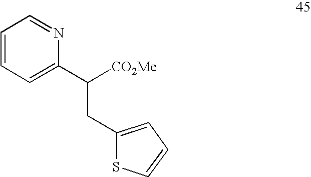

To a solution of ester 44 (2.66 g, 10.1 mmol) in THF (100 mL) and water (20 mL) was added a solution of sodium hydroxide (1M, 10.1 mL, 10.1 mmol), and the two-phase mixture heated to 40° C. for 16 h. The mixture was evaporated to dryness and the residue azeotroped three times with THF to remove residual solvent, before being dried further under high vacuum to afford the title compound as a white solid; 2.39 g, 93%; MS m/e 234 (MH)⁺.

### Preparation of 2-(2-Pyridin-2-yl-3-thiphen-2-yl-propionyl)-malonic acid diethyl ester 47

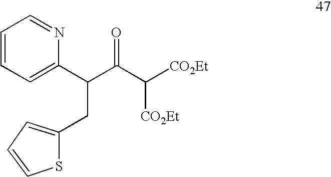

Thionyl chloride (2 mL) was added to solid sodium salt 45 (500 mg, 1.96 mmol) and the mixture stirred for 15 min until a red solution formed. The thionyl chloride was evaporated and the residue azeotroped three times from anhydrous THF to afford the acid chloride 46.

To a stirred mixture of diethyl malonate (314 mg, 1.96 mmol), anhydrous MgCl₂ (186 mg, 1.96 mmol) and triethylamine (0.540 mL, 3.92 mmol) in anhydrous MeCN (5.0 mL) at 0° C. was added a solution of the acid chloride 46 in MeCN (4 mL), dropwise over 5 min. The mixture was allowed to warm to rt and stirred for 72 h. The solvent was evaporated, EtOAc (20 mL) added and the organic phase washed with 10% citric acid (2×10 mL), dried (Na₂SO₄), the mixture filtered and the filtrate evaporated to dryness to afford the title compound (about 50% pure judging by LCMS) which was used without further purification; 853 mg crude; MS m/e 376 (MH)⁺.

### Preparation of 4-Hydroxy-2-oxo-1-thiophen-2-ylmethyl-2H-quinazoline-3-carboxylic acid ethyl ester 48

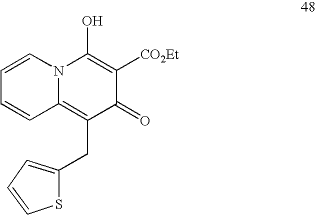

A solution of diester 47 (853 mg, 2.26 mmol) was dissolved in DMSO (2 mL) and the solution heated at 120° C. for 2 h. The solution was allowed to cool to rt, water added (100 mL) and the mixture extracted with EtOAc (3×100 mL). The combined organic extracts dried (Na₂SO₄), the mixture filtered and the filtrate evaporated to dryness to afford an orange solid which was chromatographed (silica, eluent 0-70% EtOAc in heptane) to afford the title compound as a bright yellow solid; 112 mg, 17% from 45; MS m/e 330 (MH)⁺.

### Preparation of N-[3-(4-Hydroxy-2-oxo-1-thiophen-2-ylmethyl-2H-quinazolin-3-yl)-1,1-dioxo-1,4-dihydro-1lambda′6′-benzo[1,2,4]thiadiazin-7-yl]-methanesulfonamide 244

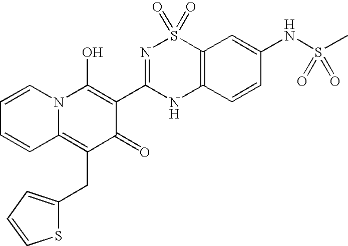

Ester 48 (112 mg, 0.339 mmol), aminosulfonamide 6 (108 mg, 0.406 mmol) and PPSE (2.5 mL) were placed in a sealable tube containing a stirrer bar. The tube was sealed and the mixture heated at 140° C. for 4 h. The solution was allowed to cool, water (50 mL) added and the mixture stirred 10 minutes, to afford a precipitate. The mixture was filtered and the solid dried in air before being dissolved in DMSO and purified by preparative HPLC (high pH method) to afford compound 244 as a yellow solid; 12.9 mg, 17%; ¹H NMR (500 MHz, DMSO-d₆) δ 14.3 (s, 1H), 14.1 (s, 1H), 10.28 (s, 1H), 9.12 (d, 1H), 8.02 (d, 1H), 7.85 (dd, 1H), 7.73 (d, 1H), 7.65 (s, 1H), 7.59 (d, 1H), 7.34 (dd, 1H), 7.29 (d, 1H), 6.96 (s, 1H), 6.90 (dd, 1H), 4.39 (s, 2H), 3.09 (s, 3H); MS m/e 531 (MH)⁺.

### Example 18

A general synthetic scheme for the preparation of polymerase inhibitors, described in this section is illustrated in Scheme 18 below and exemplified by the following description of the synthesis of compound 245.

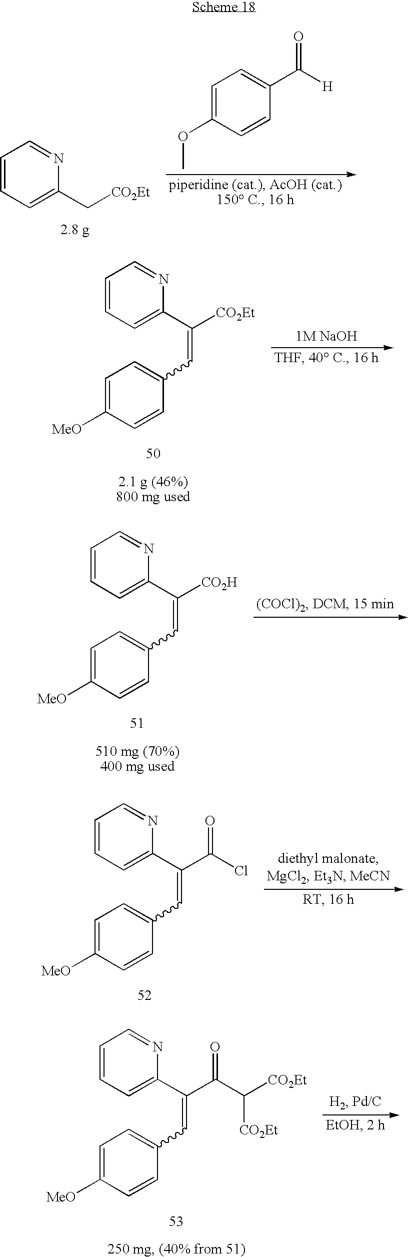

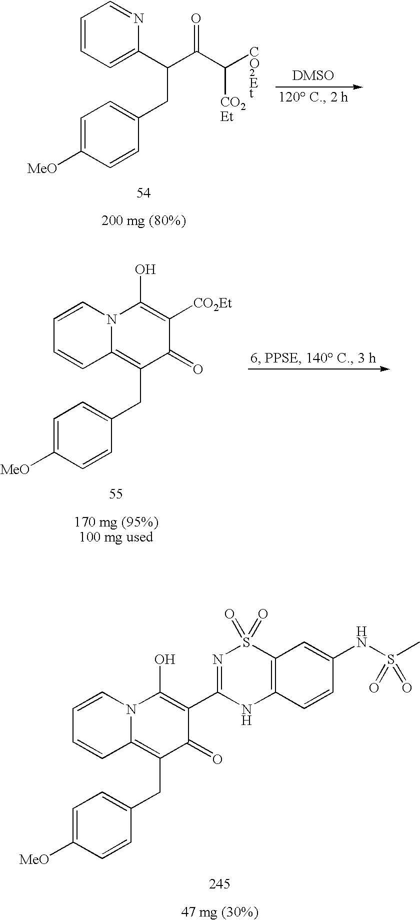

### Preparation of 3-(4-methoxy-phenyl)-2-pyridin-2-yl-acrylic acid ethyl ester 50

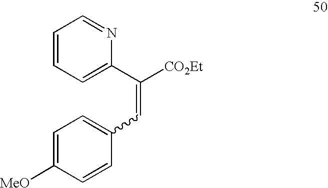

Compound 50 was prepared as a mixture of regioisomers in a similar manner to 43 of Scheme 17; 46%; MS m/e 284 (MH)⁺.

### Preparation of 3-(4-methoxy-phenyl)-2-pyridin-2-yl-acrylic acid 51

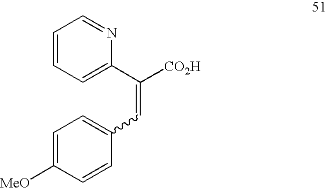

To a solution of ester 50 (800 mg, 2.82 mmol) in THF (10 mL) and water (3 mL) was added a solution of sodium hydroxide (1M, 2.82 mL, 2.82 mmol) and the mixture stirred at 40° C. for 16 h. The THF was evaporated, more water (20 mL) added and the solution acidified to pH 4 with 1M HCl and sodium hydrogen carbonate. The mixture was then extracted with TBME (3×20 mL) and DCM (2×35 mL) and the combined organic layers dried (Na₂SO₄), the mixture filtered and the filtrate evaporated to dryness to afford compound 51 as a yellow solid; 510 mg, 70%; MS m/e 256 (MH)⁺.

### Preparation of 2-[3-(4-methoxy-phenyl)-2-pyridin-2-yl-acryloyl]-malonic acid diethyl ester 53

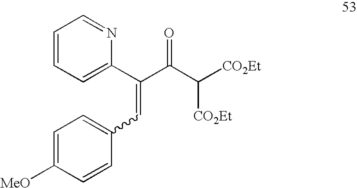

A suspension of acid 51 (400 mg, 1.57 mmol) in DCM (8 mL) was cooled to 4° C. Oxalyl chloride (0.332 mL, 3.93 mmol) was added with stirring and the orange solution stirred until gas evolution ceased (ca. 20 min). The solvent was evaporated and the acid chloride azeotroped twice with DCM to afford acid chloride 52.

A solution of diethyl malonate (0.238 mL, 1.57 mmol), anhydrous MgCl₂ (149 mg, 1.57 mmol) and triethylamine (0.437 mL, 3.14 mmol) in anhydrous MeCN (8 mL) was cooled to 4° C. with stirring under nitrogen. To this mixture was added a solution of acid chloride 52 in anhydrous MeCN and the resultant mixture stirred for 16 h at rt. The solvent was evaporated, EtOAc (15 mL) added and the organic layer washed with 10% aqueous citric acid solution (pH 4, adjusted if necessary with phosphate buffer), dried (Na₂SO₄), the mixture filtered and the filtrate evaporated to dryness. The residue was chromatographed on silica (eluent 0-10% EtOAc in heptane) to afford the title compound as a yellow oil; 250 mg, 40%; MS m/e 398 (MH)⁺.

### Preparation of 2-[3-(4-methoxy-phenyl)-2-pyridin-2-yl-propionyl]-malonic acid diethyl ester 54

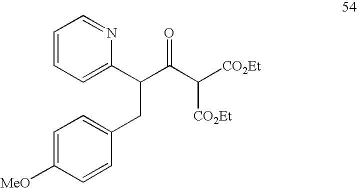

To a solution of olefin 53 (250 mg, 0.63 mmol) in EtOH (15 mL) was added 10% palladium on charcoal (50% wet, 50 mg) and the mixture stirred while purging repeatedly with nitrogen. The mixture was then stirred under an atmosphere of hydrogen (1 atm) for 2 h, the atmosphere replaced with nitrogen, the mixture filtered through Celite and the filtrate evaporated to dryness to afford the title compound as a yellow oil which was used without further purification; 200 mg, 80%; MS m/e 400 (MH)⁺.

### Preparation of 4-Hydroxy-1-(4-methoxy-benzyl)-oxo-2H-quinazoline-3-carboxylic acid ethyl ester 55

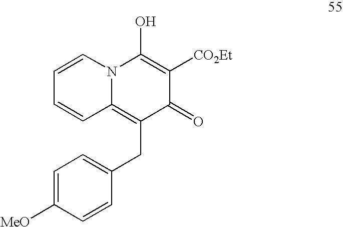

Compound 55 was prepared in a similar manner to 48, except that chromatography was unnecessary; 170 mg, 95%; MS m/e 354 (MH)⁺.

### Preparation of N-{3-[4-Hydroxy-1-(4-methoxy-benzyl)-2-oxo-2H-quinazolin-3-yl]-1,1-dioxo-1,4-dihydro-1lambda′6′-benzo[1,2,4]thiadiazin-7-yl}-methanesulfonamide 245

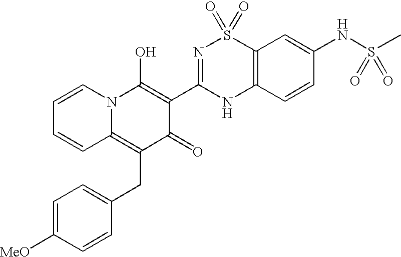

Compound 245 was prepared in a similar manner to compound 244; 47 mg, 30%; ¹H NMR (500 MHz, DMSO-d₆) δ 14.27 (s, 1H), 14.26 (s, 1H), 10.27 (s, 1H), 9.11 (d, 1H), 7.91 (d, 1H), 7.79 (dd, 1H), 7.72 (d, 1H), 7.64 (s, 1H), 7.59 (d, 1H), 7.30 (dd, 1H), 7.18 (d, 2H), 6.82 (d, 2H), 4.14 (s, 2H), 3.68 (s, 3H), 3.09 (s, 3H); MS m/e 555 (MH)⁺.

### Preparation of 3-Furan-3-yl-2-pyridin-2-yl-acrylic acid ethyl ester 57

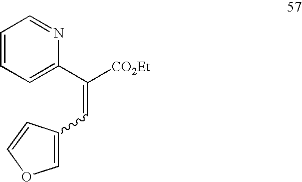

Using a variation of the Knoevenagel procedure described for the preparation of 50, ethyl 2-pyridylacetate (500 mg, 3.02 mmol) was dissolved in anhydrous THF (8 mL) and the solution cooled to −78° C. under a nitrogen atmosphere. Lithium bis(trimethylsilyl)amide (1M in THF, 3.00 mL, 3.00 mmol) was added via syringe over 10 min followed by 3-furaldehyde (290 mg, 3.02 mmol) and the yellow solution allowed to warm to −40° C., with further stirring at this temperature for 2 h. Acetic anhydride (616 mg, 6.04 mmol) was added and the reaction allowed to warm to rt. Triethylamine (610 mg, 6.04 mmol) was added and the solution stirred overnight at rt and then at 55° C. for 3 h or until elimination was complete (as determined by LCMS). The solvent was evaporated and the brown solid chromatographed on silica (eluent 30% EtOAc in DCM) to afford the title compound as mixture of regioisomers which were separately isolated during the chromatography step; combined yield 540 mg, 73%); MS m/e 244 (MH)⁺.

### Preparation of 3-(4-Dimethylamino-phenyl)-2-pyridin-2-yl-acrylic acid ethyl ester 58

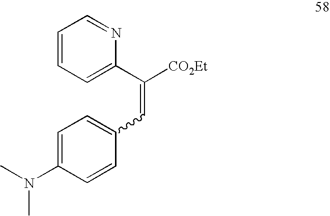

Compound 58 was prepared in a similar manner to 43 of Scheme 17; 19%; MS m/e 297 (MH)⁺.

### Preparation of 3-Furan-3-yl-2-pyridin-2-yl-acrylic acid 59

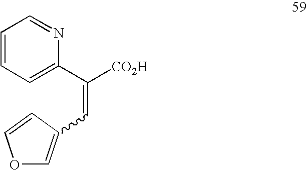

Compound 59 was prepared in a similar manner to 51; 68%; MS m/e 216 (MH)⁺.

### Preparation of 3-(4-Dimethylamino-phenyl)-2-pyridin-2-yl-acrylic acid, sodium salt 60

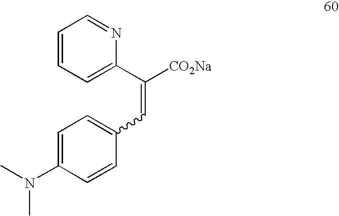

Compound 60 was prepared in a similar manner to 51, except that the sodium salt was isolated directly by evaporation of the reaction mixture; 99%; MS m/e 269 (MH)⁺.

### Preparation of 2-(3-Furan-3-yl-2-pyridin-2-yl-acryloyl)-malonic acid diethyl ester 61

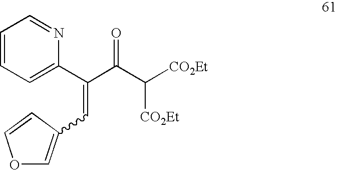

Compound 61 was prepared in a similar manner to 53, except that the material was used in the next step without purification; 55%; MS m/e 358 (MH)⁺.

### Preparation of 2-[3-(4-Dimethylamino-phenyl)-2-pyridin-2-yl-acryloyl]-malonic acid diethyl ester 62

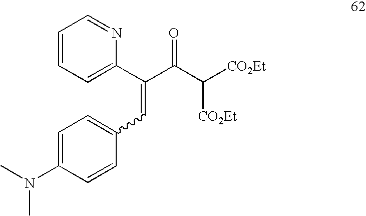

Compound 62 was prepared in a similar manner to 4 Scheme 16 (i.e. by the action of thionyl chloride on sodium salt 60, followed by reaction with sodium diethyl malonate); 23%; MS m/e 411 (MH)⁺.

### Preparation of 2-(3-Furan-3-yl-2-pyridin-2-yl-propioyl)-malonic acid diethyl ester 63

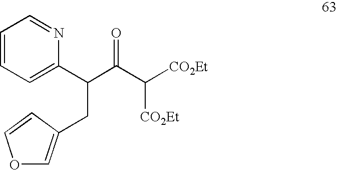

Compound 63 was prepared in a similar manner to 54; 78%; MS m/e 360 (MH)⁺.

### Preparation of 2-[3-(4-Dimethylamino-phenyl)-2-pyridin-2-yl-propionyl]-malonic acid diethyl ester 64

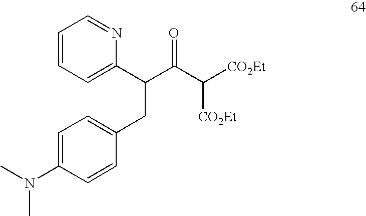

Compound 64 was prepared in a similar manner to 54; 67%; MS m/e 413 (MH)⁺.

### Preparation of 1-Furan-3-ylmethyl-4-Hydroxy-oxo-2H-quinazoline-3-carboxylic acid ethyl ester 65

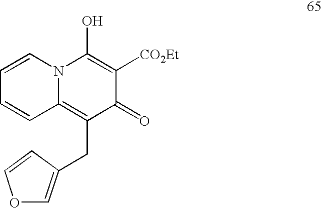

Compound 65 was prepared in a similar manner to 48 of Scheme 17; 30%; MS m/e 314 (MH)⁺.

### Preparation of 1-(4-Dimethylamino-benzyl)-4-hydroxy-2-oxo-2H-quinolizine-3-carboxylic acid ethyl ester 66

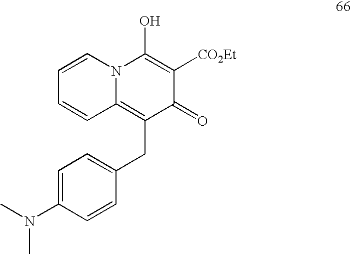

Compound 66 was prepared in a similar manner to 48 of Scheme 17; 99%; MS m/e 367 (MH)⁺.

### Preparation of N-[3-(1-Furan-3-ylmethyl-4-Hydroxy-2-oxo-2H-quinazolin-3-yl)-1,1-dioxo-1,4-dihydro-1lambda′6′-benzo[1,2,4]thiadiazin-7-yl]-methanesulfonamide 246

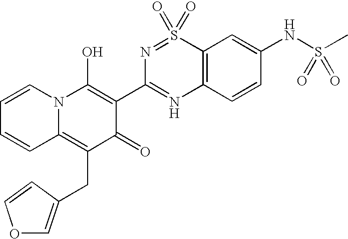

Compound 246 was prepared in a similar manner to compound 244; 17%; ¹H NMR (500 MHz, DMSO-d₆) δ 14.27 (s, 1H), 14.23 (s, 1H), 10.28 (s, 1H), 9.10 (d, 1H), 7.94 (d, 1H), 7.82 (dd, 1H), 7.72 (d, 1H), 7.64 (s, 1H), 7.58 (d, 1H), 7.53 (s, 1H), 7.47 (s, 1H), 7.33 (dd, 1H), 6.40 (s, 1H), 3.97 (s, 2H), 3.09 (s, 3H); MS m/e 515 (MH)⁺.

### Preparation of N-{3-[1-(4-Dimethylamino-benzyl)-4-hydroxy-2-oxo-2H-quinolizin-3-yl]-1,1-dioxo-1,4-dihydro-1lambda*6*-benzo[1,2,4]thiadiazin-7-yl}-methanesulfonamide 247

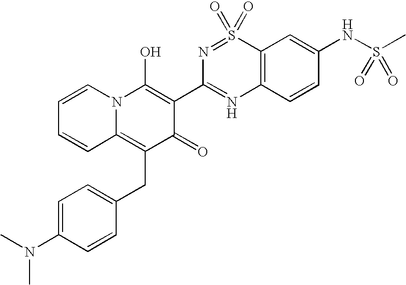

Compound 247 was prepared in a similar manner to compound 244; 3%; ¹H NMR (500 MHz, DMSO-d₆) δ 14.28 (s, 1H), 14.22 (s, 1H), 10.28 (s, 1H), 9.09 (bs, 1H), 7.89 (bs, 1H), 7.78 (bs, 1H), 7.71 (bs, 1H), 7.66-7.55 (m, 2H), 7.29 (bs), 7.06 (d, 2H), 6.61 (d, 2H), 4.08 (bs, 2H), 3.08 (s, 3H), 2.80 (s, 6H); MS m/e 568 (MH)⁺.

### Example 19

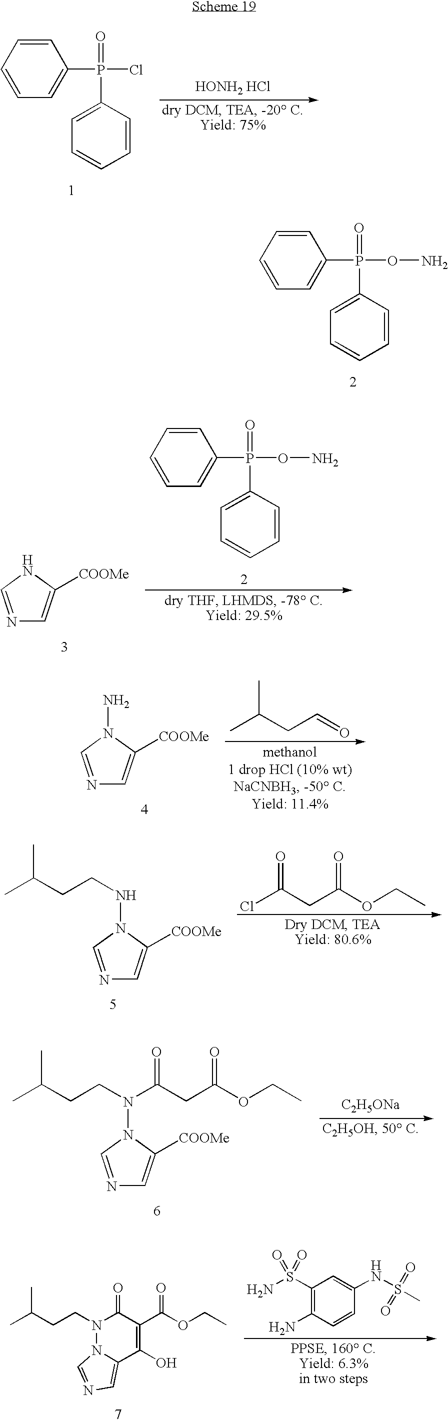

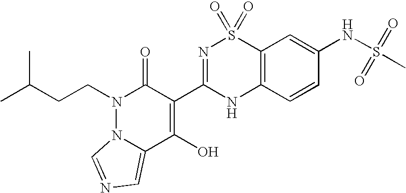

### Preparation of Compound 2

A solution of hydroxylamine hydrochloride (12.9 g, 186 mmol) in dry DCM (500 mL) was added dry TEA (34.3 g, 340 mmol). The reaction mixture was cooled to −20° C., followed by adding dropwise of compound 1 (40 g, 170 mmol) in dry DCM (50 mL). The solution was maintained at −20° C. for another 1.5 h. The reaction was allowed to warm to rt overnight. The reaction was filtered, the solid was diluted with 500 mL of water, then filtered to give desired compound 2 (29.8 g, yield: 75%).

### Preparation of Compound 4

A solution of compound 3 (1.0 g, 7.93 mmol) in dry THF (20 mL) was added dropwise of LHMDS (10 mL, 10 mmol) at −78° C. and stirred for 1 h at this temperature. Then the reaction was slowly warmed to −10° C. for 30 minutes. Compound 2 (1.85 g, 7.93 mmol) was added to the result solution in one portion. The reaction mixture was allowed to warm to room temperature overnight. The solution was diluted with DCM (50 mL), filtered. The organic phase was quenched by adding water, dried over Na₂SO₄. Filtered and concentrated, the residue was purified by Prep-HPLC to give compound 4 (330 mg, yield: 29.5%). MS-ESI: m/z=142 [M+1]⁺.

### Preparation of Compound 5

To a solution of compound 4 (846 mg, 6 mmol) in methanol (25 mL) was added 3-methylbutanyl (670 mg, 7.8 mmol) and 1 drop of HCl solution (10% wt). The reaction mixture was stirred at rt for 30 minutes. After which NaCNBH₃ (252 mg, 4 mmol) was added and the resulting mixture was heated to 50° C., stirred overnight. The reaction mixture was cooled down, diluted with water, concentrated to remove solvent methanol. The mixture was extracted with DCM, washed with NaHCO₃ solution, the combined organic phase was dried over Na₂SO₄, concentrated, the residue was purified by TLC to give compound 5 (145 mg, yield: 11.4%) MS-ESI: m/z=212 [M+1]⁺.

### Preparation of Compound 6

A solution of compound 5 (145 mg, 0.687 mmol) in dry DCM (10 mL) was added TEA (104 mg, 1.03 mmol), followed by adding chlorocarbonyl-acetic acid ethyl ester (154 mg, 1.03 mmol). The mixture was stirred overnight at room temperature. The solution was concentrated in vacuo, the residue was purified by TLC to give compound 6 (180 mg, yield: 80.6%). MS-ESI: m/z=326 [M+1]⁺.

### Preparation of Compound 7

To a solution of compound 6 (280 mg, 0.86 mmol) in dry ethanol (4 mL) was added sodium ethoxide (175 mg, 2.58 mmol). The reaction mixture was flushed with N₂, heated to 60° C. for 9 hours. The reaction mixture was cooled down, purified by TLC (DCM CH₃OH=10:1) to give compound 7 (150 mg contained silical gel). MS-ESI: m/z=294 [M+1]⁺.

### Preparation of Compound 248

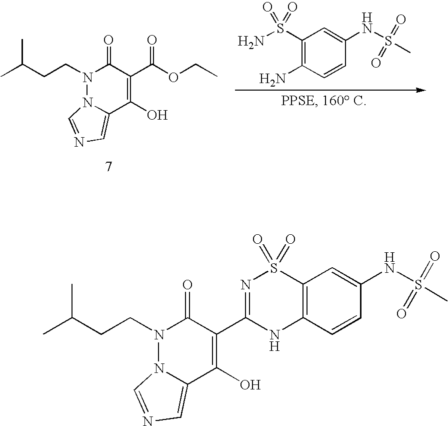

Compound 7 (approx. 94 mg (crude), 0.32 mmol) and sulfonamide (85 mg, 0.32 mmol) in PPSE (3 mL) was flushed with N₂, heated to 160° C. and stirred for 2 h. The reaction mixture was cooled down, diluted with EA (20 mL), quenched by adding water. The mixture was extracted with EA. The organic phase was concentrated, the residue was purified by pre-HPLC□ Column style: YMC-Pack ODS-AQ, 150*30 mml.D. s-5 um. Mobile phase: water+0.075% TFA, CAN+0.075% TFA (ratio: 45:55:75:100) to give compound 248 (TFA salt) (10 mg, yield: 6.3% in two steps, purity: 93.1% in LC-Ms). ¹H NMR (400 MHz, DMSO): 0.923 (d, 6H, J=6.4), 1.511 (m, 2H), 1.650 (m, 1H), 3.025 (s, 3H), 4.289 (t, 2H, J=6.8), 7.482 (s, 2H), 7.537 (s, 1H), 7.976 (s, 1H), 8.923 (s, 1H), 10.094 (s, 1H), 13.796 (s, 1H). MS-ESI: m/z=495 [M+1]⁺.

### Example 20

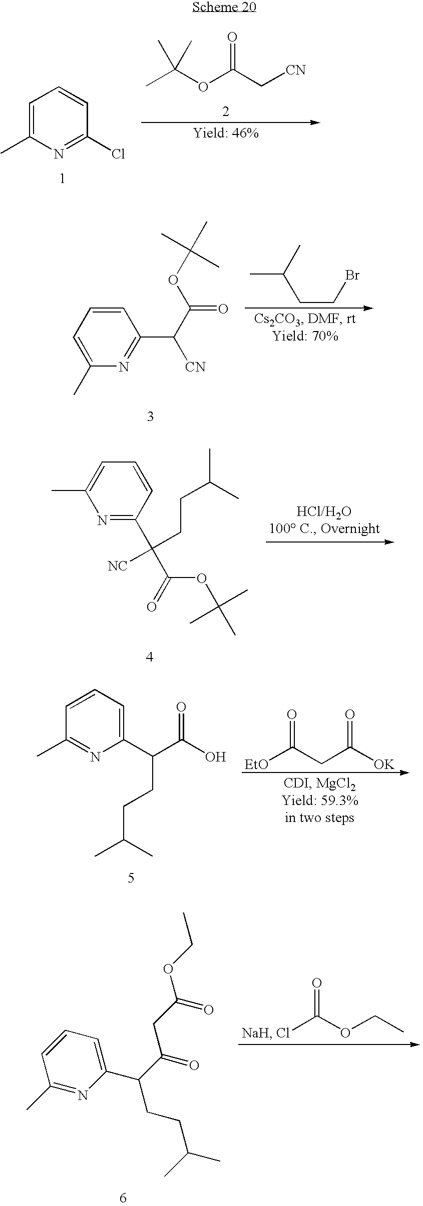

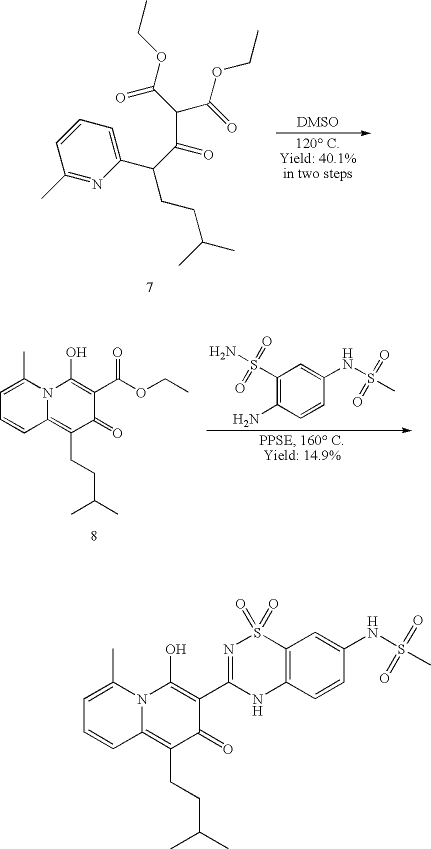

### Preparation of Compound 3

To a solution of compound 1 (10 g, 78 mmol) added t-BuOK (17 g, 156 mmol) in dioxane (150 ml), followed by Pd(PPh₃)₄ (2 g, 0.02 eq.). The reaction mixture was heated to 70° C. and stirred overnight at this temperature. After overnight, large quantity of solid was precipitate, diluted with water and extracted with EtOAc. Combined the organic layer and dried over Na₂SO₄ then removed the solvent to get yellow solid which was washed with ether then filtered. (8.4 g, yield: 46%). MS-ESI: m/z=233.0 [M+1]⁺.

### Preparation of Compound 4

To a solution of compound 3 (1 g, 4 mmol) in 8 ml of DMF was added 1-bromo-3-methylbutane (1.43 g, 8 mmol), followed by adding Cs₂CO₃ (2.8 g, 8 mmol). The mixture was stirred overnight at r.t, then diluted with water and extracted with EtOAc, dried over with Na₂SO₄, concentrated to give the crude which was purified by chromatography to give compound 4 (0.9 g, yield: 70%). MS-ESI: m/z=303.0 [M+1]⁺.

### Preparation of Compound 5

To a solution of compound 4 (20 g, 66 mmol) in water (70 ml) was added con. HCl (70 ml). The mixture was heated to 100° C. and refluxed overnight at this temperature. Then the mixture was cooled in an ice bath and neutralized with 1N HCl to pH˜4. The solution was freeze-dried to give the mixture of compound 5 and NaCl salt which was used directly for the next step. MS-ESI: m/z=222.0 [M+1]⁺.

### Preparation of Compound 6

A solution of crude compound 5 (57.9 mmol) in anhydrous tetrahydrofuran (THF) (150 mL) was cooled in salt-ice bath, and N,N′-carbonyldiimidazole (9.38 g, 57.9 mmol) was added in small portions under vigorous stirring. After evolution of gas, the mixture was stirred at room temperature for 3 h and then cooled in an ice bath. To a suspension of monoethyl malonate potassium salt (21.6 g, 127.3 mmol) in THF (150 mL) in ice bath was added Et₃N (18.1 g, 179.4 mmol) followed by anhydrous MgCl₂ (14.8 g, 156.3 mmol). The mixture was stirred at room temperature for 3 h, then cooled in salt-ice bath and the above solution of the activated ester previously prepared in THF was added dropwise slowly. The mixture was allowed to stir for 39 h at room temperature, quenched with aqueous citric acid and extracted with ethyl acetate. The organic layers were washed with saturated NaHCO₃ solution and brine, dried (Na₂SO₄) and concentrated in vacuo and purified by chromatography to give compound 6 as yellow oil (10 g, yield: 59.3% in two steps) [symbol] MS-ESI: m/z=292.0 [M+1]⁺.

### Preparation of Compound 7

Compound 6 (10 g, 34.3 mmol) was dissolved in anhydrous THF (100 mL) and cooled to 0° C. NaH (60% in oil, 2.7 g, 68.6 mmol) was added and the mixture stirred for 45 min at room temperature. After cooling again to 0° C., a solution of ethyl chloroformate (5.6 g, 51.4 mmol) in anhydrous THF (2 mL) was slowly added with a seringe. The solution was stirring at room temperature for 2 h, treated with water, acidified to pH˜3 by addition of citric acid and extracted with ethyl acetate. The organic layer was dried over Na₂SO₄ and concentrated in vacuo to give crude product 7 which was used directly for the next step. MS-ESI: m/z=364.0 [M+1]⁺.

### Preparation of Compound 8

The crude compound 7 (500 mg, 1.4 mmol) was dissolved in DMSO (10 mL) and heated to 120° C. for 2.5 h. Then it was poured into water and extracted with ethyl acetate. The organic layer was washed with water, dried on Na₂SO₄ and concentrated in vacuo. The product was purified by prep-TLC to give compound 8 as brown solid (100 mg, yield: 40.1% in two steps). MS-ESI: m/z=318.0 [M+1]⁺.

### Preparation of Compound 249

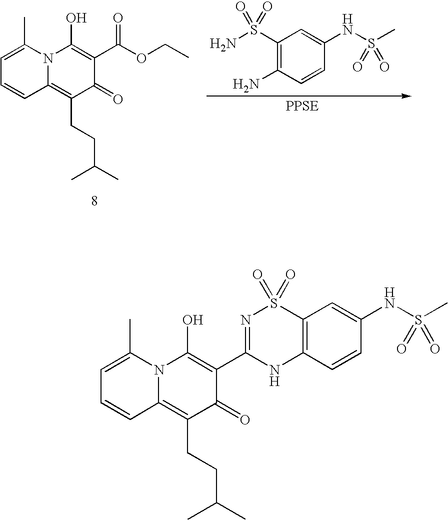

Compound 8 (100 mg, 0.31 mmol) was added at 160° C. to PPSE (5 mL) which and then 2-amino-5-(methylsulfonamido)benzenesulfonamide (85 mg, 0.31 mmol) was added. The solution stirred for 2 h at 160° C. The cooled mixture was poured into water and the precipitate was collected and washed with MeOH for several times. Then it was dried to give compound 249 as a dark yellow green solid (24 mg, yield: 14.9%, purity: 98.3% in LC-Ms). ¹H NMR (400 MHz, DMSO): 0.923 (d, 6H, J=6.4 Hz), 1.272 (m, 2H), 1.586 (m, 2H), 2.895 (s, 3H), 3.095 (s, 3H), 6.857 (m, 1H,), 7.545 (m, 5H), 10.213 (s, 1H), 14.105 (s, 1H), 13.956 (s, 1H), 14.108 (s, 1H). MS-ESI: m/z=519.1 [M+1]⁺.

### Example 21

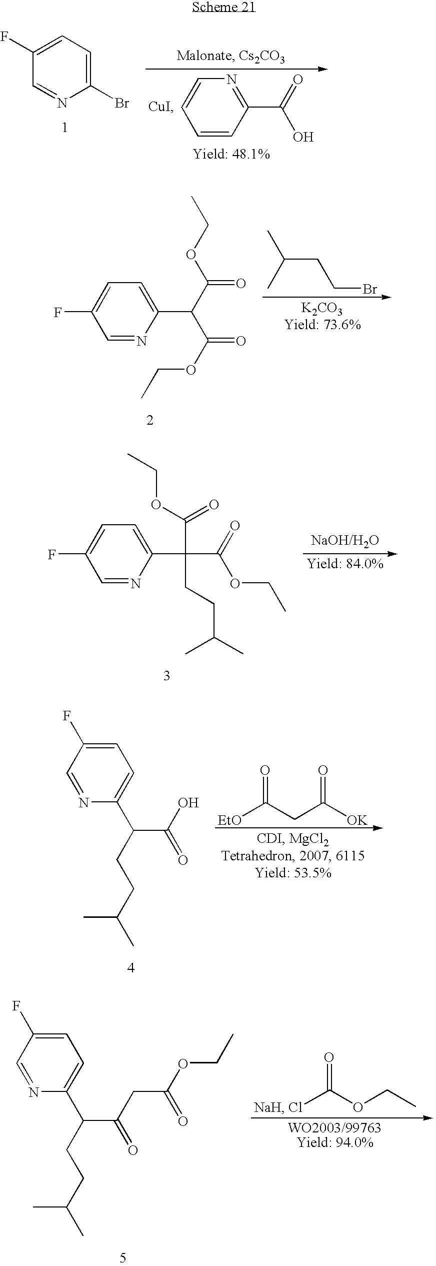

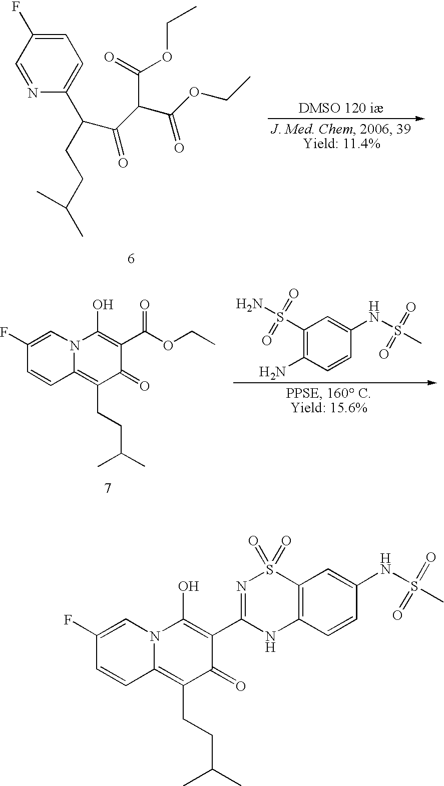

### Preparation of Compound 2

To a solution of compound 1 (20 g, 114.3 mmol) and picolinic acid (11.2 g, 91.4 mmol) in 1,4-dioxane (600 ml) was added CuI (8.7 g, 45.7 mmol) and Cs₂CO₃ (111.8 g, 342.9 mmol). After that, diethyl malonate (73.2 g, 457.2 mmol) was added to the solution and stirred at 100° C. for overnight, then quenched with water and extracted with ethyl acetate. The organic layer was dried over Na₂SO₄ and concentrated. The product was purified by chromatography on silica gel (EA/PE 1:100-1:30) to give compound 2 (14 g, yield: 48.1%) as white oil. ¹H NMR (400 MHz, CDCl₃): 1.278 (d, 6H, J=1.8 Hz), 4.228 (m, 4H), 4.928 (s, 1H), 7.435 (m, 1H), 7.528 (m, 1H), 8.408 (d, 1H, J=2.8 Hz). MS-ESI: m/z=256 [M+1]⁺.

### Preparation of Compound 3

Compound 2 (10 g, 39.2 mmol) was dissolved in DMF (200 ml), then K₂CO₃(21.6 g, 156.8 mmol) was added followed by 1-bromo-3-methylbutane (35.3 g, 235.2 mmol). Immerse the flask in an oil bath and heat slowly so that the temperature reaches 50-60° C. for overnight. The reaction mixture was partitioned between EtOAc (1500 ml) and water (1000 ml). After quenching the reaction, the reaction mixture was poured into separatory funnel and separated. The organic layer was dried on Na₂SO₄ and concentrated. The crude product was chromatographed on silica gel (EA/PE 1:100-1:30) to give compound 3 (9.4 g, 73.6%) as a white liquid. ¹H NMR (300 MHz, CDCl₃): 0.762 (d, 6H, J=2.4 Hz), 0.971 (m, 2H), 1.165 (m, 6H), 1.460 (m, 1H), 2.267 (m, 2H), 4.156 (m, 4H), 7.317 (m, 1H), 7.688 (m, 1H), 8.321 (d, 1H, J=2.8 Hz). MS-ESI: m/z=326 [M+1]⁺.

### Preparation of Compound 4

Compound 3 (7.5 g, 23 mmol) was added to the solution of 40 ml of 1M NaOH and stirred at 100° C. for 1 h. Then the mixture was cooled in an ice bath and neutralized with 1N HCl to pH˜1. The solution was extracted with ethyl acetate and the organic layer was separated. The combined organic layer was dried over Na₂SO₄ and concentrated to give compound 4 (4.3 g, yield: 84%) which was used directly for the next step. MS-ESI: m/z=226 [M+1]⁺.

### Preparation of Compound 5

A solution of compound 4 (4.3 g, 19.1 mmol) in anhydrous tetrahydrofuran (THF) (100 ml) was cooled in salt-ice bath, and N,N′-carbonyldiimidazole (5.64 g, 34.4 mmol) was added in small portions under vigorous stirring. After evolution of gas, the mixture was stirred at room temperature for 3 h and then cooled in an ice bath. To a suspension of monoethyl malonate potassium salt (14.36 g, 84 mmol) in THF (80 ml) in ice bath was added Et₃N (13.5 g, 133.7 mmol) followed by anhydrous MgCl₂ (9.96 g, 104.8 mmol). The mixture was stirred at room temperature for 3 h, then cooled in salt-ice bath and the above solution of the activated ester previously prepared in THF was added dropwise slowly. The mixture was allowed to stir for 24 h at room temperature, quenched with aqueous citric acid and extracted with ethyl acetate. The organic layers were washed with saturated NaHCO₃ solution and brine, dried (Na₂SO₄) and concentrated in vacuo and purified by chromatography on silica gel (EA/PE 1:100-1:20) to give compound 5 (3 g, yield: 53.5%). ¹H NMR (400 MHz, CDCl₃): 0.762 (d, 6H, J=1.8 Hz), 0.919 (m, 1H), 1.076 (m, 1H), 1.215 (m, 4H), 1.452 (m, 1H), 1.758 (m, 1H), 2.031 (m, 1H), 3.449 (m, 2H), 3.943 (m, 1H), 4.123 (q, 2H), 7.169 (m, 1H), 7.327 (m, 1H), 8.360 (d, 1H, J=2.8 Hz). MS-ESI: m/z=296 [M+1]⁺.

### Preparation of Compound 6

Compound 5 (3 g, 10.2 mmol) was dissolved in anhydrous THF (40 mL) and cooled to 0° C. NaH (60% in oil, 1.2 g, 30.6 mmol) was added and the mixture stirred for 45 min at room temperature. After cooling again to 0° C., a solution of ethyl chloroformate (2.2 g, 20.2 mmol) in anhydrous THF (5 mL) was slowly added with a seringe. The solution was stirring at room temperature for 2 h, treated with water, acidified to pH˜3 by addition of citric acid and extracted with ethyl acetate. The organic layer was dried over Na₂SO₄ and concentrated in vacuo to give crude product 6 (3.5 g, yield: 94%) which was used directly for the next step. MS-ESI: m/z=368 [M+1]⁺.

### Preparation of Compound 7

The crude compound 6 (2 g, 5.5 mmol) was dissolved in Dowtherm oil (20 mL) and heated to 230° C. for 20 mins. Then it was cooled and purified by pre-HPLC (EA/PE 1:3) to give compound 7 as brown solid (0.2 g, yield: 11.4%). ¹H NMR (400 MHz, CDCl₃): 0.991 (d, 6H, J=2.2 Hz), 1.41 (m, 2H), 1.492 (t, 3H, J=6.4 Hz), 1.598 (m, 1H), 2.67 (m, 2H), 4.464 (q, 2H, J=1.8 Hz), 7.284 (m, 1H), 7.440 (m, 1H), 8.975 (d, 1H, J=2.8 Hz), 13.43 (s, 1H). MS-ESI: m/z=322.1 [M+1]⁺.

### Preparation of Compound 250

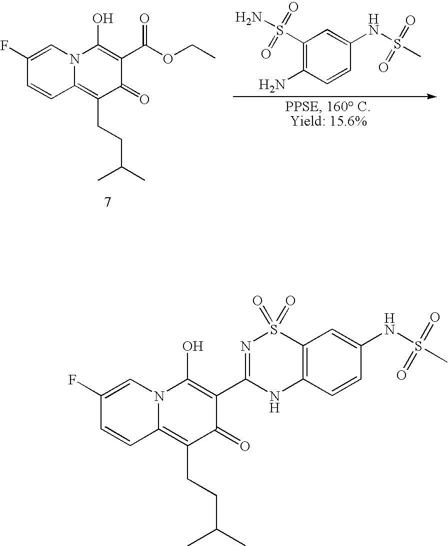

Compound 7 (200 mg, 0.62 mmol) was added to PPSE (0.5 mL) which and then 2-amino-5-(methylsulfonamido)benzenesulfonamide (500 mg, 1.86 mmol) was added. The solution stirred for 2 h at 180° C. The cooled mixture was poured into water and extracted with ethyl acetate. The combined organic layer was dried over Na₂SO₄ and concentrated. Then the residue was re-crystallized in ethyl acetate to give compound 250 as a yellow solid (50 mg, yield: 15.6%. Purity: 98.2% in LC-Ms). ¹H NMR (400 MHz, DMSO): 0.968 (d, 6H, J=6.8 Hz), 1.357 (m, 2H,), 1.661 (m, 1H), 2.824 (m, 2H), 3.171 (s, 3H), 7.647 (m, 3H), 7.974 (m, 2H,), 9.02 (d, 1H, J=5.6 Hz), 10.296 (s, 1H), 14.154 (s, 1H), 14.209 (s, 1H). MS-ESI: m/z=523 [M+1]⁺.

### Preparation of Compound 251

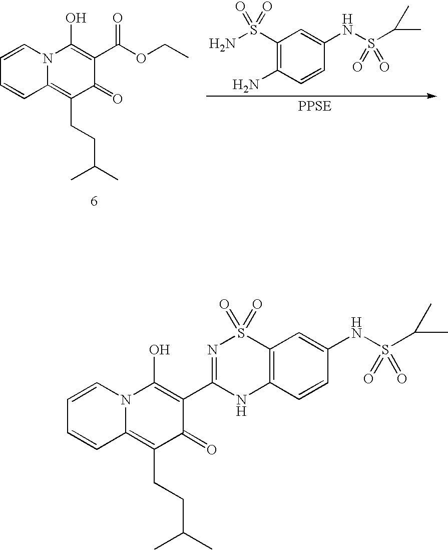

Compound 6 (200 mg, 0.66 mmol) was added at 160° C. to PPSE (3 mL) which and then 2-amino-5-(isopropanesulfonamido)benzenesulfonamide (192 mg, 0.66 mmol) was added. The solution stirred for 1.5 h at 160° C. The cooled mixture was poured into water extracted with ethyl acetate. The organic layer was dried on Na₂SO₄ and concentrated. The product was purified by prep-TLC to give the compound 251 as a yellow solid (36.7 mg, yield: 15.8%. Purity: 97.8% in LC-Ms). ¹H NMR (400 MHz, DMSO): 0.973 (d, 6H, J=6.8 Hz), 1.291 (d, 6H, J=6.8 Hz), 1.361 (m, 2H), 1.672 (m, 1H), 2.787 (t, 2H, J=7.8 Hz), 3.310 (m, 1H), 7.286 (t, 1H, J=6.8 Hz), 7.638 (m, 3H), 7.823 (m, 2H), 9.056 (d, 1H, J=7.2 Hz), 10.333 (s, 1H), 14.135 (s, 1H), 14.275 (s, 1H). MS-ESI: m/z=551.0 [M+23+1]⁺.

### Example 22

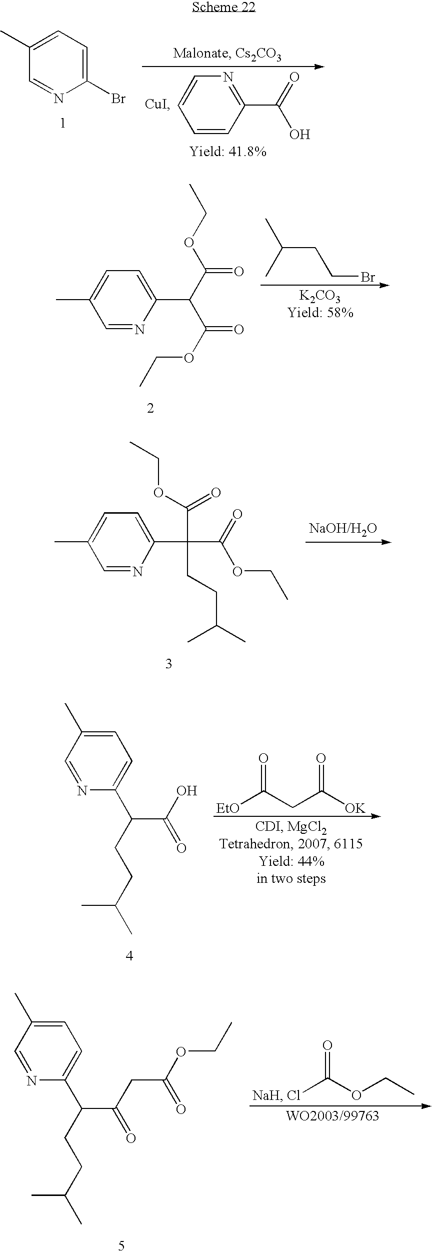

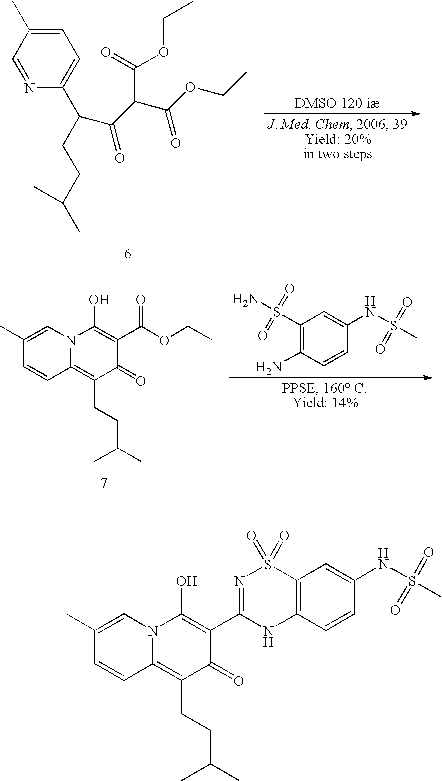

### Preparation of Compound 2

To a solution of compound 1 (10 g, 58 mmol) and picolinic acid (5.7 g, 46 mmol) in 1,4-dioxane (200 ml) was added CuI (4.43 g, 23 mmol) and Cs₂CO₃ (56 g, 174 mmol). After that, diethyl malonate (37.24 g, 232.53 mmol) was added to the solution and stirred at 100° C. for overnight, then quenched with water and extracted with ethyl acetate. The organic layer was dried over Na₂SO₄ and concentrated. The product was purified by chromatography on silica gel (EA/PE 1:100-1:50) to give compound 2 (6 g, yield: 41.8%) as white oil. ¹H NMR (400 MHz, CDCl₃): 1.256 (m, 6H), 2.316 (s, 3H), 4.210 (m, 4H), 7.367 (d, 1H, J=8.4 Hz), 7.510 (d, 1H, J=8 Hz), 8.376 (d, 1H, J=1.6 Hz). MS-ESI: m/z=252 [M+1]⁺

### Preparation of Compound 3

Compound 2 (6 g, 23.88 mmol) was dissolved in DMF (20 ml), then K₂CO₃ (6.6 g, 47.76 mmol) was added followed by 1-bromo-3-methylbutane (4.33 g, 28.65 mmol). Immerse the flask in an oil bath and heat slowly so that the temperature reached 50-60° C. overnight. The reaction mixture was partitioned between EtOAc (500 ml) and water (500 ml). The reaction mixture was poured into separatory funnel and separated. The organic layer was dried over Na₂SO₄ and concentrated. The crude product was chromatographed on silica gel (EA/PE 1:60-1:30) to give compound 3 (4.5 g, 58%) as a colorless liquid. ¹H NMR (400 MHz, CDCl₃): 0.777 (d, 6H, J=6.8 Hz), 1.016 (m, 1H), 1.158 (m, 6H), 1.440 (m, 1H), 2.245 (s, 1H), 2.276 (m, 2H), 4.160 (m, 4H), 7.416 (d, 1H, J=1.6 Hz), 7.513 (d, 1H, J=8 Hz), 8.321 (s, 1H). MS-ESI: m/z=322 [M+1]⁺

### Preparation of Compound 4

Compound 3 (4.5 g, 14 mmol) was added to the solution of 20 ml of 1M NaOH and stirred at 100° C. for 1 h. Then the mixture was cooled in an ice bath and neutralized with 1N HCl to pH˜1. The solution was freeze-dried to give the mixture of compound 4 with NaCl salt which was used directly for the next step. MS-ESI: m/z=222 [M+1]⁺

### Preparation of Compound 5

A solution of crude compound 4 (14 mmol) in anhydrous tetrahydrofuran (THF) (50 ml) was cooled in salt-ice bath, and N,N′-carbonyldiimidazole (3.41 g, 21 mmol) was added in small portions under vigorous stirring. After evolution of gas, the mixture was stirred at room temperature for 3 h and then cooled in an ice bath. To a suspension of monoethyl malonate potassium salt (7.15 g g, 42 mmol) in THF (80 ml) in ice bath was added Et₃N (10 ml) followed by anhydrous MgCl₂ (4.8 g, 42.03 mmol). The mixture was stirred at room temperature for 3 h, then cooled in salt-ice bath and the above solution of the activated ester previously prepared in THF was added dropwise slowly. The mixture was allowed to stir for 39 h at room temperature, quenched with aqueous citric acid and extracted with ethyl acetate. The organic layers were washed with saturated NaHCO₃ solution and brine, dried (Na₂SO₄) and concentrated in vacuo and purified by chromatography on silica gel (EA/PE 1:50-1:3) to give compound 5 (1.8 g, yield: 44% in two steps). ¹H NMR (400 MHz, CDCl₃): 0.777 (m, 6H,), 0.949 (m, 1H), 1.084 (m, 1H), 1.155 (m, 4H), 1.479 (m, 1H), 1.575 (m, 1H), 2.242 (m, 1H), 2.255 (s, 1H), 3.365 (dd, 2H, J1=48 Hz, J2=13.6 Hz), 3.860 (t, 1H, J=7.4 Hz), 4.046 (q, 2H, J2=6.4 Hz), 7.041 (d, 1H, J=8 Hz), 7.692 (d, 1H, J=8 Hz), 8.325 (s, 1H). MS-ESI: m/z=292 [M+1]⁺

### Preparation of Compound 6

Compound 5 (1.8 g, 6.18 mmol) was dissolved in anhydrous THF (20 mL) and cooled to 0° C. NaH (60% in oil, 500 mg, 12.35 mmol) was added and the mixture stirred for 45 min at room temperature. After cooling again to 0° C., a solution of ethyl chloroformate (871.51 mg, 8.03 mmol) in anhydrous THF (0.5 mL) was slowly added with a seringe. The solution was stirring at room temperature for 2 h, treated with water, acidified to pH˜3 by addition of citric acid and extracted with ethyl acetate. The organic layer was dried over Na₂SO₄ and concentrated in vacuo to give crude product 6 which was used directly for the next step. MS-ESI: m/z=364 [M+1]⁺

### Preparation of Compound 7

The crude compound 6 (6.18 mmol) was dissolved in DMSO (20 mL) and heated to 120° C. for 8 h. Then it was poured into water and extracted with ethyl acetate. The organic layer was washed with water, dried over Na₂SO₄ and concentrated in vacuo. The product was purified by chromatography on silica gel (EA/PE 1:50-1:3) to give compound 7 as brown solid (0.4 g, yield: 20% in two steps). ¹H NMR (400 MHz, CDCl₃): 0.995 (d, 6H, J=6.8 Hz), 1.391 (m, 2H), 1.466 (t, 3H, J=7.2 Hz), 1.673 (m, 1H), 2.327 (s, 1H), 2.739 (m, 2H), 4.497 (q, 2H, J2=6.8 Hz), 7.319 (d, 1H, J=1.6 Hz), 7.432 (d, 1H, J=9.2 Hz), 8.957 (s, 1H), 13.405 (s, 1H). MS-ESI: m/z=318.1 [M+1]⁺

### Preparation of Compound 252

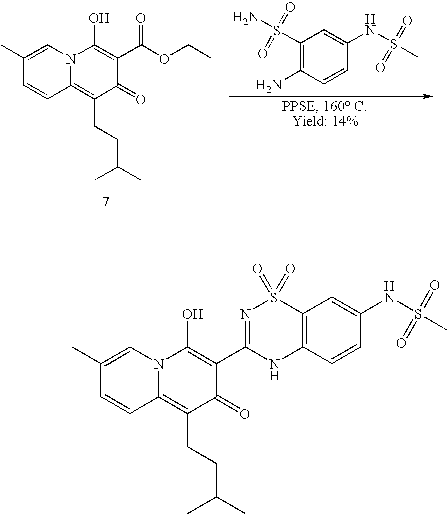

Compound 7 (50 mg, 0.157 mmol) was added at 160° C. to PPSE (0.5 mL) which and then 2-amino-5-(methylsulfonamido)benzenesulfonamide (41 mg, 0.157 mmol) was added. The solution stirred for 1 h at 160° C. The cooled mixture was poured into water and the precipitate was collected and washed with MeOH for several times. Then it was dried to give compound 252 as a green solid (11 mg, yield: 14%. Purity: 95.3% in LC-Ms). ¹H NMR (400 MHz, DMSO): 1.031 (d, 6H, J=6.8 Hz), 1.420 (m, 2H,), 1.729 (m, 1H), 2.445 (s, 3H,), 2.850 (t, 2H, J=8 Hz), 3.158 (s, 3H), 7.647 (d, 1H, J=2.8 Hz), 7.729 (m, 3H,), 7.891 (d, 1H, J=9.2 Hz), 8.960 (s, 1H), 10.349 (s, 1H), 14.079 (s, 1H), 14.477 (s, 1H). MS-ESI: m/z=519 [M+1]⁺.

### Example 23

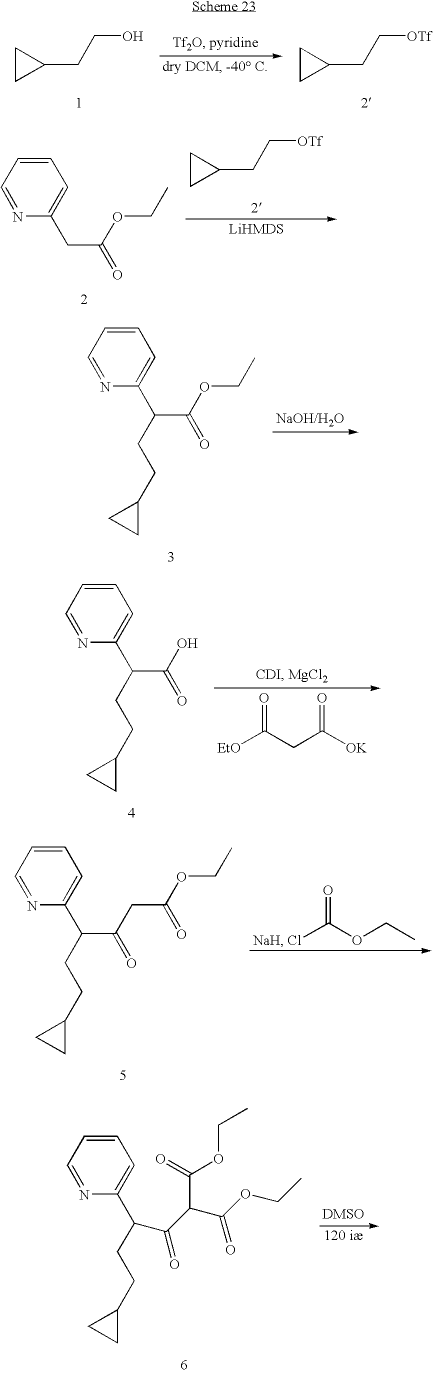

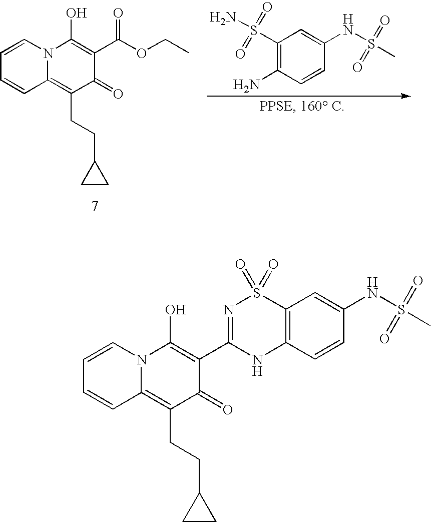

### Preparation of Compound 2

A solution of compound 1 (2.17 g, 25.2 mmol) in dry DCM (40 mL) was added dry pyridine (2.4 g, 30.3 mmol). The reaction mixture was cooled to −40° C., followed by adding dropwise of trifluoromethanesulfonic anhydride (8.5 g, 30.3 mmol). The solution was allowed to stirred for 30 minutes at −40° C., then the reaction mixture was allowed to warm to room temperature. The reaction mixture was diluted with PE (100 mL), concentrated to removes solvent DCM, filtrated and the organic phase was concentrated to give crude compound 2′ (4.72 g, 85.9%).

### Preparation of Compound 3

A solution of compound 2 (3.58 g, 22 mmol) in dry THF (30 mL) was added dropwise of LiHMDS (24 mL, 24 mmol) at −78° C. and stirred for 3 h at this temperature. Then the reaction was slowly warmed to 0° C. for 10 minutes. The reaction mixture was cooled to −78° C., compound 2′ (4.8 g, 22 mmol) added dropwise to the mixture at −78° C. The reaction mixture was allowed to warm to room temperature overnight, quenched with water and extracted with ethyl acetate. The organic layer was dried over Na₂SO₄ and concentrated. The product was purified by chromatography to give compound 3 (4.78 g, yield: 93.2%) as light oil. MS-ESI: m/z=234 [M+1]⁺

### Preparation of Compound 4

The same procedure used to prepare compound 68b in Scheme 10 was used to prepare compound 4 (4.0 g, yield: 95%) MS-ESI: m/z=206 [M+1]⁺

### Preparation of Compound 5

The same procedure used to prepare compound 69b in Scheme 10 was used to prepare compound 5 (440 mg, yield: 32.8%). MS-ESI: m/z=276 [M+1]⁺

### Preparation of Compound 6

The same procedure used to prepare compound 70b in Scheme 10 was used to prepare compound 6 (550 mg, yield: 90.0%). MS-ESI: m/z=348 [M+1]⁺

### Preparation of Compound 7

The same procedure used to prepare compound 71b in Scheme 10 was used to prepare compound 7 (100 mg, yield: 30.0%). MS-ESI: m/z=302 [M+1]⁺

### Preparation of Compound 253

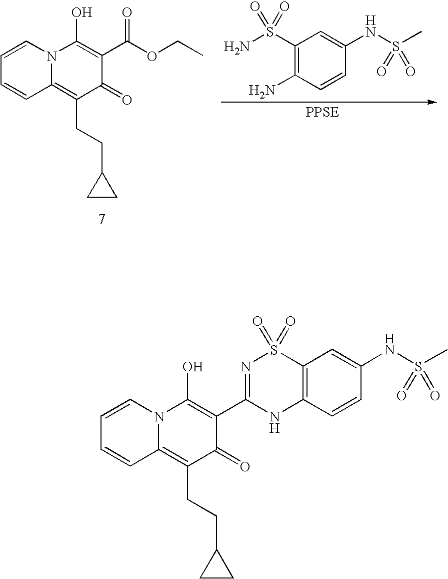

The same procedure used to prepare compound 225 was used to give compound 253 as a yellow solid (4.0 mg, yield: 1.2%). ¹H NMR (400 MHz, DMSO): 0.96 (t, 3H, J=7.2 Hz), 1.62 (m, 3H), 1.66 (m, 1H), 2.18 (m, 1H), 2.67 (m, 1H), 2.91 (m, 1H), 3.05 (s, 3H), 4.12 (m, 1H), 7.24 (t, 1H, J=6.0 Hz), 7.37 (d, 1H, J=8.8 Hz), 7.56 (m, 2H), 7.79 (m, 2H), 9.06 (d, 1H, J=7.6 Hz). MS-ESI: m/z=503 [M+1]⁺

### Example 24

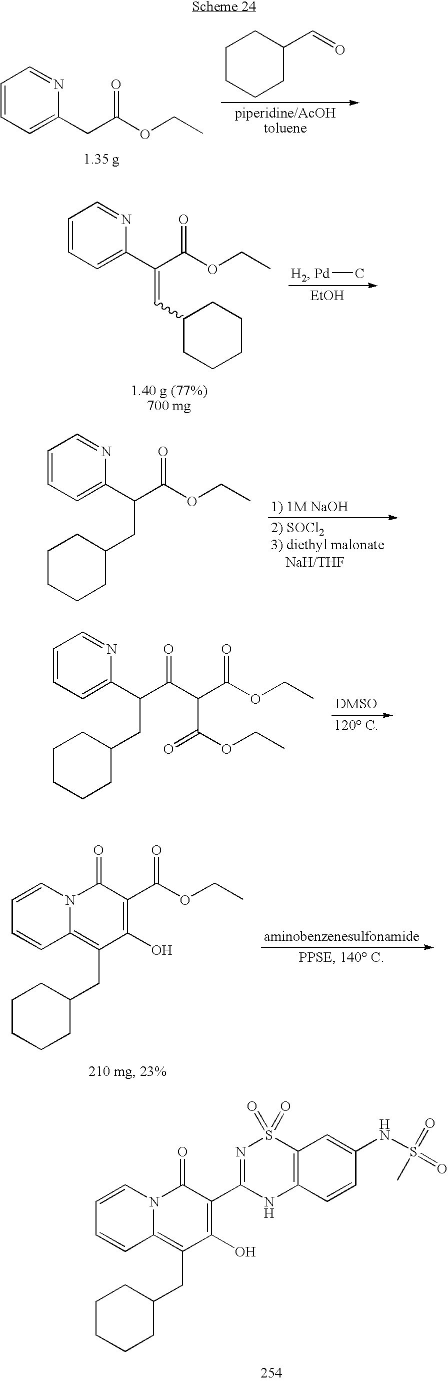

### Preparation of Compound 254

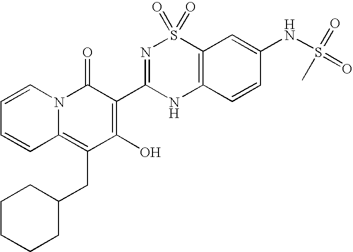

### Preparation of Compound 254 is shown above in Scheme 24

### Example 25

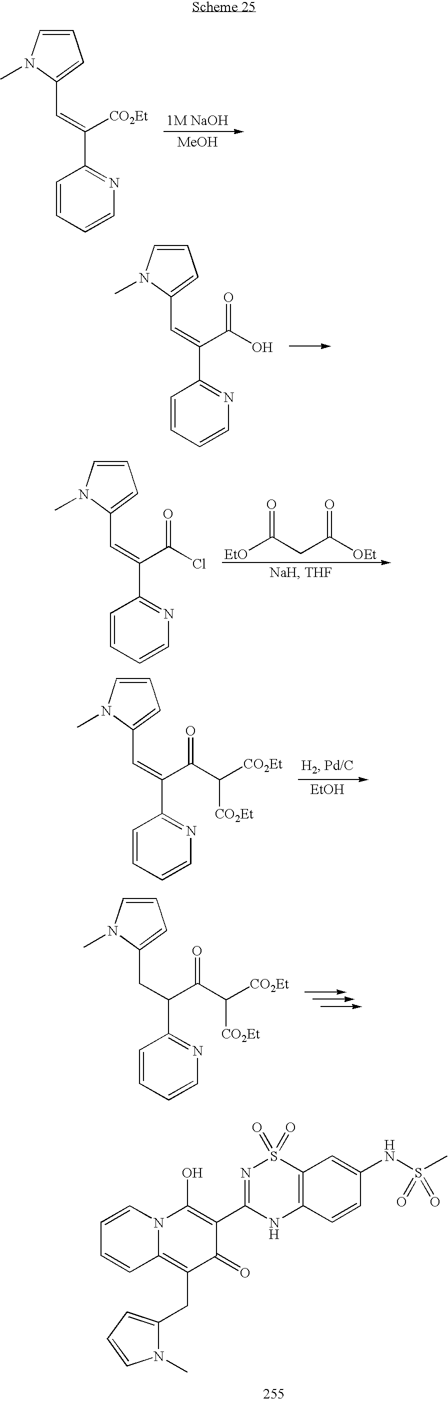

### Preparation of Compound 255

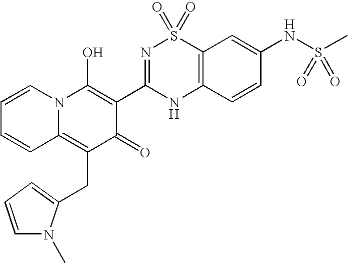

### Preparation of Compound 255 is shown herein in Scheme 25

### Activities of NS5B Inhibitors

The compounds were tested in the Replizyme HCV heterotemplate radioactive RNA-dependent RNA-polymerase (RdRp) assay. The test compounds were pre-incubated with the RNA template and NS5B polymerase protein at 37° C. for 30 minutes. The RdRp reaction was initiated with the addition of the NTPs to the buffer-NS5B-compound mix, and was allowed to proceed for 90 minutes at 37° C. Control reactions included: no enzyme, 5% DMSO (test compound solvent), no compound/solvent, Cordycepin-TP and HCV-796 (IC₅₀ values used as a reference inhibition). Radioactive products were collected by applying the stopped reaction to DE-81 paper, air dried prior to washing with buffer comprising NaH₂PO₄ and sodium pyrophosphate to remove unincorporated ³²P-GTP in the NTP mix, and rinsed with dH₂O followed by 100% ethanol. The DE-81 paper was air dried, squares cut out and placed in scintillation tubes for counting.

TABLE 1

| | HCV Replicon | HCV NS5B inhibition |
|---|---|---|
| Compound | inhibition EC₅₀ (μM) | EC₅₀ (μM) |
| 101 | D | D |
| 102 | C | C |
| 103 | C | D |
| 104 | B | C |
| 105 | B | B |
| 201 | A | A |
| 202 | A | A |
| 203 | E | C |
| 204 | A | C |
| 205 | A | A |
| 206 | E | B |
| 207 | E | B |
| 208 | E | C |
| 209 | E | C |
| 210 | A | C |
| 211 | A | B |
| 212 | E | B |
| 213 | A | D |
| 214 | C | D |
| 215 | | B |
| 216 | D | B |
| 218 | D | B |
| 220 | C | D |
| 221 | E | C |
| 222 | C | D |
| 223 | B | D |
| 224 | D | A |
| 225 | D | D |
| 226 | D | D |
| 227 | D | D |
| 228 | D | D |
| 229 | D | C |
| 230 | D | B |
| 231 | D | A |
| 232 | D | E |
| 233 | D | B |
| 234 | D | A |
| 235 | D | B |
| 236 | D | D |
| 237 | D | D |
| 238 | D | D |
| 242 | | A |
| 244 | | D |
| 245 | D | C |
| 249 | D | C |
| 250 | D | D |
| 251 | D | C |
| 252 | D | D |
| 253 | C | B |

A indicates an EC₅₀ or IC₅₀ between 10 and 50 μM
B indicates an EC₅₀ or IC₅₀ between 1 and 10 μM
C indicates an EC₅₀ or IC₅₀ between 0.1 and 1 μM
D indicates an EC₅₀ or IC₅₀ of less than 0.1 μM
E indicates an EC₅₀ or IC₅₀ of greater than 50 μM

### CONCLUSION

Potent small molecule inhibitors of the HCV NS5B polymerase have been developed.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A compound having the structure of Formula I: 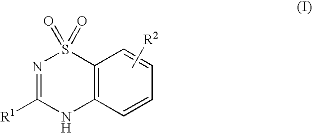
or a pharmaceutically acceptable salt or prodrug thereof wherein:
R¹ is selected from the group consisting of: 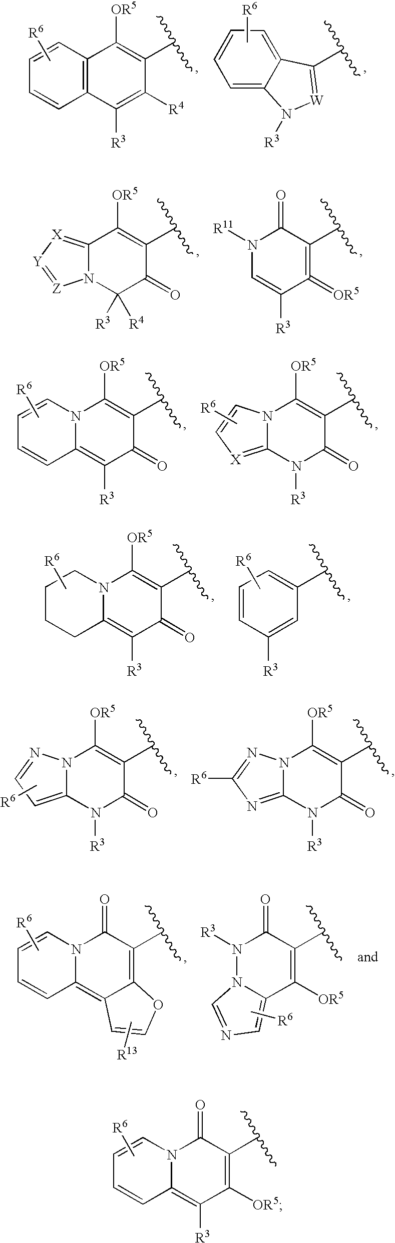
X, Y, and Z are each N or CR⁷, wherein each R⁷ is independently selected from the group consisting of hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino;
W is N or CR¹², wherein R¹² is selected from the group consisting of hydrogen, hydroxyl, optionally substituted alkyl, optionally substituted alkoxy and optionally substituted amino;
R² is present from 0 to 4 times, wherein each R² is independently selected from the group consisting of hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted amino, and —NH(SO₂R⁸), each R⁸ is independently selected from the group consisting of optionally substituted alkyl and optionally substituted cycloalkyl;
R³ is selected from the group consisting of hydrogen, halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, optionally substituted arylalkyl, optionally substituted heteroarylalkyl, optionally substituted amino and haloalkyl;
R⁴ is selected from the group consisting of hydrogen, hydroxyl, optionally substituted alkyl, optionally substituted alkoxy and optionally substituted amino;
R⁵ is selected from the group consisting of hydrogen and optionally substituted alkyl;
R⁶ is present from 0 to 4 times, wherein each R⁶ is independently selected from the group consisting of halogen, hydroxy, cyano, nitro, optionally substituted alkyl, optionally substituted alkoxy, optionally substituted cycloalkyl, optionally substituted heterocyclyl, optionally substituted aryl, optionally substituted heteroaryl, and optionally substituted amino;
R¹¹ is selected from the group consisting of an optionally substituted aryl, an optionally substituted heteroaryl, an optionally substituted alicyclyl, an optionally substituted heterocyclyl, an optionally substituted alkyl, an optionally substituted alkenyl, an optionally substituted alkynyl, alkyl-CO—, and alkenyl-CO—;
R¹³ is selected from the group consisting of hydrogen, hydroxyl, optionally substituted alkyl, optionally substituted alkoxy and optionally substituted amino; and
with the proviso that Formula I cannot be

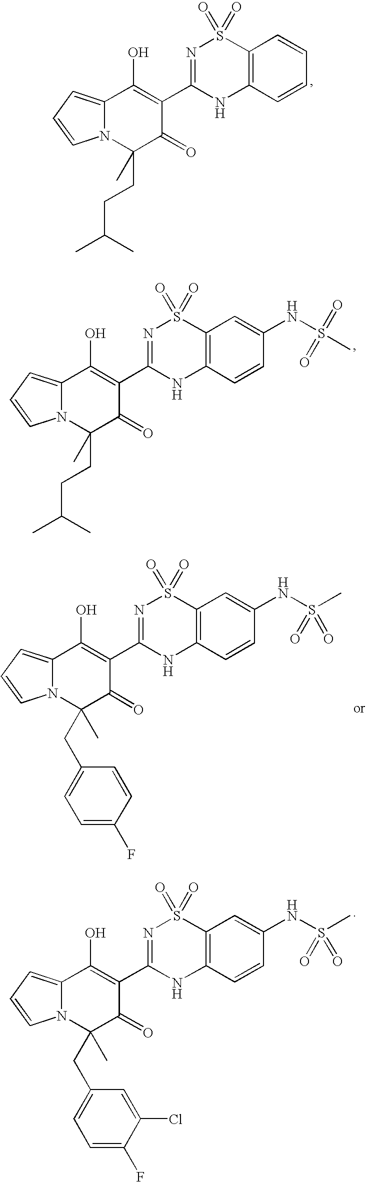

2. The compound of claim 1, wherein when X, Y and Z are CH, R³ and R⁴ cannot both be optionally substituted alkyl.

3. The compound of claim 1, wherein R³ is —NR⁹R¹⁰, wherein R⁹ and R¹⁰ are independently selected from the group consisting of hydrogen and optionally substituted alkyl.

4. The compound of claim 1, wherein R³ is selected from the group consisting of halogen, optionally substituted arylalkyl, and optionally substituted alkyl.

5. The compound of claim 1, wherein R⁶ is not present.

6. The compound of claim 1, wherein R² is not present.

7. The compound of claim 1, wherein R¹ is

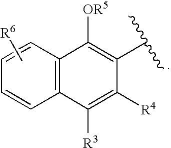

8. (canceled)

9. (canceled)

10. (canceled)

11. (canceled)

12. (canceled)

13. (canceled)

14. (canceled)

15. (canceled)

16. (canceled)

17. (canceled)

18. The compound of claim 1, wherein R¹ is

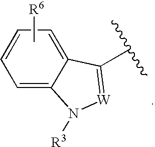

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. The compound of claim 1, wherein R¹ is

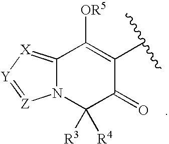

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. The compound of claim 1, wherein R¹ is

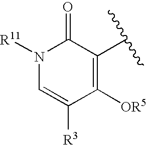

44. (canceled)

45. (canceled)

46. (canceled)

47. (canceled)

48. (canceled)

49. (canceled)

50. The compound of claim 1, wherein R¹ is

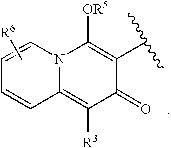

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. The compound of claim 1, wherein R¹ is

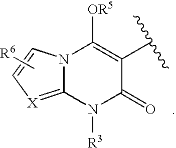

65. (canceled)

66. (canceled)

67. (canceled)

68. (canceled)

69. (canceled)

70. (canceled)

71. (canceled)

72. The compound of claim 1, wherein R¹ is

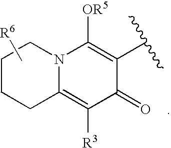

73. (canceled)

74. (canceled)

75. (canceled)

76. (canceled)

77. The compound of claim 1, wherein R¹ is

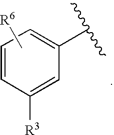

78. (canceled)

79. (canceled)

80. (canceled)

81. (canceled)

82. The compound of claim 1, wherein R¹ is

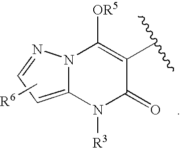

83. (canceled)

84. (canceled)

85. (canceled)

86. (canceled)

87. (canceled)

88. (canceled)

89. (canceled)

90. (canceled)

91. (canceled)

92. (canceled)

93. The compound of claim 1, wherein R¹ is

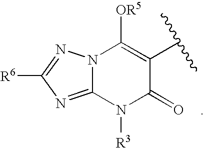

94. (canceled)

95. (canceled)

96. (canceled)

97. (canceled)

98. (canceled)

99. The compound of claim 1, wherein R¹ is

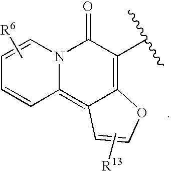

100. (canceled)

101. (canceled)

102. (canceled)

103. (canceled)

104. The compound of claim 1, wherein R¹ is

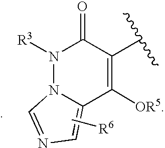

105. (canceled)

106. (canceled)

107. (canceled)

108. (canceled)

109. (canceled)

110. The compound of claim 1, wherein R¹ is

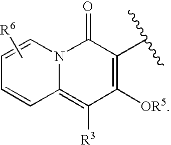

111. (canceled)

112. (canceled)

113. (canceled)

114. (canceled)

115. (canceled)

116. (canceled)

117. The compound of claim 1 having one of the following structures selected from the group consisting of:

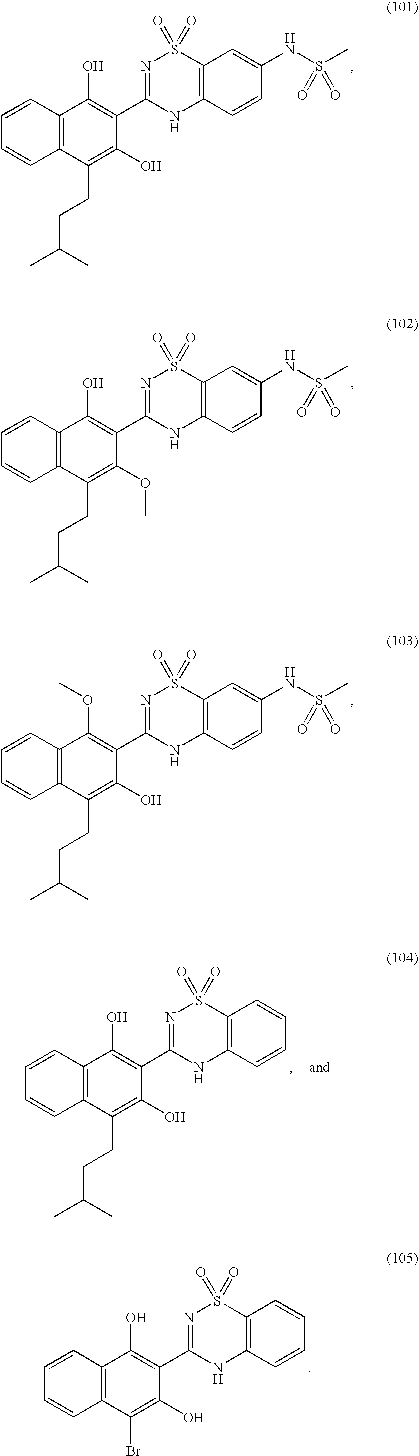

118. The compound of claim 1 having one of the following structures selected from the group consisting of:

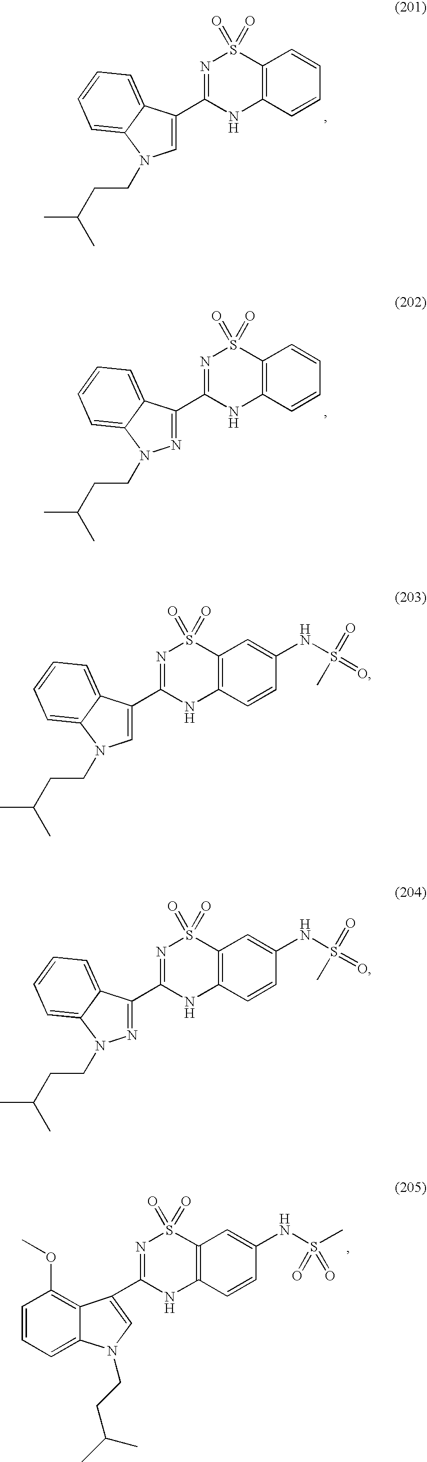

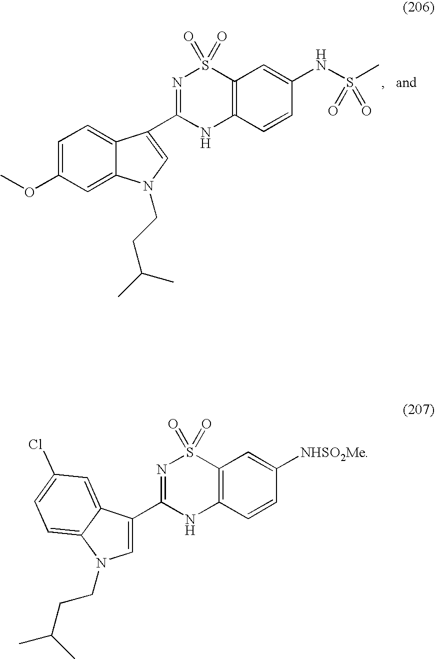

119. The compound of claim 1 having one of the following structures selected from the group consisting of:

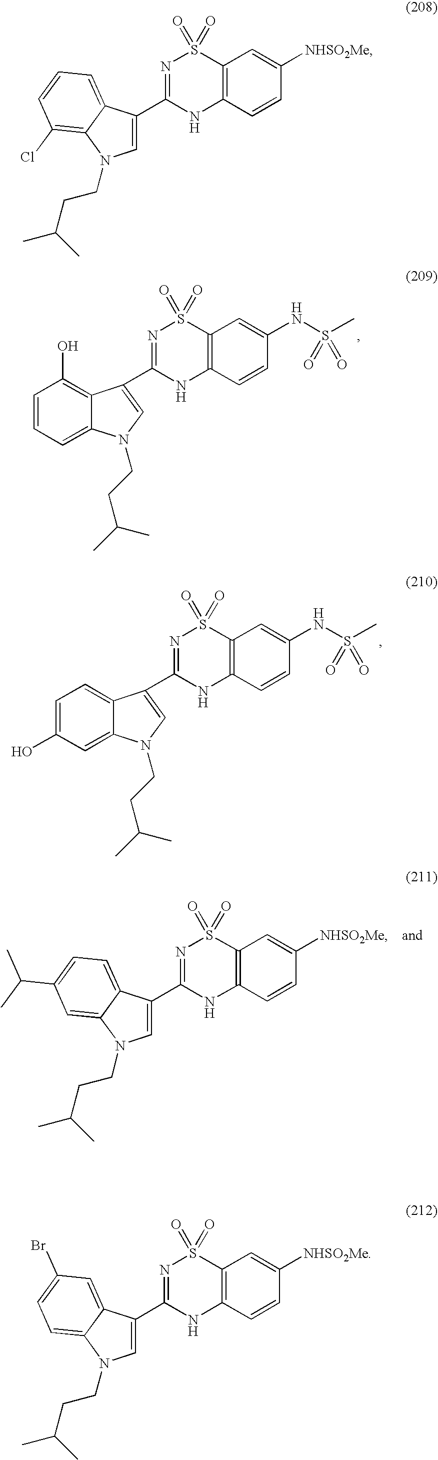

120. The compound of claim 1 having one of the following structures selected from the group consisting of:

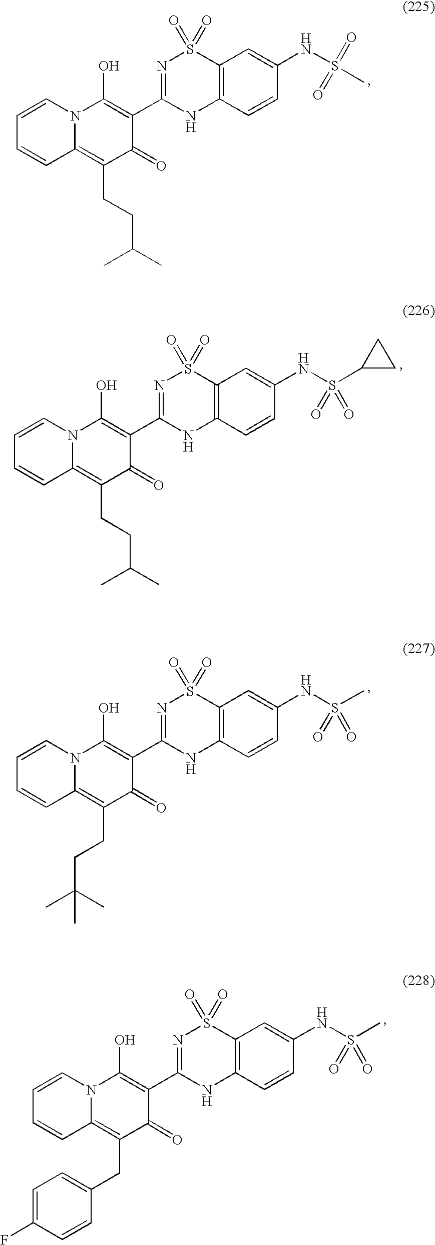

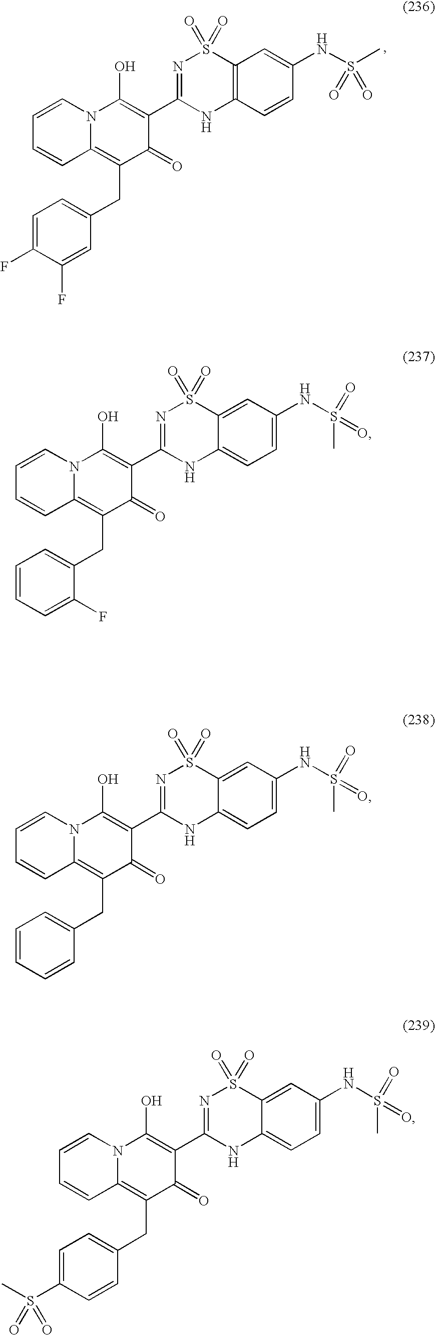

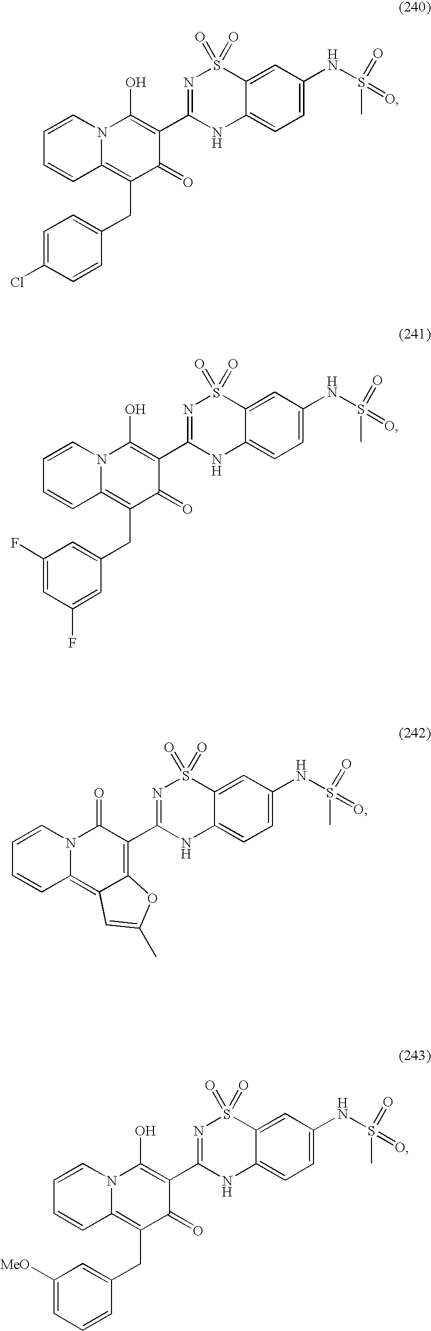

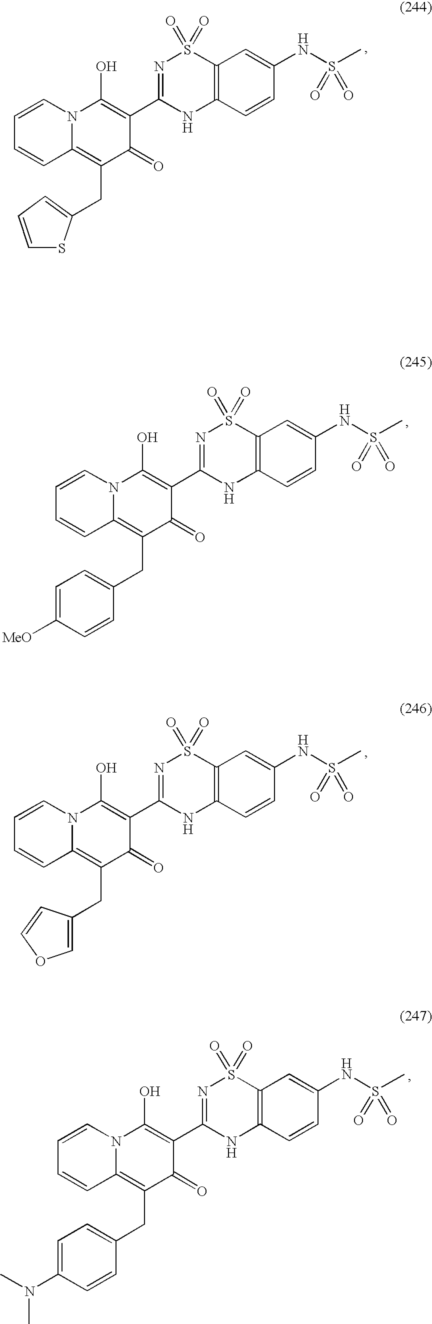

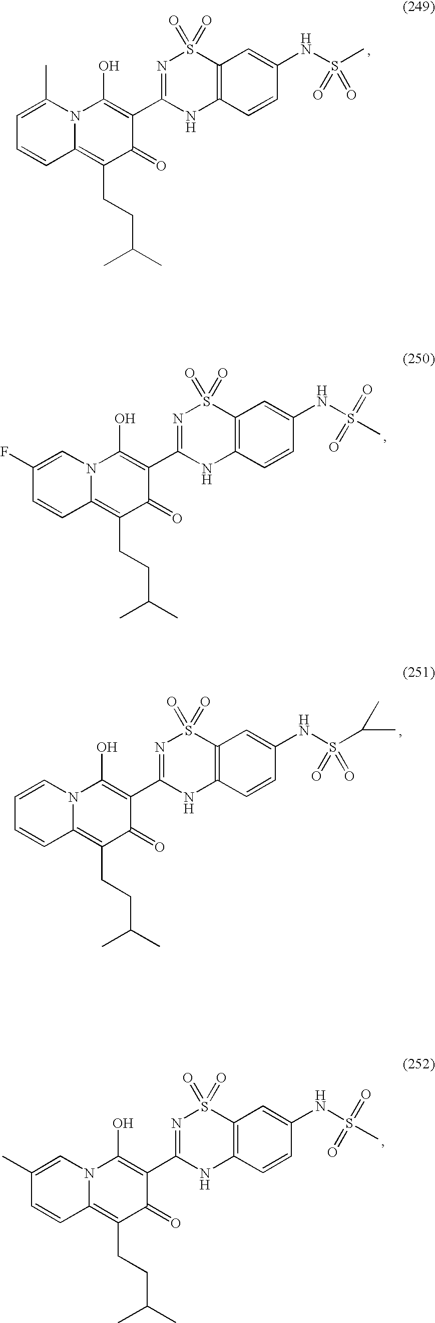

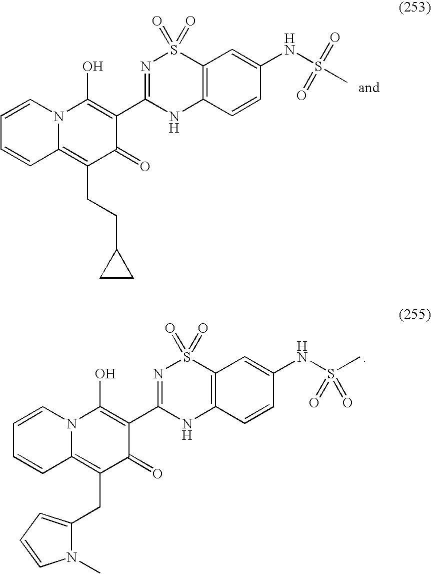

121. The compound of claim 1 having one of the following structures selected from the group consisting of:

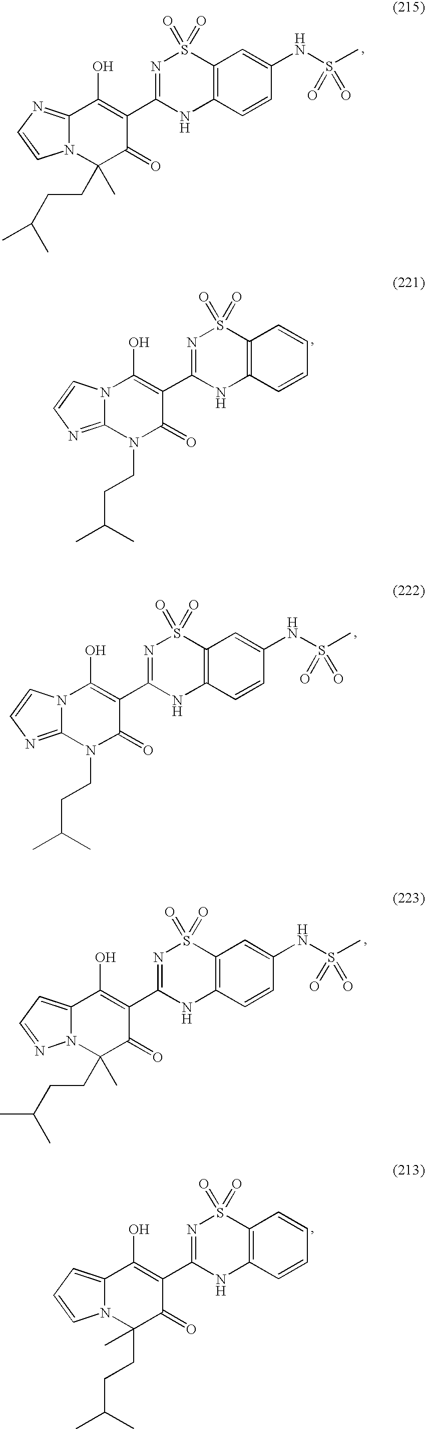

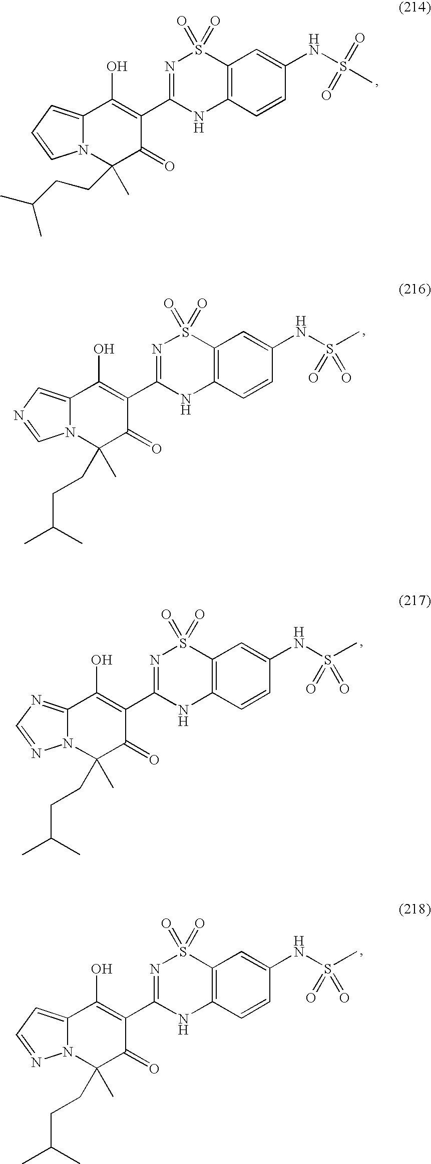

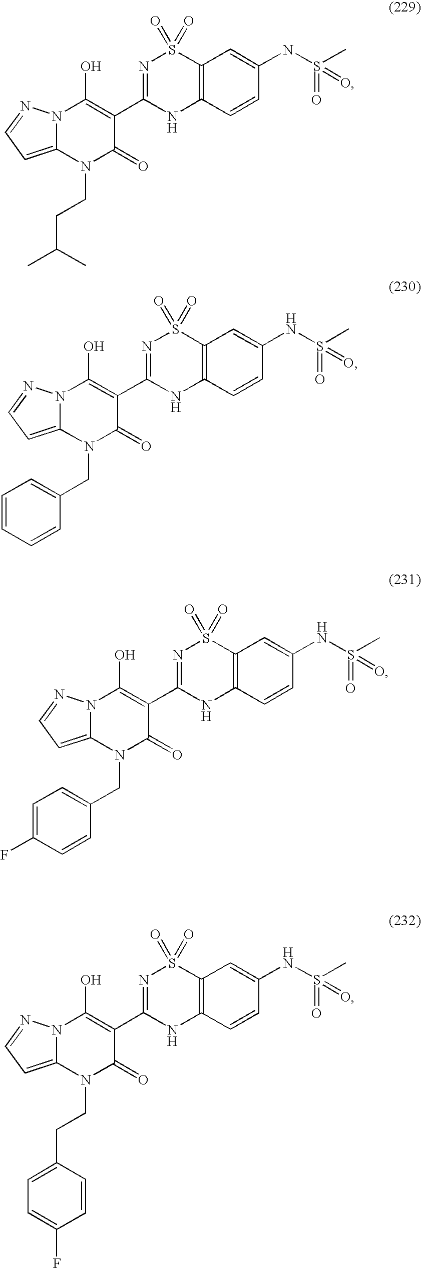

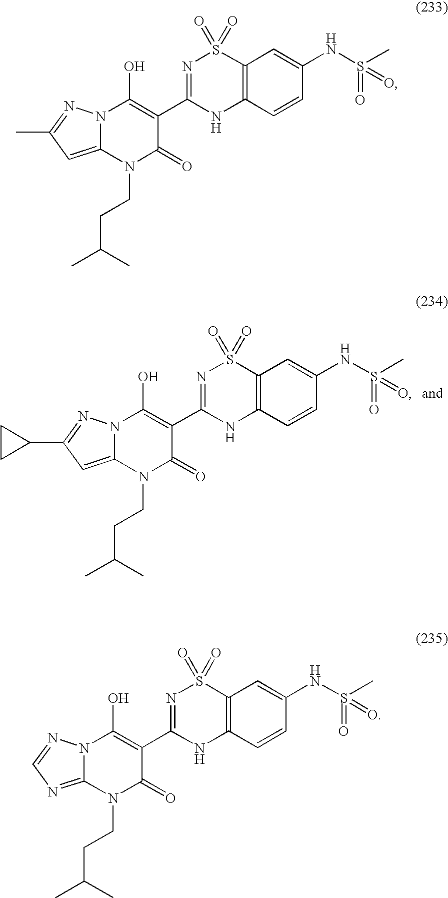

122. The compound of claim 1 having one of the following structures selected from the group consisting of:

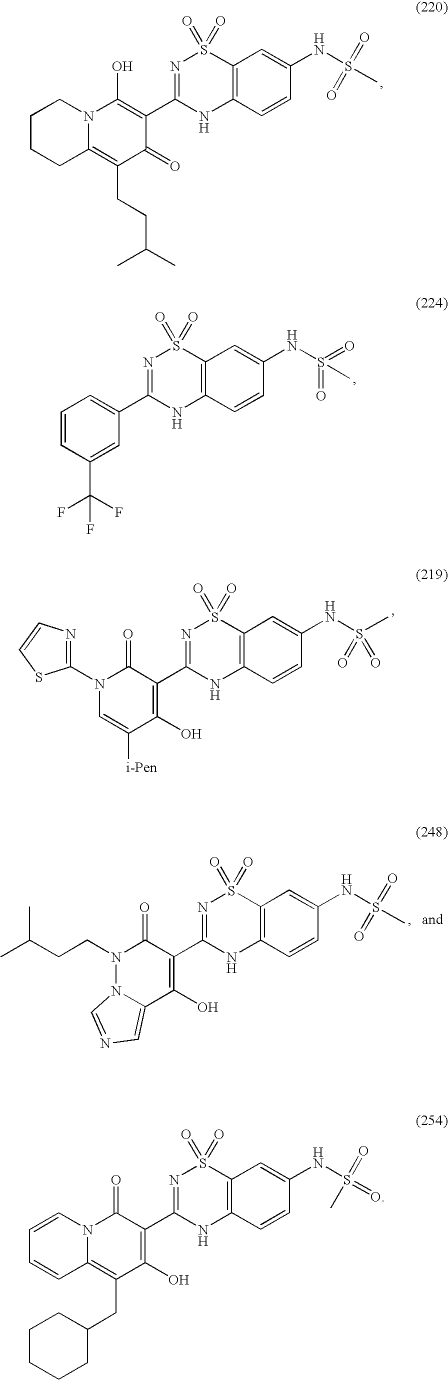

123. A pharmaceutical composition comprising a pharmaceutically acceptable excipient and one of more compounds of claim 1.

124. A method of inhibiting NS5B polymerase activity comprising contacting a NS5B polymerase with a compound of claim 1.

125. The method of claim 124 in which the contacting is conducted in vivo.

126. The method of claim 125, further comprising identifying a subject suffering from a hepatitis C infection and administering the compound to the subject in an amount effective to treat the infection.

127. The method of claim 126, wherein the method further comprises administering to the individual an effective amount of a nucleoside analog.

128. The method of claim 127, wherein the nucleoside analog is selected from ribavirin, levovirin, viramidine, an L-nucleoside, and isatoribine.

129. The method of claim 126, wherein the method further comprises administering to the individual an effective amount of a human immunodeficiency virus 1 protease inhibitor.

130. The method of method of claim 129, wherein the protease inhibitor is ritonavir.

131. The method of claim 126, wherein the method further comprises administering to the individual an effective amount of an NS3 protease inhibitor.

132. The method of claim 126, wherein the method further comprises administering to the individual an effective amount of interferon-gamma (IFN-γ).

133. The method of claim 132, wherein the IFN-γ is administered subcutaneously in an amount of from about 10 μg to about 300 μg.

134. The method of claim 126, wherein the method further comprises administering to the individual an effective amount of interferon-alpha (IFN-α).

135. The method of claim 134, wherein the IFN-α is monoPEG-ylated consensus IFN-α administered at a dosing interval of every 8 days to every 14 days.

136. The method of claim 134, wherein the IFN-α is monoPEG-ylated consensus IFN-α administered at a dosing interval of once every 7 days.

137. The method of claim 134, wherein the IFN-α is INFERGEN consensus IFN-α.

138. The method of claim 126, further comprising administering an effective amount of an agent selected from 3′-azidothymidine, 2′,3′-dideoxyinosine, 2′,3′-dideoxycytidine, 2′,3′-didehydro-2′,3′-dideoxythymidine, combivir, abacavir, adefovir dipoxil, cidofovir, and an inosine monophosphate dehydrogenase inhibitor.

139. The method of claim 126, wherein a sustained viral response is achieved.

140. The method of claim 124, in which the contacting is conducted ex vivo.

141. A method of treating liver fibrosis in an individual, the method comprising administering to the individual an effective amount of a compound of claim 1.

142. The method of claim 141, wherein the method further comprises administering to the individual an effective amount of a nucleoside analog.

143. The method of claim 142, wherein the nucleoside analog is selected from ribavirin, levovirin, viramidine, an L-nucleoside, and isatoribine.

144. The method of claim 141, wherein the method further comprises administering to the individual an effective amount of a human immunodeficiency virus 1 protease inhibitor.

145. The method of method of claim 144, wherein the protease inhibitor is ritonavir.

146. The method of claim 141, wherein the method further comprises administering to the individual an effective amount of an NS3 protease inhibitor.

147. The method of claim 141, wherein the method further comprises administering to the individual an effective amount of interferon-gamma (IFN-γ).

148. The method of claim 147, wherein the IFN-γ is administered subcutaneously in an amount of from about 10 μg to about 300 μg.

149. The method of claim 141, wherein the method further comprises administering to the individual an effective amount of interferon-alpha (IFN-α).

150. The method of claim 149, wherein the IFN-α is monoPEG-ylated consensus IFN-α administered at a dosing interval of every 8 days to every 14 days.

151. The method of claim 149, wherein the IFN-α is monoPEG-ylated consensus IFN-α administered at a dosing interval of once every 7 days.

152. The method of claim 149, wherein the IFN-α is INFERGEN consensus IFN-α.

153. The method of claim 141, further comprising administering an effective amount of an agent selected from 3′-azidothymidine, 2′,3′-dideoxyinosine, 2′,3′-dideoxycytidine, 2′,3′-didehydro-2′,3′-dideoxythymidine, combivir, abacavir, adefovir dipoxil, cidofovir, and an inosine monophosphate dehydrogenase inhibitor.

154. A method of increasing liver function in an individual having a hepatitis C virus infection, the method comprising administering to the individual an effective amount of a compound of claim 1.

155. The method of claim 154, wherein the method further comprises administering to the individual an effective amount of a nucleoside analog.

156. The method of claim 155, wherein the nucleoside analog is selected from ribavirin, levovirin, viramidine, an L-nucleoside, and isatoribine.

157. The method of claim 154, wherein the method further comprises administering to the individual an effective amount of a human immunodeficiency virus 1 protease inhibitor.

158. The method of method of claim 157, wherein the protease inhibitor is ritonavir.

159. The method of claim 154, wherein the method further comprises administering to the individual an effective amount of an NS3 protease inhibitor.

160. The method of claim 154, wherein the method further comprises administering to the individual an effective amount of interferon-gamma (IFN-γ).

161. The method of claim 160, wherein the IFN-γ is administered subcutaneously in an amount of from about 10 μg to about 300 μg.

162. The method of claim 154, wherein the method further comprises administering to the individual an effective amount of interferon-alpha (IFN-α).

163. The method of claim 162, wherein the IFN-α is monoPEG-ylated consensus IFN-α administered at a dosing interval of every 8 days to every 14 days.

164. The method of claim 162, wherein the IFN-α is monoPEG-ylated consensus IFN-α administered at a dosing interval of once every 7 days.

165. The method of claim 162, wherein the IFN-α is INFERGEN consensus IFN-α.

166. The method of claim 154, further comprising administering an effective amount of an agent selected from 3′-azidothymidine, 2′,3′-dideoxyinosine, 2′,3′-dideoxycytidine, 2′,3′-didehydro-2′,3′-dideoxythymidine, combivir, abacavir, adefovir dipoxil, cidofovir, and an inosine monophosphate dehydrogenase inhibitor.
